(19) United States
(12) Patent Application Publication
(10) Pub. No.: US 2024/0391926 A1
(43) Pub. Date: Nov. 28, 2024

(54) SPIROINDOLINONE COMPOUNDS AS Kv1.3 POTASSIUM SHAKER CHANNEL BLOCKERS

(71) Applicant: D.E. Shaw Research, LLC, Indianapolis, IN (US)

(72) Inventors: Fabrizio GIORDANETTO, New York, NY (US); Morten Østergaard JENSEN, Copenhagen (DK); Vishwanath JOGINI, Telengana (IN); Roger John SNOW, Danbury, CT (US)

(21) Appl. No.: 18/564,752

(22) PCT Filed: May 27, 2022
(86) PCT No.: PCT/US2022/031229

§ 371 Date: Nov. 28, 2023

Related U.S. Application Data
(60) Provisional application No. 63/194,599, filed on May 28, 2021.

(51) Int. Cl. C07D 487/10; A61K 31/407; A61K 31/4155; A61K 31/4245; A61K 31/497

(52) U.S. Cl. CPC C07D 487/10; A61K 31/407; A61K 31/4155; A61K 31/4245; A61K 31/497

(57) ABSTRACT

A compound of Formula (I) or a pharmaceutically-acceptable salt thereof, is described, wherein the substituents are as defined herein. Pharmaceutical compositions comprising the same and method of using the same are also described.

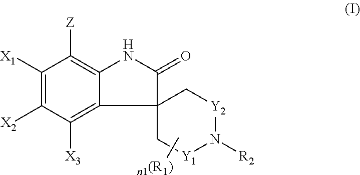

This application claims the benefit of and priority to U.S. Provisional Patent Application No. 63/194,599, filed on May 28, 2021, the content of which is hereby incorporated by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals as potassium channel blockers.

### BACKGROUND

Voltage-gated Kv1.3 potassium (K⁺) channels are expressed in lymphocytes (T and B lymphocytes), the central nervous system, and other tissues, and regulate a large number of physiological processes such as neurotransmitter release, heart rate, insulin secretion, and neuronal excitability. Kv1.3 channels can regulate membrane potential and thereby indirectly influence calcium signaling in human effector memory T cells. Effector memory T cells are mediators of several conditions, including multiple sclerosis, type I diabetes mellitus, psoriasis, spondylitis, parodontitis, and rheumatoid arthritis. Upon activation, effector-memory T cells increase expression of the Kv1.3 channel. Amongst human B cells, naive and early memory B cells express small numbers of Kv1.3 channels when they are quiescent. In contrast, class-switched memory B cells express high numbers of Kv1.3 channels. Furthermore, the Kv1.3 channel promotes the calcium homeostasis required for T-cell receptor-mediated cell activation, gene transcription, and proliferation (Panyi, G., et al., 2004, Trends Immunol., 565-569). Blockade of Kv1.3 channels in effector memory T cells suppresses activities like calcium signaling, cytokine production (interferon-gamma, interleukin 2), and cell proliferation.

Autoimmune disease is a family of disorders resulting from tissue damage caused by attack from the body's own immune system. Such diseases may affect a single organ, as in multiple sclerosis and type I diabetes mellitus, or may involve multiple organs, as in the case of rheumatoid arthritis and systemic lupus erythematosus. Treatment is generally palliative, with anti-inflammatory and immunosuppressive drugs, which can have severe side effects. A need for more effective therapies has led to a search for drugs that can selectively inhibit the function of effector memory T cells, known to be involved in the etiology of autoimmune diseases. These inhibitors are thought to be able to ameliorate autoimmune diseases symptoms without compromising the protective immune response. Effector memory T cells (“TEMs”) express high numbers of the Kv1.3 channel and depend on these channels for their function. In vivo, Kv1.3 channel blockers paralyze TEMs at the sites of inflammation and prevent their reactivation in inflamed tissues. Kv1.3 channel blockers do not affect the motility within lymph nodes of naive and central memory T cells. Suppressing the function of these cells by selectively blocking the Kv1.3 channel offers the potential for effective therapy of autoimmune diseases with minimal side effects.

Multiple sclerosis (“MS”) is caused by autoimmune damage to the central nervous system (“CNS”). Symptoms include muscle weakness and paralysis, which severely affect quality of life for patients. MS progresses rapidly and unpredictably and eventually leads to death. The Kv1.3 channel is also highly expressed in auto-reactive TEMs from MS patients (Wulff H., et al., 2003, J. Clin. Invest., 1703-1713; Rus H., et al., 2005, PNAS, 11094-11099). Animal models of MS have been successfully treated using blockers of the Kv1.3 channel.

Compounds which are selective Kv1.3 channel blockers are thus potential therapeutic agents as immunosuppressants or immune system modulators. The Kv1.3 channel is also considered as a therapeutic target for the treatment of obesity and for enhancing peripheral insulin sensitivity in patients with type 2 diabetes mellitus. These compounds can also be utilized in the prevention of graft rejection and the treatment of immunological (e.g., autoimmune) and inflammatory disorders.

Tubulointerstitial fibrosis is a progressive connective tissue deposition on the kidney parenchyma, leading to renal function deterioration, is involved in the pathology of chronic kidney disease, chronic renal failure, nephritis, and inflammation in glomeruli, and is a common cause of end-stage renal failure. Overexpression of Kv1.3 channels in lymphocytes can promote their proliferation, leading to chronic inflammation and overstimulation of cellular immunity, which are involved in the underlying pathology of these renal diseases and are contributing factors in the progression of tubulointerstitial fibrosis. Inhibition of the lymphocyte Kv1.3 channel currents suppress proliferation of kidney lymphocytes and ameliorate the progression of renal fibrosis (Kazama I., et al., 2015, Mediators Inflamm., 1-12).

Kv1.3 channels also play a role in gastroenterological disorders including inflammatory bowel diseases (“IBDs”) such as ulcerative colitis (“UC”) and Crohn's disease. UC is a chronic IBD characterized by excessive T cell infiltration and cytokine production. UC can impair quality of life and can lead to life-threatening complications. High levels of Kv1.3 channels in CD4 and CD8 positive T cells in the inflamed mucosa of UC patients have been associated with production of pro-inflammatory compounds in active UC. Kv1.3 channels are thought to serve as a marker of disease activity and pharmacological blockade might constitute a novel immunosuppressive strategy in UC, as demonstrated in a humanized rodent model of UC (Unterweger A., et al. 2021, J. Crohns Colitis, available at https://academic.oup.com/ecco-icc/advance-article/doi/10.1093/ecco-icc/jjab078/6247959). Present treatment regimens for UC, including corticosteroids, salicylates, and anti-TNF-α reagents, are insufficient for many patients (Hansen L. K., et al., 2014, J. Crohns Colitis, 1378-1391). Crohn's disease is a type of IBD which may affect any part of the gastrointestinal tract. Crohn's disease is thought to be the result of intestinal inflammation due to a T cell-driven process initiated by normally safe bacteria. Thus, Kv1.3 channel inhibition can be utilized in treating the Crohn's disease.

In addition to T cells, Kv1.3 channels are also expressed in microglia, where the channel is involved in inflammatory cytokine and nitric oxide production and in microglia-mediated neuronal killing. In humans, strong Kv1.3 channel expression has been found in microglia in the frontal cortex of patients with Alzheimer's disease and on CD68⁺ cells in MS brain lesions. It has been suggested that Kv1.3 channel blockers might be able to preferentially target detrimental proinflammatory microglia functions. Kv1.3 channels are expressed on activated microglia in infarcted rodent and human brain. Higher Kv1.3 channel current densities are observed in acutely isolated microglia from the infarcted hemisphere than in microglia isolated from the contralateral hemisphere of a mouse model of stroke (Chen Y. J., et al., 2017, Ann. Clin. Transl. Neurol., 147-161).

Expression of Kv1.3 channels is elevated in microglia of human Alzheimer's disease brains, suggesting that Kv1.3 channel is a pathologically relevant microglial target in Alzheimer's disease (Rangaraju S., et al., 2015, J. Alzheimers Dis., 797-808). Soluble AβO enhances microglial Kv1.3 channel activity. Kv1.3 channels are required for AβO-induced microglial pro-inflammatory activation and neurotoxicity. Kv1.3 channel expression/activity is upregulated in transgenic Alzheimer's disease animals and human Alzheimer's disease brains. Pharmacological targeting of microglial Kv1.3 channels can affect hippocampal synaptic plasticity and reduce amyloid deposition in APP/PS1 mice. Thus, Kv1.3 channel may be a therapeutic target for Alzheimer's disease.

Kv1.3 channel blockers could be also useful for ameliorating pathology in cardiovascular disorders such as ischemic stroke, where activated microglia significantly contributes to the secondary expansion of the infarct.

Kv1.3 channel expression is associated with the control of proliferation in multiple cell types, apoptosis, and cell survival. These processes are crucial for cancer progression. In this context, Kv1.3 channels located in the inner mitochondrial membrane can interact with the apoptosis regulator Bax (Serrano-Albarras, A., et al., 2018, Expert Opin. Ther. Targets, 101-105). Thus, inhibitors of Kv1.3 channels may be used as anticancer agents.

A number of peptide toxins with multiple disulfide bonds from spiders, scorpions, and anemones are known to block Kv1.3 channels. A few selective, potent peptide inhibitors of the Kv1.3 channel have been developed. A synthetic derivative of stichodactyla toxin (“shk”) with an unnatural amino acid (shk-186) is the most advanced peptide toxin. Shk has demonstrated efficacy in preclinical models and is currently in a phase I clinical trial for treatment of psoriasis. Shk can suppress proliferation of TEMs, resulting in improved condition in animal models of multiple sclerosis. Unfortunately, Shk also binds to the closely-related Kvi channel subtype found in CNS and heart. There is a need for Kv1.3 channel-selective inhibitors to avoid potential cardio- and neuro-toxicity. Additionally, small peptides like shk-186 are rapidly cleared from the body after administration, resulting in short circulating half-lives and frequent administration events. Thus, there is a need for the development of long-acting, selective Kv1.3 channel inhibitors for the treatment of chronic inflammatory diseases.

Thus, there remains a need for development of novel Kv1.3 channel blockers as pharmaceutical agents.

### SUMMARY OF THE INVENTION

In one aspect, compounds useful as potassium channel blockers having a structure of Formula I

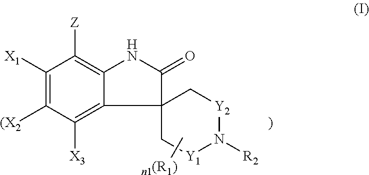

are described, where the various substituents are defined herein. The compounds of Formula I disclosed herein can block Kv1.3 potassium (K⁺) channels and be used in the treatment of a variety of conditions. Methods for synthesizing these compounds are also described herein. Pharmaceutical compositions and methods of using these compositions described herein are useful for treating conditions in vitro and in vivo. Such compounds, pharmaceutical compositions, and methods of treatment have a number of clinical applications, including as pharmaceutically active agents and methods for treating cancer, an immunological disorder, a CNS disorder, an inflammatory disorder, a gastroenterological disorder, a metabolic disorder, a cardiovascular disorder, a kidney disease, or a combination thereof.

In one aspect, a compound of Formula I or a pharmaceutically-acceptable salt thereof is described,

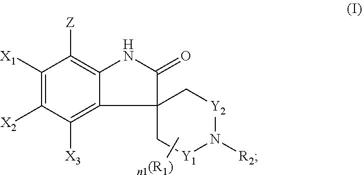

wherein:
X₁, X₂, and X₃ are each independently H, halogen, CN, alkyl, cycloalkyl, halogenated alkyl, halogenated cycloalkyl, OH, SH, alkoxy, halogenated alkoxy, alkylthio, or halogenated alkylthio;
or alternatively X₁ and X₂ and the carbon atoms they are connected to taken together form a 5- or 6-membered aryl;
or alternatively X₂ and X₃ and the carbon atoms they are connected to taken together form a 5- or 6-membered aryl;
Z is H, alkyl, halogenated alkyl, heteroalkyl, cycloalkyl, heterocycle, aryl, heteroaryl, halogen, CN, CF₃, OCF₃, ORₐ, NRₐR_{b}, or NRₐ(C═O)R_{b};
Y₁ is absent or C(R₁)₂;
Y₂ is absent, C(R₁)₂, C(R₁)₂(C═O), C(R₁)₂C(R₁)₂ or C(R₁)₂C(R₁)₂(C═O);
each occurrence of R₁ is independently H, halogen, alkyl, cycloalkyl, saturated heterocycle, aryl, heteroaryl, (CR₄R₅)ₙ₃OR_{c}, or (CR₄R₅)ₙ₃NR_{c}R_{d};
R₂ is alkyl, heteroalkyl, cycloalkyl, heterocycle, bicycloalkyl, spiroalkyl, heterobicycloalkyl, heterospiroalkyl, alkylaryl, alkylheteroaryl, aryl, heteroaryl, (CR₄R₅)ₙ₂(C═O)R₃, (CR₄R₅)ₙ₂(C═O)N(R₄)R₃, SO₂R₃, or SO₂NR_{c}R_{d};
each occurrence of R₃ is H, independently alkyl, cycloalkyl, heterocycle, bicycloalkyl, spiroalkyl, heterobicycloalkyl, heterospiroalkyl, alkylaryl, alkylheteroaryl, aryl, or heteroaryl;
each occurrence of R₄ and R₅ is independently H, alkyl, cycloalkyl, heterocycle, aryl, or heteroaryl;
each occurrence of Rₐ and R_{b} is independently H, alkyl, cycloalkyl, heterocycle, aryl, or heteroaryl;
or alternatively Rₐ and R_{b} together with the nitrogen atom that they are connected to form a 3-7-membered heterocycle;
each occurrence of R_{c} and R_{d} is independently H, alkyl, cycloalkyl, heterocycle, aryl, or heteroaryl;
or alternatively R_{c} and R_{d} together with the nitrogen atom that they are connected to form a 3-7-membered heterocycle;
each heterocycle comprises 1-3 heteroatoms each independently selected from the group consisting of N, O and S;
each of alkyl, cycloalkyl, heteroalkyl, heterocycle, aryl, and heteroaryl in X₁, X₂, X₃, Z, R₁, R₂, or R₃, where applicable, are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)R_{d}, where valence permits;
n₁ is an integer from 0-4;
n₂ is an integer from 0-4; and
n₃ is an integer from 0-4.

In one aspect, a compound of Formula I or a pharmaceutically-acceptable salt thereof is described,

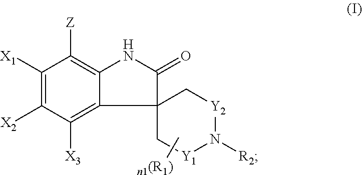

wherein:
X₁, X₂, and X₃ are each independently H, halogen, CN, alkyl, cycloalkyl, halogenated alkyl, halogenated cycloalkyl, OH, SH, alkoxy, halogenated alkoxy, alkylthio, or halogenated alkylthio;
or alternatively X₁ and X₂ and the carbon atoms they are connected to taken together form a 5- or 6-membered aryl;
or alternatively X₂ and X₃ and the carbon atoms they are connected to taken together form a 5- or 6-membered aryl;
Z is H, alkyl, halogenated alkyl, heteroalkyl, cycloalkyl, heterocycle, aryl, heteroaryl, halogen, CN, CF₃, OCF₃, ORₐ, NRₐR_{b}, or NRₐ(C═O)R_{b};
Y₁ is absent or C(R₁)₂;
Y₂ is absent, C(R₁)₂, C(R₁)₂(C═O), C(R₁)₂C(R₁)₂ or C(R₁)₂C(R₁)₂(C═O);
each occurrence of R₁ is independently H, halogen, alkyl, cycloalkyl, saturated heterocycle, aryl, heteroaryl, (CR₄R₅)ₙ₃OR_{c}, or (CR₄R₅)ₙ₃NR_{c}R_{d};
R₂ is alkyl, heteroalkyl, cycloalkyl, heterocycle, bicycloalkyl, spiroalkyl, heterobicycloalkyl, heterospiroalkyl, alkylaryl, alkylheteroaryl, aryl, heteroaryl, (CR₄R₅)ₙ₂(C═O)R₃, (CR₄R₅)ₙ₂(C═O)N(R₄)R₃, SO₂R_{c}, or SO₂NR_{c}R_{d};
each occurrence of R₃ is H, independently alkyl, cycloalkyl, heterocycle, bicycloalkyl, spiroalkyl, heterobicycloalkyl, heterospiroalkyl, alkylaryl, alkylheteroaryl, aryl, or heteroaryl;
each occurrence of R₄ and R₅ is independently H, alkyl, cycloalkyl, heterocycle, aryl, or heteroaryl;
each occurrence of Rₐ and R_{b} is independently H, alkyl, cycloalkyl, heterocycle, aryl, or heteroaryl;
or alternatively Rₐ and R_{b} together with the nitrogen atom that they are connected to form a 3-7-membered heterocycle;
each occurrence of R_{c} and R_{d} is independently H, alkyl, cycloalkyl, heterocycle, aryl, or heteroaryl;
or alternatively R_{c} and R_{d} together with the nitrogen atom that they are connected to form a 3-7-membered heterocycle;
each heterocycle comprises 1-3 heteroatoms each independently selected from the group consisting of N, O and S;
each of alkyl, cycloalkyl, heteroalkyl, heterocycle, aryl, and heteroaryl in X₁, X₂, X₃, Z, R₁, R₂, or R₃, where applicable, are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)R_{d}, where valence permits;
n₁ is an integer from 0-4;
n₂ is an integer from 0-4; and
n₃ is an integer from 0-4.

In any one of the embodiments described herein, X₁, X₂, and X₃ are each independently H, halogen, CN, alkyl, or halogenated alkyl.

In any one of the embodiments described herein, X₁, X₂, and X₃ are each independently cycloalkyl or halogenated cycloalkyl.

In any one of the embodiments described herein, X₁, X₂, and X₃ are each independently H, F, Cl, Br, CN, CH₃, or CF₃.

In any one of the embodiments described herein, X₁, X₂, and X₃ are each independently H or Cl.

In any one of the embodiments described herein, Z is H, halogen, alkyl, or halogenated alkyl.

In any one of the embodiments described herein, Z is H, F, Cl, Br, CH₃, or CF₃.

In any one of the embodiments described herein, Z is H or Cl.

In any one of the embodiments described herein, Z is ORₐ or NRₐR_{b}.

In any one of the embodiments described herein, each occurrence of Rₐ and R_{b} is independently H or alkyl.

In any one of the embodiments described herein, each occurrence of Rₐ and R_{b} is cycloalkyl or heterocycle.

In any one of the embodiments described herein, each occurrence of Rₐ and R_{b} is aryl or heteroaryl.

In any one of the embodiments described herein, at least two of Z, X₁, X₂, and X₃ are not H.

In any one of the embodiments described herein, the structural moiety

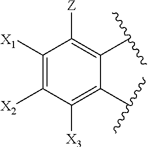

has the structure of

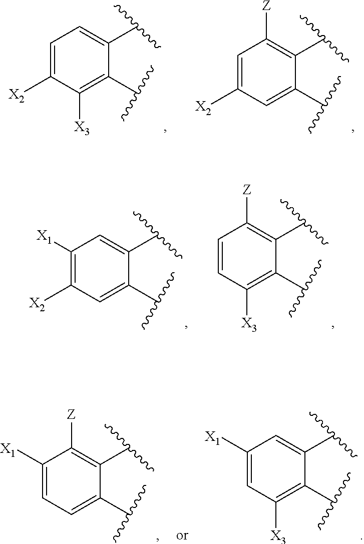

In any one of the embodiments described herein, the structural moiety

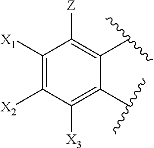

has the structure of

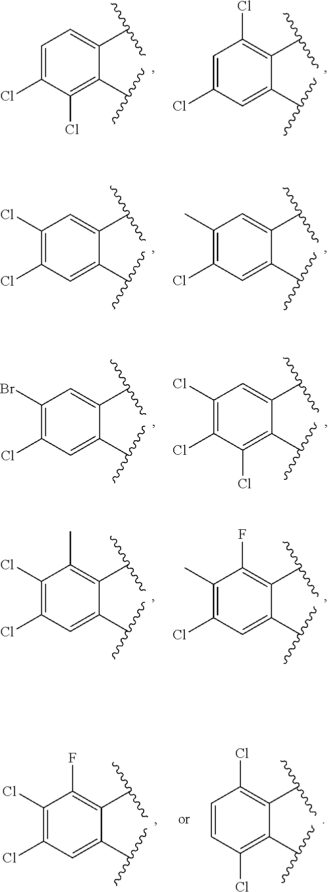

In any one of the embodiments described herein, the structural moiety

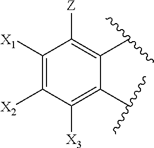

has the structure of

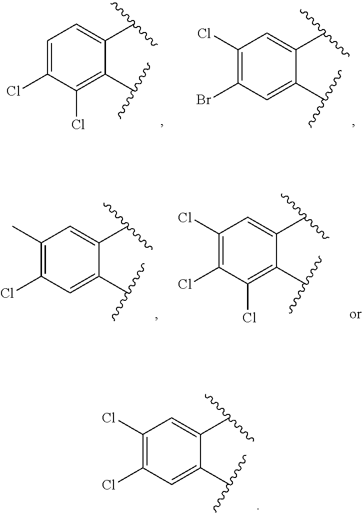

In any one of the embodiments described herein, the structural moiety

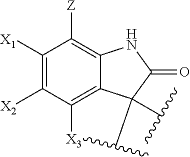

has the structure of

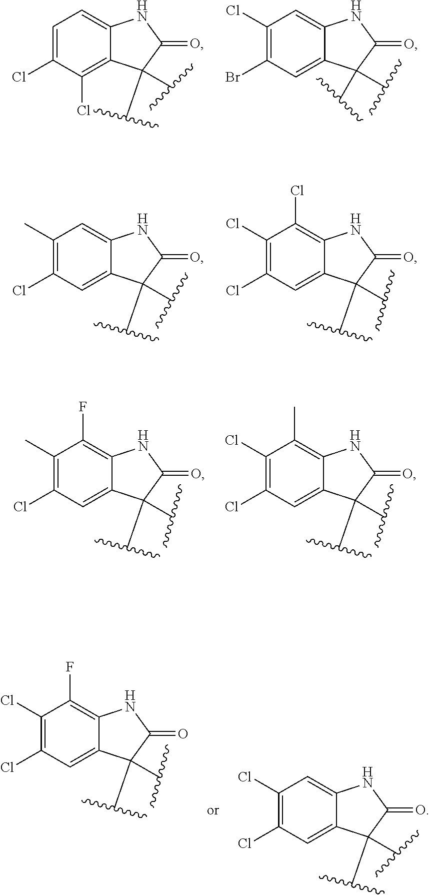

In any one of the embodiments described herein, Y₁ and Y₂ are each independently absent or C(R₁)₂.

In any one of the embodiments described herein, Y₁ is absent and Y₂ is C(R₁)₂.

In any one of the embodiments described herein, Y₁ is C(R₁)₂ and Y₂ is C(R₁)₂.

In any one of the embodiments described herein, the structural moiety

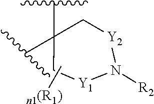

has the structure of

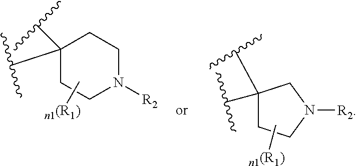

In any one of the embodiments described herein, the structural moiety

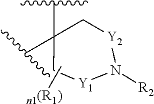

has the structure of

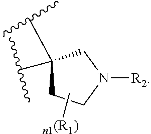

In any one of the embodiments described herein, at least one occurrence of R₁ is H, alkyl, or cycloalkyl.

In any one of the embodiments described herein, at least one occurrence of R₁ is halogen, (CR₄R₅)ₙ₃OR_{c}, or (CR₄R₅)ₙ₃NR_{c}R_{d}.

In any one of the embodiments described herein, at least one occurrence of R₁ is saturated heterocycle, aryl, or heteroaryl.

In any one of the embodiments described herein, at least one occurrence of R₁ is H or CH₃.

In any one of the embodiments described herein, the compound has Formula Ia:

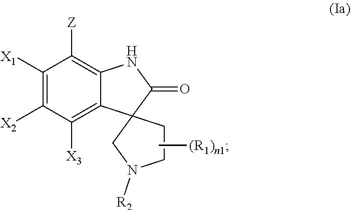

wherein:
X₁, X₂, and X₃ are each independently H, halogen, or alkyl;
Z is H, halogen, alkyl, halogenated alkyl, CN, ORₐ, or NRₐR_{b}; and
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d}.

In any one of the embodiments described herein, Z is H, halogen, alkyl, or halogenated alkyl.

In any one of the embodiments described herein, Z is H, F, Cl, Br, CH₃, or CF₃.

In any one of the embodiments described herein, Z is H.

In any one of the embodiments described herein, Z is CN, ORₐ, or NRₐR_{b}.

In any one of the embodiments described herein, each occurrence of Rₐ and R_{b} is independently H or alkyl.

In any one of the embodiments described herein, each occurrence of Rₐ and R_{b} is cycloalkyl or heterocycle.

In any one of the embodiments described herein, each occurrence of Rₐ and R_{b} is aryl or heteroaryl.

In any one of the embodiments described herein, at least one occurrence of R₁ is alkyl or cycloalkyl.

In any one of the embodiments described herein, at least one occurrence of R₁ is halogen, (CR₄R₅)ₙ₃OR_{c}, or (CR₄R₅)ₙ₃NR_{c}R_{d}.

In any one of the embodiments described herein, at least one occurrence of R₁ is saturated heterocycle, aryl, or heteroaryl.

In any one of the embodiments described herein, n₁ is 0 or 1.

In any one of the embodiments described herein, the compound has Formula Ib:

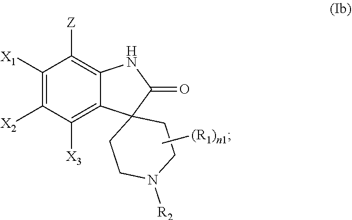

wherein:
X₁, X₂, and X₃ are each independently H, alkyl, or halogen;
Z is H, halogen, alkyl, halogenated alkyl, CN, ORₐ, or NRₐR_{b}; and
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d}.

In any one of the embodiments described herein, Z is H, halogen, alkyl, or halogenated alkyl.

In any one of the embodiments described herein, Z is H, F, Cl, Br, CH₃, or CF₃.

In any one of the embodiments described herein, Z is H.

In any one of the embodiments described herein, Z is CN, ORₐ, or NRₐR_{b}.

In any one of the embodiments described herein, each occurrence of Rₐ and R_{b} is independently H or alkyl.

In any one of the embodiments described herein, each occurrence of Rₐ and R_{b} is cycloalkyl or heterocycle.

In any one of the embodiments described herein, each occurrence of Rₐ and R_{b} is aryl or heteroaryl.

In any one of the embodiments described herein, at least one occurrence of R₁ is alkyl or cycloalkyl.

In any one of the embodiments described herein, at least one occurrence of R₁ is halogen, (CR₄R₅)ₙ₃OR_{c}, or (CR₄R₅)ₙ₃NR_{c}R_{d}.

In any one of the embodiments described herein, at least one occurrence of R₁ is saturated heterocycle, aryl, or heteroaryl.

In any one of the embodiments described herein, n₁ is 0 or 1.

In any one of the embodiments described herein, R₂ is alkyl, cycloalkyl, or heteroalkyl.

In any one of the embodiments described herein, R₂ is heterocycle, aryl, heteroaryl, alkylaryl, or alkylheteroaryl.

In any one of the embodiments described herein, R₂ is bicycloalkyl, spiroalkyl, heterobicycloalkyl, or heterospiroalkyl.

In any one of the embodiments described herein, R₂ is SO₂R_{c} or SO₂NR_{c}R_{d}.

In any one of the embodiments described herein, R₂ is (CR₄R₅)ₙ₂OR_{c}, (CR₄R₅)ₙ₂(CR₄)((CR₄R₅)ₙ₃OR_{c})₂, (C═O)(CR₄R₅)ₙ₂OR_{c}, (C═O)(CR₄R₅)ₙ₂(CR₄)((CR₄R₅)ₙ₃OR_{c})₂, (CR₄R₅)ₙ₂COOR_{c}, (C═O)(CR₄R₅)ₙ₂NR_{c}R_{d}, or (CR₄R₅)ₙ₂NR_{c}(C═O)R_{d}.

In any one of the embodiments described herein, R₂ is (CR₄R₅)ₙ₂(C═O)R₃ or (CR₄R₅)ₙ₂(C═O)NR₃R₄.

In any one of the embodiments described herein, each occurrence of R₃ is alkyl or cycloalkyl.

In any one of the embodiments described herein, each occurrence of R₃ is heterocycle, aryl, or heteroaryl.

In any one of the embodiments described herein, each occurrence of R₃ is alkylaryl or alkylheteroaryl.

In any one of the embodiments described herein, each occurrence of R₃ is bicycloalkyl, spiroalkyl, heterobicycloalkyl, or heterospiroalkyl.

In any one of the embodiments described herein, each occurrence of R₄ and R₅ is independently H, alkyl, cycloalkyl, or heterocycle.

In any one of the embodiments described herein, each occurrence of R₄ and R₅ is independently aryl or heteroaryl.

In any one of the embodiments described herein, each occurrence of R_{c} and R_{d} is independently H, alkyl, or cycloalkyl.

In any one of the embodiments described herein, each occurrence of R_{c} and R_{d} is independently heterocycle, aryl, or heteroaryl.

In any one of the embodiments described herein, each occurrence of n₂ and n₃ is independently 0, 1, or 2.

In any one of the embodiments described herein, each occurrence of n₂ and n₃ is each independently 3 or 4.

In any one of the embodiments described herein, the compound has Formula Ic:

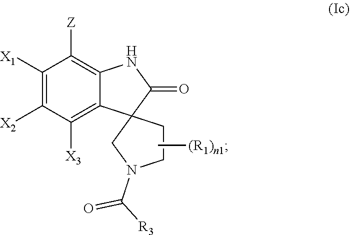

wherein:
X₁, X₂, and X₃ are each independently H, halogen, or alkyl;
Z is H, halogen, alkyl, halogenated alkyl, CN, ORₐ, or NRₐR_{b};
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d}; and
R₃ is alkyl, cycloalkyl, heterocycle, aryl, heteroaryl, bicycloalkyl, spiroalkyl, heterobicycloalkyl, or heterospiroalkyl; and wherein the alkyl, cycloalkyl, heterocycle, aryl, or heteroaryl is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, Z is H, halogen, alkyl, or halogenated alkyl.

In any one of the embodiments described herein, Z is H, F, Cl, Br, CH₃, or CF₃.

In any one of the embodiments described herein, Z is H or Cl.

In any one of the embodiments described herein, at least one occurrence of R₁ is H, alkyl or cycloalkyl.

In any one of the embodiments described herein, n₁ is 0 or 1.

In any one of the embodiments described herein, R₃ is alkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, R₃ is cycloalkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, R₃ is heterocycle that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, R₃ is aryl or heteroaryl each optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, R₃ is bicycloalkyl, spiroalkyl, heterobicycloalkyl, or heterospiroalkyl.

In any one of the embodiments described herein, the compound has Formula Id:

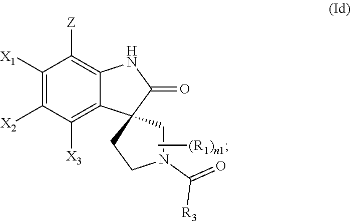

wherein:
X₁, X₂, and X₃ are each independently H, halogen, or alkyl;
Z is H, halogen, alkyl, halogenated alkyl, CN, ORₐ, or NRₐR_{b};
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d}; and
R₃ is alkyl, cycloalkyl, heterocycle, aryl, heteroaryl, bicycloalkyl, spiroalkyl, heterobicycloalkyl, or heterospiroalkyl; and wherein the alkyl, cycloalkyl, heterocycle, aryl, or heteroaryl is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, Z is H, halogen, alkyl, or halogenated alkyl.

In any one of the embodiments described herein, Z is H, F, Cl, Br, CH₃, or CF₃.

In any one of the embodiments described herein, Z is H or Cl.

In any one of the embodiments described herein, at least one occurrence of R₁ is H, alkyl or cycloalkyl.

In any one of the embodiments described herein, n₁ is 0 or 1.

In any one of the embodiments described herein, R₃ is alkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, R₃ is cycloalkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, R₃ is heterocycle that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, R₃ is aryl or heteroaryl each optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In any one of the embodiments described herein, R₃ is bicycloalkyl, spiroalkyl, heterobicycloalkyl, or heterospiroalkyl.

In any one of the embodiments described herein, R₂ is

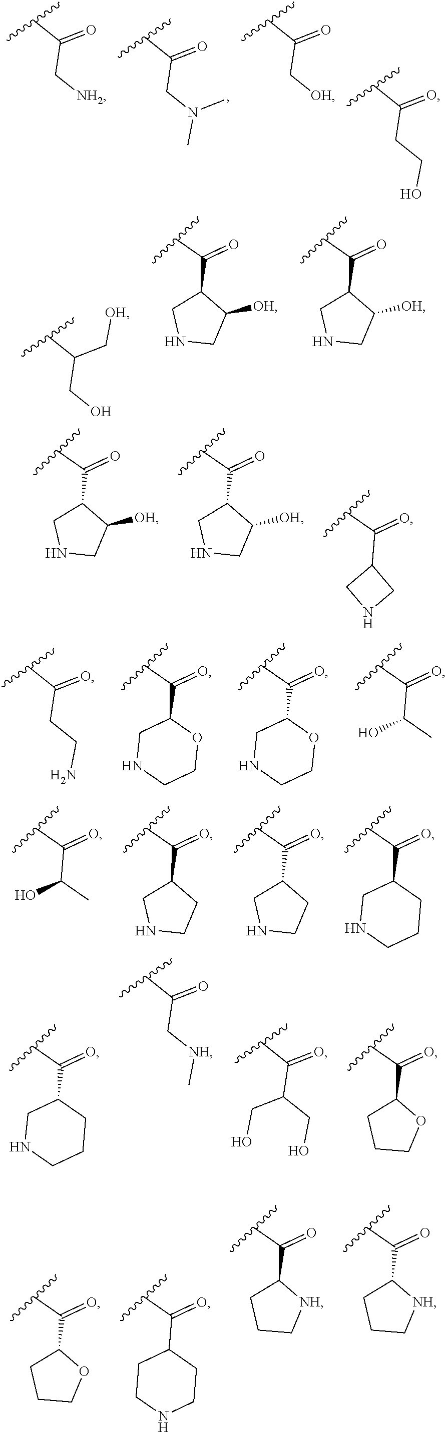

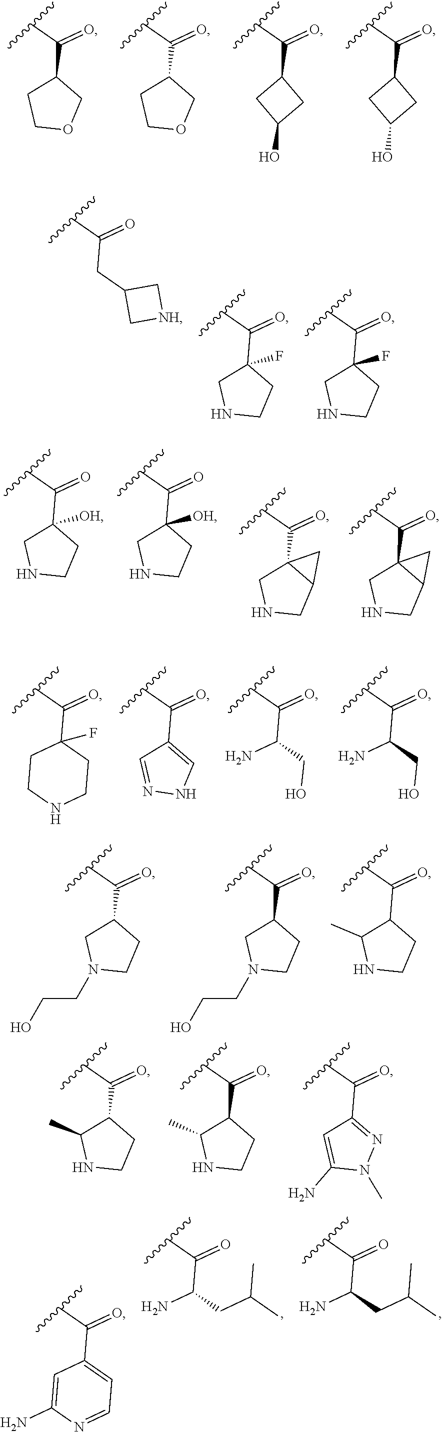

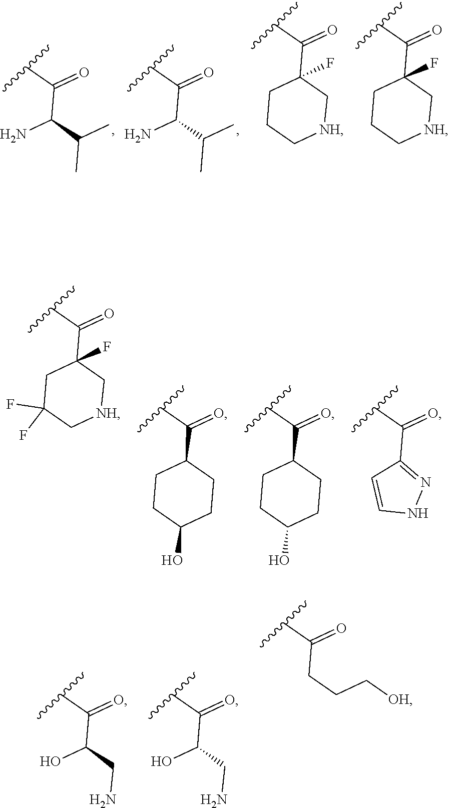

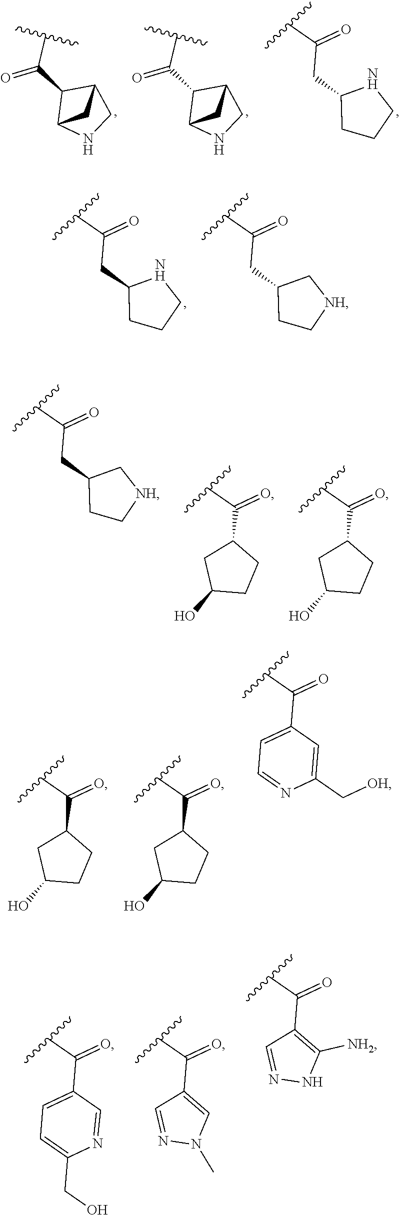

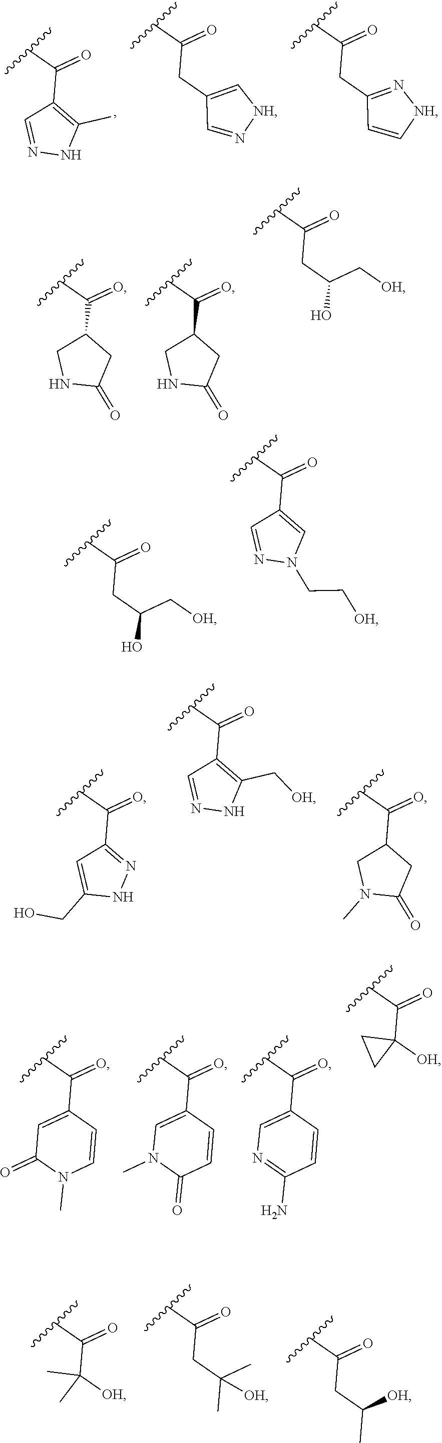

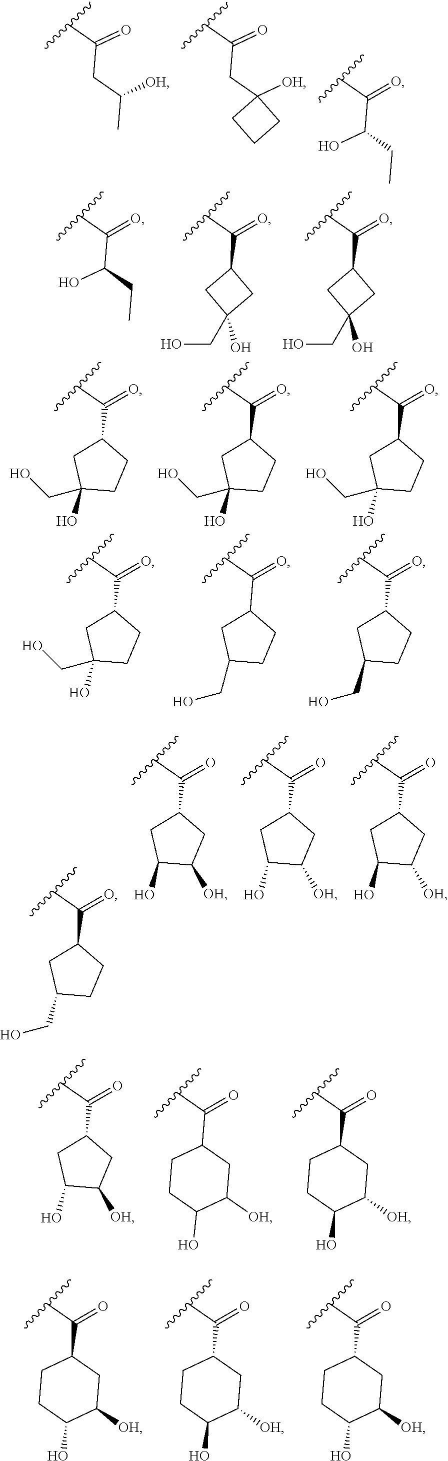

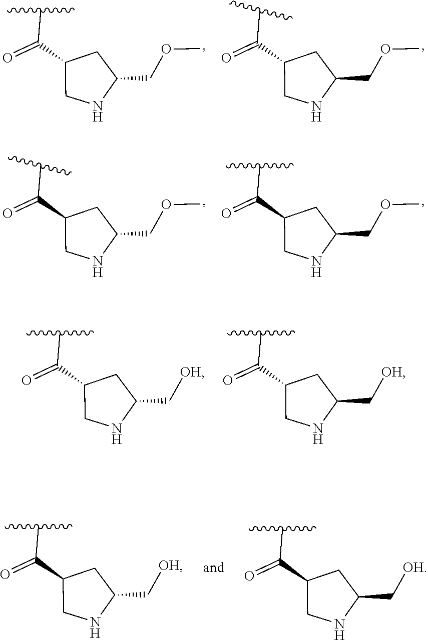

In any one of the embodiments described herein, R₂ is

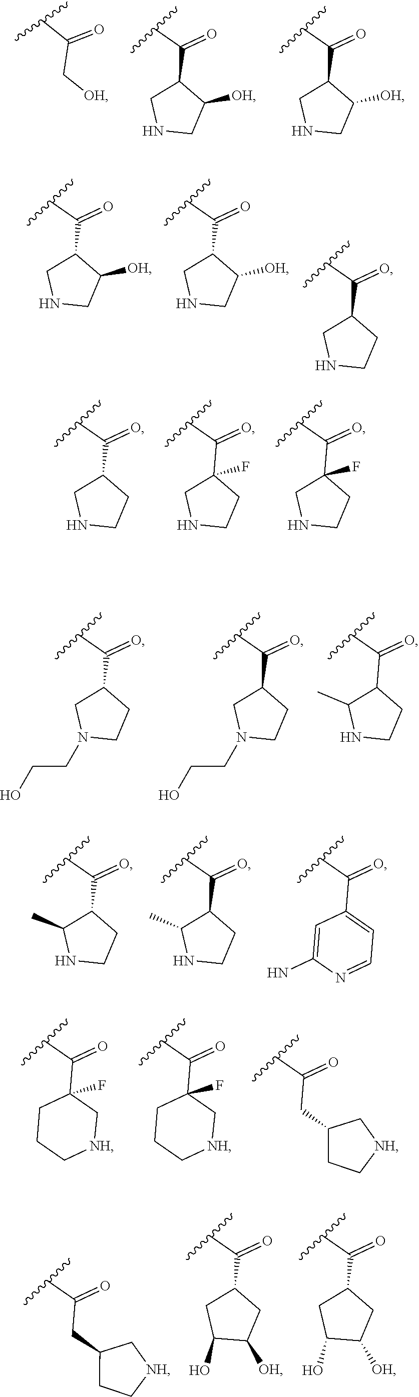

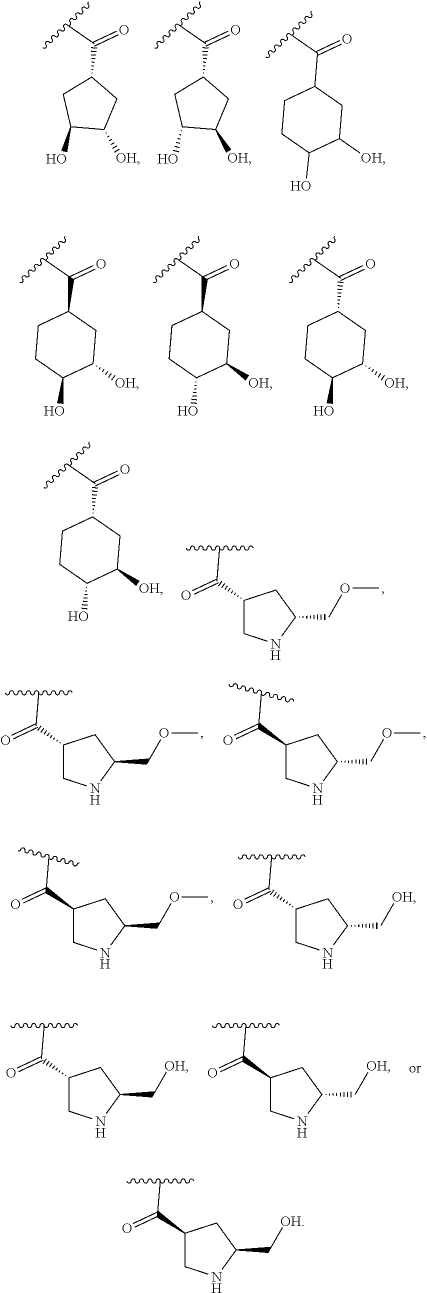

In any one of the embodiments described herein, R₂ is

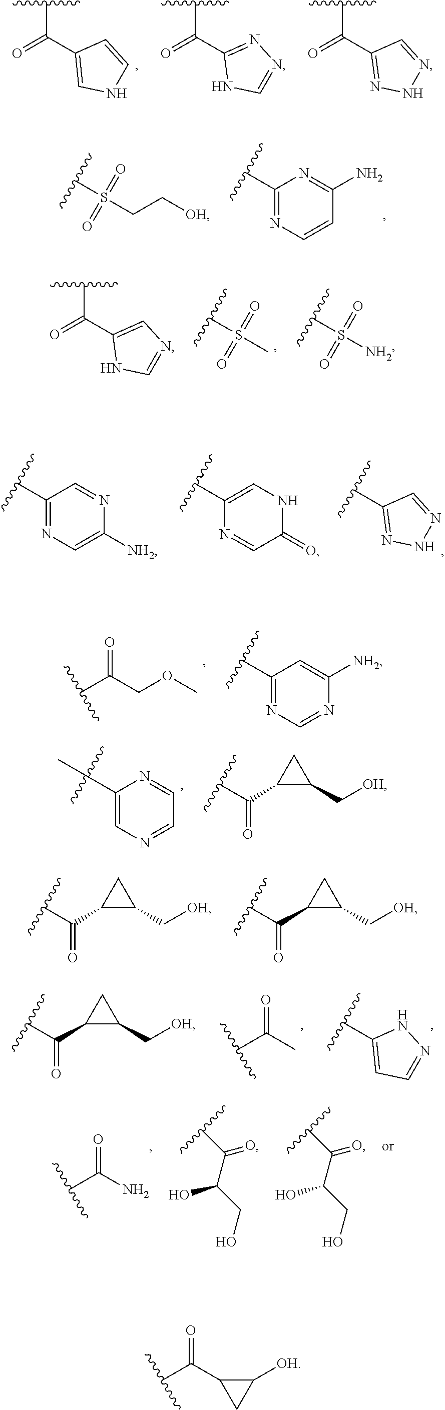

In any one of the embodiments described herein, the compound is selected from the group consisting of compounds 1-159 as shown in Table 1.

In another aspect, a pharmaceutical composition is described, including at least one compound according to any one of the embodiments described herein or a pharmaceutically-acceptable salt thereof and a pharmaceutically-acceptable carrier or diluent.

In yet another aspect, a method of treating a condition in a mammalian species in need thereof is described, including administering to the mammalian species a therapeutically effective amount of at least one compound according to any one of the embodiments described herein, or a pharmaceutically-acceptable salt thereof, or a pharmaceutical composition thereof, where the condition is selected from the group consisting of cancer, an immunological disorder, a central nervous system disorder, an inflammatory disorder, a gastroenterological disorder, a metabolic disorder, a cardiovascular disorder, and a kidney disease.

In any one of the embodiments described herein, the immunological disorder is transplant rejection or an autoimmune disease.

In any one of the embodiments described herein, the autoimmune disease is rheumatoid arthritis, multiple sclerosis, systemic lupus erythematosus, or type I diabetes mellitus.

In any one of the embodiments described herein, the Central Nerve System (CNS) disorder is Alzheimer's disease.

In any one of the embodiments described herein, the inflammatory disorder is an inflammatory skin condition, arthritis, psoriasis, spondylitis, parodontitits, or an inflammatory neuropathy.

In any one of the embodiments described herein, the gastroenterological disorder is an inflammatory bowel disease.

In any one of the embodiments described herein, the metabolic disorder is obesity or type II diabetes mellitus.

In any one of the embodiments described herein, the cardiovascular disorder is an ischemic stroke.

In any one of the embodiments described herein, the kidney disease is chronic kidney disease, nephritis, or chronic renal failure.

In any one of the embodiments described herein, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

In any one of the embodiments described herein, the mammalian species is human.

In yet another aspect, a method of blocking Kv1.3 potassium channel in a mammalian species in need thereof is described, including administering to the mammalian species a therapeutically effective amount of at least one compound according to any one of the embodiments described herein, or a pharmaceutically-acceptable salt thereof, or a pharmaceutical composition thereof.

In any one of the embodiments described herein, the mammalian species is human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂R_{c}, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle, and each occurrence of R_{c} is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle, and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited to, one or more of the following groups: hydrogen, halogen, alkyl, halogenated alkyl (i.e., an alkyl group bearing a single halogen substituent or multiple halogen substituents such as CF₃ or CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂R_{c}, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of R_{c} is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂R_{c}, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally to form a heterocycle; and each occurrence of R_{c} is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂R_{c}, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally to form a heterocycle; and each occurrence of R_{c} is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)OR_{c}, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂R_{c}, wherein each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of R_{c} is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings wherein two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)OR_{c}, NRₐC(═O)NR_{b}R_{c}, NR_{d}S(═O)₂NR_{b}R_{c}, NRₐP(═O)₂NR_{b}R_{c}, NR_{b}C(═O)Rₐ, or NR_{b}P(═O)₂R_{c}, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of R_{c} is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms and sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, indolinyl, isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂R_{c}, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of R_{c} is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “bicycloalkyl” or “spiroalkyl” refers to a compound containing at least one cycloalkyl ring that shares one or more ring atoms with at least one other cycloalkyl ring. The term “heterobicycloalkyl” or “heterospiroalkyl” refers to a bicycloalkyl group in which at least one, preferably from 1-3, carbon atoms in at least one ring are replaced with a heteroatom selected from the group consisting of N, S, O, or P. The heteroatom may occupy a terminal position or a bridging position (i.e., a connection point between two rings). Exemplary bicycloalkyl groups include adamantyl, bicyclo[1.1.1]pentyl, bicyclo[2.2.1]heptyl, bicyclo[3.1.1]heptyl, bicyclo[2.1.1]hexyl, octahydropentalenyl, bicyclo[3.2.1]octyl, bicyclo[3.3.3]undecanyl, decahydronaphthalenyl, bicyclo[3.2.0]heptyl, octahydro-1H-indenyl, bicyclo[4.2.1]nonanyl, and the like. Exemplary spiro bicycloalkyl groups include spiro[4.4]nonyl, spiro[3.3]heptyl, spiro[5.5]undecyl, spiro[3.5]nonyl, spiro[4.5]decyl, and the like. “Substituted bicycloalkyl”, “substituted spiroalkyl”, “substituted heterobicycloalkyl”, and “substituted heterospiroalkyl” refer to a bicycloalkyl, spiroalkyl, heterobicycloalkyl, or heterospiroalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, bicycloalkyl, spiroalkyl, alkenyl, cycloalkenyl, alkynyl, heterocycle, aryl, ORₐ, SRₐ, S(═O)R_{c}, S(═O)₂R_{c}, —N═S(═O)(Rₐ), —RₐS(═O)(═NRₐ), S(═O)(═NRₐ)(═N(Rₐ)₂) (linked to the molecule via Rₐ or N), P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂R_{c}, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally to form a heterocycle; and each occurrence of R_{c} is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “oxo” refers to

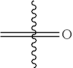 [SMILES: *=O]

substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be rearranged to satisfy the valence requirement. For instance, a pyridine with a 2-oxo substituent group may have the structure of

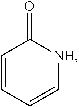

which also includes its tautomeric form of

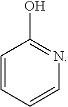

The term “alkylamino” refers to a group having the structure —NHR′, wherein R′ is alkyl, substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,2,4-triazolyl, and tetrazolyl.

The term “alkoxy” refers to a group having the structure —OR′, wherein R′ is alkyl, substituted alkyl, cycloalkyl or substituted cycloalkyl, as defined herein. Examples of alkoxy groups include, but are not limited to, methoxy, ethoxy, n-propoxy, iso-propoxy, cyclopropoxy, n-butoxy, tert-butoxy, neopentyloxy, n-pentyloxy, hexyloxy, cyclohexyloxy, and the like.

The term “alkylthio” refers to a group having the structure —SR′, wherein R′ is alkyl, substituted alkyl, cycloalkyl or substituted cycloalkyl, as defined herein. Examples of alkylthio groups include, but are not limited to, methythio, ethythio, n-propylthio, iso-propylthio, cyclopropylthio, n-butylthio, tert-butylthio, neopentylthio, n-pentylthio, hexylthio, cyclohexylthio, and the like.

The terms “halogen” or “halo” refer to chlorine, bromine, fluorine, or iodine.

The term “substituted” refers to the embodiments in which a molecule, molecular moiety, or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂R_{c}, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of R_{c} is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a phenol or carboxylic acid, zwitterions (“inner salts”) may be formed and are included within the term “salt(s)” as used herein. Pharmaceutically-acceptable (i.e., non-toxic, physiologically-acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates, or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid; for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a phenol or carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides, and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention. As used herein, any depicted structure of the compound includes the tautomeric forms thereof.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to or greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999), the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O ³¹P, ³²P, ³⁵S, ¹⁸F, and ³⁶Cl, respectively. Compounds of the present invention, or an enantiomer, diastereomer, tautomer, or pharmaceutically-acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements, and hence may be preferred in some circumstances. Isotopically-labeled compounds can generally be prepared by carrying out the procedures disclosed in the Schemes and/or in the Examples below, by substituting a readily-available isotopically-labeled reagent for a non-isotopically-labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; cardio-facio-cutaneous syndrome; hereditary gingival fibromatosis type 1; autoimmune lymphoproliferative syndrome; and capillary malformation-arterovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

### Compounds

Novel compounds as Kv1.3 potassium channel blockers are described. Applicants have surprisingly discovered that the compounds disclosed herein exhibit potent Kv1.3 potassium channel-inhibiting properties. Additionally, Applicants have surprisingly discovered that the compounds disclosed herein selectively block the Kv1.3 potassium channel and do not block the hERG channel and thus have desirable cardiovascular safety profiles.

In one aspect, a compound of Formula I or a pharmaceutically-acceptable salt thereof is described,

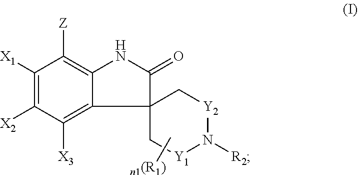

wherein:
X₁, X₂, and X₃ are each independently H, halogen, CN, alkyl, cycloalkyl, halogenated alkyl, halogenated cycloalkyl, OH, SH, alkoxy, halogenated alkoxy, alkylthio, or halogenated alkylthio;
or alternatively X₁ and X₂ and the carbon atoms they are connected to taken together form a 5- or 6-membered aryl;
or alternatively X₂ and X₃ and the carbon atoms they are connected to taken together form a 5- or 6-membered aryl;
Z is H, alkyl, halogenated alkyl, heteroalkyl, cycloalkyl, heterocycle, aryl, heteroaryl, halogen, CN, CF₃, OCF₃, ORₐ, NRₐR_{b}, or NRₐ(C═O)R_{b};
Y₁ is absent or C(R₁)₂;
Y₂ is absent, C(R₁)₂, C(R₁)₂(C═O), C(R₁)₂C(R₁)₂ or C(R₁)₂C(R₁)₂(C═O);
each occurrence of R₁ is independently H, halogen, alkyl, cycloalkyl, saturated heterocycle, aryl, heteroaryl, (CR₄R₅)ₙ₃OR_{c}, or (CR₄R₅)ₙ₃NR_{c}R_{d};
R₂ is alkyl, heteroalkyl, cycloalkyl, heterocycle, bicycloalkyl, spiroalkyl, heterobicycloalkyl, heterospiroalkyl, alkylaryl, alkylheteroaryl, aryl, heteroaryl, (CR₄R₅)ₙ₂(C═O)R₃, (CR₄R₅)ₙ₂(C═O)N(R₄)R₃, SO₂R_{c}, or SO₂NR_{c}R_{d};
each occurrence of R₃ is independently H, alkyl, cycloalkyl, heterocycle, bicycloalkyl, spiroalkyl, heterobicycloalkyl, heterospiroalkyl, alkylaryl, alkylheteroaryl, aryl, or heteroaryl;
each occurrence of R₄ and R₅ is independently H, alkyl, cycloalkyl, heterocycle, aryl, or heteroaryl;
each occurrence of Rₐ and R_{b} is independently H, alkyl, cycloalkyl, heterocycle, aryl, or heteroaryl;
or alternatively Rₐ and R_{b} together with the nitrogen atom that they are connected to form a 3-7-membered heterocycle;
each occurrence of R_{c} and R_{d} is independently H, alkyl, cycloalkyl, heterocycle, aryl, or heteroaryl;
or alternatively R_{c} and R_{d} together with the nitrogen atom that they are connected to form a 3-7-membered heterocycle;
each heterocycle comprises 1-3 heteroatoms each independently selected from the group consisting of N, O and S;
each of alkyl, cycloalkyl, heteroalkyl, heterocycle, aryl, and heteroaryl in X₁, X₂, X₃, Z, R₁, R₂, or R₃, where applicable, are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)Rₐ, where valence permits;
n₁ is an integer from 0-4;
n₂ is an integer from 0-4; and
n₃ is an integer from 0-4.

In some embodiments, X₁ is H, halogen, CN, alkyl, halogenated alkyl, cycloalkyl, or halogenated cycloalkyl. In some embodiments, X₁ is OH, SH, alkoxy, halogenated alkoxy, alkylthio, or halogenated alkylthio. In some embodiments, X₁ is H, halogen, fluorinated alkyl, or alkyl. In some embodiments, X₁ is H or halogen. In other embodiments, X₁ is fluorinated alkyl or alkyl. In other embodiments, X₁ is cycloalkyl. In some embodiments, X₁ is H, F, Cl, Br, Me, CF₂H, CF₂Cl, or CF₃. In some embodiments, X₁ is H, F, or Cl. In some embodiments, X₁ is F or Cl. In some embodiments, X₁ is H or Cl. In some embodiments, X₁ is F. In some embodiments, X₁ is Cl. In some embodiments, X₁ is CH₃. In some embodiments, X₁ is CF₃ or CF₂H. In some embodiments, X₁ is CF₂Cl. In some embodiments, X₁ is H.

In some embodiments, X₂ is H, halogen, CN, alkyl, halogenated alkyl, cycloalkyl, or halogenated cycloalkyl. In some embodiments, X₂ is OH, SH, alkoxy, halogenated alkoxy, alkylthio, or halogenated alkylthio. In some embodiments, X₂ is H, halogen, fluorinated alkyl, or alkyl. In some embodiments, X₂ is H or halogen. In other embodiments, X₂ is fluorinated alkyl or alkyl. In other embodiments, X₂ is cycloalkyl. In some embodiments, X₂ is H, F, Cl, Br, Me, CF₂H, CF₂Cl, or CF₃. In some embodiments, X₂ is H, F, or Cl. In some embodiments, X₂ is F or Cl. In some embodiments, X₂ is H or Cl. In some embodiments, X₂ is F. In some embodiments, X₂ is Cl. In some embodiments, X₂ is CH₃. In some embodiments, X₂ is CF₃ or CF₂H. In some embodiments, X₂ is CF₂Cl. In some embodiments, X₂ is H.

In some embodiments, X₃ is H, halogen, CN, alkyl, halogenated alkyl, cycloalkyl, or halogenated cycloalkyl. In some embodiments, X₃ is OH, SH, alkoxy, halogenated alkoxy, alkylthio, or halogenated alkylthio. In some embodiments, X₃ is H, halogen, fluorinated alkyl, or alkyl. In some embodiments, X₃ is H or halogen. In other embodiments, X₃ is fluorinated alkyl or alkyl. In other embodiments, X₃ is cycloalkyl. In some embodiments, X₃ is H, F, Cl, Br, Me, CF₂H, CF₂Cl, or CF₃. In some embodiments, X₃ is H, F, or Cl. In some embodiments, X₃ is F or Cl. In some embodiments, X₃ is H or Cl. In some embodiments, X₃ is F. In some embodiments, X₃ is Cl. In some embodiments, X₃ is CH₃. In some embodiments, X₃ is CF₃ or CF₂H. In some embodiments, X₃ is CF₂Cl. In some embodiments, X₃ is H.

In some embodiments, Z is H, halogen, CN, alkyl, halogenated alkyl, cycloalkyl, or halogenated cycloalkyl. In some embodiments, Z is H, halogen, fluorinated alkyl, or alkyl. In some embodiments, Z is H or halogen. In other embodiments, Z is fluorinated alkyl or alkyl. In other embodiments, Z is cycloalkyl. In some embodiments, Z is H, F, Cl, Br, Me, CF₂H, CF₂Cl, or CF₃. In some embodiments, Z is H, F, or Cl. In some embodiments, Z is F or Cl. In some embodiments, Z is H or Cl. In some embodiments, Z is F. In some embodiments, Z is Cl. In some embodiments, Z is CH₃. In some embodiments, Z is CF₃ or CF₂H. In some embodiments, Z is CF₂Cl. In some embodiments, Z is H.

In some embodiments, Z is ORₐ. In some embodiments, Z is OH or O—(C₁-C₄ alkyl). In some embodiments, Z is OH, OMe, OCF₃, OEt, OPr, Oi-Pr, OBu, Oi-Bu, Osec-Bu, or Ot-Bu. In some embodiments, Z is OH. In some embodiments, Z is NRₐR_{b} or NRₐ(C═O)R_{b}. In some embodiments, Z is NH₂, NHMe, NHEt, or NMe₂. In some embodiments, Z is NHCOMe, NMeCOEt, or NHCOEt.

In some embodiments, at least two of Z, X₁, X₂, and X₃ are not H. In some embodiments, X₁ and Z are not H. In some embodiments, X₂ and Z are not H. In some embodiments, X₃ and Z are not H. In some embodiments, X₁ and X₂ are not H. In some embodiments, X₁ and X₃ are not H. In some embodiments, X₂ and X₃ are not H. In some embodiments, Z, X₁, and X₂ are not H. In some embodiments, Z, X₁, and X₃ are not H. In some embodiments, Z, X₂, and X₃ are not H. In some embodiments, X₁, X₂, and X₃ are not H.

In some embodiments, at least two of Z, X₁, X₂, and X₃ are not H and are each selected from the group consisting of alkyl, halogen, halogenated alkyl, and cycloalkyl. In some embodiments, X₁ and Z are not H and are each selected from the group consisting of alkyl, halogen, halogenated alkyl, and cycloalkyl. In some embodiments, X₂ and Z are not H and are each selected from the group consisting of alkyl, halogen, halogenated alkyl, and cycloalkyl. In some embodiments, X₃ and Z are not H and are each selected from the group consisting of alkyl, halogen, halogenated alkyl, and cycloalkyl. In some embodiments, X₁ and X₂ are not H and are each selected from the group consisting of alkyl, halogen, halogenated alkyl, and cycloalkyl. In some embodiments, X₁ and X₃ are not H and are each selected from the group consisting of alkyl, halogen, halogenated alkyl, and cycloalkyl. In some embodiments, X₂ and X₃ are not H and are each selected from the group consisting of alkyl, halogen, halogenated alkyl, and cycloalkyl. In some embodiments, Z, X₁, and X₂ are not H and are each selected from the group consisting of alkyl, halogen, halogenated alkyl, and cycloalkyl. In some embodiments, Z, X₁, and X₃ are not H and are each selected from the group consisting of alkyl, halogen, halogenated alkyl, and cycloalkyl. In some embodiments, Z, X₂, and X₃ are not H and are each selected from the group consisting of alkyl, halogen, halogenated alkyl, and cycloalkyl. In some embodiments, X₁, X₂, and X₃ are not H and are each selected from the group consisting of alkyl, halogen, halogenated alkyl, and cycloalkyl.

In some embodiments, at least two of Z, X₁, X₂, and X₃ are not H and are each selected from the group consisting of halogen and alkyl. In some embodiments, X₁ and Z are not H and are each selected from the group consisting of halogen and alkyl. In some embodiments, X₂ and Z are not H and are each selected from the group consisting of halogen and alkyl. In some embodiments, X₃ and Z are not H and are each selected from the group consisting of halogen and alkyl. In some embodiments, X₁ and X₂ are not H and are each selected from the group consisting of halogen and alkyl. In some embodiments, X₁ and X₃ are not H and are each selected from the group consisting of halogen and alkyl. In some embodiments, X₂ and X₃ are not H and are each selected from the group consisting of halogen and alkyl. In some embodiments, Z, X₁, and X₂ are not H and are each selected from the group consisting of halogen and alkyl. In some embodiments, Z, X₁, and X₃ are not H and are each selected from the group consisting of halogen and alkyl. In some embodiments, Z, X₂, and X₃ are not H and are each selected from the group consisting of halogen and alkyl. In some embodiments, X₁, X₂, and X₃ are not H and are each selected from the group consisting of halogen and alkyl.

In some embodiments, at least two of Z, X₁, X₂, and X₃ are each independently Cl, Br, or methyl. In some embodiments, X₁ and Z are each independently Cl, Br, or methyl. In some embodiments, X₂ and Z are each independently Cl, Br, or methyl. In some embodiments, X₃ and Z are each independently Cl, Br, or methyl. In some embodiments, X₁ and X₂ are each independently Cl, Br, or methyl. In some embodiments, X₁ and X₃ are each independently Cl, Br, or methyl. In some embodiments, X₂ and X₃ are each independently Cl, Br, or methyl. In some embodiments, Z, X₁, and X₂ are each independently Cl, Br, or methyl. In some embodiments, Z, X₁, and X₃ are each independently Cl, Br, or methyl. In some embodiments, Z, X₂, and X₃ are each independently Cl, Br, or methyl. In some embodiments, X₁, X₂, and X₃ are each independently Cl, Br, or methyl.

In some embodiments, at least two of Z, X₁, X₂, and X₃ are each Cl. In some embodiments, X₁ and Z are each Cl. In some embodiments, X₂ and Z are each Cl. In some embodiments, X₃ and Z are each Cl. In some embodiments, X₁ and X₂ are each Cl. In some embodiments, X₁ and X₃ are each Cl. In some embodiments, X₂ and X₃ are each Cl. In some embodiments, Z, X₁, and X₂ are each Cl. In some embodiments, Z, X₁, and X₃ are each Cl. In some embodiments, Z, X₂, and X₃ are each Cl. In some embodiments, X₁, X₂, and X₃ are each Cl.

In some embodiments, the structural moiety

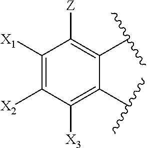

has the structure of

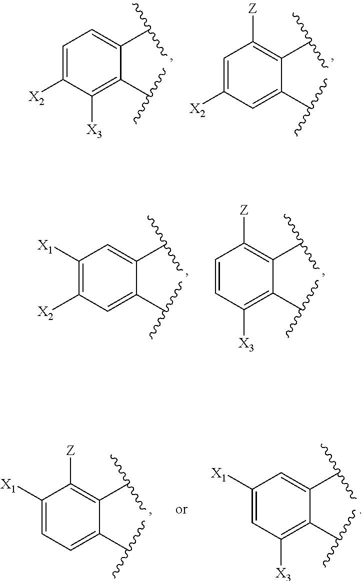

In some embodiments, the structural moiety

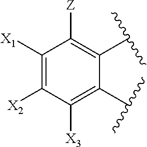

has the structure of

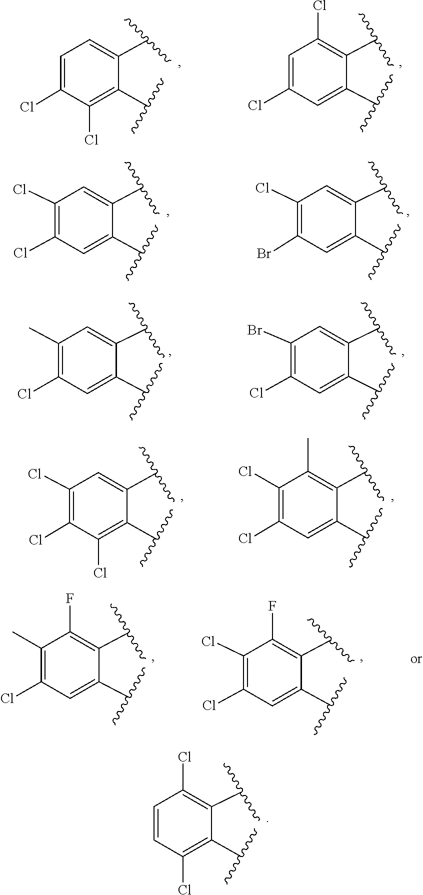

In some embodiments, the structural moiety

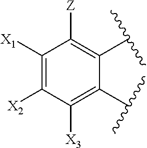

has the structure of

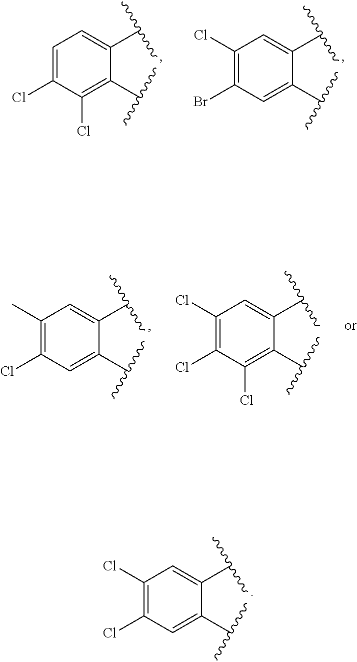

In some embodiments, the structural moiety

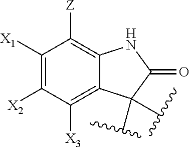

has the structure of

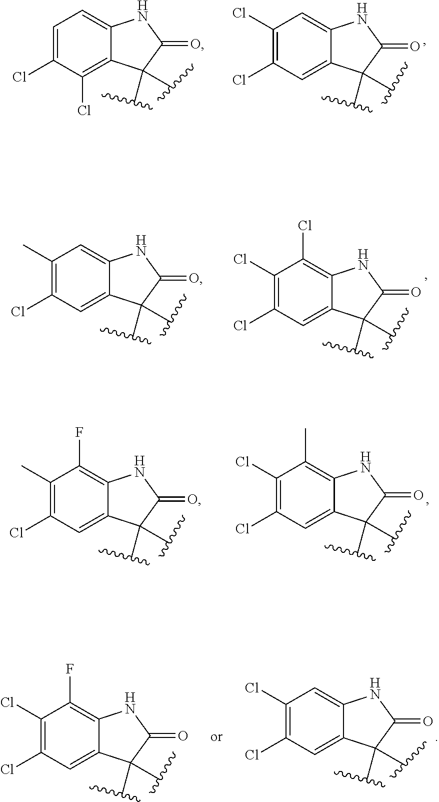

In some embodiments, Y₁ is absent. In some embodiments, Y₁ is C(R₁)₂.

In some embodiments, Y₂ is absent. In some embodiments, Y₂ is C(R₁)₂. In some embodiments, Y₂ is C(R₁)₂C(R₁)₂. In some embodiments, Y₂ is C(R₁)₂(C═O) or C(R₁)₂C(R₁)₂(C═O).

In some embodiments, Y₁ and Y₂ are each independently absent or C(R₁)₂. In some embodiments, Y₁ is absent and Y₂ is C(R₁)₂. In some embodiments, Y₁ is C(R₁)₂ and Y₂ is C(R₁)₂.

In some embodiments, the structural moiety

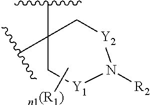

has the structure of

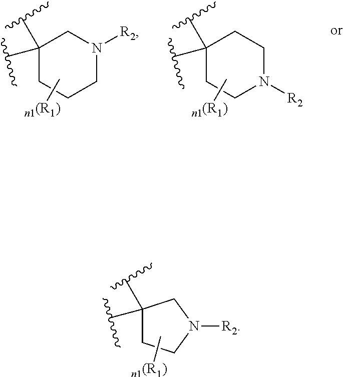

In some embodiments, the structural moiety

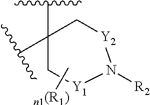

has the structure of

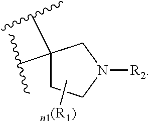

In some embodiments, the structural moiety

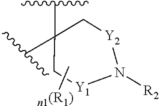

has the structure of

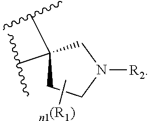

In some embodiments, n₁ is 0. In some embodiments, n₁ is an integer from 1-3. In some embodiments, n₁ is 2 or 3. In some embodiments, n₁ is 1 or 2. In some embodiments, n₁ is 0 or 1. In some embodiments, n₁ is 1. In some embodiments, n₁ is 2. In some embodiments, n₁ is 3.

In some embodiments, at least one occurrence of R₁ is H, alkyl, cycloalkyl, aryl, or heteroaryl. In some embodiments, at least one occurrence of R₁ is halogen, saturated heterocycle, (CR₄R₅)ₙ₃OR_{c}, or (CR₄R₅)ₙ₃NR_{c}R_{d}. In some embodiments, at least one occurrence of R₁ is H, alkyl, or cycloalkyl. In some embodiments, at least one occurrence of R₁ is H or alkyl. Non-limiting examples of alkyl include methyl, ethyl, propyl, isopropyl, n-butyl, iso-butyl, sec-butyl, pentyl, hexyl, heptyl, and octyl. In some embodiments, at least one occurrence of R₁ is a cycloalkyl. Non-limiting examples of cycloalkyl include cyclopropyl, cyclobutyl, cyclopentyl, cyclohexyl, and cycloheptyl. In some embodiments, at least one occurrence of R₁ is halogen. Non-limiting examples of halogen include F, Cl, Br, and I.

In some embodiments, one or more occurrences of R₁ are (CR₄R₅)ₙ₃OR_{c} or (CR₄R₅)ₙ₃NR_{c}R_{d}. In some embodiments, one or more occurrences of R₁ are OR_{c}, NR_{c}R_{d}, —CH₂OR_{c}, —CH₂NR_{c}R_{d}, —CH₂CH₂OR_{c}, or —CH₂CH₂NR_{c}R_{d}. In some specific embodiments, at least one occurrence of R₁ is NH₂, CH₂NH₂, or CH₂CH₂NH₂. In other specific embodiments, at least one occurrence of R₁ is OH, CH₂OH or CH₂NH₂.

In still other embodiments, at least one occurrence of R₁ is an optionally substituted 4-, 5-, 6- or 7-membered heterocycle containing 1-3 heteroatoms each selected from the group consisting of N, O, and S. In some embodiments, at least one occurrence of R₁ is heteroaryl. In some embodiments, at least one occurrence of R₁ is aryl. In some embodiments, at least one occurrence of R₁ selected from the group consisting of

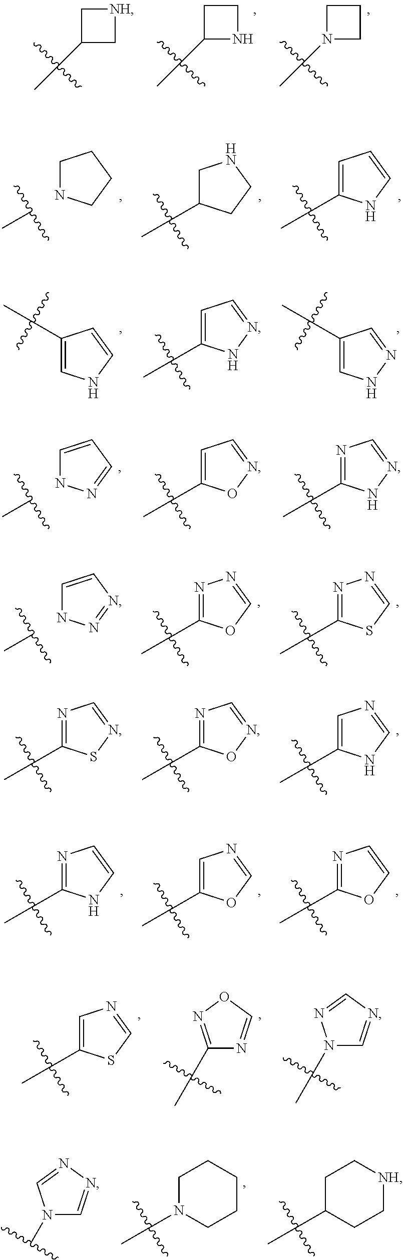

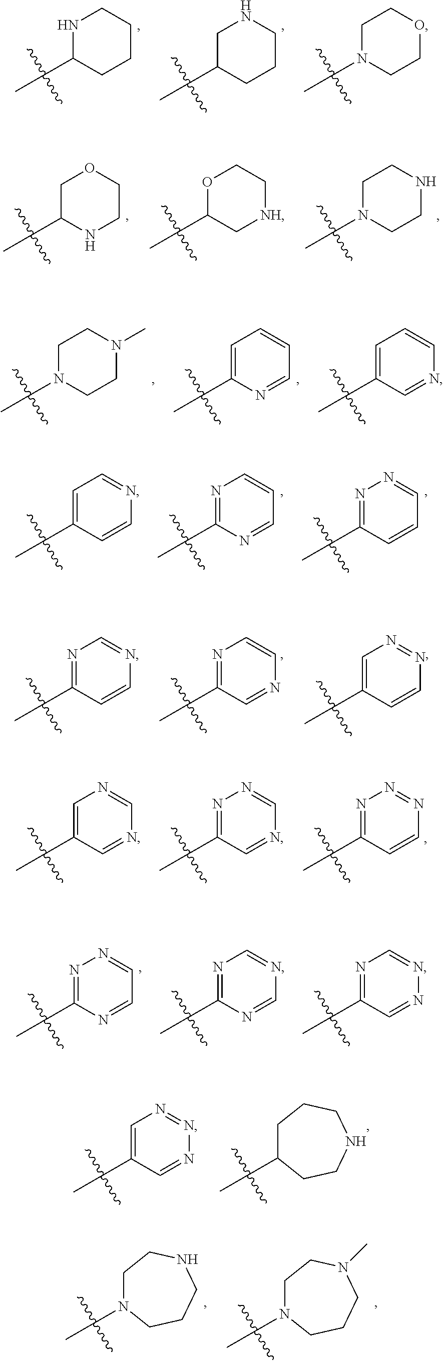

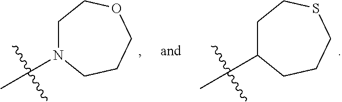 [SMILES: *N1CCCOCC1 ; *C1CCCSCC1]

In some embodiments, the compound of Formula I has a structure of Formula Ia

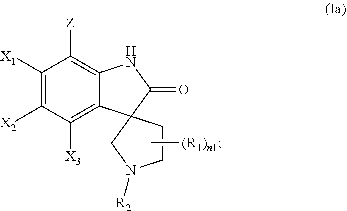

wherein
X₁, X₂, and X₃ are each independently H, halogen, or alkyl;
Z is H, halogen, alkyl, halogenated alkyl, CN, ORₐ, or NRₐR_{b}; and
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d}.

In some embodiments, Z is H, halogen, alkyl, or halogenated alkyl. In some embodiments, Z is H, F, Cl, Br, CH₃, or CF₃. In some embodiments, Z is H. In some embodiments, Z is CN, ORₐ, or NRₐR_{b}. In some embodiments, each occurrence of Rₐ and R_{b} is independently H or alkyl. In some embodiments, each occurrence of Rₐ and R_{b} is cycloalkyl or heterocycle. In some embodiments, each occurrence of Rₐ and R_{b} is aryl or heteroaryl.

In some specific embodiments, at least one occurrence of R₁ is alkyl or cycloalkyl. In some embodiments, at least one occurrence of R₁ is halogen, (CR₄R₅)ₙ₃OR_{c}, or (CR₄R₅)ₙ₃NR_{c}R_{d}. In some embodiments, at least one occurrence of R₁ is saturated heterocycle, aryl, or heteroaryl. In some embodiments, n₁ is 0 or 1.

In some embodiments, the compound of Formula I has a structure of Formula Ib

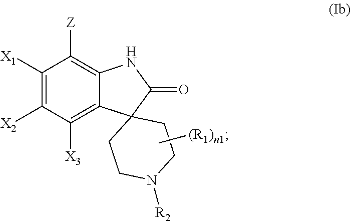

wherein:
X₁, X₂, and X₃ are each independently H, halogen, or alkyl;
Z is H, halogen, alkyl, halogenated alkyl, CN, ORₐ, or NRₐR_{b}; and
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d}.

In some embodiments, Z is H, halogen, alkyl, or halogenated alkyl. In some embodiments, Z is H, F, Cl, Br, CH₃, or CF₃. In some embodiments, Z is H. In some embodiments, Z is CN, ORₐ, or NRₐR_{b}. In some embodiments, each occurrence of Rₐ and R_{b} is independently H or alkyl. In some embodiments, each occurrence of Rₐ and R_{b} is cycloalkyl or heterocycle. In some embodiments, each occurrence of Rₐ and R_{b} is aryl or heteroaryl.

In some specific embodiments, at least one occurrence of R₁ is alkyl or cycloalkyl. In some embodiments, at least one occurrence of R₁ is halogen, (CR₄R₅)ₙ₃OR_{c}, or (CR₄R₅)ₙ₃NR_{c}R_{d}. In some embodiments, at least one occurrence of R₁ is saturated heterocycle, aryl, or heteroaryl. In some embodiments, n₁ is 0 or 1.

In some embodiments, R₂ is alkyl, cycloalkyl, or heteroalkyl. In some embodiments, R₂ is alkyl. Non-limiting examples of alkyl include methyl, ethyl, propyl, isopropyl, n-butyl, iso-butyl, sec-butyl, pentyl, hexyl, heptyl, and octyl. In some embodiments, R₂ is a cycloalkyl. Non-limiting examples of cycloalkyl include cyclopropyl, cyclobutyl, cyclopentyl, cyclohexyl, and cycloheptyl.

In some embodiments, R₂ is heterocycle, aryl, heteroaryl, alkylaryl, or alkylheteroaryl. In some embodiments, R₂ is an optionally substituted 4-, 5-, 6- or 7-membered heterocycle containing 1-3 heteroatoms each selected from the group consisting of N, O, and S. In some embodiments, R₂ is heteroaryl. In some embodiments, R₂ is aryl. In some embodiments, R₂ selected from the group consisting of

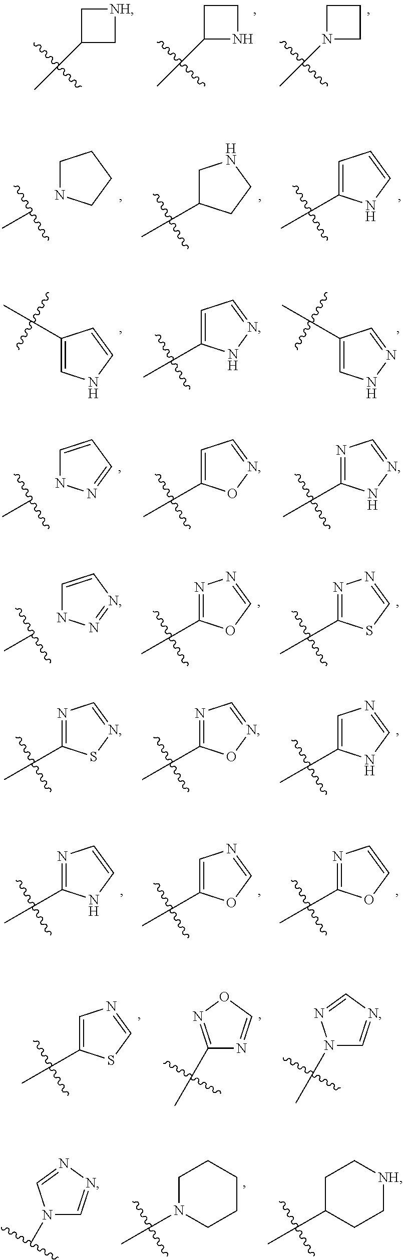

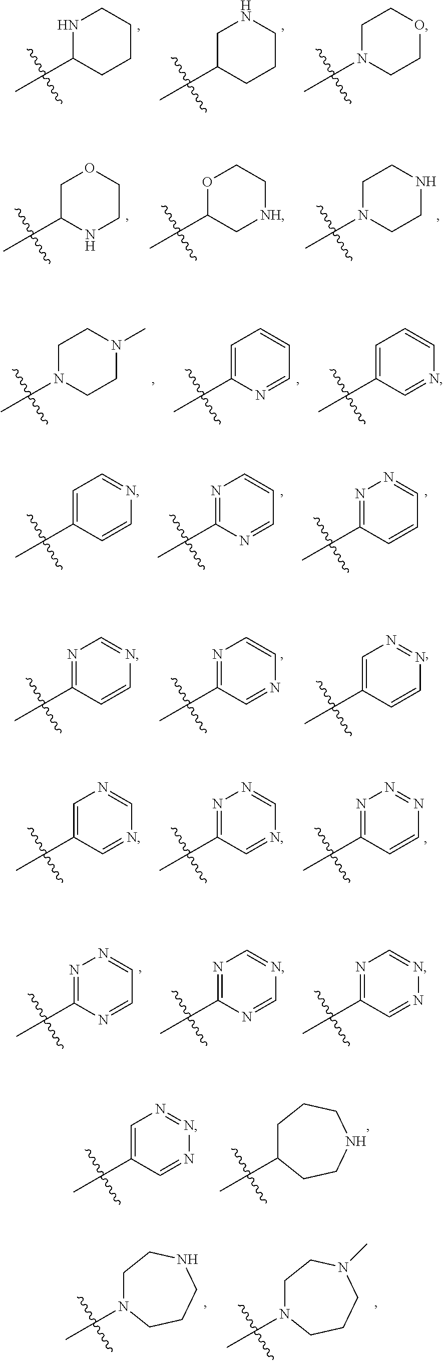

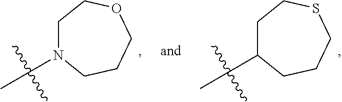 [SMILES: *N1CCCOCC1 ; *C1CCCSCC1]

wherein the heterocycle or heteroaryl of R₂ is optionally substituted by alkyl, OH, oxo, or (C═O)C₁₋₄alkyl where valence permits.

In some embodiments, R₂ is bicycloalkyl, spiroalkyl, heterobicycloalkyl, or heterospiroalkyl. Exemplary bicycloalkyl groups include, but not limited to, adamantyl, bicyclo[1.1.1]pentyl, bicyclo[2.2.1]heptyl, bicyclo[3.1.1]heptyl, bicyclo[2.1.1]hexyl, octahydropentalenyl, bicyclo[3.2.1]octyl, bicyclo[3.3.3]undecanyl, decahydronaphthalenyl, bicyclo[3.2.0]heptyl, octahydro-1H-indenyl, bicyclo[4.2.1]nonanyl, and the like. Exemplary spiro bicycloalkyl groups include, but not limited to, spiro[4.4]nonyl, spiro[3.3]heptyl, spiro[5.5]undecyl, spiro[3.5]nonyl, spiro[4.5]decyl, and the like. The term “heterobicycloalkyl,” as used herein, refers to a bicycloalkyl group, as defined herein, in which one or more of the constituent carbon atoms have been replaced by nitrogen, oxygen, or sulfur. The term “heterospiroalkyl,” as used herein, refers to a spiroalkyl group, as defined herein, in which one or more of the constituent carbon atoms have been replaced by nitrogen, oxygen, or sulfur. In some embodiments, R₂ is selected from the group consisting of

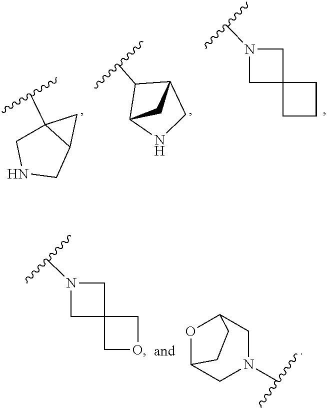 [SMILES: *C12CNCC1C2 ; *C1[C@@H]2CN[C@H]1C2 ; *N1CC2(CCC2)C1 ; *N1CC2(COC2)C1 ; *N1CC2CCC(C1)O2]

In still other embodiments, R₂ is (CR₄R₅)ₙ₂(C═O)R₃ or (CR₄R₅)ₙ₂(C═O)NR₃R₄. In some embodiments, R₂ is CH₂(C═O)R₃, CH₂CH₂(C═O)R₃, (C═O)R₃, CH₂(C═O)NR₃R₄ or (C═O)NR₃R₄.

In some embodiments, R₃ is H, alkyl or cycloalkyl. In some embodiments, R₃ is alkyl. Non-limiting examples of alkyl include methyl, ethyl, propyl, isopropyl, n-butyl, iso-butyl, sec-butyl, pentyl, hexyl, heptyl, and octyl. In some embodiments, R₃ is a cycloalkyl. Non-limiting examples of cycloalkyl include cyclopropyl, cyclobutyl, cyclopentyl, cyclohexyl, and cycloheptyl.

In some embodiments, R₃ is heterocycle, aryl, heteroaryl, alkylaryl, or alkylheteroaryl. In some embodiments, R₃ is an optionally substituted 4-, 5-, 6- or 7-membered heterocycle containing 1-3 heteroatoms each selected from the group consisting of N, O, and S. In some embodiments, R₃ is heteroaryl. In some embodiments, R₃ is aryl. In some embodiments, R₃ selected from the group consisting of

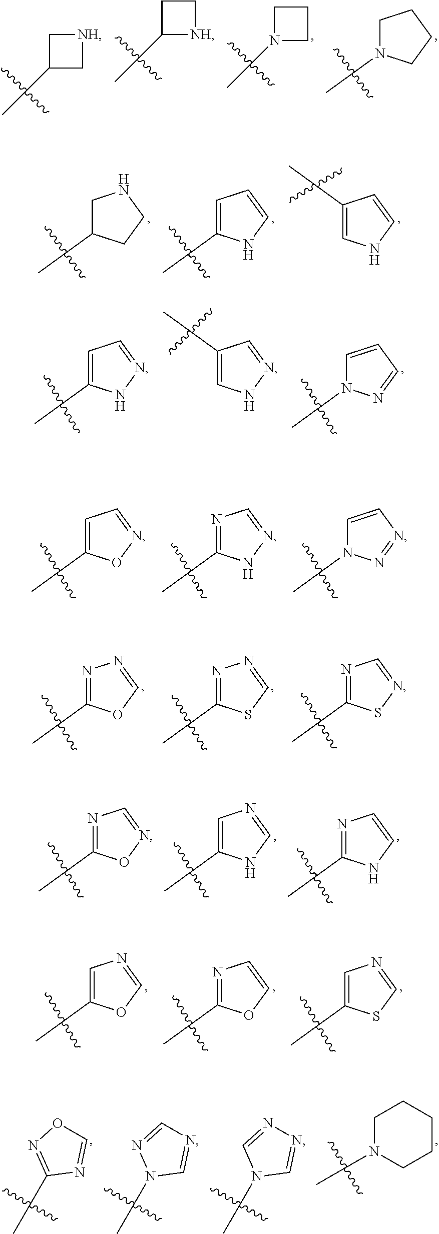

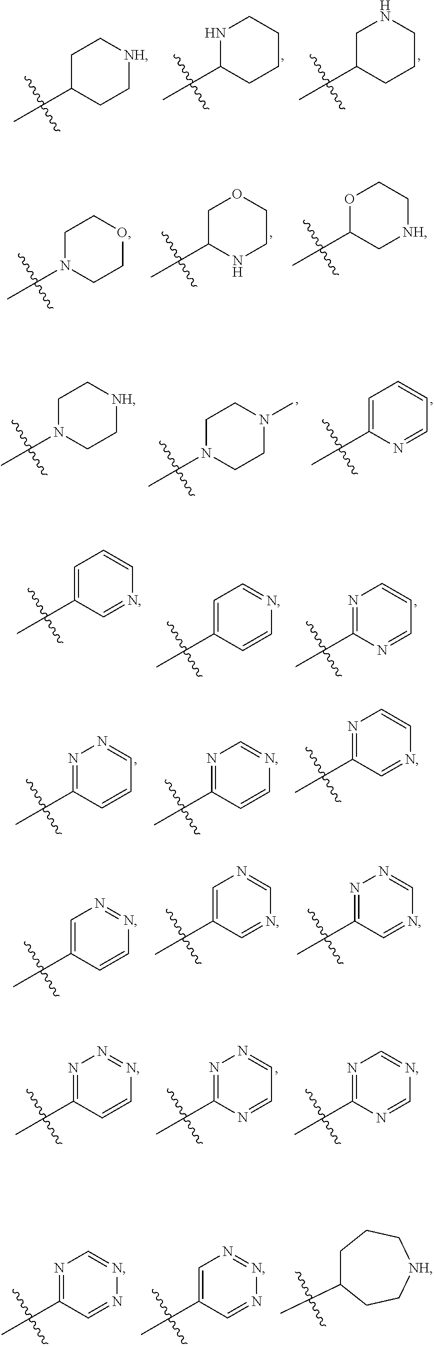

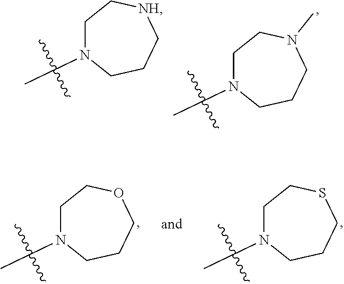

wherein the heterocycle or heteroaryl of R₃ is optionally substituted by alkyl, OH, oxo, or (C═O)C₁₋₄alkyl where valence permits.

In some embodiments, R₃ is bicycloalkyl, spiroalkyl, heterobicycloalkyl, or heterospiroalkyl. Exemplary bicycloalkyl groups include, but not limited to, adamantyl, bicyclo[1.1.1]pentyl, bicyclo[2.2.1]heptyl, bicyclo[3.1.1]heptyl, bicyclo[2.1.1]hexyl, octahydropentalenyl, bicyclo[3.2.1]octyl, bicyclo[3.3.3]undecanyl, decahydronaphthalenyl, bicyclo[3.2.0]heptyl, octahydro-1H-indenyl, bicyclo[4.2.1]nonanyl, and the like. Exemplary spiro bicycloalkyl groups include, but not limited to, spiro[4.4]nonyl, spiro[3.3]heptyl, spiro[5.5]undecyl, spiro[3.5]nonyl, spiro[4.5]decyl, and the like. The term “heterobicycloalkyl,” as used herein, refers to a bicycloalkyl group, as defined herein, in which one or more of the constituent carbon atoms have been replaced by nitrogen, oxygen, or sulfur. The term “heterospiroalkyl,” as used herein, refers to a spiroalkyl group, as defined herein, in which one or more of the constituent carbon atoms have been replaced by nitrogen, oxygen, or sulfur. In some embodiments, R₃ is selected from the group consisting of

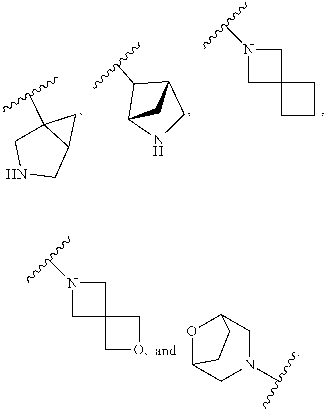 [SMILES: *C12CNCC1C2 ; *C1[C@@H]2CN[C@H]1C2 ; *N1CC2(CCC2)C1 ; *N1CC2(COC2)C1 ; *N1CC2CCC(C1)O2]

In some embodiments, each of alkyl, cycloalkyl, heteroalkyl, heterocycle, aryl, and heteroaryl in X₁, X₂, X₃, Z, R₁, R₂, or R₃, where applicable, are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)Rₐ, where valence permits.

In some embodiments, R₂ is SO₂R_{c} or SO₂NR_{c}R_{d}. In some embodiments, R₂ is (CR₄R₅)ₙ₂OR_{c}, (CR₄R₅)ₙ₂(CR₄)((CR₄R₅)ₙ₃OR_{c})₂, (C═O)(CR₄R₅)ₙ₂OR_{c}, (C═O)(CR₄R₅)ₙ₂(CR₄)((CR₄R₅)ₙ₃OR_{c})₂, (CR₄R₅)ₙ₂COOR_{c}, (C═O)(CR₄R₅)ₙ₂NR_{c}R_{d}, or (CR₄R₅)ₙ₂NR_{c}(C═O)Rₐ. In some specific embodiments, R₂ is selected from the group consisting of SO₂Me, SO₂NHMe, SO₂NMe₂, SO₂NHEt, CH₂OH, CH₂CH₂OH, CH₂OMe, CH₂CH(CH₂OH)₂, CH₂CH(CH₂CH₂OH)₂, CH₂CH₂CH(CH₂OH)₂, (C═O)CH₂OH, (C═O)CH₂CH₂OH, (C═O)CH₂CH(CH₂OH)₂, (C═O)CH₂CH(CH₂CH₂OH)₂, (C═O)CH₂CH₂CH(CH₂OH)₂, CH₂COOH, CH₂CH₂COOH, CH₂COOMe, (C═O)CH₂NH₂, (C═O)NH₂, (C═O)CH₂NHMe, (C═O)NMe₂, CH₂NH(C═O)Me, and NH(C═O)Et.

In some embodiments, each occurrence of R₄ and R₅ is independently H, alkyl, cycloalkyl, or heterocycle. In some specific embodiments, each occurrence of R₄ and R₅ is independently H, CH₃, or CH₂CH₃. In other specific embodiments, each occurrence of R₄ and R₅ is independently H and H, H and Me, Me and Me, H and Et, Me and Et, or Et and Et. In some embodiments, at least one occurrence of R₄ or R₅ is independently aryl or heteroaryl.

In some embodiments, each occurrence of Rₐ and R_{b} is independently H or alkyl. In some specific embodiments, each occurrence of Rₐ and R_{b} is independently H, CH₃, or CH₂CH₃. In some embodiments, each occurrence of Rₐ and R_{b} is independently cycloalkyl or saturated heterocycle. In some embodiments, each occurrence of Rₐ and R_{b} is independently aryl or heteroaryl.

In some embodiments, Rₐ and R_{b} taken together with the nitrogen atom they are connected to form a heterocycle comprising the nitrogen atom and 0-3 additional heteroatoms each selected from the group consisting of N, O, and S. In some embodiments, Rₐ and R_{b} taken together with the nitrogen atom they are connected to form a 4-, 5-, or 6-membered heterocycle. Non-limiting examples of 4-, 5-, or 6-membered heterocycle include azetidine, pyrrolidine, piperidine, and piperazine. In some specific embodiments, the 4-, 5-, or 6-membered heterocycle is

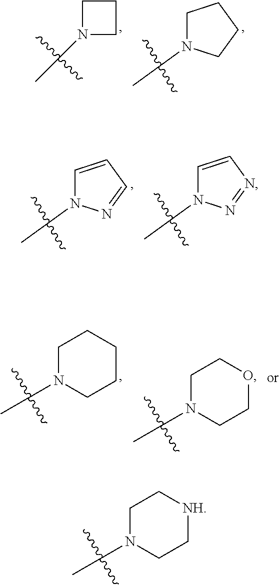

In some embodiments, each occurrence of R_{c} and R_{d} is independently H or alkyl. In some specific embodiments, each occurrence of R_{c} and R_{d} is independently H, CH₃, or CH₂CH₃. In some embodiments, each occurrence of R_{c} and R_{d} is independently cycloalkyl or heterocycle. In some embodiments, each occurrence of R_{c} and R_{d} is independently aryl or heteroaryl.

In some embodiments, R_{c} and R_{d} taken together with the nitrogen atom they are connected to form a heterocycle comprising the nitrogen atom and 0-3 additional heteroatoms each selected from the group consisting of N, O, and S. In some embodiments, R_{c} and R_{d} taken together with the nitrogen atom they are connected to form a 4-, 5-, or 6-membered heterocycle. Non-limiting examples of 4-, 5-, or 6-membered heterocycle include azetidine, pyrrolidine, piperidine, and piperazine. In some specific embodiments, the 4-, 5-, or 6-membered heterocycle is

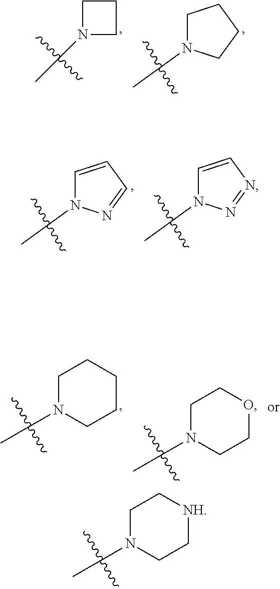

In some embodiments, n₂ is an integer from 0-3. In some embodiments, n₂ is an integer from 1-3. In some embodiments, n₂ is 0. In some embodiments, n₂ is 1 or 2. In some embodiments, n₂ is 1. In some embodiments, n₂ is 3 or 4.

In some embodiments, n₃ is an integer from 0-3. In some embodiments, n₃ is an integer from 1-3. In some embodiments, n₃ is 0. In some embodiments, n₃ is 1 or 2. In some embodiments, n₃ is 1. In some embodiments, n₃ is 3 or 4.

In some embodiments, the compound of Formula I has a structure of Formula Ic:

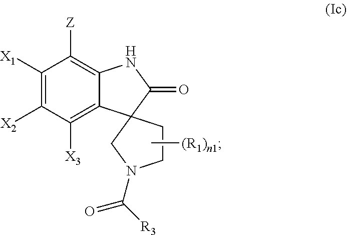

wherein:
X₁, X₂, and X₃ are each independently H, halogen, or alkyl;
Z is H, halogen, alkyl, halogenated alkyl, CN, ORₐ, or NRₐR_{b};
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d}; and
R₃ is alkyl, cycloalkyl, heterocycle, aryl, heteroaryl, bicycloalkyl, spiroalkyl, heterobicycloalkyl, or heterospiroalkyl; and wherein the alkyl, cycloalkyl, heterocycle, aryl, or heteroaryl is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In some embodiments, Z is H, halogen, alkyl, or halogenated alkyl. In some embodiments, Z is H, F, Cl, Br, CH₃, or CF₃. In some embodiments, Z is H. In some embodiments, Z is CN, ORₐ, or NRₐR_{b}. In some embodiments, each occurrence of Rₐ and R_{b} is independently H or alkyl. In some embodiments, each occurrence of Rₐ and R_{b} is cycloalkyl or heterocycle. In some embodiments, each occurrence of Rₐ and R_{b} is aryl or heteroaryl.

In some specific embodiments, at least one occurrence of R₁ is alkyl or cycloalkyl. In some embodiments, at least one occurrence of R₁ is halogen, (CR₄R₅)ₙ₃OR_{c}, or (CR₄R₅)ₙ₃NR_{c}R_{d}. In some embodiments, at least one occurrence of R₁ is saturated heterocycle, aryl, or heteroaryl. In some embodiments, n₁ is 0 or 1.

In some specific embodiments, R₃ is alkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits. In some specific embodiments, R₃ is cycloalkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits. In some specific embodiments, R₃ is heterocycle that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits. In some specific embodiments, R₃ is aryl or heteroaryl each optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits. In some specific embodiments, R₃ is bicycloalkyl, spiroalkyl, heterobicycloalkyl, or heterospiroalkyl.

In some embodiments, the compound of Formula I has a structure of Formula Id:

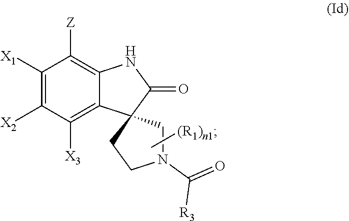

wherein:
X₁, X₂, and X₃ are each independently H, halogen, or alkyl;
Z is H, halogen, alkyl, halogenated alkyl, CN, ORₐ, or NRₐR_{b};
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d}; and
R₃ is alkyl, cycloalkyl, heterocycle, aryl, heteroaryl, bicycloalkyl, spiroalkyl, heterobicycloalkyl, or heterospiroalkyl; and wherein the alkyl, cycloalkyl, heterocycle, aryl, or heteroaryl is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

In some embodiments, Z is H, halogen, alkyl, or halogenated alkyl. In some embodiments, Z is H, F, Cl, Br, CH₃, or CF₃. In some embodiments, Z is H. In some embodiments, Z is CN, ORₐ, or NRₐR_{b}. In some embodiments, each occurrence of Rₐ and R_{b} is independently H or alkyl. In some embodiments, each occurrence of Rₐ and R_{b} is cycloalkyl or heterocycle. In some embodiments, each occurrence of Rₐ and R_{b} is aryl or heteroaryl.

In some specific embodiments, at least one occurrence of R₁ is alkyl or cycloalkyl. In some embodiments, at least one occurrence of R₁ is halogen, (CR₄R₅)ₙ₃OR_{c}, or (CR₄R₅)ₙ₃NR_{c}R_{d}. In some embodiments, at least one occurrence of R₁ is saturated heterocycle, aryl, or heteroaryl. In some embodiments, n₁ is 0 or 1.

In some specific embodiments, R₃ is alkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits. In some specific embodiments, R₃ is cycloalkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits. In some specific embodiments, R₃ is heterocycle that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits. In some specific embodiments, R₃ is aryl or heteroaryl each optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits. In some specific embodiments, R₃ is bicycloalkyl, spiroalkyl, heterobicycloalkyl, or heterospiroalkyl.

In some specific embodiments, R₂ is selected from the group consisting of

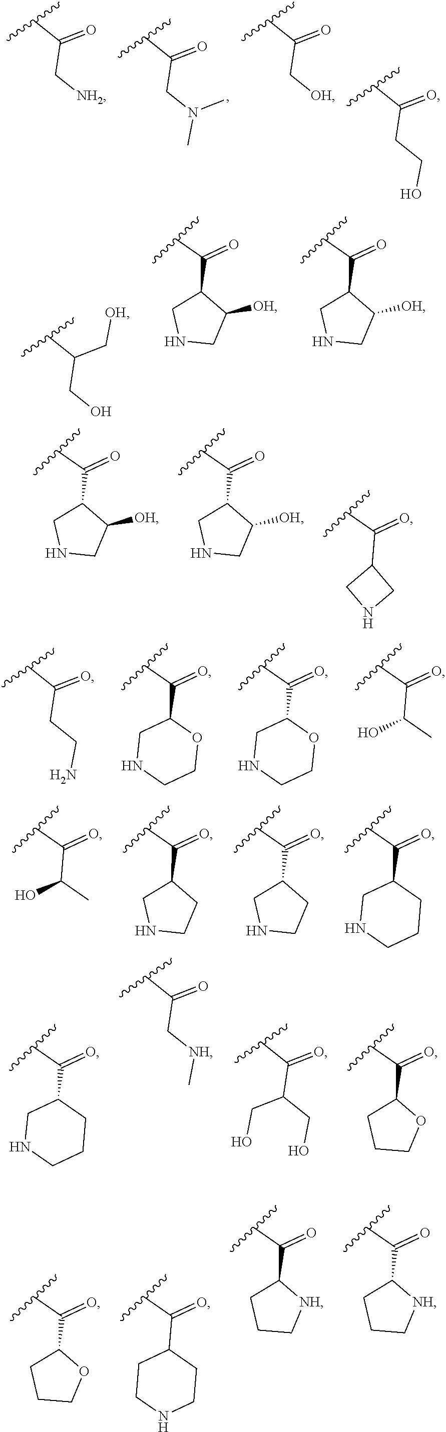

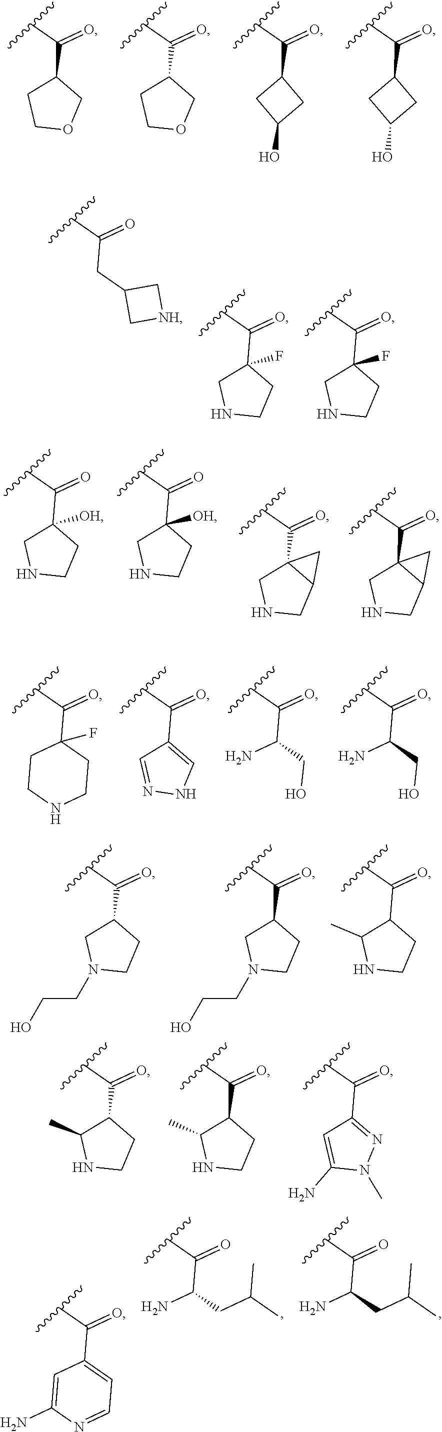

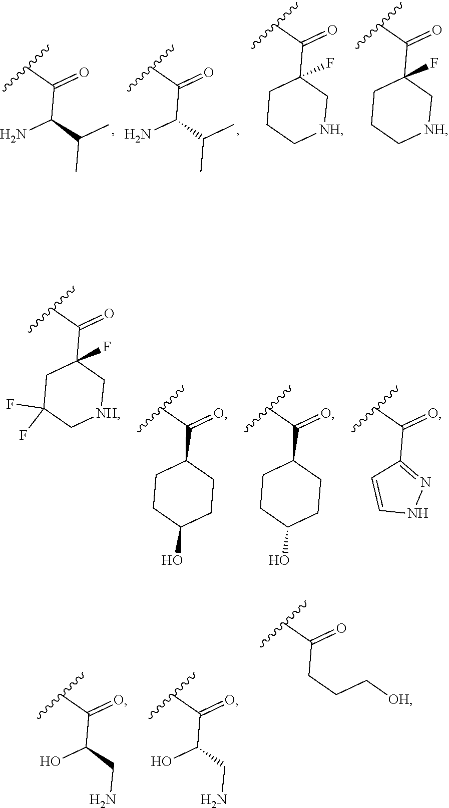

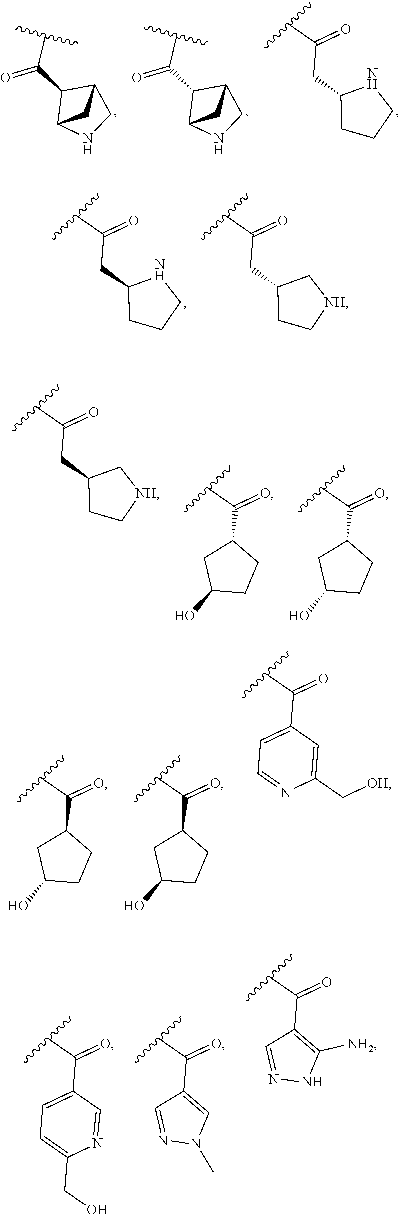

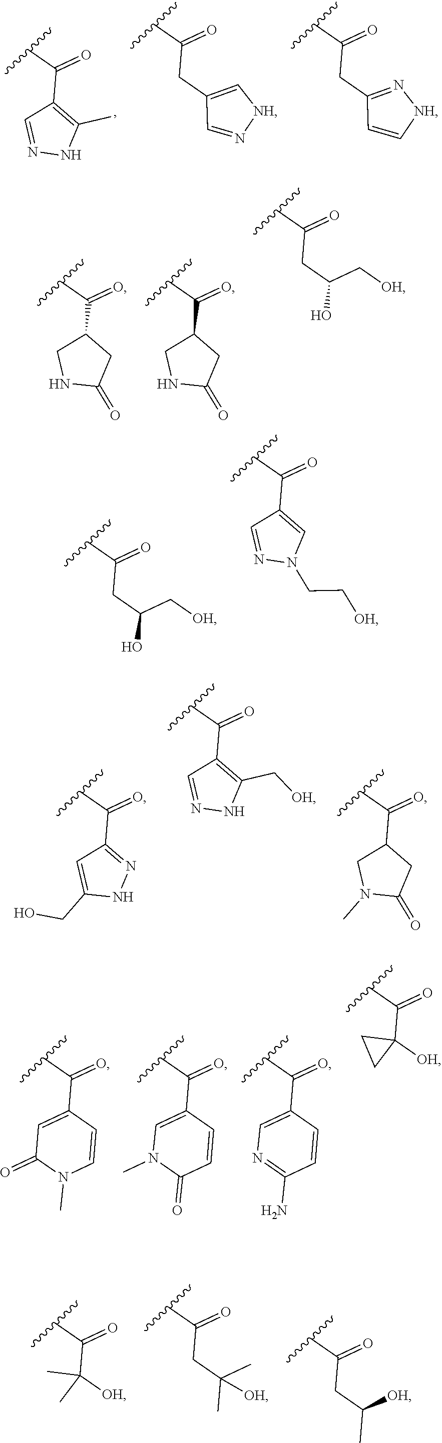

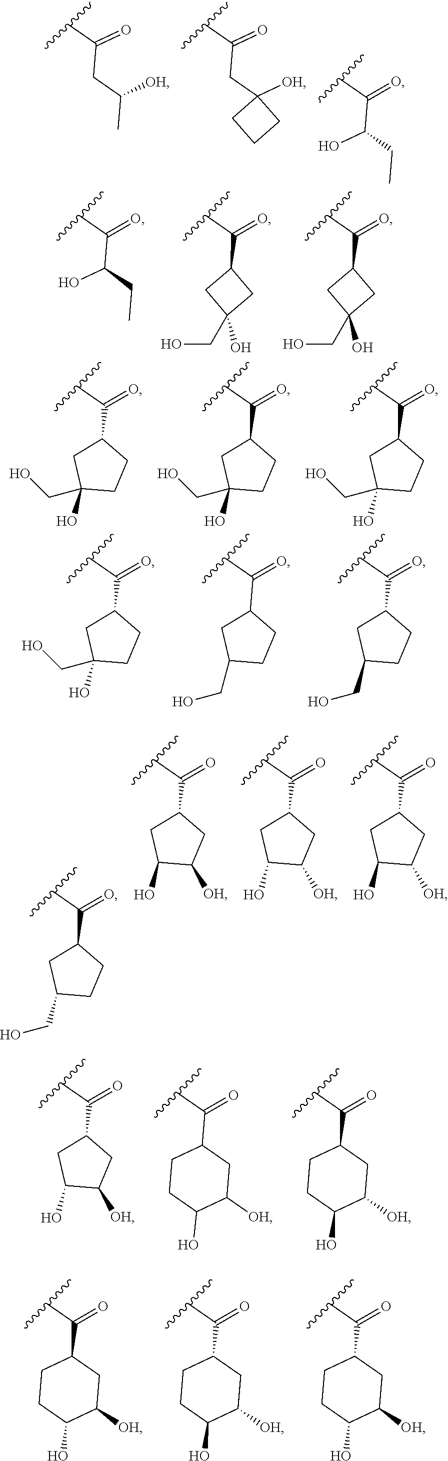

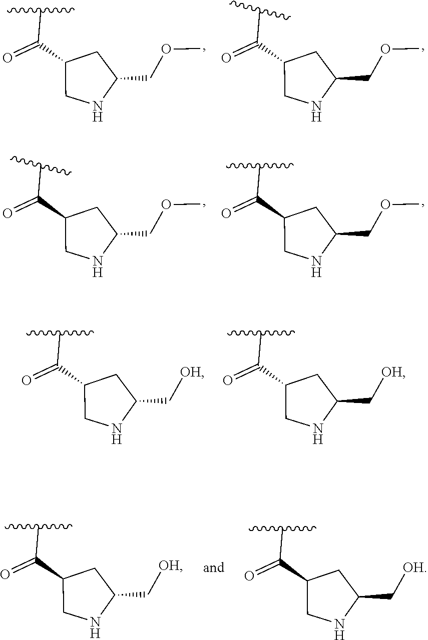

In some specific embodiments, R₂ is selected from the group consisting of

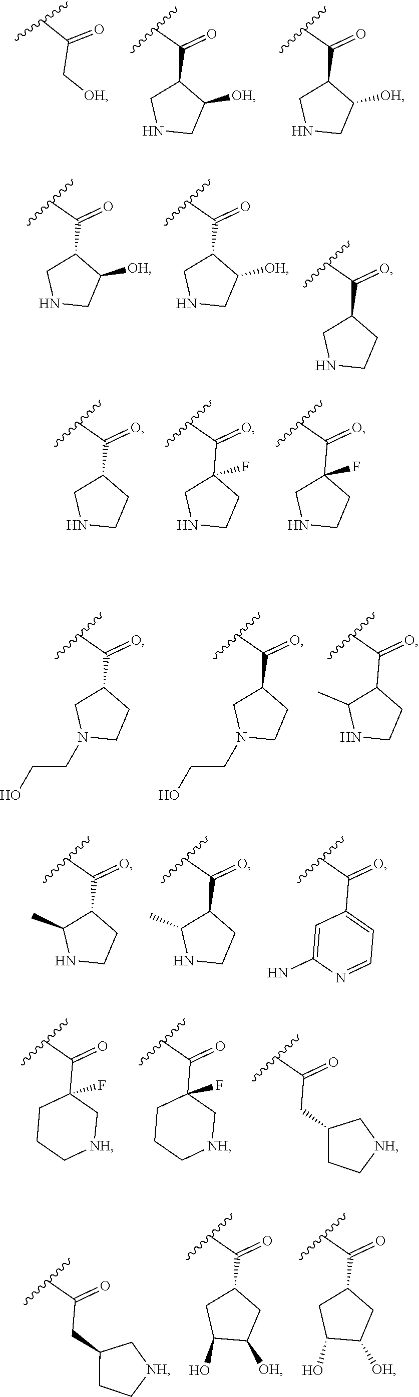

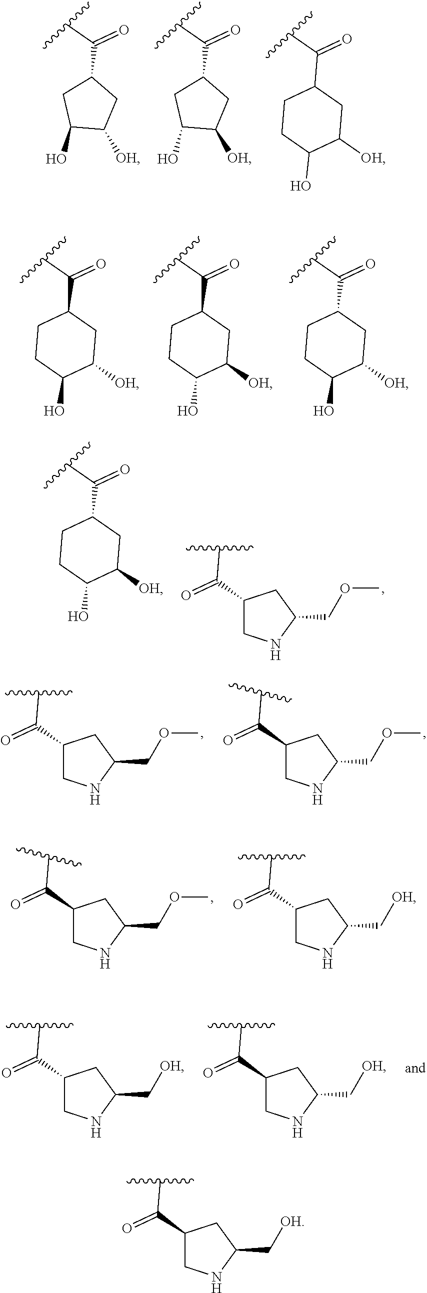

In some specific embodiments, R₂ is selected from the group consisting of

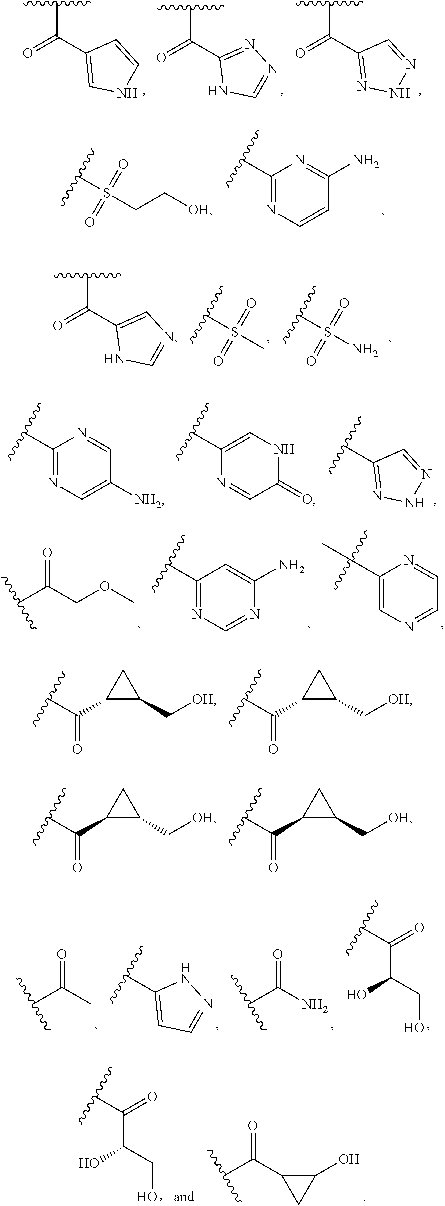

In some embodiments, the alkyl, cycloalkyl, and heteroalkyl in X₁, X₂, and X₃ are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)Rₐ, where valence permits. In some embodiments, the alkyl, cycloalkyl, heteroalkyl, heterocycle, aryl, and heteroaryl in Z are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)Rₐ, where valence permits. In some embodiments, the alkyl, cycloalkyl, heterocycle, aryl, and heteroaryl in R₁ are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)Rₐ, where valence permits. In some embodiments, the alkyl, cycloalkyl, heterocycle, aryl, and heteroaryl in R₂ are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)Rₐ, where valence permits. In some embodiments, the alkyl, cycloalkyl, heterocycle, aryl, and heteroaryl in R₃ is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)Rₐ, where valence permits.

In some embodiments, the compound of Formula I is selected from the group consisting of compounds 1-159 as shown in Table 1 below.

### Abbreviations

ACN Acetonitrile
Boc or boc Tert-butyloxycarbonyl
DBU 1,8-Diazabicyclo[5.4.0]undec-7-ene
DCM Dichloromethane
DMF Dimethyl formamide
DMSO Dimethyl sulfoxide
EA Ethyl acetate
EDCI 1-Ethyl-3-(3-dimethylaminopropyl)carbodiimide
HOBT Hydroxybenzotriazole
MeOH Methanol
NMO N-Methylmorpholine N-oxide
PE Petroleum ether
PMB Paramethoxybenzyl
TEA Triethylamine
TFA Trifluoroacetic acid
THF Tetrahydrofuran

### Methods of Preparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations, but not limitations of the preparation of some of the starting materials and compounds disclosed herein.

Schemes 1-6 below describe synthetic routes which may be used for the synthesis of compounds of the present invention, e.g., compounds having a structure of Formula I or a precursor thereof. Various modifications to these methods may be envisioned by those skilled in the art to achieve similar results to that of the inventions given below. In the embodiments below, the synthetic route is described using compounds having the structure of Formula I or a precursor thereof as examples. The general synthetic routes described in Schemes 1-6 and examples described in the Example section below illustrate methods used for the preparation of the compounds described herein.

Compound I-1 as shown in Scheme 1 can be prepared by any method known in the art and/or is commercially available. Substituents shown in Scheme 1 are defined herein. Isatin I-1 can be reacted with trimethylsilylmethyl Grignard to give I-2. I-2 can undergo elimination in the presence of a Lewis acid such as boron trifluoride etherate to form the methylene indolinone I-3. 1,3-dipolar cycloaddition of I-3 with dipole precursor I-4 provides the spirocyclic system I-5. If R₁ is not H, a mixture of regioisomers is obtained. Removal of the N-benzyl group can be achieved using 1-chloroethylchloroformate or by catalytic hydrogenolysis to give I-6, the precursor to many compounds of the invention. In some cases, it is necessary to protect the indolinone nitrogen to carry out reactions on the basic nitrogen. This can be achieved by reacting I-5 with a protecting reagent such as paramethoxybenzyl chloride and a base such as potassium carbonate, optionally in the presence of potassium iodide to give I-5a. The pyrrolidine nitrogen of I-5a is then deprotected in the same way as for I-5 to give I-6a.

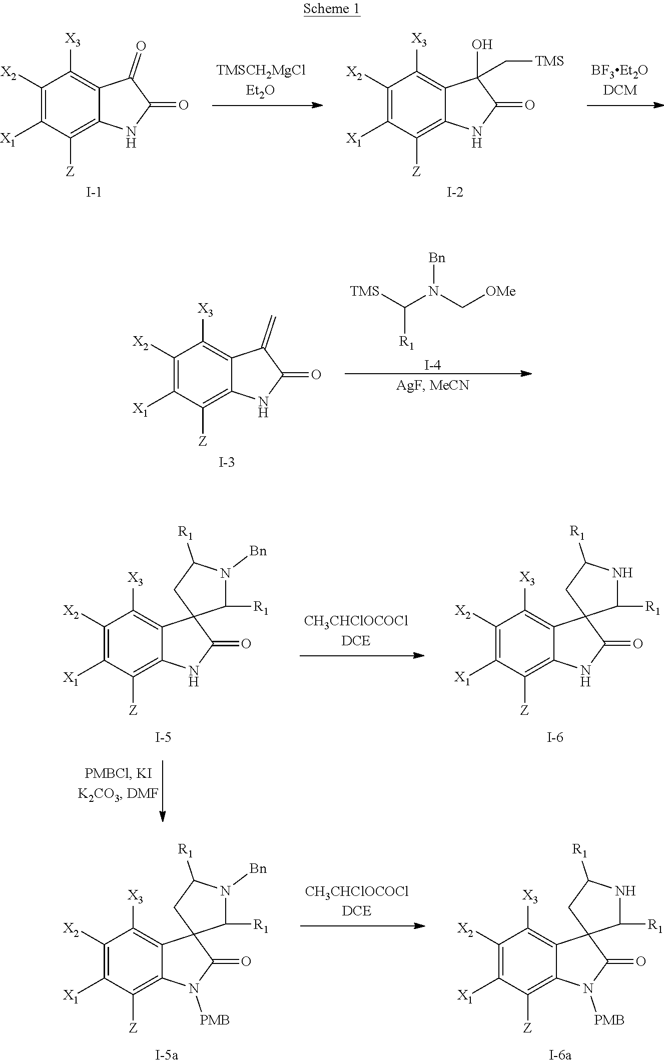

Alternatively, I-6 can be prepared by the route shown in Scheme 2. Compound I-7 as shown in Scheme 2 can be prepared by any method known in the art and/or is commercially available. Substituents shown in Scheme 2 are defined herein. Phenylacetic ester 7 can be reacted with a nitrating reagent such as a mixture of sulfuric and nitric acid gives I-8. I-8 is converted to the unsaturated ester I-9 by heating with aqueous formaldehyde and a base such as potassium carbonate. Cycloaddition of I-9 with the 1,3-dipole precursor I-4 and an acid such as TFA in an aprotic solvent such as THE yields the pyrrolidine I-10. If R₁ is not H, a mixture of regioisomers is obtained. The nitro group of I-10 is reduced by reacting with a reduction reagent such as zinc and hydrochloric acid to result in cyclization to the spiroindolinone I-5. Removal of the N-benzyl group as described in Scheme 1 using 1-chloroethylchloroformate provides I-6.

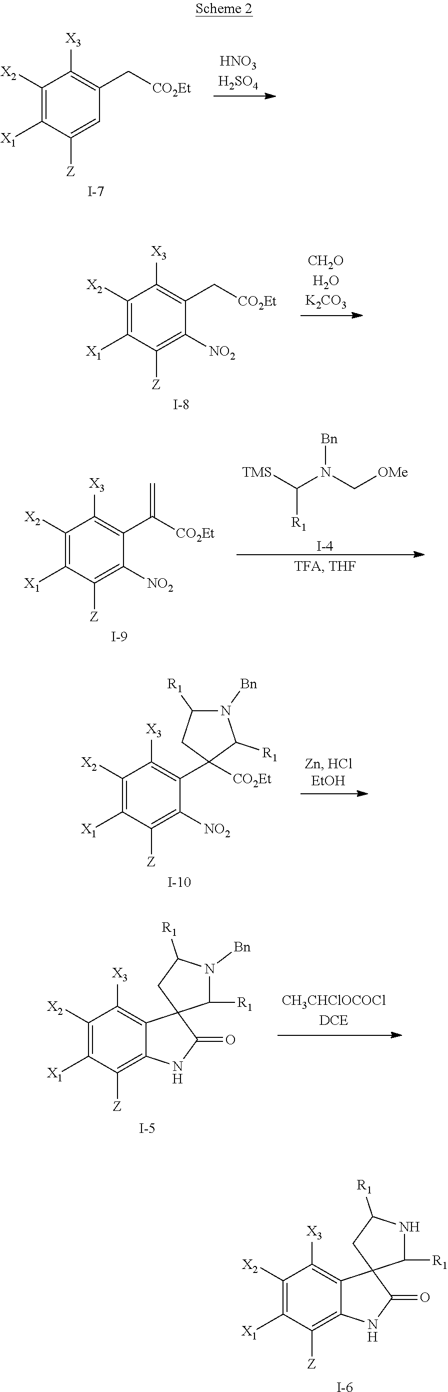

A third route to the spirocyclic system shown in Scheme 3 can also provide access to compounds substituted at each of the carbons in the pyrrolidine ring starting from a suitably substituted indole I-11. Compound I-11 as shown in Scheme 3 can be prepared by any method known in the art and/or is commercially available. Substituents shown in Scheme 3 are defined herein. Formylation of I-11 under Villsmeier conditions with DMF and phosphorus oxychloride gives I-12. I-12 is condensed with a nitoralkane, which also acts as solvent, in the presence of a catalyst such as ammonium acetate to form the nitroalkene I-13a (R₁ is a substituted group such as alkyl). Similarly, reaction using nitromethane yields the unsubstituted nitroalkene I-13b. Reduction of I-13a and I-13b is carried out in two steps, first reduction of the double bond with sodium borohydride followed by reduction of the nitro group with a metal such as zinc in an acidic solvent such as acetic acid to form the tryptamines I-15a or I-15b. Substitution at the carbon attached to the indole ring is achieved by reacting I-13b with a Grignard reagent R₁MgBr in an ether solvent such as THE to form I-14. Reduction of I-14 with zinc and acetic acid gives tryptamine I-15c. Pictet-Spengler cyclization of I-15a or I-15c with formaldehyde followed by protection of the amine such as Boc provides the β-carbolines I-16a and I-16c, respectively. Cyclization of I-15b with an aldehyde R₁CHO, optionally with an acid catalyst such as sulfuric acid, followed by Boc protection gives I-16b. Treatment of the β-carbolines I-16a, I-16b, I-16c with N-bromosuccinimide in water and acetic acid brings about rearrangement to the spirocyclic system. After removal of the protecting group, the substituted spiropyrrolidines I-6b, I-6c, I-6d are obtained, in each case as a single regioisomer. Pyrrolidines with substitution at two or three different carbons may be obtained by combining these routes shown in Scheme 3.

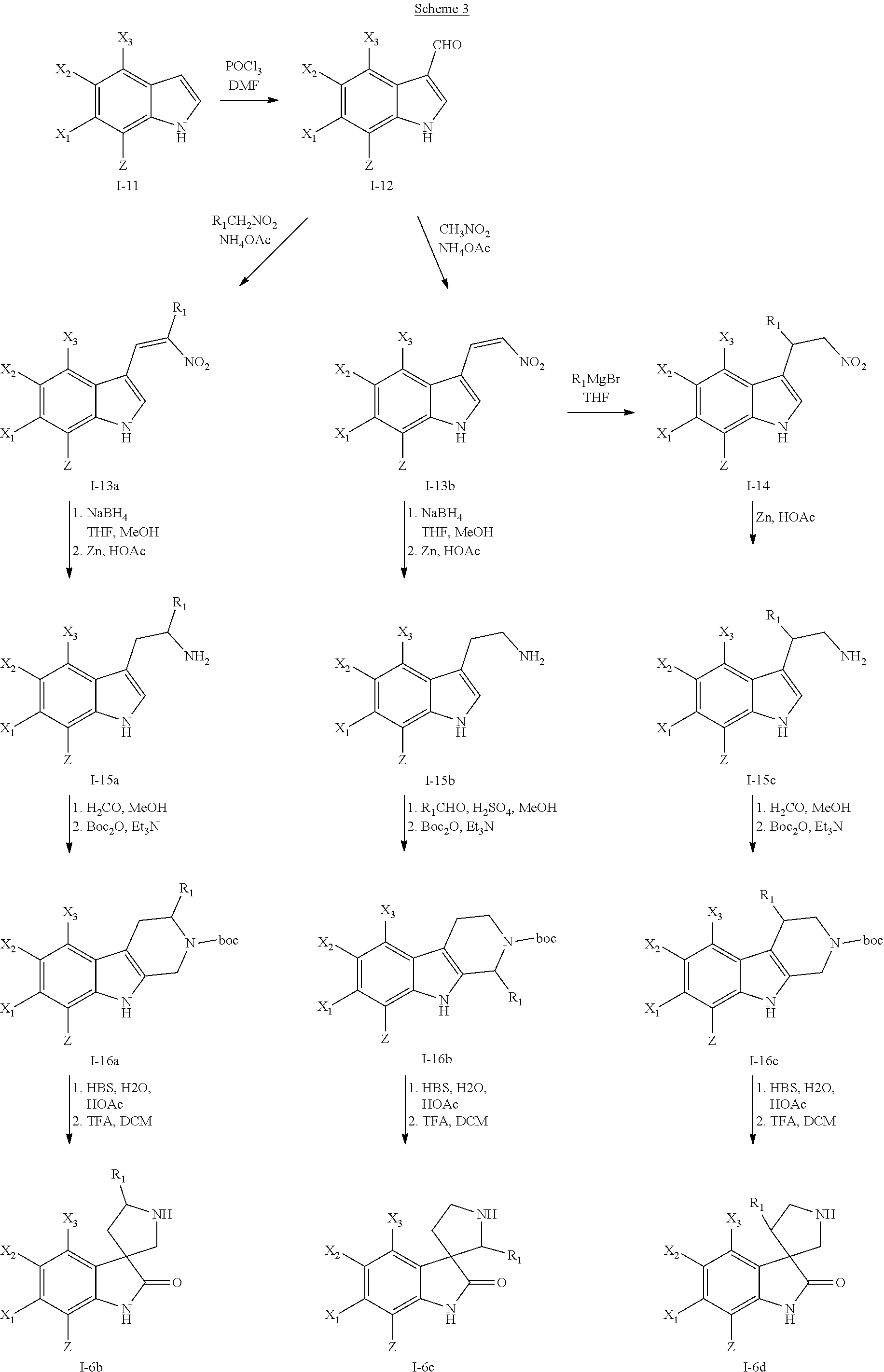

An enantioselective synthesis of the spiroindolinone core can be carried out using the method described in Mukaiyama et al, Chem. Eur. J. 2014, 20, 13583-13588 as shown in Scheme 4. Compound I-1 as shown in Scheme 4 can be prepared by any method known in the art and/or is commercially available. Substituents shown in Scheme 4 are defined herein. Suitably substituted isatin I-1 is first protected on nitrogen with a group such as benzyl or PMB to give I-17. Reaction of I-17 with acetaldehyde and a base such as DBU in a solvent such as THE under cooling to a low temperature such as −25° C. produces the aldol product I-18. Dehydration of I-18 under acid conditions such as sulfuric acid, in a solvent mixture containing acetic acid, water, and THE gives the enal I-19. Asymmetric Michael addition of nitromethane to I-19, using the R chiral auxiliary I-21 in isopropanol containing water provides I-20 enriched in the S enantiomer. Treatment of I-20 with zinc in acetic acid and ethanol brings about reduction of the nitro group, cyclization to an imine and further reduction to yield the spirocyclic pyrrolidine I-5aS as the S enantiomer. Removal of the PMB protecting group may be carried out either before or after further elaboration of the amine under acid conditions such as using a mixture of trifluromethanesulfonic acid and trifluoroacetic acid to yield I-5S.

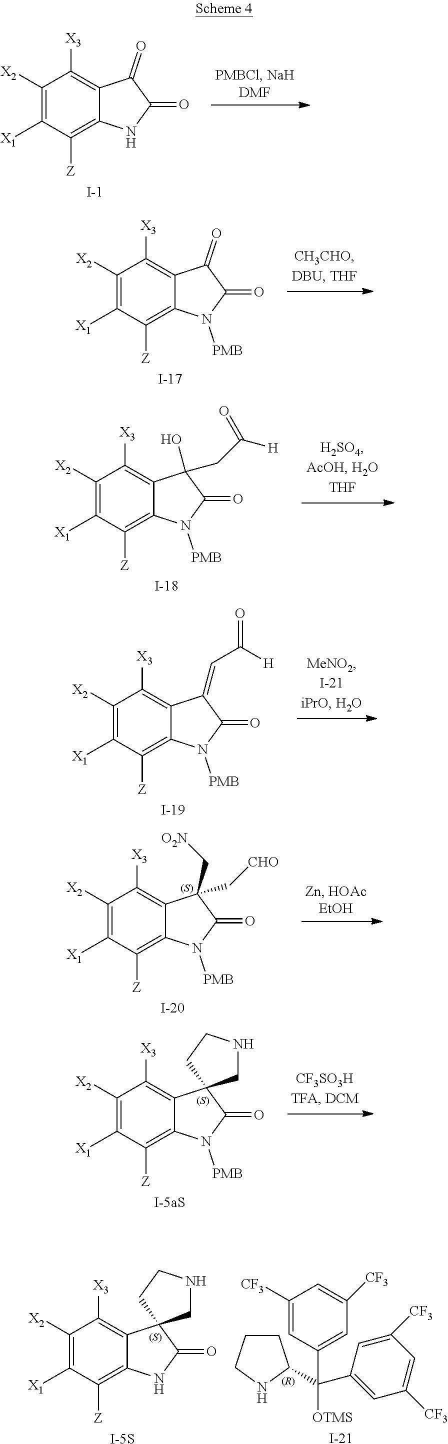

Spiropiperidines are synthesized from imdolinones, such as compound I-22, that are either commercially available or can be prepared by literature methods, as shown in Scheme 5. Substituents shown in Scheme 5 are defined herein. A suitably substituted indolinone I-22 is reacted with N-boc bis(2-chloroethyl)amine in the presence of a base such as sodium hydride in an inert solvent such as THE to form the spiropiperidine I-23. Removal of the Boc group under standard conditions provides I-24.

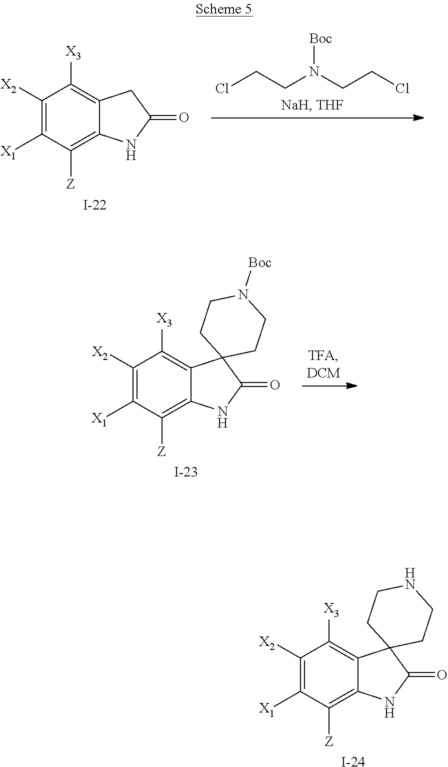

Compounds with a spiropyrrolidone ring rather than spiropyrrolidine can be prepared by the reaction sequence shown in Scheme 6. Substituents shown in Scheme 6 are defined herein. A suitably substituted isatin I-1 is first protected on nitrogen with a group such a benzyl or p-methoxybenzyl. The protected isatin I-17 is condensed with a cyanoacetate such as methyl cyanoacetate and an amine base such as piperidine to give I-25. Michael addition of nitromethane, that is used as solvent, in the presence of a base such as piperidine provides I-26. Heating I-26 with an alkali such as potassium hydroxide in water and alcohol causes hydrolysis and decarboxylation to the nitrile I-27. Hydrolysis of I-27 to the primary amide I-28 is carried out using acetamide and palladium chloride in aqueous THF. Reduction of the nitro group in I-28 with zinc in acetic acid results in cyclization to the spiropyrrolidone I-29. I-29 may be N-alkylated with R₃X under standard conditions and deprotected to provide I-30.

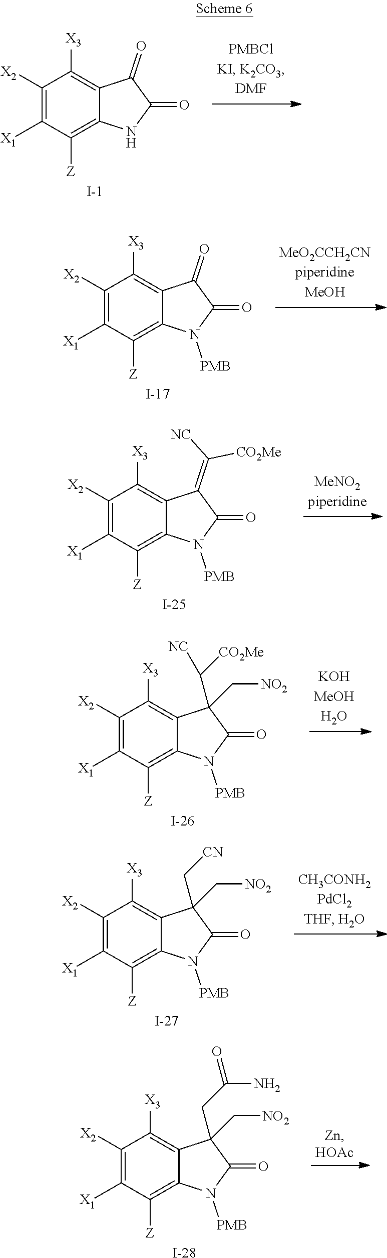

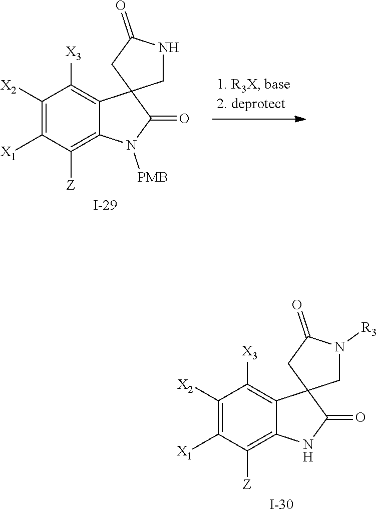

### Pharmaceutical Compositions

This invention also provides a pharmaceutical composition comprising at least one of the compounds as described herein or a pharmaceutically-acceptable salt or solvate thereof, and a pharmaceutically-acceptable carrier or diluent.

In yet another aspect, the present invention provides a pharmaceutical composition comprising at least one compound selected from the group consisting of compounds of Formula I as described herein and a pharmaceutically-acceptable carrier or diluent.

In certain embodiments, the composition is in the form of a hydrate, solvate or pharmaceutically-acceptable salt. The composition can be administered to the subject by any suitable route of administration, including, without limitation, oral and parenteral.

The phrase “pharmaceutically-acceptable carrier” as used herein means a pharmaceutically-acceptable material, composition or vehicle, such as a liquid or solid filler, diluent, excipient, solvent, or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as butylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically-acceptable salts. The term “pharmaceutically-acceptable salt,” in this respect, refers to the relatively non-toxic, inorganic and organic acid salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. See, e.g., Berge et al., (1977) “Pharmaceutical Salts”, J. Pharm. Sci. 66:1-19 (incorporated herein by reference in its entirety).

The pharmaceutically-acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like. See, e.g., Berge et al. (supra).

Wetting agents, emulsifiers, and lubricants, such as sodium lauryl sulfate, magnesium stearate, and polyethylene oxide-polybutylene oxide copolymer, as well as coloring agents, release agents, coating agents, sweetening, flavoring and perfuming agents, preservatives, and antioxidants can also be present in the compositions.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions, to provide the desired release profile, other polymer matrices, liposomes, and/or microspheres. They may be sterilized by, for example, filtration through a bacteria-retaining filter, or by incorporating sterilizing agents in the form of sterile solid compositions, which can be dissolved in sterile water or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically-acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar, and tragacanth, and mixtures thereof.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving or dispersing the pharmaceutical agents in the proper medium. Absorption enhancers can also be used to increase the flux of the pharmaceutical agents of the invention across the skin. The rate of such flux can be controlled, by either providing a rate-controlling membrane or dispersing the compound in a polymer matrix or gel.

Ophthalmic formulations, eye ointments, powders, solutions, and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions, or emulsions; or sterile powders which may be reconstituted into sterile injectable solutions or dispersions just prior to use, which may contain antioxidants, buffers, bacteriostats, or solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polypropylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot-injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically-acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), racehorses, birds, lizards, and any other organism which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

### Administration to a Subject

In yet another aspect, the present invention provides a method for treating a condition in a mammalian species in need thereof, the method comprising administering to the mammalian species a therapeutically effective amount of at least one compound selected from the group consisting of compounds of Formula I, or a pharmaceutically-acceptable salt thereof or a pharmaceutical composition thereof, wherein the condition is selected from the group consisting of cancer, an immunological disorder, a CNS disorder, an inflammatory disorder, a gastroenterological disorder, a metabolic disorder, a cardiovascular disorder, and a kidney disease.

In some embodiments, the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

In some embodiments, the inflammatory disorder is an inflammatory skin condition, arthritis, psoriasis, spondylitis, parodontitits, or an inflammatory neuropathy. In some embodiments, the gastroenterological disorder is an inflammatory bowel disease such as Crohn's disease or ulcerative colitis.

In some embodiments, the immunological disorder is transplant rejection or an autoimmune disease (e.g., rheumatoid arthritis, MS, systemic lupus erythematosus, or type I diabetes mellitus). In some embodiments, the CNS disorder is Alzheimer's disease.

In some embodiments, the metabolic disorder is obesity or type II diabetes mellitus. In some embodiments, the cardiovascular disorder is an ischemic stroke. In some embodiments, the kidney disease is chronic kidney disease, nephritis, or chronic renal failure.

In some embodiments, the mammalian species is human.

In some embodiments, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, inflammatory bowel disease, obesity, type II diabetes mellitus, ischemic stroke, chronic kidney disease, nephritis, chronic renal failure, and a combination thereof.

In yet another aspect, a method of blocking Kv1.3 potassium channel in a mammalian species in need thereof is described, including administering to the mammalian species a therapeutically effective amount of at least one compound of Formula I, or a pharmaceutically-acceptable salt or pharmaceutical composition thereof.

In some embodiments, the compounds described herein is selective in blocking the Kv1.3 potassium channels with minimal or no off-target inhibition activities against other potassium channels, or against calcium or sodium channels. In some embodiments, the compounds described herein do not block the hERG channels and therefore have desirable cardiovascular safety profiles.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically-acceptable solutions, which may routinely contain pharmaceutically-acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants; even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops, or preparations with protracted release of active compounds in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33, which is incorporated herein by reference in its entirety.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, different doses may be necessary depending on activity of the compound, manner of administration, purpose of the administration (i.e., prophylactic or therapeutic), nature and severity of the disorder, age and body weight of the patient. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically-acceptable salt. When used in medicine the salts should be pharmaceutically acceptable, but non-pharmaceutically-acceptable salts can conveniently be used to prepare pharmaceutically-acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, PA; incorporated herein by reference in its entirety.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained-release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids, or neutral fats such as mono- di- and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974, and 5,407,686. In addition, pump-based hardware delivery systems can be used, some of which are adapted for implantation.

### Assays for Effectiveness of Kv1.3 Potassium Channel Blockers

In some embodiments, the compounds as described herein are tested for their activities against Kv1.3 potassium channel. In some embodiments, the compounds as described herein are tested for their Kv1.3 potassium channel electrophysiology. In some embodiments, the compounds as described herein are tested for their hERG electrophysiology.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

Examples 1-6 describe various intermediates used in the syntheses of representative compounds of Formula I disclosed herein.

### Example 1. Intermediate 1S ((3S)-5,6-dichloro-1H-spiro[indole-3,3′-pyrrolidin]-2-one) and Intermediate 1R ((3R)-5,6-dichloro-1H-spiro[indole-3,3′-pyrrolidin]-2-one)

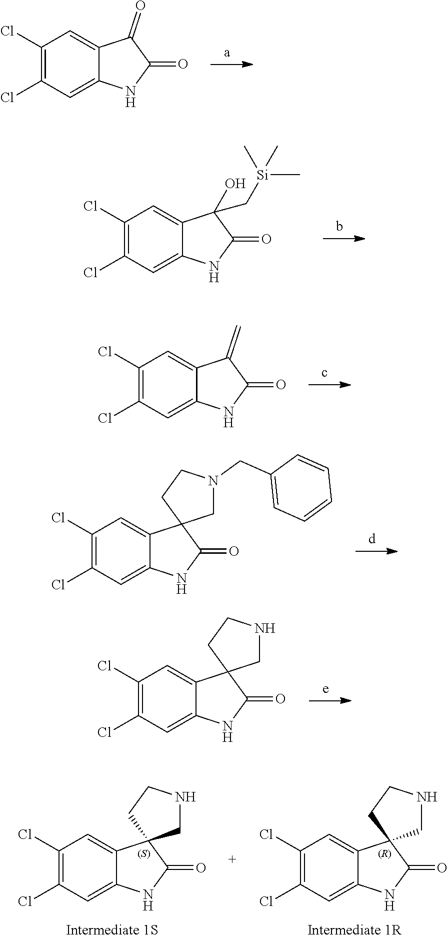

### Step a:

To a stirred solution of 5,6-dichloro-1H-indole-2,3-dione (50.0 g, 231 mmol) in THE (3.50 L) was added (trimethylsilyl)methylmagnesium chloride (600 mL, 4.08 mol, 1.3 Min THF) at −78° C. under nitrogen atmosphere. The reaction mixture was stirred for 2 h, quenched with saturated aq. NH₄Cl (1 L) at 0° C. and extracted with EA (3×1 L). The combined organic layers were washed with brine (2×500 mL) and dried over anhydrous Na₂SO₄. After filtration, the filtrate was concentrated under reduced pressure. The residue was suspended in PE and stirred for 15 min. The solids were collected by filtration and washed with PE (3×1 L) to afford 5,6-dichloro-3-hydroxy-3-[(trimethylsilyl)methyl]-1H-indol-2-one as a yellow solid (52.0 g, crude), which was used in the next step without purification: LCMS (ESI) calc'd for C₁₂H₁₅Cl₂NO₂Si [M−H]⁻: 302, 304 (3:2), found 302, 304 (3:2); ¹H NMR (300 MHz, DMSO-d₆) δ 10.53 (s, 1H), 7.49 (s, 1H), 7.01 (s, 1H), 5.96 (s, 1H), 1.53-1.08 (m, 2H), −0.27 (s, 9H).

### Step b:

To a stirred mixture of 5,6-dichloro-3-hydroxy-3-[(trimethylsilyl)methyl]-1H-indol-2-one (52.0 g, 171 mmol) in DCM (520 mL) was added BF₃ Et₂O (140 mL, 1.10 mol) at −78° C. under nitrogen atmosphere. The reaction mixture was stirred at room temperature for 2 h. The precipitated solid was collected by filtration and washed with DCM (3×1 L) to afford 5,6-dichloro-3-methylidene-1H-indol-2-one as a yellow solid (50.0 g, crude), which was used in the next step without purification: LCMS (ESI) calc'd for C₉H₅Cl₂NO [M−H]⁻: 212, 214 (3:2), found 212, 214 (3:2); ¹H NMR (300 MHz, DMSO-d₆) δ 10.73 (s, 1H), 7.91 (s, 1H), 7.01 (s, 1H), 6.47 (s, 1H), 6.28 (s, 1H).

### Step c:

To a stirred solution of 5,6-dichloro-3-methylidene-1H-indol-2-one (50.0 g, 233 mmol) and benzyl(methoxymethyl)[(trimethylsilyl)methyl]amine (83.2 g, 350 mmol) in THF (700 mL) was added TFA (26.0 mL) at room temperature under nitrogen atmosphere. The reaction mixture was stirred for 2 h and concentrated under reduced pressure. The residue was purified by reverse phase flash chromatography, eluting with 70% ACN in water (plus 10 mM NH₄HCO₃) to afford 1′-benzyl-5,6-dichloro-1H-spiro[indole-3,3′-pyrrolidin]-2-one as a yellow solid (20.0 g, 25% over three steps): LCMS (ESI) calc'd for C₁₈H₁₆Cl₂N₂O [M+H]⁺: 347, 349 (3:2), found 347, 349 (3:2); ¹H NMR (300 MHz, DMSO-d₆) δ 10.61 (s, 1H), 7.54 (s, 1H), 7.42-7.12 (m, 5H), 7.00 (s, 1H), 3.71 (s, 2H), 3.31 (s, 1H), 3.08 (td, J=8.3, 4.3 Hz, 1H), 2.84-2.59 (m, 2H), 2.27-1.85 (m, 2H).

### Step d:

A solution of 1′-benzyl-5,6-dichloro-1H-spiro[indole-3,3′-pyrrolidin]-2-one (20.0 g, 57.6 mmol) and chloroethyl chloroformate (32.9 g, 230 mmol) in DCE (200 mL) was stirred at 60° C. for 2 h. The resulting mixture was concentrated under reduced pressure. The residue was dissolved in MeOH (200 mL) and stirred at 60° C. for 30 min. The crude was purified by reverse phase flash chromatography, eluting with 30% ACN in water (plus 10 mM NH₄HCO₃) to afford 5,6-dichloro-1H-spiro[indole-3,3′-pyrrolidin]-2-one as an off-white solid (9.60 g, 54%): LCMS(ESI) calc'd for C₁₁H₁₀Cl₂N₂O [M+H]⁺: 257, 259 (3:2), found 257, 259 (3:2); ¹H NMR (300 MHz, DMSO-d₆) δ 10.46 (s, 1H), 7.65 (s, 1H), 7.01 (s, 1H), 3.26-3.19 (m, 1H), 3.06-2.94 (m, 3H), 2.19-1.82 (m, 2H).

### Step e:

5,6-dichloro-1H-spiro[indole-3,3-pyrrolidin]-2-one (10.0 g, 38.9 mmol) was separated by prep chiral SFC with the following conditions: Column: CHIRALPAK IG, 3×25 cm, 5 μm; Mobile Phase A: CO₂, Mobile Phase B: MeOH (0.1% 2 M NH₃-MEOH); Flow rate: 70 mL/min; Gradient: 60% B; Column Temperature: 34° C.; Back Pressure: 100 bar; Detector: UV 220 nm; Retention Time 1: 4.99 min; Retention Time 2: 9.00 min; Injection Volumn: 2 ml; Number Of Runs: 100. The faster-eluting enantiomer was obtained (3S)-5,6-dichloro-1H-spiro[indole-3,3-pyrrolidin]-2-one at 4.99 min as an off-white solid (2.50 g, 24%): LCMS(ESI) calc'd for C₁₁H₁₀Cl₂N₂O [M+H]⁺: 257, 259 (3:2), found 257, 259 (3:2); ¹H NMR (300 MHz, DMSO-d₆) δ 10.56 (s, 1H), 7.63 (s, 1H), 6.99 (s, 1H), 3.25-3.10 (m, 1H), 2.98 (dd, J=11.8, 1.6 Hz, 3H), 2.10 (ddd, J=13.3, 8.0, 5.5 Hz, 1H), 1.88 (ddd, J=12.7, 7.9, 6.5 Hz, 1H). The slower-eluting enantiomer was obtained (3R)-5,6-dichloro-1H-spiro[indole-3,3-pyrrolidin]-2-one at 9.00 min as an off-white solid (2.90 g, 26%): LCMS(ESI) calc'd for C₁₁H₁₀Cl₂N₂O [M+H]⁺: 257, 259 (3:2), found 257, 259 (3:2); ¹H NMR (300 MHz, DMSO-d₆) δ 11.02 (s, 1H), 7.96 (d, J=1.2 Hz, 1H), 7.09 (s, 1H), 3.62-3.35 (m, 4H), 2.36-2.08 (m, 2H).

### Example 2. Intermediate 2 (5,6,7-trichloro-1H-spiro[indole-3,3′-pyrrolidin]-2-one)

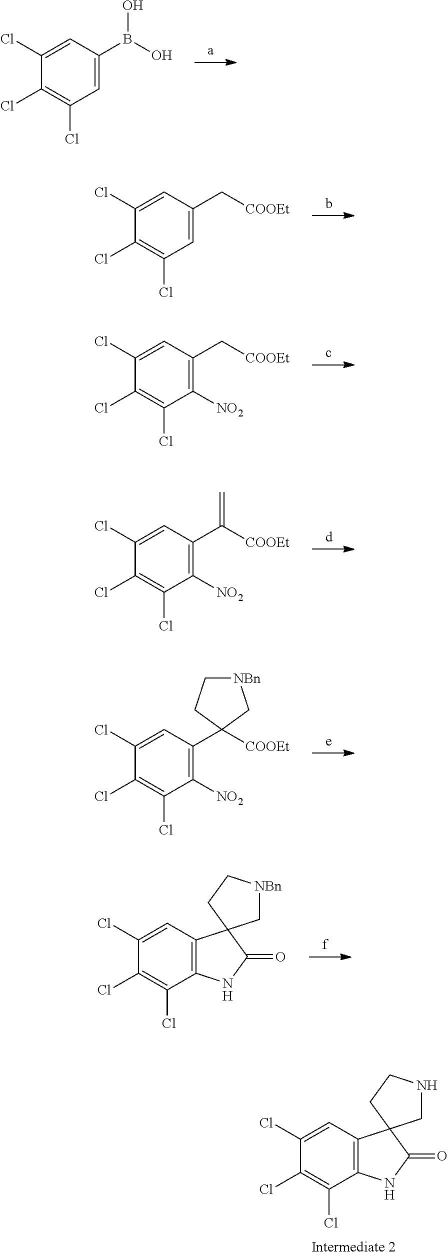

### Step a:

A mixture of 3,4,5-trichlorophenylboronic acid (2.00 g, 8.88 mmol), glycine ethyl ester hydrochloride (1.90 g, 13.6 mmol), NaNO₂ (1.10 g, 16.0 mmol) and NH₄Cl (1.90 g, 35.5 mmol) in toluene (20 mL) and H₂O (1 mL) was stirred at 100° C. for 16 h, diluted with EA (50 mL) and washed with brine (10 mL). The organic phase was dried over anhydrous Na₂SO₄. After filtration, the filtrate was concentrated under reduced pressure. The residue was purified by silica gel column chromatography, eluting with PE/EA (5/1) to afford ethyl 2-(3,4,5-trichlorophenyl)acetate as a light yellow oil (1.65 g, 70%): ¹H NMR (400 MHz, CDCl₃) δ 7.35 (s, 2H), 4.20 (q, J=7.1 Hz, 2H), 3.57 (s, 2H), 1.29 (t, J=7.1 Hz, 3H).

### Step b:

To a solution of ethyl 2-(3,4,5-trichlorophenyl)acetate (2.00 g, 7.48 mmol) in conc. H₂SO₄ (20 mL) was added conc. HNO₃ (0.600 g, 9.50 mmol) dropwise at −10° C. to 0° C. over 15 min. The reaction mixture was allowed to warm to room temperature over 1 h and stirred for an additional 1 h. The resulting mixture was poured into ice-water (50 mL). The precipitate was filtered, the filter cake washed with water (100 mL) and dried under reduced pressure to afford ethyl 2-(3,4,5-trichloro-2-nitrophenyl)acetate as an off-white solid (1.70 g, 73%): ¹H NMR (400 MHz, CDCl₃) δ 7.53 (s, 1H), 4.21 (q, J=7.1 Hz, 2H), 3.64 (s, 2H), 1.29 (t, J=7.1 Hz, 3H).

### Step c:

To a solution of ethyl 2-(3,4,5-trichloro-2-nitrophenyl)acetate (1.80 g, 5.76 mmol) in HCHO (10 mL, 30% in H₂O) was added a solution of K₂CO₃ (1.19 g, 8.64 mmol) in H₂O (4 mL) at room temperature. The reaction mixture was stirred at 60° C. for 2 h and filtered. The filter cake was washed with water (10 mL) and dried under reduced pressure to afford ethyl 2-(3,4,5-trichloro-2-nitrophenyl)prop-2-enoate as an off-white solid (1.70 g, 91%): ¹H NMR (400 MHz, CDCl₃) δ 7.46 (s, 1H), 6.66 (s, 1H), 5.96 (s, 1H), 4.27 (q, J=7.1 Hz, 2H), 1.31 (t, J=7.1 Hz, 3H).

### Step d:

To a stirred solution of ethyl 2-(3,4,5-trichloro-2-nitrophenyl)prop-2-enoate (1.60 g, 4.93 mmol) and benzyl(methoxymethyl)[(trimethylsilyl)methyl]amine (1.29 g, 5.42 mmol) in THE (20 mL) was added TFA (0.620 g, 5.42 mmol) at room temperature. The reaction mixture was stirred for 2 h, basified with aq. saturated NaHCO₃ to pH 8 and extracted with EA (2×20 mL). The combined organic layers were washed with brine (20 mL) and dried over anhydrous Na₂SO₄. After filtration, the filtrate was concentrated under reduced pressure. The residue was purified by silica gel column chromatography, eluting with PE/EA (5/1) to afford ethyl 1-benzyl-3-(3,4,5-trichloro-2-nitrophenyl)pyrrolidine-3-carboxylate as a light yellow foam (1.70 g, 75%): LCMS (ESI) calc'd for C₂₀H₁₉C₁₃N₂O₄ [M+H]⁺: 457, 459, 461 (3:3:1), found 457, 459, 461 (3:3:1); ¹H NMR (400 MHz, CDCl₃) δ 7.69 (s, 1H), 7.41-7.34 (m, 4H), 7.34-7.32 (m, 1H), 4.27-4.17 (m, 1H), 4.16-4.04 (m, 1H), 3.79 (d, J=12.9 Hz, 1H), 3.56 (d, J=13.0 Hz, 1H), 3.18 (d, J=9.9 Hz, 1H), 3.00-2.88 (m, 2H), 2.82 (d, J=10.0 Hz, 1H), 2.70 (q, J=8.0 Hz, 1H), 2.13-2.00 (m, 1H), 1.23 (t, J=7.1 Hz, 3H).

### Step e:

To a solution of ethyl 1-benzyl-3-(3,4,5-trichloro-2-nitrophenyl)pyrrolidine-3-carboxylate (0.400 g, 0.870 mmol) in EtOH (12 mL) and aq. HCl (8 mL, 3 M) was added Zn (1.20 g, 18.4 mmol) in portions at room temperature. The reaction mixture was stirred at 80° C. for 16 h and filtered through Celite, washing with water (2×20 mL). The filtrate was basified with saturated aq. NaHCO₃ to pH 9 and extracted with EA (3×20 mL). The combined organic layers were washed with brine (2×20 mL) and dried over anhydrous Na₂SO₄. After filtration, the filtrate was concentrated under reduced pressure to afford 1′-benzyl-5,6,7-trichloro-1H-spiro[indole-3,3′-pyrrolidin]-2-one as a light yellow solid (0.250 g, 75%): LCMS (ESI) calc'd for C₁₈H₁₅Cl₃N₂O [M+H]⁺: 381, 383, 385 (3:3:1), found 381, 383, 385 (3:3:1); ¹H NMR (400 MHz, DMSO-d₆) δ 11.13 (s, 1H), 7.57 (s, 1H), 7.40-7.30 (m, 4H), 7.28-7.21 (m, 1H), 3.71 (s, 2H), 3.14-3.01 (m, 1H), 2.84 (d, J=9.2 Hz, 1H), 2.72-2.53 (m, 2H), 2.28-2.16 (m, 1H), 2.06-1.94 (m, 1H).

### Step f:

To a solution of 1′-benzyl-5,6,7-trichloro-1H-spiro[indole-3,3′-pyrrolidin]-2-one (0.220 g, 0.580 mmol) in DCE (5 mL) was added chloroethyl chloroformate (0.390 g, 2.69 mmol) at room temperature. The reaction mixture was stirred at 60° C. for 2 h and concentrated under reduced pressure. The residue was dissolved in MeOH (5 mL), stirred at 60° C. for 2 h and concentrated under reduced pressure. The residue was purified by reverse phase chromatography, eluting with 55% ACN in water (plus 10 mM NH₄HCO₃) to afford 5,6,7-trichloro-1H-spiro[indole-3,3′-pyrrolidin]-2-one as an off-white solid (0.120 g, 71%): LCMS (ESI) calc'd for C₁₁H₉Cl₃N₂O [M+H]⁺: 291, 293, 295 (3:3:1), found 291, 293, 295 (3:3:1); ¹H NMR (400 MHz, CDCl₃) δ 7.97 (s, 1H), 7.29 (s, 1H), 3.53-3.37 (m, 2H), 3.32-3.17 (m, 1H), 3.05 (d, J=11.9 Hz, 1H), 2.55-1.96 (m, 3H).

### Example 3. Intermediate 3 (5,6-dichloro-5′-methyl-1H-spiro[indole-3,3′-pyrrolidin]-2-one)

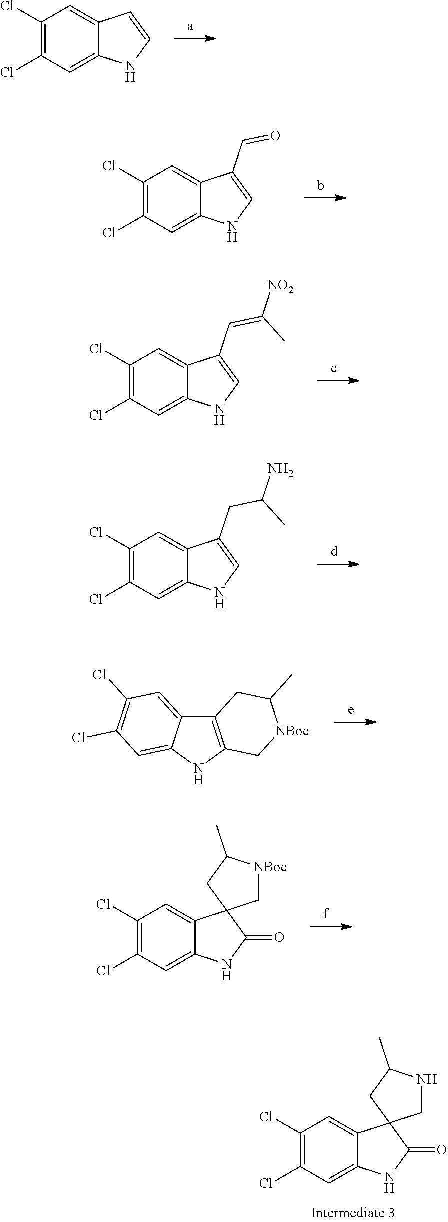

### Step a:

To DMF (20 mL) was added POCl₃ (2.47 g, 16.1 mmol) dropwise at −20° C. under nitrogen atmosphere. The solution was stirred for 0.5 h and then a solution of 5,6-dichloro-1H-indole (2.00 g, 10.8 mmol) in DMF (5 mL) was added, and stirred for an additional 1 h. The mixture was poured into ice-water (50 mL), stirred for 15 min and extracted with EA (3×50 mL). The aqueous solution was basified with aq. KOH (20%) to pH 8 and extracted with EA (3×50 mL). The combined organic solutions were washed with brine (3×50 mL) and dried over anhydrous Na₂SO₄. After filtration, the filtrate was concentrated under reduced pressure to afford 5,6-dichloro-1H-indole-3-carbaldehyde as an orange solid (1.80 g, 78%): LCMS (ESI) calc'd for C₉H₅Cl₂NO [M+H]⁺: 214, 216 (3:2) found 214, 216 (3:2); ¹H NMR (300 MHz, DMSO-d₆) δ 12.36 (s, 1H), 9.94 (s, 1H), 8.42 (s, 1H), 8.23 (s, 1H), 7.80 (s, 1H).

### Step b:

A mixture of 5,6-dichloro-1H-indole-3-carbaldehyde (1.80 g, 8.41 mmol) and ammonium acetate (0.780 g, 10.1 mmol) in CH₃NO₂ (15 mL) was stirred at 90° C. for 1 h and concentrated under reduced pressure. The residue was purified by silica gel chromatography, eluting with PE/EA (3/1) to afford 5,6-dichloro-3-[(1E)-2-nitroprop-1-en-1-yl]-1H-indole as an orange solid (1.50 g, 66%): LCMS (ESI) calc'd for C₁₁H₈Cl₂N₂O₂ [M−H]⁻: 269, 271 (3:2) found 269, 271 (3:2); ¹H NMR (400 MHz, CDCl₃) δ 8.74 (s, 1H), 8.36 (s, 1H), 7.91 (s, 1H), 7.60 (s, 1H), 7.59 (d, J=2.8 Hz, 1H), 2.55 (s, 3H).

### Step c:

To a solution of 5,6-dichloro-3-[(1E)-2-nitroprop-1-en-1-yl]-1H-indole (0.900 g, 3.32 mmol) in MeOH (10 mL) and THE (10 mL) was added NaBH₄ (0.500 g, 13.3 mmol) at room temperature. The reaction mixture was stirred for 2 h, quenched with water (50 mL) and extracted with EA (3×50 mL). The combined organic layers were washed with brine (2×20 mL) and dried over anhydrous Na₂SO₄. After filtration, the residue was concentrated under reduced pressure and dissolved in AcOH (8 mL). Zn (1.30 g, 19.9 mmol) was added and the reaction mixture was stirred for 16 h and filtered. The filtrate was concentrated under reduced pressure and the residue was purified by reverse phase chromatography, eluting with 45% ACN in water (plus 10 mM NH₄HCO₃) to afford 1-(5,6-dichloro-1H-indol-3-yl)propan-2-amine as a light yellow foam (0.400 g, 49%): LCMS (ESI) calc'd for C₁₁H₁₂Cl₂N₂ [M+H]⁺: 243, 245 (3:2) found 243, 245 (3:2); ¹H NMR (400 MHz, CDCl₃) δ 8.34 (s, 1H), 7.68 (s, 1H), 7.47 (s, 1H), 7.09 (s, 1H), 3.37-3.22 (m, 1H), 2.84 (dd, J=14.3, 5.1 Hz, 1H), 2.66 (dd, J=14.3, 8.2 Hz, 1H), 1.20 (d, J=6.3 Hz, 3H).

### Step d:

To a stirred solution of 1-(5,6-dichloro-1H-indol-3-yl)propan-2-amine (0.300 g, 1.23 mmol) in HFIP (5 mL) was added HCHO (0.120 g, 1.48 mmol, 37% aqueous solution) at room temperature. The reaction mixture was stirred for 1.5 h and concentrated under reduced pressure. The residue was dissolved in DCM (8 mL) and TEA (0.370 g, 3.70 mmol) and Boc₂O (0.320 g, 1.48 mmol) were added. The reaction mixture was stirred for 1 h, diluted with water (50 mL) and extracted with EA (3×30 mL). The combined organic layers were washed with brine (2×20 mL) and dried over anhydrous Na₂SO₄. After filtration, the filtrate was concentrated under reduced pressure to afford tert-butyl 6,7-dichloro-3-methyl-1,3,4,9-tetrahydro-2H-pyrido[3,4-b]indole-2-carboxylate as a yellow solid (0.450 g, crude), which was used to next step without purification: LCMS (ESI) calc'd for C₁₇H₂₀Cl₂N₂O₂ [M−H]⁻: 353, 355 (3:2) found 353, 355 (3:2).

### Step e:

To a solution of tert-butyl 6,7-dichloro-3-methyl-1,3,4,9-tetrahydro-2H-pyrido[3,4-b]indole-2-carboxylate (0.450 g, 1.27 mmol) in THE (8 mL), H₂O (4 mL) and AcOH (0.8 mL) was added NBS (0.250 g, 1.39 mmol) at room temperature. The reaction mixture was stirred for 1 h and concentrated under reduced pressure to afford tert-butyl 5,6-dichloro-5′-methyl-2-oxo-1H-spiro[indole-3,3′-pyrrolidine]-1′-carboxylate as a brown oil (0.450 g, crude), which was used in the next step without purification: LCMS (ESI) calc'd for C₁₇H₂₀Cl₂N₂O₃ [M−H]⁻: 369, 371 (3:2) found 369, 371 (3:2).

### Step f:

To a stirred mixture of tert-butyl 5,6-dichloro-5′-methyl-2-oxo-1H-spiro[indole-3,3′-pyrrolidine]-1′-carboxylate (0.450 g, 1.21 mmol) in DCM (2 mL) was added TFA (2 mL) at room temperature. The reaction mixture was stirred for 1 h and concentrated under reduced pressure. The residue was purified by reverse phase chromatography, eluting with 20% ACN in water (plus 0.05% TFA) to afford 5,6-dichloro-5′-methyl-1H-spiro[indole-3,3′-pyrrolidin]-2-one as a yellow solid (0.300 g, 64%): LCMS (ESI) calc'd for C₁₂H₁₂Cl₂N₂O [M+H]⁺: 271, 273 (3:2) found 271, 273 (3:2); ¹H NMR (300 MHz, CD₃OD) δ 7.72 (d, J=2.1 Hz, 1H), 7.12 (d, J=5.6 Hz, 1H), 4.37-4.11 (m, 1H), 3.83-3.43 (m, 2H), 2.62-2.39 (m, 1H), 2.27-2.09 (m, 1H), 1.57 (dd, J=6.5, 3.7 Hz, 3H).

### Example 4. Intermediate 3b (5,6-dichloro-2′-methyl-1H-spiro[indole-3,3′-pyrrolidin]-2-one)

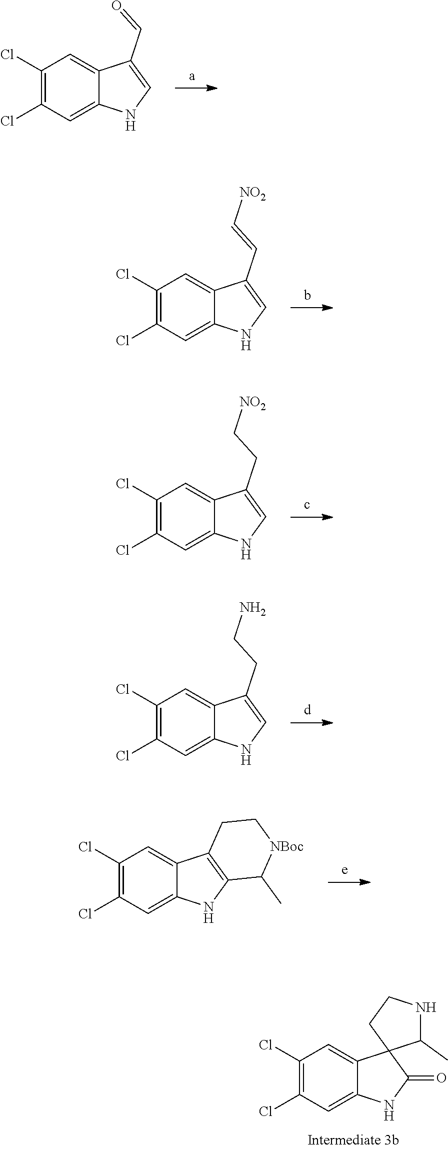 [SMILES: O=Cc1c[nH]c2cc(Cl)c(Cl)cc12 ; O=[N+]([O-])/C=C/c1c[nH]c2cc(Cl)c(Cl)cc12 ; O=[N+]([O-])CCc1c[nH]c2cc(Cl)c(Cl)cc12 ; NCCc1c[nH]c2cc(Cl)c(Cl)cc12 ; CC1c2[nH]c3cc(Cl)c(Cl)cc3c2CCN1C(=O)OC(C)(C)C ; CC1NCCC12C(=O)Nc1cc(Cl)c(Cl)cc12]

### Step a:

To a stirred solution of 5,6-dichloro-1H-indole-3-carbaldehyde (2.00 g, 9.34 mmol) in CH₃NO₂ (60.0 mL) was added CH₃COONH₄ (0.860 g, 11.2 mmol) at room temperature. The reaction mixture was stirred at 90° C. for 1 h and concentrated under reduced pressure. The residue was purified by silica gel chromatography, eluting with PE/EA (1/1) to afford 5,6-dichloro-3-[(E)-2-nitroethenyl]-1H-indole as an orange solid (1.10 g, 46%): LCMS (ESI) calc'd for C₁₀H₆Cl₂N₂O₂ [M−H]⁻: 255, 257 (3:2) found 255, 257 (3:2); ¹H NMR (300 MHz, DMSO-d₆) δ 12.38 (s, 1H), 8.43-8.35 (m, 2H), 8.33 (s, 1H), 8.16 (d, J=13.5 Hz, 1H), 7.78 (s, 1H).

### Step b:

To a stirred solution of 5,6-dichloro-3-[(E)-2-nitroethenyl]-1H-indole (1.10 g, 4.28 mmol) in MeOH (10 mL) and THE (10 mL) was added NaBH₄ (0.320 g, 8.56 mmol) in portions at room temperature. The reaction mixture was stirred for 30 min, quenched with water (10 mL) and concentrated under reduced pressure. The residue was purified by silica gel chromatography, eluting with PE/EA (2/1) to afford 5,6-dichloro-3-(2-nitroethyl)-1H-indole as a yellow solid (0.730 g, 66%): LCMS (ESI) calc'd for C₁₀H₈Cl₂N₂O₂ [M−H]⁻: 257, 259 (3:2) found 257, 259 (3:2); ¹H NMR (300 MHz, CDCl₃) δ 8.11 (s, 1H), 7.66 (s, 1H), 7.50 (s, 1H), 7.11 (dd, J=2.3, 1.0 Hz, 1H), 4.73-4.62 (m, 2H), 3.50-3.40 (m, 2H).

### Step c:

To a stirred solution of 5,6-dichloro-3-(2-nitroethyl)-1H-indole (0.730 g, 2.82 mmol) in HOAc (10.0 mL) was added Zn (1.84 g, 28.2 mmol) at room temperature. The reaction mixture was stirred for 16 h and filtered. The filter cake was washed with EA (3×20 mL) and the filtrate was concentrated under reduced pressure. The residue was purified by reverse phase chromatography, eluting with 40% ACN in water (plus 10 mM NH₄HCO₃) to afford 2-(5,6-dichloro-1H-indol-3-yl)ethanamine as a colorless oil (0.550 g, 85%): LCMS (ESI) calc'd for C₁₀H₁₀Cl₂N₂ [M−H]⁻: 227, 229 (3:2) found 227, 229 (3:2); ¹H NMR (300 MHz, CDCl₃) 8.05 (s, 1H), 7.70 (s, 1H), 7.49 (s, 1H), 7.10 (s, 1H), 3.04 (t, J=6.7 Hz, 2H), 2.87 (t, J=6.7 Hz, 2H).

### Step d:

To a stirred solution of 2-(5,6-dichloro-1H-indol-3-yl)ethanamine (0.450 g, 1.96 mmol) in MeOH (10 mL) and H₂O (2 mL) were added acetaldehyde (0.129 g, 2.95 mmol) and conc. H₂SO₄ (19.26 mg, 0.196 mmol) at room temperature. The reaction mixture was stirred at 60° C. for 6 h and concentrated under reduced pressure. The crude product was dissolved in DCM (10 mL) and TEA (0.596 g, 5.89 mmol) and Boc₂O (0.643 g, 2.95 mmol) was added at room temperature. The resulting mixture was stirred for 1 h and concentrated under reduced pressure. The residue was purified by reverse phase chromatography, eluting with 78% ACN in water (plus 10 mM NH₄HCO₃) to afford tert-butyl 6,7-dichloro-1-methyl-1H,3H,4H,9H-pyrido[3,4-b]indole-2-carboxylate as a yellow solid (0.250 g, 36%): LCMS (ESI) calc'd for C₁₇H₂₀Cl₂N₂O₂ [M−H]⁻: 353, 355 (3:2) found 353, 355 (3:2); ¹H NMR (300 MHz, CDCl₃) δ 7.55 (s, 1H), 7.42 (s, 1H), 5.47-5.15 (m, 1H), 4.57-4.21 (m, 1H), 3.25-2.99 (m, 1H), 2.87-2.59 (m, 2H), 1.54 (s, 9H), 1.49 (d, J=6.77 Hz, 3H).

### Step e:

To a stirred solution of tert-butyl 6,7-dichloro-1-methyl-1H,3H,4H,9H-pyrido[3,4-b]indole-2-carboxylate (0.250 g, 0.704 mmol) in H₂O (1.00 mL), THE (2.00 mL) and AcOH (0.200 mL) was added NBS (0.250 g, 1.41 mmol) at room temperature. The reaction mixture was stirred for 2 h and concentrated under reduced pressure. The crude product was dissolved in DCM (2 mL) and TFA (0.500 mL) was added into the solution. The resulting mixture was stirred for 1 h and concentrated under reduced pressure. The residue was purified by reverse phase chromatography, eluting with 45% ACN in water (plus 10 mM NH₄HCO₃) to afford 5,6-dichloro-2′-methyl-1H-spiro[indole-3,3′-pyrrolidin]-2-one as a yellow oil (0.150 g, 79%): LCMS (ESI) calc'd for C₁₂H₁₂Cl₂N₂O [M+H]⁺: 271, 273 (3:2) found 271, 273 (3:2): ¹H NMR (400 MHz, CDCl₃) δ 8.21 (s, 1H), 7.28 (d, J=5.47 Hz, 1H), 7.05 (d, J=7.60 Hz, 1H), 3.64-3.12 (m, 3H), 2.61-2.31 (m, 1H), 2.31-2.02 (m, 1H), 0.98 (dd, J=69.74, 6.49 Hz, 3H).

### Example 5. Intermediate 3c (5,6-dichloro-4′-methyl-1H-spiro[indole-3,3′-pyrrolidin]-2-one)

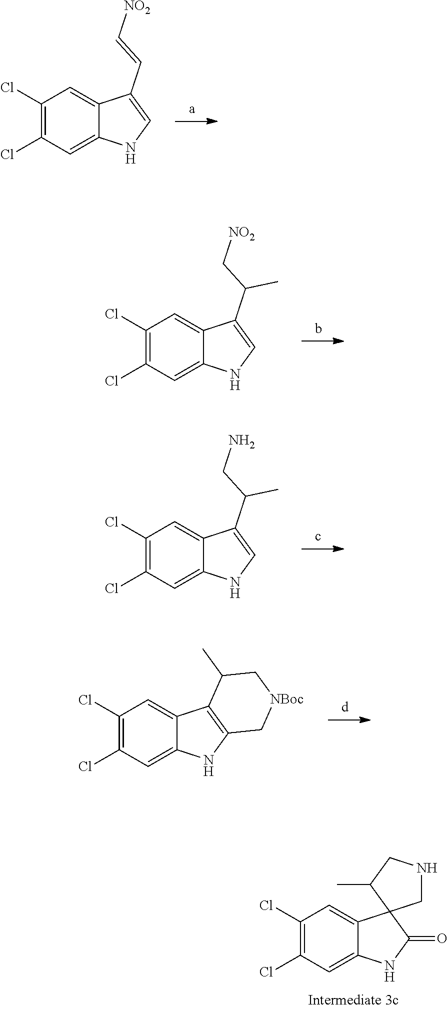 [SMILES: O=[N+]([O-])/C=C/c1c[nH]c2cc(Cl)c(Cl)cc12 ; CC(C[N+](=O)[O-])c1c[nH]c2cc(Cl)c(Cl)cc12 ; CC(CN)c1c[nH]c2cc(Cl)c(Cl)cc12 ; CC1CN(C(=O)OC(C)(C)C)Cc2[nH]c3cc(Cl)c(Cl)cc3c21 ; CC1CNCC12C(=O)Nc1cc(Cl)c(Cl)cc12]

### Step a:

To a stirred solution of 5,6-dichloro-3-[(E)-2-nitroethenyl]-1H-indole (1.10 g, 4.28 mmol) in THE (30 mL) was added CH₃MgBr (10.7 mL, 10.7 mmol, 1 Min THF) dropwise at −60° C. under nitrogen atmosphere. The reaction mixture was stirred for 3 h, quenched with saturated aq. NH₄Cl (20 mL) and extracted with EA (3×50 mL). The combined organic layers were washed with brine (3×30 mL) and dried over anhydrous Na₂SO₄. After filtration, the filtrate was concentrated under reduced pressure. The residue was purified by silica gel chromatography, eluting with PE/EA (3/1) to afford 5,6-dichloro-3-(1-nitropropan-2-yl)-1H-indole as a yellow oil (0.950 g, 81%): LCMS (ESI) calc'd for C₁₁H₁₀Cl₂N₂O₂ [M−H]⁻: 271, 273 (3:2) found 271, 273 (3:2); ¹H NMR (400 MHz, DMSO-d₆) δ 11.27 (s, 1H), 7.91 (s, 1H), 7.61 (s, 1H), 7.40 (d, J=2.4 Hz, 1H), 4.89-4.78 (m, 2H), 3.82 (q, J=7.2 Hz, 1H), 1.35 (d, J=7.0 Hz, 3H).

### Step b:

To a stirred solution of 5,6-dichloro-3-(1-nitropropan-2-yl)-1H-indole (1.00 g, 3.66 mmol) in AcOH (15.0 mL) was added Zn (2.39 g, 36.6 mmol) at room temperature. The reaction mixture was stirred for 16 h and filtered. The filter cake was washed with EA (3×20 mL) and the filtrate was concentrated under reduced pressure. The residue was purified by reverse phase chromatography, eluting with 60% ACN in water (plus 10 mM NH₄HCO₃) to afford 2-(5,6-dichloro-1H-indol-3-yl)propan-1-amine as a yellow oil (0.80 g, 90%): LCMS (ESI) calc'd for C₁₁H₁₂Cl₂N₂ [M−H]⁻: 241, 243 (3:2) found 241, 243 (3:2); ¹H NMR (300 MHz, CDCl₃) δ 8.20 (s, 1H), 7.73 (s, 1H), 7.48 (s, 1H), 7.05 (d, J=2.3 Hz, 1H), 3.06 (dd, J=12.9, 6.4 Hz, 1H), 3.05-2.88 (m, 2H), 1.37 (d, J=6.7 Hz, 2H), 1.32 (s, 3H).

### Step c:

To a stirred solution of 2-(5,6-dichloro-1H-indol-3-yl)propan-1-amine (0.400 g, 1.65 mmol) in HFIP (8.00 mL) was added HCHO (0.260 g, 3.29 mmol) at room temperature. The reaction mixture was stirred for 2 h and concentrated under reduced pressure. The crude product was dissolved in DCM (5 mL) and TEA (0.332 g, 3.29 mmol) and Boc₂O (0.538 g, 2.47 mmol) were added. The resulting reaction mixture was stirred for 4 h and concentrated under reduced pressure. The residue was purified by reverse phase chromatography, eluting with 60% ACN in water (plus 10 mM NH₄HCO₃) to afford tert-butyl 6,7-dichloro-4-methyl-1H,3H,4H,9H-pyrido[3,4-b]indole-2-carboxylate as a yellow oil (0.160 g, 27%): LCMS (ESI) calc'd for C₁₇H₂₀Cl₂N₂O₂ [M−H]⁻: 353, 355 (3:2) found 353, 355 (3:2).

### Step d:

To a stirred solution of tert-butyl 6,7-dichloro-4-methyl-1H,3H,4H,9H-pyrido[3,4-b]indole-2-carboxylate (0.160 g, 0.450 mmol) in H₂O (1.00 mL), THE (2.00 mL) and AcOH (0.200 mL) was added NBS (0.160 g, 0.900 mmol) at room temperature. The reaction mixture was stirred for 2 h and concentrated under reduced pressure. The crude product was dissolved in DCM (2 mL) and TFA (0.5 mL) was added. The resulting reaction mixture was stirred for 1 h and concentrated under reduced pressure. The residue was purified by reverse phase chromatography, eluting with 35% ACN in water (plus 0.05% TFA) to afford 5,6-dichloro-4′-methyl-1H-spiro[indole-3,3′-pyrrolidin]-2-one as a yellow oil (60.0 mg, 49%): LCMS (ESI) calc'd for C₁₂H₁₂Cl₂N₂O [M−H]⁻: 269, 271 (3:2) found 269, 271 (3:2).

### Example 6. Intermediate 4 (5,6-dichloro-1-[(4-methoxyphenyl)methyl]spiro[indole-3,3′-pyrrolidin]-2-one)

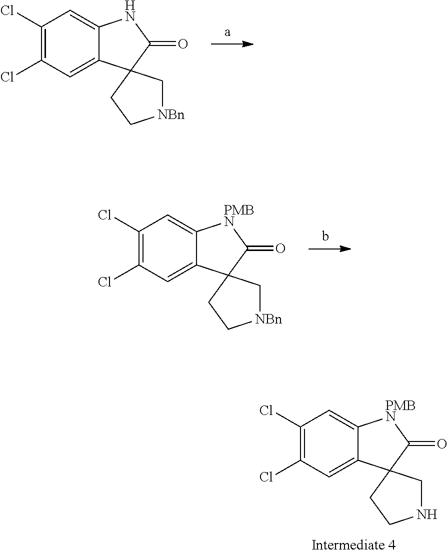

### Step a:

To a stirred solution of 1′-benzyl-5,6-dichloro-1H-spiro[indole-3,3′-pyrrolidin]-2-one (8.50 g, 24.5 mmol) in DMF (100 mL) was added NaH (1.96 g, 49.0 mmol, 60% in oil) and PMBCl (4.60 g, 29.4 mmol) at 0° C. under nitrogen atmosphere. The reaction mixture was stirred for 2 h, diluted with water (50 mL) and extracted with EA (3×50 mL). The combined organic layers were washed with brine (3×50 mL) and dried over anhydrous Na₂SO₄. After filtration, the filtrate was concentrated under reduced pressure. The residue was purified by silica gel chromatography, eluting with PE/EA (5/1) to afford 1′-benzyl-5,6-dichloro-1-[(4-methoxyphenyl)methyl]spiro[indole-3,3′-pyrrolidin]-2-one as a light yellow oil (11.0 g, 96%): LCMS (ESI) calc'd for C₂₆H₂₄Cl₂N₂O₂ [M+H]⁺: 467, 469 (3:2) found 467, 469 (3:2); ¹H NMR (400 MHz, DMSO-d₆) δ 7.61 (s, 1H), 7.40-7.29 (m, 4H), 7.28-7.19 (m, 4H), 6.92-6.84 (m, 2H), 4.83 (s, 2H), 3.75-3.68 (m, 5H), 3.15-3.07 (m, 1H), 2.82 (d, J=9.07 Hz, 1H), 2.70 (d, J=9.08 Hz, 1H), 2.61 (q, J=8.29 Hz, 1H), 2.30-2.21 (m, 1H), 2.07-1.98 (m, 1H).

### Step b:

To a solution of 1′-benzyl-5,6-dichloro-1-[(4-methoxyphenyl)methyl]spiro[indole-3,3′-pyrrolidin]-2-one (9.00 g, 19.3 mmol) in DCE (90 mL) was added chloroethyl chloroformate (11.3 g, 78.8 mmol) at room temperature. The reaction mixture was stirred at 60° C. for 2 h and concentrated under reduced pressure. The residue was dissolved in MeOH (90.0 mL), stirred at 60° C. for 1 h and concentrated under reduced pressure. The residue was suspended in MTBE (30.0 mL) and filtered. The filter cake was dried under vacuum to afford 5,6-dichloro-1-[(4-methoxyphenyl)methyl]spiro[indole-3,3′-pyrrolidin]-2-one as a light yellow solid (5.00 g, 69%): LCMS (ESI) calc'd for C₁₉H₁₈Cl₂N₂O₂ [M+H]⁺: 377, 379 (3:2) found 377, 379 (3:2); ¹H NMR (400 MHz, DMSO-d₆) δ 7.72 (s, 1H), 7.28-7.18 (m, 3H), 6.94-6.84 (m, 2H), 4.84 (s, 2H), 3.71 (s, 3H), 3.27-3.15 (m, 2H), 3.10-2.99 (m, 2H), 2.22-2.11 (m, 1H), 2.03-1.88 (m, 1H).

Examples 7-23 describe the syntheses of representative compounds of Formula I disclosed herein.

### Example 7. Compound 1 ((3S)-5,6-dichloro-1′-[4-hydroxypyrrolidine-3-carbonyl]-1H-spiro[indole-3,3′-pyrrolidin]-2-one isomer 1), Compound 2 ((3S)-5,6-dichloro-1′-[4-hydroxypyrrolidine-3-carbonyl]-1H-spiro[indole-3,3′-pyrrolidin]-2-one isomer 2), Compound 3 ((3S)-5,6-dichloro-1′-[4-hydroxypyrrolidine-3-carbonyl]-1H-spiro[indole-3,3′-pyrrolidin]-2-one isomer 3), and Compound 4 ((3S)-5,6-dichloro-1′-[4-hydroxypyrrolidine-3-carbonyl]-1H-spiro[indole-3,3′-pyrrolidin]-2-one isomer 4)

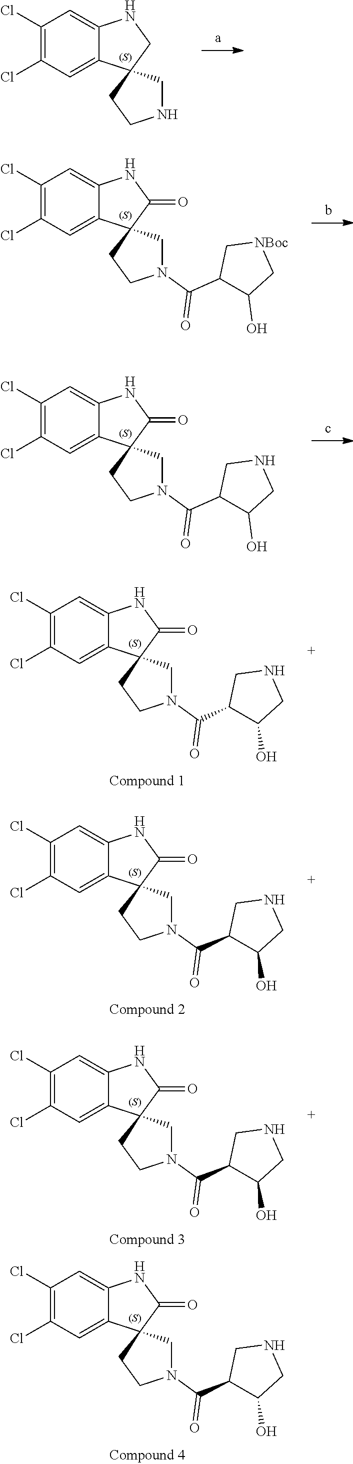

### Step a:

To a stirred solution of (3S)-5,6-dichloro-1H-spiro[indole-3,3-pyrrolidin]-2-one (0.100 g, 0.390 mmol) and 1-(tert-butoxycarbonyl)-4-hydroxypyrrolidine-3-carboxylic acid (99.0 mg, 0.430 mmol) in DMF (2 mL) were added HOBT (68.0 mg, 0.510 mmol), EDCI (97.0 mg, 0.510 mmol) and TEA (0.120 g, 1.17 mmol) at room temperature. The reaction mixture was stirred for 1 h, diluted with water (20 mL) and extracted with EA (3×20 mL). The combined organic layers were washed with brine (5×20 mL) and dried over anhydrous Na₂SO₄. After filtration, the filtrate was concentrated under reduced pressure to afford tert-butyl 3-[[(3S)-5,6-dichloro-2-oxo-1H-spiro[indole-3,3-pyrrolidin]-1-yl]carbonyl]-4-hydroxypyrrolidine-1-carboxylate as a yellow oil (0.200 g, crude), which was used directly in the next step without purification: LCMS (ESI) calc'd for C₂₁H₂₅Cl₂N₃O₅ [M+H]⁺470, 472 (3:2), found 470, 472 (3:2).

### Step b:

To a stirred solution of tert-butyl 3-[[(3S)-5,6-dichloro-2-oxo-1H-spiro[indole-3,3-pyrrolidin]-1-yl]carbonyl]-4-hydroxypyrrolidine-1-carboxylate (0.200 g, crude) in DCM (4 mL) was added TFA (1 mL) at room temperature. The reaction mixture was stirred for 1 h and concentrated under reduced pressure. The residue was purified by Prep-HPLC with the following conditions: Column: XBridge Prep C18 OBD Column, 19×150 mm 5 μm; Mobile Phase A: Water (plus 10 mM NH₄HCO₃), Mobile Phase B: ACN; Flow rate: 20 mL/min; Gradient: 30% B to 50% B in 5.3 min, 50% B; Wavelength: UV 254/210 nm; Retention Time: 5.20 min. The fractions containing the desired product were collected and concentrated under reduced pressure to afford (3S)-5,6-dichloro-1-(4-hydroxypyrrolidine-3-carbonyl)-1H-spiro[indole-3,3-pyrrolidin]-2-one as an off-white solid (84.2 mg, 58% overall two steps): LCMS (ESI) calc'd for C₁₆H₁₇Cl₂N₃O₃ [M+H]⁺: 370, 372 (3:2), found 370, 372 (3:2); ¹H NMR (300 MHz, CD₃OD) δ 7.66-7.37 (m, 1H), 7.12-7.08 (m, 1H), 4.76-4.46 (m, 1H), 4.25-3.65 (m, 4H), 3.63-3.36 (m, 1H), 3.28-2.74 (m, 4H), 2.55-2.16 (m, 2H).
Step c:

(3S)-5,6-dichloro-1′-(4-hydroxypyrrolidine-3-carbonyl)-1H-spiro[indole-3,3′-pyrrolidin]-2-one (80.0 mg, 0.220 mmol) was separated by Prep Chiral HPLC with the following conditions: Column: CHIRALPAK IE, 2×25 cm, 5 μm; Mobile Phase A: Hex (plus 0.3% IPA)-HPLC, Mobile Phase B: EtOH-HPLC; Flow rate: 16 mL/min; Gradient: 50% B to 50% B in 25 min; WavelengthWavelength: UV 220/254 nm; Retention Time 1: 11.61 min; Retention Time 2: 21.49 min; Sample Solvent: EtOH-HPLC; Injection Volume: 1 mL. Two peaks were isolated, each containing two isomers, the faster eluting peak 1 at 11.61 min and the slower eluting peak 2 at 21.49 min, were obtained. Peak 1 was further separated by Prep-Chiral HPLC with the following conditions: Column: CHIRALPAK AD-H, 2×25 cm, 5 μm; Mobile Phase A: Hex (plus 0.5% 2M NH₃-MeOH)-HPLC, Mobile Phase B: EtOH-HPLC; Flow rate: 20 mL/min; Gradient: 30% B to 30% B in 28 min; Wavelength: UV 220/254 nm; Retention Time 1: 10.95 min; Retention Time 2: 21.24 min; Sample Solvent: EtOH-HPLC; Injection Volume: 2 mL; Number Of Runs: 3. The faster eluting isomer at 10.95 min was obtained (3S)-5,6-dichloro-1′-[4-hydroxypyrrolidine-3-carbonyl]-1H-spiro[indole-3,3′-pyrrolidin]-2-one isomer 1 as an off-white solid (8.30 mg, 10%): LCMS (ESI) calc'd for C₁₆H₁₇Cl₂N₃O₃ [M+H]⁺: 370, 372 (3:2), found 370, 372 (3:2); ¹H NMR (400 MHz, CD₃OD) δ 7.47 (d, J=19.8 Hz, 1H), 7.11 (d, J=4.7 Hz, 1H), 4.59-4.44 (m, 1H), 4.27-3.61 (m, 4H), 3.43-3.36 (m, 0.5H), 3.30-3.25 (m, 0.5H), 3.23-2.97 (m, 3H), 2.90-2.76 (m, 1H), 2.49-2.18 (m, 2H). And the slower eluting isomer at 21.24 min was obtained (3S)-5,6-dichloro-1′-[4-hydroxypyrrolidine-3-carbonyl]-1H-spiro[indole-3,3′-pyrrolidin]-2-one isomer 2 as an off-white solid (8.50 mg, 10%): LCMS (ESI) calc'd for C₁₆H₁₇Cl₂N₃O₃ [M+H]⁺: 370, 372 (3:2), found 370, 372 (3:2); ¹H NMR (400 MHz, CD₃OD) δ 7.48 (d, J=35.7 Hz, 1H), 7.11 (d, J=3.7 Hz, 1H), 4.58-4.40 (m, 1H), 4.15-3.66 (m, 4H), 3.41-3.35 (m, 0.5H), 3.30-3.23 (m, 0.5H), 3.22-3.01 (m, 3H), 2.90-2.78 (m, 1H), 2.50-2.20 (m, 2H). Peak 2 from the first separation was further separated by Prep-Chiral HPLC with the following conditions: Column: CHIRALPAK ID, 2×25 cm, 5 μm; Mobile Phase A: MtBE (0.5% 2M NH₃-MeOH)-HPLC, Mobile Phase B: EtOH-HPLC; Flow rate: 20 mL/min; Gradient: 30% B to 30% B in 24 min; Wavelength: UV 220/254 nm; Retention Time 1: 12.97 min; Retention Time 2: 22.25 min; Sample Solvent: EtOH-HPLC; Injection Volume: 1 mL; Number Of Runs: 3. The faster eluting isomer at 12.97 min was obtained (3S)-5,6-dichloro-1′-[4-hydroxypyrrolidine-3-carbonyl]-1H-spiro[indole-3,3′-pyrrolidin]-2-one isomer 3 as an off-white solid (2.40 mg, 3%): LCMS (ESI) calc'd for C₁₆H₁₇Cl₂N₃O₃ [M+H]⁺: 370, 372 (3:2), found 370, 372 (3:2); ¹H NMR (300 MHz, CD₃OD) δ 7.52 (d, J=80.3 Hz, 1H), 7.10 (d, J=3.7 Hz, 1H), 4.74-4.45 (m, 1H), 4.23-3.65 (m, 4H), 3.56-3.44 (m, 1H), 3.23-2.86 (m, 4H), 2.56-2.14 (m, 2H). And the slower eluting isomer at 22.25 min was obtained (3S)-5,6-dichloro-1′-[4-hydroxypyrrolidine-3-carbonyl]-1H-spiro[indole-3,3′-pyrrolidin]-2-one isomer 4 as an off-white solid (3.90 mg, 4%): LCMS (ESI) calc'd for C₁₆H₁₇Cl₂N₃O₃ [M+H]⁺: 370, 372 (3:2), found 370, 372 (3:2); ¹H NMR (300 MHz, CD₃OD) δ 7.52 (d, J=80.3 Hz, 1H), 7.11 (d, J=3.7 Hz, 1H), 4.77-4.49 (m, 1H), 4.17-3.66 (m, 4H), 3.58-3.40 (m, 1H), 3.23-2.85 (m, 4H), 2.56-2.14 (m, 2H).

The compounds in Table 1A below were prepared in an analogous fashion to that described for Compound 1, starting from Intermediate 1S and the corresponding carboxylic acids, which were available from commercial sources.

TABLE 1A

| Compound | | Chemical | |
|---|---|---|---|
| Number | Structure | Name | MS: (M + H)+ & 1H MNR |
| 5 | 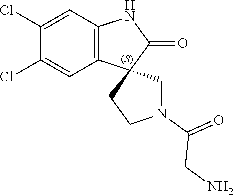 | (S)-5,6- dichloro-1′- glycylspiro[indo- line-3,3′- pyrrolidin]-2- one | [M + H]+: 314, 316 (3:2); 1H NMR (400 MHz, CD3OD) δ 7.46 (d, J = 25.5 Hz, 1H), 7.10 (s, 1H), 3.99- 3.76 (m, 3H), 3.75-3.67 (m, 1H), 3.65-3.56 (m, 1H), 3.53-3.49 (m, 1H), 2.51-2.18 (m, 2H). |
| 6 | 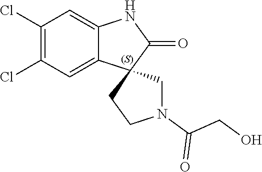 | (S)-5,6- dichloro-1′-(2- hydroxyacetyl)spi- ro[indoline- 3,3′-pyrrolidin]- 2-one | [M + H]+: 315, 317 (3:2); 1H NMR (300 MHz, CD3OD) δ 7.44 (d, J = 19.5 Hz, 1H), 7.09 (s, 1H), 4.39- 4.21 (m, 1H), 4.19-4.16 (m, 1H), 3.98-3.64 (m, 4H), 2.49-2.10 (m, 1H). |
| 7 | 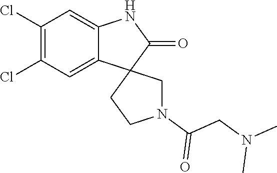 [SMILES: CN(C)CC(=O)N1CCC2(C1)C(=O)Nc1cc(Cl)c(Cl)cc12] | 5,6-dichloro-1′- (dimethylglycyl) spiro[indoline- 3,3′-pyrrolidin]- 2-one | [M + H]+: 342, 344 (3:2); 1H NMR (300 MHz, CD3OD) δ 7.43 (d, J = 24.0 Hz, 1H), 7.09 (s, 1H), 4.03- 3.63 (m, 4H), 3.26-3.12 (m, 2H), 2.51-2.12 (m, 8H). |
| 8 | 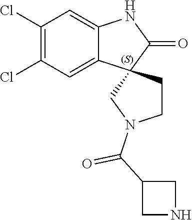 | (S)-1′- (azetidine-3- carbonyl)-5,6- dichlorospiro[in- doline-3,3′- pyrrolidin]-2- one | [M + H]+: 340, 342 (3:2); 1H NMR (400 MHz, CD3OD) δ 7.38 (d, J = 26.1 Hz, 1H), 7.01 (s, 1H), 4.34- 4.05 (m, 4H), 4.04-3.52 (m, 5H), 2.36-2.13 (m, 2H). |
| 9 | 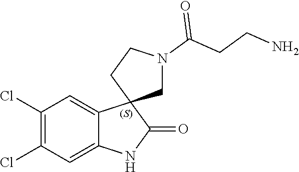 | (S)-1′-(3- aminopropanoyl)- 5,6- dichlorospiro[in- doline-3,3′- pyrrolidin]-2- one | [M + H]+: 328, 330, (3:2); 1H NMR (300 MHz, CD3OD) δ 7.46 (d, J = 17.1 Hz, 1H), 7.10 (d, J = 2.0 Hz, 1H), 4.08-3.60 (m, 4H), 3.09-2.91 (m, 2H), 2.75-2.50 (m, 2H), 2.50-2.18 (m, 2H). |
| 10 | 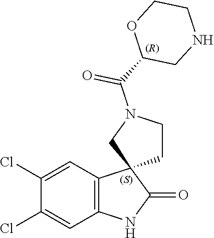 | (S)-5,6- dichloro-1′- ((R)- morpholine-2- carbonyl)spiro[in- doline-3,3′- pyrrolidin]-2- one | [M + H]+: 370, 372 (3:2); 1H NMR (300 MHz, CD3OD) δ 7.44 (d, J = 21.5 Hz, 1H), 7.10 (d, J = 2.3 Hz, 1H), 4.47-4.14 (m, 1H), 4.06-3.56 (m, 6H), 3.19-2.82 (m, 4H), 2.47-2.14 (m, 2H). |
| 11 | 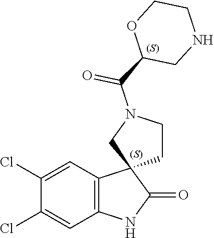 | (S)-5,6- dichloro-1′- ((S)- morpholine-2- carbonyl)spiro[in- doline-3,3′- pyrrolidin]-2- one | [M + H]+: 370, 372 (3:2); 1H NMR (300 MHz, CD3OD) δ 7.42 (d, J = 10.4 Hz, 1H), 7.11 (d, J = 2.5 Hz, 1H), 4.73-4.54 (m, 1H), 4.24-3.97 (m, 3H), 3.97-3.80 (m, 2H), 3.77-3.65 (m, 1H), 3.48-3.39 (m, 2H), 3.28-3.17 (m, 2H), 2.54-2.14 (m, 2H). |
| 12 | 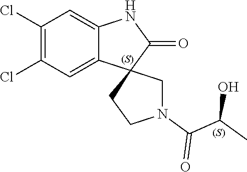 | (S)-5,6- dichloro-1′- ((S)-2- hydroxypropan- oyl)spiro[indoline- 3,3′- pyrrolidin]-2- one | [M + H]+: 3329, 331 (3:2); 1H NMR (400 MHz, CD3OD) δ 7.45 (d, J = 37.7 Hz, 1H), 7.10 (d, J = 2.5 Hz, 1H), 4.61-4.35 (m, 1H), 4.11-3.91 (m, 2H), 3.88-3.70 (m, 2H), 2.51-2.18 (m, 2H), 1.37 (dd, J = 27.5, 6.6 Hz, 3H). |
| 13 | 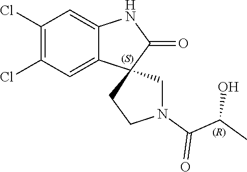 | (S)-5,6- dichloro-1′- ((R)-2- hydroxypropan- oyl)spiro[indoline- 3,3′- pyrrolidin]-2- one | [M + H]+: 329, 331 (3:2); 1H NMR (400 MHz, CD3OD) δ 7.42 (d, J = 31.6 Hz, 1H), 7.11 (d, J = 2.6 Hz, 1H), 4.59-4.39 (m, 1H), 4.19-3.60 (m, 4H), 2.49-2.16 (m, 2H), 1.38 (dd, J = 35.7, 6.6 Hz, 3H). |
| 14 | 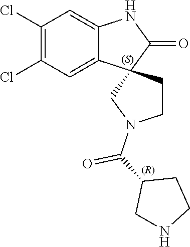 | (S)-5,6- dichloro-1′- ((R)- pyrrolidine-3- carbonyl)spiro[in- doline-3,3′- pyrrolidin]-2- one | [M + H]+: 354, 356 (3:2); 1H NMR (400 MHz, CD3OD) δ 7.46 (d, J = 30.3 Hz, 1H), 7.11 (d, J = 5.6 Hz, 1H), 4.14-3.56 (m, 4H), 3.40-3.36 (m, 1H), 3.30-2.94 (m, 4H), 2.48-1.95 (m, 4H). |
| 15 | 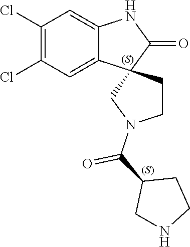 | (S)-5,6- dichloro-1′- ((S)- pyrrolidine-3- carbonyl)spiro[in- doline-3,3′- pyrrolidin]-2- one | [M + H]+: 354, 356 (3:2); 1H NMR (400 MHz, CD3OD) δ 7.45 (d, J = 17.3 Hz, 1H), 7.10 (d, J = 4.2 Hz, 1H), 4.11-3.97 (m, 1H), 3.96-3.78 (m, 2H), 3.66 (d, J = 12.3 Hz, 1H), 3.30-2.83 (m, 5H), 2.47-1.89 (m, 4H). |
| 16 | 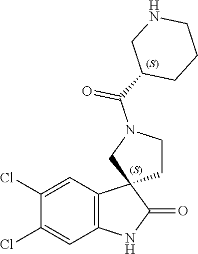 | (S)-5,6- dichloro-1′- ((S)-piperidine- 3- carbonyl)spiro[in- doline-3,3′- pyrrolidin]-2- one | [M + H]+: 368, 370 (3:2); 1H NMR (400 MHz, CD3OD) δ 7.44 (d, J = 23.7 Hz, 1H), 7.11 (d, J = 6.0 Hz, 1H), 4.15-3.76 (m, 4H), 3.64 (d, J = 12.4 Hz, 1H), 3.22- 2.64 (m, 4H), 2.47-2.18 (m, 2H), 2.08-1.47 (m, 4H). |
| 17 | 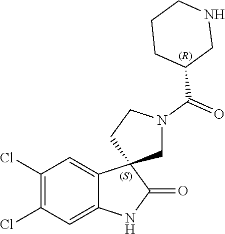 | (S)-5,6- dichloro-1′- ((R)-piperidine- 3- carbonyl)spiro[in- doline-3,3′- pyrrolidin]-2- one | [M + H]+: 368, 370 (3:2); 1H NMR (400 MHz, CD3OD) δ 7.45 (d, J = 32.5 Hz, 1H), 7.11 (d, J = 6.6 Hz, 1H), 4.10-3.95 (m, 1H), 3.95-3.78 (m, 2H), 3.66 (d, J = 12.4 Hz, 1H), 3.23-2.72 (m, 5H), 2.49-2.19 (m, 2H), 2.10-1.56 (m, 4H). |
| 18 | 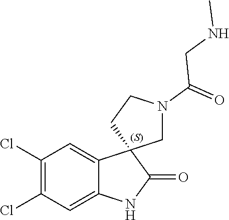 | (S)-5,6- dichloro-1′- (methylglycyl)spi- ro[indoline- 3,3′-pyrrolidin]- 2-one | [M + H]+: 328, 330 (3:2); 1H NMR (300 MHz, CD3OD) δ 7.46 (d, J = 18.6 Hz, 1H), 7.10 (s, 1H), 4.09- 3.54 (m, 6H), 2.61-2.16 (m, 5H). |
| 19 | 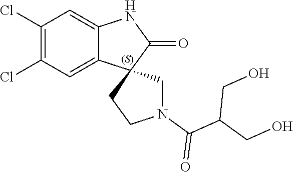 | (S)-5,6- dichloro-1′-(3- hydroxy-2- (hydroxymethyl) propanoyl)spiro [indoline-3,3′- pyrrolidin]-2- one | [M + H]+: 359, 361 (3:2); 1H NMR (300 MHz, CD3OD) δ 7.52 (d, J = 50.3 Hz, 1H), 7.10 (d, J = 1.6 Hz, 1H), 4.24-4.05 (m, 1H), 4.02-3.97 (m, 1H), 3.95-3.55 (m, 6H), 3.29-2.99 (m, 1H), 2.58-2.11 (m, 2H). |
| 20 | 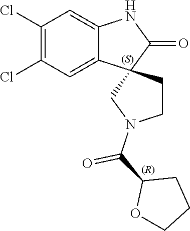 | (S)-5,6- dichloro-1′- ((R)- tetrahydrofuran- 2- carbonyl)spiro[in- doline-3,3′- pyrrolidin]-2- one | [M + H]+: 355, 357 (3:2); 1H NMR (400 MHz, CD3OD) δ 7.43 (d, J = 22.1 Hz, 1H), 7.10 (d, J = 1.4 Hz, 1H), 4.75-4.52 (m, 1H), 4.19-3.64 (m, 6H), 2.49-2.29 (m, 2H), 2.27-2.14 (m, 1H), 2.14-1.89 (m, 3H). |
| 21 | 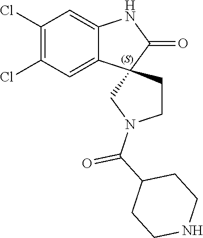 | (S)-5,6- dichloro-1′- (piperidine-4- carbonyl)spiro[in- doline-3,3′- pyrrolidin]-2- one | [M + H]+: 368, 370 (3:2); 1H NMR (400 MHz, CD3OD) δ 7.40 (d, J = 28.1 Hz, 1H), 7.08 (d, J = 5.1 Hz, 1H), 4.11-3.93 (m, 1H), 3.93-3.59 (m, 3H), 3.18-3.03 (m, 2H), 2.85-2.56 (m, 3H), 2.45-2.16 (m, 2H), 1.90-1.79 (m, 1H), 1.79-1.62 (m, 3H). |
| 22 | 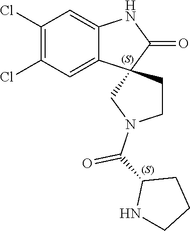 | (S)-1′-(L- prolyl)-5,6- dichlorospiro[in- doline-3,3′- pyrrolidin]-2- one | [M + H]+: 354, 356 (3:2); 1H NMR (400 MHz, CD3OD) δ 7.47 (d, J = 42.8 Hz, 1H), 7.10 (d, J = 4.9 Hz, 1H), 4.04-3.87 (m, 3H), 3.87-3.69 (m, 3H), 3.26-3.13 (m, 1H), 2.89-2.75 (m, 1H), 2.50-2.34 (m, 1H), 2.34-2.12 (m, 1H), 1.97-1.67 (m, 3H). |
| 23 | 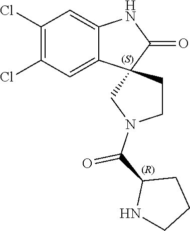 | (S)-1′-(D- prolyl)-5,6- dichlorospiro[in- doline-3,3′- pyrrolidin]-2- one | [M + H]+: 354, 356 (3:2); 1H NMR (400 MHz, CD3OD) δ 7.44 (d, J = 28.6 Hz, 1H), 7.10 (d, J = 3.1 Hz, 1H), 3.78-4.07 (m, 3H), 3.56-3.78 (m, 2H), 3.19-3.13 (m, 1H), 2.97-3.07 (m, 1H), 2.18-2.47 (m, 2H), 2.03-2.18 (m, 2H), 1.72-1.8 (m, 1H), 1.6-1.69 (m, 1H). |
| 24 | 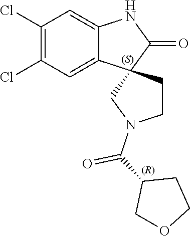 | (S)-5,6- dichloro-1′- ((R)- tetrahydrofuran- 3- carbonyl)Spiro[in- doline-3,3′- pyrrolidin]-2- one | [M + H]+: 355, 357 (3:2); 1H NMR (300 MHz, CD3OD) δ 7.46 (d, J = 21.3 Hz, 1H), 7.10 (d, J = 3.7 Hz, 1H), 4.02-4.16 (m, 1H), 3.74-4.02 (m, 6H), 3.63-3.71 (m, 1H), 3.38-3.48 (m, 1H), 2.29-2.47 (m, 2H), 2.08-2.28 (m, 2H). |
| 25 | 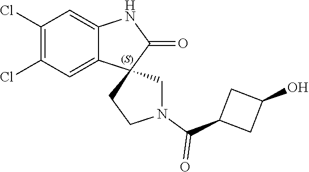 | (S)-5,6- dichloro-1′- ((1s,3R)-3- hydroxycyclobu- tane-1- carbonyl)spiro[in- doline-3,3′- pyrrolidin]-2- one | [M + H ]+: 355, 357 (3:2); 1H NMR (300 MHz, DMSO- d6) δ 10.79-10.73 (brs, 1H), 7.52 (d, J = 26.5 Hz, 1H), 7.05 (d, J = 3.9 Hz, 1H), 5.09 (dd, J = 12.8, 7.0 Hz, 1H), 3.84-4.11 (m, 1H), 3.7-3.84 (m, 1H), 3.53-3.7 (m, 3H), 2.56-2.84 (m, 1H), 2.35-2.47 (m, 1H), 2.2-2.34 (m, 2H), 2.12-2.2 (m, 1H), 1.86-2.07 |
| | | | (m, 2H). |
| 26 | 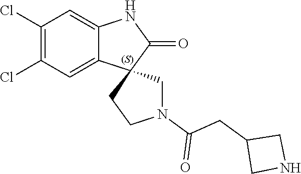 | (S)-1′-(2- (azetidin-3- yl)acetyl)-5,6- dichlorospiro[in- doline-3,3′- pyrrolidin]-2- one | [M + H]+: 354, 356 (3:2); 1H NMR (300 MHz, CD3OD) δ 7.44 (d, J = 1.68 Hz, 1H), 7.10 (d, J = 3.9 Hz, 1H), 3.96-4.12 (m, 1H), 3.83-3.96 (m, 3H), 3.65-3.83 (m, 2H), 3.45-3.65 (m, 2H), 3.07-3.27 (m, 1H), 2.83 (d, J = 7.5 Hz, 1H), 2.72 (d, J = 7.7 Hz, 1H), 2.13-2.49 (m, 2H). |
| 27 | 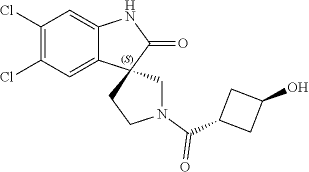 | (S)-5,6- dichloro-1′- ((1r,3S)-3- hydroxycyclobu- tane-1- carbonyl)spiro[in- doline-3,3′- pyrrolidin]-2- one | [M + H]+: 355, 357 (3:2); 1H NMR (400 MHz, CD3OD) δ 7.42 (d, J = 24.8 Hz, 1H), 7.09 (d, J = 2.2 Hz, 1H), 4.35-4.49 (m, 1H), 3.85-3.96 (m, 1H), 3.62-3.85 (m, 3H), 3.14-3.23 (m, 1H), 2.46-2.66 (m, 2H), 2.28-2.43 (m, 2H), 2.12-2.28 (m, 2H). |
| 28 | 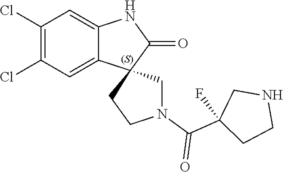 | (S)-5,6- dichloro-1′-(3- fluoropyrrolidine- 3- carbonyl)spiro[in- doline-3,3′- pyrrolidin]-2- one isomer 1 | [M + H]+: 372, 374 (3:2); 1H NMR (300 MHz, CD3OD) δ 7.44 (d, J = 14.1 Hz, 1H), 7.10 (s, 1H), 4.09- 4.26 (m, 1H), 3.81-4.09 (m, 2H), 3.75 (d, J = 12.6 Hz, 1H), 3.35-3.52 (m, 2H), 2.99-3.29 (m, 2H), 2.16-2.62 (m, 4H); 19F NMR (282 MHz, CD3OD) δ −150.76 (s), −151.48 (s). |
| 29 | 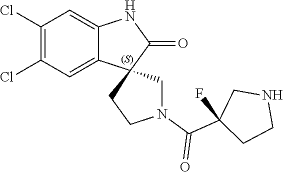 | (S)-5,6- dichloro-1′-(3- fluoropyrrolidine- 3- carbonyl)spiro[in- doline-3,3′- pyrrolidin]-2- one isomer 1 | [M + H]+: 372, 374 (3:2); 1H NMR (300 MHz, CD3OD) δ 7.43 (d, J = 21.3 Hz, 1H), 7.10 (d, J = 1.6 Hz, 1H), 4.07-4.27 (m, 1H), 3.83-4.07 (m, 3H), 3.67-3.77 (m, 1H), 3.37-3.56 (m, 1H), 3.0-3.3 (m, 2H), 2.16-2.59 (m, 4H); 19F NMR (282 MHz, CD3OD) δ −150.77 (s), −151.48 (s). |
| 30 | 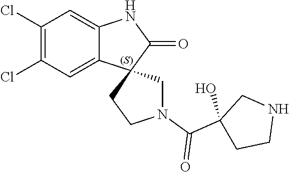 | (S)-5,6- dichloro-1′-(3- hydroxypyrroli- dine-3- carbonyl)spiro[in- doline-3,3′- pyrrolidin]-2- one isomer 1 | [M + H]+: 370, 372 (3:2); 1H NMR (400 MHz, CD3OD) δ 7.41 (d, J = 67.3 Hz, 1H), 7.11 (d, J = 2.6 Hz, 1H), 4.26 (t, J = 7.1 Hz, 1H), 4.01-4.18 (m, 1H), 3.65-3.96 (m, 2H), 3.48 (dd, J = 29.3, 12.2 Hz, 1H), 2.91-3.26 (m, 3H), 2.21-2.51 (m, 2H), 1.89-2.21 (m, 2H). |
| 31 | 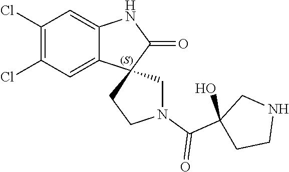 | (S)-5,6- dichloro-1′-(3- hydroxypyrroli- dine-3- carbonyl)spiro[in- doline-3,3′- pyrrolidin]-2- one isomer 2 | [M + H]+: 370, 372 (3:2); 1H NMR (40 0MHz, CD3OD) δ 7.43 (d, J = 47.9 Hz, 1H), 7.11 (s, 1H), 4.15- 4.35 (m, 1H), 4.10-4.02 (m, 1H), 3.69-3.99 (m, 2H), 3.35-3.46 (m, 1H), 2.84-3.26 (m, 3H), 2.25-2.5 (m, 2H), 1.90-2.23 (m, 2H). |
| 32 | 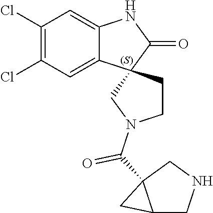 | (3S)-1′-(3- azabicyclo[3.1.0] hexane-1- carbonyl)-5,6- dichlorospiro[in- doline-3,3′- pyrrolidin]-2- one isomer 1 | [M + H]+: 366, 368 (3:2); 1H NMR (400 MHz, CD3OD) δ 7.40 (d, J = 60.4 Hz, 1H), 7.11 (s, 1H), 3.54- 4.24 (m, 4H), 2.84-3.23 (m, 4H), 2.15-2.47 (m, 2H), 1.96 (s, 1H), 0.72-1.19 (m, 2H). |
| 33 | 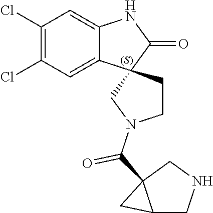 | (3S)-1′-(3- azabicyclo[3.1.0] hexane-1- carbonyl)-5,6- dichlorospiro[in- doline-3,3′- pyrrolidin]-2- one isomer 2 | [M + H]+: 366, 368 (3:2); 1H NMR (400 MHz, CD3OD) δ 7.44 (s, 1H), 7.10 (s, 1H), 3.96-4.16 (m, 2H), 3.65-3.96 (m, 2H), 2.90-3.17 (m, 4H), 2.26-2.45 (m, 2H), 1.88-2.06 (m, 1H), 0.79-1.17 (m, 2H). |
| 34 | 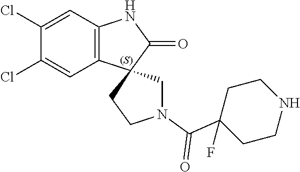 | (S)-5,6- dichloro-1′-(4- fluoropiperidine- 4- carbonyl)spiro[in- doline-3,3′- pyrrolidin]-2- one | [M + H]+: 386, 388 (3:2); 1H NMR (400 MHz, CD3OD) δ 7.46 (d, J = 19.8 Hz, 1H), 7.11 (d, J = 0.7 Hz, 1H), 4.06-4.28 (m, 2H), 3.84-4.05 (m, 2H), 3.73 (d, J = 12.7 Hz, 1H), 3.36-3.55 (m, 2H), 3.20-3.31 (m, 1H), 2.37-2.57 (m, 4H), 2.28-2.37 (m, 1H), 2.19-2.28 (m, 1H). |
| 35 | 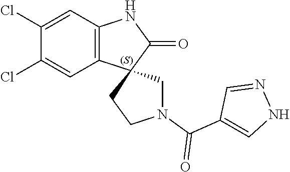 | (S)-5,6- dichloro-1′- (1H-pyrazole-4- carbonyl)spiro[in- doline-3,3′- pyrrolidin]-2- one | [M + H]+: 351, 353 (3:2); 1H NMR (400 MHz, CD3OD) δ 8.11 (s, 2H), 7.47 (d, J = 29.9 Hz, 1H), 7.10 (d, J = 8.3 Hz, 1H), 4.15-4.34 (m, 1H), 3.93-4.15 (m, 2H), 3.86 (d, J = 12.5 Hz, 1H), 2.24-2.55 (m, 2H). |
| 36 | 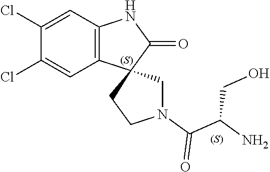 | (S)-1′-(L- Seryl)-5,6- dichlorospiro[in- doline-3,3′- pyrrolidin]-2- one | [M + H]+: 344, 346 (3:2); 1H NMR (400 MHz, CD3OD) δ 7.50 (d, J = 47 .1 Hz, 1H), 7.10 (d, J = 1.4 Hz, 1H), 3.91-4.2 (m, 3H), 3.8- 3.89 (m, 1H), 3.70-3.80 (m, 2H), 3.58-3.66 (m, 1H), 2.16-2.49 (m, 2H). |
| 37 | 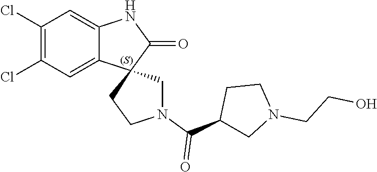 | (S)-5,6- dichloro-1′-(1- (2- hydroxyethyl)py- rrolidine-3- carbonyl)spiro[in- doline-3,3′- pyrrolidin]-2- one isomer 1 | [M + H]+: 398, 400 (3:2); 1H NMR (400 MHz, CD3OD) δ 7.44 (d, J = 25.6 Hz, 1H), 7.10 (d, J = 4.7 Hz, 1H), 3.93-4.1 (m, 1H), 3.77- 3.93 (m, 2H), 3.63-3.75 (m, 3H), 3.35-3.4 (m, 0.5H), 2.98-3.27 (m, 1.5H), 2.81- 2.96 (m, 1H), 2.58-2.81 (m, 4H), 2.00-2.47 (m, 4H). |
| 38 | 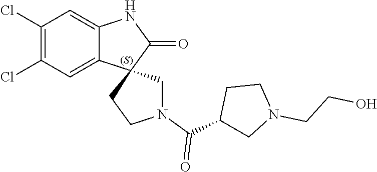 | (S)-5,6- dichloro-1′-(1- (2- hydroxyethyl)py- rrolidine-3- carbonyl)spiro[in- doline-3,3′- pyrrolidin]-2- one isomer 2 | [M + H]+: 398, 400 (3:2); 1H NMR (400 MHz, CD3OD) δ 7.44 (d, J = 27.6 Hz, 1H), 7.10 (d, J = 4.3 Hz, 1H), 3.94-4.09 (m, 1H), 3.78-3.94 (m, 2H), 3.63-3.75 (m, 3H), 3.35-3.41 (m, 0.5H), 2.95-3.26 (m, 1.5H), 2.81-2.95 (m, 1H), 2.55-2.78 (m, 4H), 2.02-2.47 (m, 4H). |
| 39 | 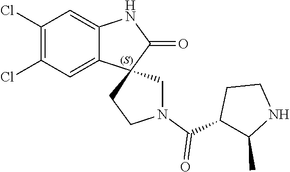 | (S)-5,6- dichloro-1′- ((2S,3R)-rel-2- methylpyrrolidine- 3- carbonyl)spiro[in- doline-3,3′- pyrrolidin]-2- one isomer 1 | [M + H]+: 368, 370 (3:2); 1H NMR (400 MHz, CD3OD) δ 7.46 (d, J = 8.7 Hz, 1H), 7.10 (s, 1H), 3.59- 4.21 (m, 4H), 3.35-3.52 (m, 2H), 3.11-3.3 (m, 1H), 2.83- 3.03 (m, 1H), 1.97-2.48 (m, 4H), 1.28 (dd, J = 33.0, 6.6 Hz, 3H). |
| 40 | 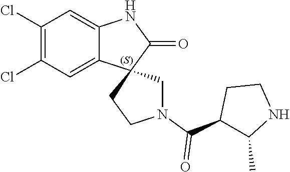 | (S)-5,6- dichloro-1′- ((2S,3R)-rel-2- methylpyrrolidine- 3- carbonyl)spiro[in- doline-3,3′- pyrrolidin]-2- one isomer 2 | [M + H]+: 368, 370 (3:2); 1H NMR (400 MHz, CD3OD) δ 7.45 (d, J = 13.1 Hz, 1H), 7.10 (d, J = 5.7 Hz, 1H), 3.64-4.18 (m, 4H), 3.36-3.53 (m, 1H), 2.98-3.19 (m, 2H), 2.60-289 (m, 1H), 1.86-2.48 (m, 4H), 1.27 (dd, J = 40.0, 6.5 Hz, 3H). |
| 41 | 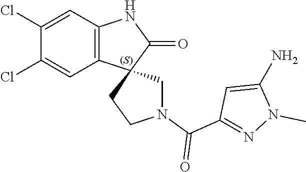 | (S)-1′-(5- amino-1- methyl-1H- pyrazole-3- carbonyl)-5,6- dichlorospiro[in- doline-3,3′- pyrrolidin]-2- one | [M + H]+: 380, 382 (3:2); 1H NMR (400 MHz, CD3OD) δ 7.44 (d, J = 50.0 Hz, 1H), 7.10 (d, J = 7.3 Hz, 1H), 5.93 (d, J = 9.8 Hz, 1H), 4.34 (t, J = 7.1 Hz, 1H), 4.26-4.22 (m, 1H), 3.80-4.10 (m, 2H), 3.67 (d, J = 35.5 Hz, 3H), 2.34-2.51 (m, 1H), 2.19-2.33 (m, 1H). |
| 42 | 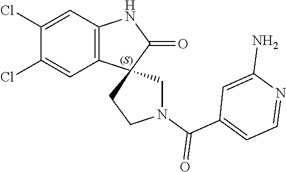 | (S)-1′-(2- aminoisonicotin- oyl)-5,6- dichlorospiro[in- doline-3,3′- pyrrolidin]-2- one | [M + H]+: 377, 379 (3:2); 1H NMR (300 MHz, DMSO- d6) δ 10.77 (d, J = 19.4 Hz, 1H), 7.98 (dd, J = 22.3, 5.0 Hz, 1H), 7.61 (dd, J = 65.3, 3.2 Hz, 1H), 7.05 (d, J = 14.5 Hz, 1H), 6.43-6.68 (m, 2H), 6.13 (d, J = 14.7 Hz, 2H), 3.53-3.95 (m, 4H), 2.20-2.34 (m, 2H). |
| 43 | 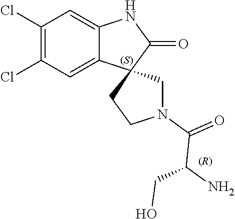 | (S)-1′-(D- seryl)-5,6- dichlorospiro[in- doline-3,3′- pyrrolidin]-2- one | [M + H]+: 344, 346 (3:2); 1H NMR (400 MHz, CD3OD) δ 7.51 (d, J = 66.9 Hz, 1H), 7.11 (d, J = 4.3 Hz, 1H), 4.06-4.15 (m, 1H), 3.85-4.00 (m, 2H), 3.72-3.81 (m, 2H), 3.56-3.72 (m, 2H), 2.33-2.55 (m, 1H), 2.13-2.29 (m, 1H). |
| 44 | 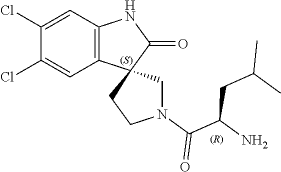 | (S)-1′-(D- leucyl)-5,6- dichlorospiro[in- doline-3,3′- pyrrolidin]-2- one | [M + H]+: 370, 372 (3:2); 1H NMR (400 MHz, DMSO- d6) δ 7.48 (d, J = 9.3 Hz, 1H), 7.06 (d, J = 3.5 Hz, 1H), 3.86-4.04 (m, 1H), 3.65-3.81 (m, 1H), 3.48-3.64 (m, 2H), 3.39 (dd, J = 9.1, 4.7 Hz, 1H), 2.05-2.31 (m, 2H), 1.71-1.88 (m, 1H), 1.14-1.45 (m, 2H), 0.94 (t, J = 7.1 Hz, 3H), 0.84 (dd, J = 15.7, 6.6 |
| | | | Hz, 3H). |
| 45 | 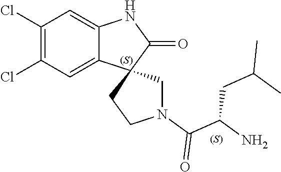 | (S)-1′-(L- leucyl)-5,6- dichlorospiro[in- doline-3,3′- pyrrolidin]-2- one | [M + H]+: 370, 372 (3:2); 1H NMR (300 MHz, CD3OD) δ 7.48 (d, J = 46.0 Hz, 1H), 7.11 (d, J = 6.0 Hz, 1H), 3.87-4.1 (m, 2H)), 3.57- 3.86 (m, 3H), 2.16-2.49 (m, 2H), 1.66-1.92 (m, 1H), 1.38-1.66 (m, 2H), 0.87-1.05 (m, 6H). |
| 46 | 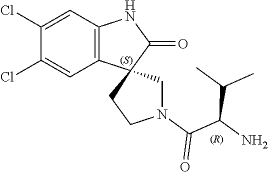 | (S)-1′-(D- valyl)-5,6- dichlorospiro[in- doline-3,3′- pyrrolidin]-2- one | [M + H]+: 356, 358 (3:2); 1H NMR (400 MHz, CD3OD) δ 7.43 (d, J = 12.,7 Hz, 1H), 7.11 (d, J = 1.5 Hz, 1H), 3.92-4.17 (m, 1H), 3.62-3.92 (m, 3H), 3.42 (dd, J = 51.8, 6.2 Hz, 1H), 2.17- 2.51 (m, 2H), 1.86-2.05 (m, 1H), 1.07 (dd, J = 15.7, 6.8 Hz, 3H), 0.98 (t, J = 6.9 Hz, 3H). |
| 47 | 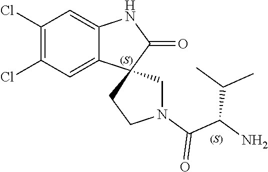 | (S)-1′-(L-valyl)- 5,6- dichlorospiro[in- doline-3,3′- pyrrolidin]-2- one | [M + H]+: 356, 358 (3:2); 1H NMR (300 MHz, CD3OD) δ 7.48 (d, J = 51.3 Hz, 1H), 7.10 (d, J = 4.6 Hz, 1H), 3.91-4.09 (m, 2H), 3.68-3.87 (m, 2H), 3.46 (dd, J = 21.1, 6.0 Hz, 1H), 2.15- 2.49 (m, 2H), 1.84-2.06 (m, 1H), 0.94-1.08 (m, 6H). |
| 48 | 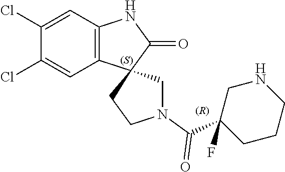 | (S)-5,6- dichloro-1′- ((R)-3- fluoropiperidine- 3- carbonyl)spiro[in- doline-3,3′- pyrrolidin]-2- one | [M + H]+: 386, 388 (3:2); 1H NMR (300 MHz, CD3OD) δ 7.43 (d, J = 14.8 Hz, 1H), 7.10 (d, J = 1.5 Hz, 1H), 4.07-4.31 (m, 1H), 4.03 (dd, J = 9.4, 4.0 Hz, 1H), 3.69-3.99 (m, 2H), 2.92-3.26 (m, 3H), 2.60-2.76 (m, 1H), 2.01-2.44 (m, 4H), 1.54-1.94 (m, 2H); 19F NMR (282 MHz, CD3OD) δ −167.47 (d, |
| | | | J = 317.1 Hz). |
| 49 | 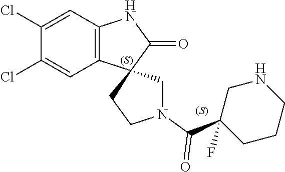 | (S)-5,6- dichloro-1′- ((S)-3- fluoropiperidine- 3- carbonyl)spiro[in- doline-3,3′- pyrrolidin]-2- one | [M + H]+: 386, 388 (3:2); 1H NMR (400 MHz, CD3OD) δ 7.41 (d, J = 39.6 Hz, 1H), 7.10 (d, J = 3.8 Hz, 1H), 4.11-4.28 (m, 1H), 3.96-4.11 (m, 1H), 3.67-3.94 (m, 2H), 3.07-3.23 (m, 2H), 2.91-3.03 (m, 1H), 2.69 (q, J = 12.0 Hz, 1H), 1.98-2.47 (m, 4H), 1.72-1.93 (m, 1H), 1.58-1.71 (m, 1H); 19F NMR |
| | | | (376 MHz, CD3OD) δ −167.37 |
| | | | (d, J = 245.0 Hz). |
| 50 | 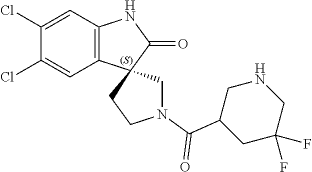 | (3S)-5,6- dichloro-1′- (5,5- difluoropiperidine- 3- carbonyl)spiro[in- doline-3,3′- pyrrolidin]-2- one | [M + H]+: 404, 406 (3:2); 1H NMR (300 MHz, CD3OD) δ 7.33-7.55 (m, 1H), 7.03-7.16 (m, 1H), 3.53-4.20 (m, 4H), 2.53-3.26 (m, 5H), 2.05-2.48 (m, 4H). |
| 51 | 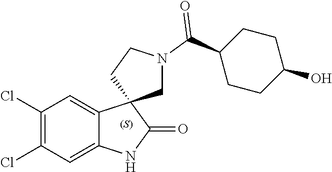 | (S)-5,6- dichloro-1′- ((1s,4R)-4- hydroxycyclohex- ane-1- carbonyl)spiro[in- doline-3,3′- pyrrolidin]-2- one | [M + H]+: 383, 385 (3:2); 1H NMR (400 MHz, CD3OD) δ 7.41 (d, J = 29.3 Hz, 1H), 7.10 (d, J = 5.3 Hz, 1H), 3.90-4.12 (m, 2H), 3.61-3.90 (m, 3H), 2.46-2.69 (m, 1H), 2.16-2.46 (m, 2H), 1.79-2.07 (m, 4H), 1.48-1.73 (m, 4H). |
| 52 | 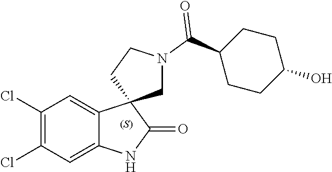 | (S)-5,6- dichloro-1′- ((1r,4S)-4- hydroxycyclohex- ane-1- carbonyl)Spiro[in- doline-3,3′- pyrrolidin]-2- one | [M + H]+: 383, 385 (3:2); 1H NMR (300 MHz, CD3OD) δ 7.42 (d, J = 21.1 Hz, 1H), 7.10 (d, J = 4.2 Hz, 1H), 3.95-4.12 (m, 1H), 3.61-3.91 (m, 3H), 3.46-3.62 (m, 1H), 2.13-2.64 (m, 3H), 1.73-2.11 (m, 4H), 1.47-1.69 (m, 2H), 1.20-1.47 (m, 2H). |
| 53 | 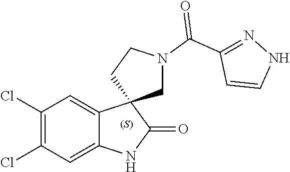 | (S)-5,6- dichloro-1′- (1H-pyrazole-3- carbonyl)spiro[in- doline-3,3′- pyrrolidin]-2- one | [M + H]+: 351, 353 (3:2); 1H NMR (400 MHz, CD3OD) δ 7.72 (dd, J = 22.4, 2.4 Hz, 1H), 7.46 (d, J = 39.1 Hz, 1H), 7.10 (d, J = 9.4 Hz, 1H), 6.83 (dd, J = 9.5, 2.4 Hz, 1H), 4.34-4.47 (m, 1H), 4.30-4.26 (m, 1H), 3.85-4.18 (m, 2H), 2.21-2.53 (m, 2H). |
| 54 | 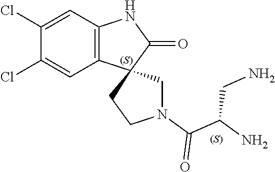 | (S)-1′-((S)-3- amino-2- hydroxypropan- oyl)-5,6- dichlorospiro[in- doline-3,3′- pyrrolidin]-2- one | [M + H]+: 344, 346 (3:2); 1H NMR (300 MHz, CD3OD) δ 7.47 (d, J = 19.5 Hz, 1H), 7.10 (d, J = 2.1 Hz, 1H), 4.23-4.46 (m, 1H), 3.88-4.11 (m, 2H), 3.68-3.88 (m, 2H), 2.74-3.03 (m, 2H), 2.17-2.50 (m, 2H). |
| 55 | 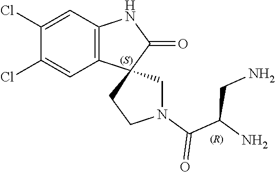 | (S)-1′-((R)-3- amino-2- hydroxypropan- oyl)-5,6- dichlorospiro[in- doline-3,3′- pyrrolidin]-2- one | [M + H]+: 344, 346 (3:2); 1H NMR (300 MHz, CD3OD) δ 7.45 (d, J = 32.9 Hz, 1H), 7.10 (d, J = 2.5 Hz, 1H), 4.24-4.46 (m, 1H), 3.95-4.23 (m, 1H), 3.64-3.95 (m, 3H), 2.73-3.05 (m, 2H), 2.14-2.51 (m, 2H). |
| 56 | 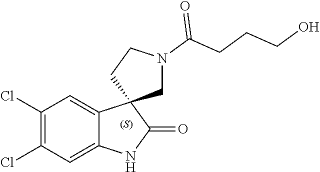 | (S)-5,6- dichloro-1′-(4- hydroxybutanoyl) spiro[indoline- 3,3′- pyrrolidin]-2- one | [M + H]+: 343, 345 (3:2); 1H NMR (300 MHz, DMSO- d6) δ 7.53 (d, J = 24.1 Hz, 1H), 7.09 (d, J = 2.2 Hz, 1H), 3.61-3.87 (m, 3H), 3.34-3.57 (m, 3H), 2.31-2.43 (m, 1H), 2.21-2.30 (m, 2H), 2.10-2.20 (m, 1H), 1.58-1.76 (m, 2H). |
| 57 | 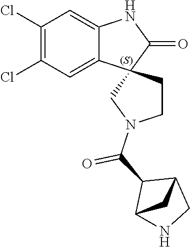 | (S)-1′-(2- azabicyclo[2.1.1] hexane-5- carbonyl)-5,6- dichlorospiro[in- doline-3,3′- pyrrolidin]-2- one isomer 1 | [M + H]+: 366, 368 (3:2); 1H NMR (400 MHz, CD3OD) δ 7.05-7.71 (m, 2H), 3.74-4.12 (m, 3H), 3.48-3.71 (m, 1H), 3.34-3.36 (m, 2H), 3.31-3.29 (m, 1H), 2.69-2.96 (m, 1H), 2.57 (d, J = 7.4 Hz, 1H), 2.46 (d, J = 7.4 Hz, 1H), 2.18-2.44 (m, 3H). |
| 58 | 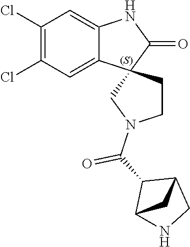 | (S)-1′-(2- azabicyclo[2.1.1] hexane-5- carbonyl)-5,6- dichlorospiro[in- doline-3,3′- pyrrolidin]-2- one isomer 2 | [M + H]+: 366, 368 (3:2); 1H NMR (400 MHz, CD3OD) δ 7.07-7.71 (m, 2H), 3.74-4.15 (m, 3H), 3.47-3.70 (m, 1H), 3.34-3.37 (m, 3H), 2.67-2.96 (m, 2H), 2.52-2.61 (m, 1H), 2.46 (d, J = 7.4 Hz, 1H), 2.17-2.44 (m, 2H). |
| 59 | 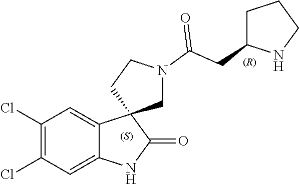 | (S)-5,6- dichloro-1′-(2- ((R)-pyrrolidin- 2- yl)acetyl)spiro[in- doline-3,3′- pyrrolidin]-2- one | [M + H]+: 368, 370 (3:2); 1H NMR (300 MHz, CD3OD) δ 7.46 (d, J = 13.9 Hz, 1H), 7.10 (d, J = 2.0 Hz, 1H), 3.87-4.07 (m, 2H), 3.63-3.86 (m, 2H), 3.42-3.63 (m, 1H), 2.89-3.13 (m, 2H), 2.59-2.82 (m, 1H), 2.51-2.61 (m, 1H), 2.15-2.52 (m, 2H), 2.01-2.14 (m, 1H), 1.76-1.96 (m, 2H), 1.38-1.65 (m, 1H). |
| 60 | 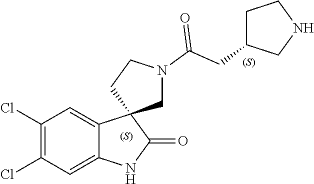 | (S)-5,6- dichloro-1′-(2- ((S)-pyrrolidin- 3- yl)acetyl)spiro[in- doline-3,3′- pyrrolidin]-2- one | [M + H]+: 368, 370 (3:2); 1H NMR (400 MHz, CD3OD) δ 7.46 (d, J = 27.1 Hz, 1H), 7.11 (d, J = 3.4 Hz, 1H), 3.87-4.04 (m, 2H), 3.64-3.87 (m, 2H), 3.54-3.64 (m, 1H), 3.37-3.47 (m, 1H), 3.21-3.3 (m, 1H), 2.90-3.02 (m, 1H), 2.58-2.83 (m, 3H), 2.20-2.58 (m, 3H), 1.65-1.85 (m, 1H). |
| 61 | 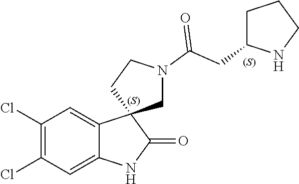 | (S)-5,6- dichloro-1′-(2- ((S)-pyrrolidin- 2- yl)acetyl)spiro[in- doline-3,3′- pyrrolidin]-2- one | [M + H]+: 368, 370 (3:2); 1H NMR (300 MHz, CD3OD) δ 7.47 (d, J = 29.2 Hz, 1H), 7.10 (d, J = 1.4 Hz, 1H), 3.65-4.06 (m, 4H), 3.46-3.61 (m, 1H), 2.88-3.12 (m, 2H), 2.55-2.71 (m, 2H), 2.17-2.52 (m, 2H), 1.97-2.14 (m, 1H), 1.72-1.97 (m, 2H), 1.38-1.60 (m, 1H). |
| 62 | 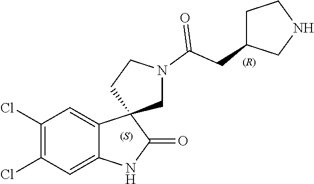 | (S)-5,6- dichloro-1′-(2- ((R)-pyrrolidin- 3- yl)acetyl)spiro[in- doline-3,3′- pyrrolidin]-2- one | [M + H]+: 368, 370 (3:2); 1H NMR (400 MHz, CD3OD) δ 7.46 (d, J = 16.8 Hz, 1H), 7.11 (d, J = 2.9 Hz, 1H), 3.75-4.04 (m, 4H), 3.56-3.70 (m, 2H), 3.36-3.48 (m, 1H), 3.21-3.31 (m, 1H), 2.91-3.03 (m, 1H), 2.49-2.83 (m, 3H), 2.19-2.47 (m, 3H). |
| 63 | 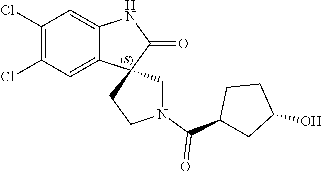 | (S)-5,6- dichloro-1′-(3- hydroxycyclopen- tane-1- carbonyl)spior[in- doline-3,3′- pyrrolidin]-2- one isomer 1 | [M + H]+: 369, 371 (3:2); 1H NMR (400 MHz, CD3OD) δ 7.43 (d, J = 32.8 Hz, 1H), 7.10 (d, J = 4.5 Hz, 1H), 4.19-4.34 (m, 1H), 3.88-4.10 (m, 2H), 3.63-3.85 (m, 2H), 2.95-3.23 (m, 1H), 2.29-2.47 (m, 2H), 1.97-2.26 (m, 3H), 1.68-1.97 (m, 3H). |
| 64 | 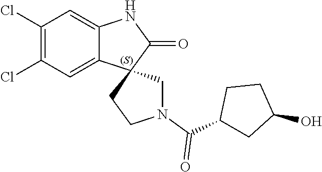 | (S)-5,6- dichloro-1′-(3- hydroxycyclopen- tane-1- carbonyl)spiro[in- doline-3,3′- pyrrolidin]-2- one isomer 2 | [M + H]+: 369, 371 (3:2); 1H NMR (400 MHz, CD3OD) δ 7.43 (d, J = 17.6 Hz, 1H), 7.10 (d, J = 3.4 Hz, 1H), 4.17-4.31 (m, 1H), 3.92-4.12 (m, 1H), 3.62-3.92 (m, 3H), 2.97-3.22 (m, 1H), 1.67-2.47 (m, 8H). |
| 65 | 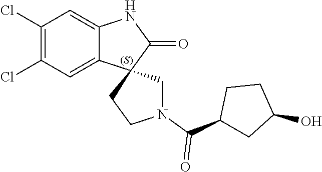 | (S)-5,6- dichloro-1′-(3- hydroxycyclopen- tane-1- carbonyl)spiro[in- doline-3,3′- pyrrolidin]-2- one isomer 3 | [M + H]+: 369, 371 (3:2); 1H NMR (400 MHz, CD3OD) δ 7.42 (d, J = 28.2 Hz, 1H), 7.10 (d, J = 5.3 Hz, 1H), 4.34-4.44 (m, 1H), 3.95-4.13 (m, 1H), 3.64-3.93 (m, 3H), 3.13-3.20 (m, 1H), 1.56-2.50 (m, 8H). |
| 66 | 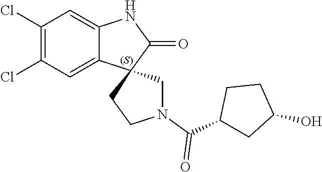 | (S)-5,6- dichloro-1′-(3- hydroxycyclopen- tane-1- carbonyl)spiro[in- doline-3,3′- pyrrolidin]-2- one isomer 4 | [M + H]+: 369, 371 (3:2); 1H NMR (400 MHz, CD3OD) δ 7.42 (d, J = 23.9 Hz, 1H), 7.10 (d, J = 5.2 Hz, 1H), 4.34-4.45 (m, 1H), 3.95-4.12 (m, 1H), 3.64-3.95 (m, 3H), 3.10-3.20 (m, 1H), 2.27-2.48 (m, 2H), 2.09-2.27 (m, 1H), 1.90-2.09 (m, 3H), 1.76-1.90 (m, 1H), 1.59-1.76 (m, 2H). |
| 67 | 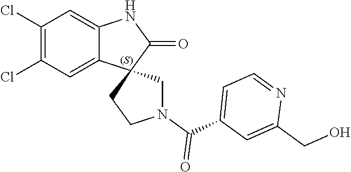 | (S)-5,6- dichloro-1′-(2- (hydroxymethyl) isonicotinoyl)spi- ro[indoline- 3,3′-pyrrolidin]- 2-one | [M + H]+: 392, 394 (3:2); 1H NMR (300 MHz, CD3OD) δ 8.61 (dd, J = 20.5, 5.0 Hz, 1H), 7.71 (d, J = 27.2 Hz, 1H), 7.39-7.61 (m, 2H), 7.09 (d, J = 18.5 Hz, 1H), 4.76 (d, J = 1 8.9 Hz, 2H), 3.74-4.19 (m, 4H), 2.28-2.5 (m, 2H). |
| 68 | 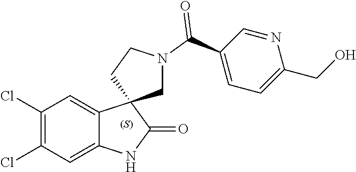 | (S)-5,6- dichloro-1′-(6- (hydroxymethyl) nicotinoyl)spiro [indoline-3,3′- pyrrolidin]-2- one | [M + H]+: 392, 394 (3:2); 1H NMR (400 MHz, CD3OD) δ 8.63-8.81 (m, 1H), 8.00-8.18 (m, 1H), 7.67 (dd, J = 29.0, 8.1 Hz, 1H), 7.52 (d, J = 64.5 Hz, 1H), 7.09 (d, J = 24.5 Hz, 1H), 4.75 (d, J = 22.7 Hz, 2H), 3.96-4.18 (m, 2H), 3.78-3.96 (m, 2H), 2.22-2.52 (m, 2H). |
| 69 | 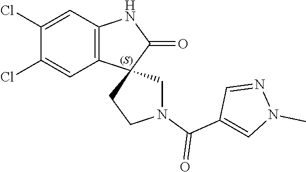 | (S)-5,6- dichloro-1′-(1- methyl-1H- pyrazole-4- carbonyl)spiro[in- doline-3,3′- pyrrolidin]-2- one | [M + H]+: 365, 367 (3:2); 1H NMR (300 MHz, DMSO- d6) δ 10.79-10.76 (brs, 1H), 8.21 (d, J = 28.09 Hz, 1H), 7.81 (d, J = 25.91 Hz, 1H), 7.59 (d, J = 23.11 Hz, 1H), 7.05 (s, 1H), 3.65-4.14 (m, 7H), 2.15-2.39 (m, 2H). |
| 70 | 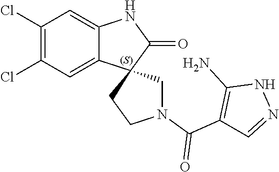 | (S)-1′-(5- amino-1H- pyrazole-4- carbonyl)-5,6- dichlorospiro[in- doline-3,3′- pyrrolidin]-2- one | [M + H]+: 366, 368 (3:2); 1H NMR (300 MHz, CD3OD) δ 8.02 (s, 1H), 7.47 (s, 1H), 7.11 (d, J = 2.04 Hz, 1H), 3.70-4.34 (m, 4H), 2.25-2.59 (m, 2H). |
| 71 | 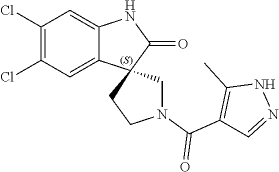 | (S)-5,6- dichloro-1′-(5- methyl-1H- pyrazol-4- carbonyl)spiro[in- doline-3,3′- pyrrolidin]-2- one | [M + H]+: 365, 367 (3:2); 1H NMR (300 MHz, CD3OD) δ 7.66-8.19 (m, 1H), 7.46 (d, J = 35.84 Hz, 1H), 7.10 (s, 1H), 3.77-4.25 (m, 4H), 2.17-2.62 (m, 5H). |
| 72 | 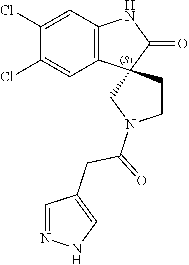 | (S)-1′-(2-(1H- pyrazol-4- yl)acetyl)-5,6- dichlorospiro[in- doline-3,3′- pyrrolidin]-2- one | [M + H]+: 365, 367 (3:2); 1H NMR (400 MHz, CD3OD) δ 7.58 (d, J = 31.38 Hz, 2H), 7.33 (d, J = 10.68 Hz, 1H), 7.09 (s, 1H), 3.93- 4.12 (m, 1H), 3.593.93 (m, 5H), 2.162.47 (m, 2H). |
| 73 | 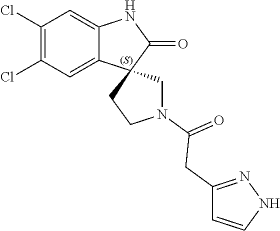 | (S)-1′-(2-(1H- pyrazol-3- yl)acetyl)-5,6- dichlorospiro[in- doline-3,3′- pyrrolidin]-2- one | [M + H]+: 365, 367 (3:2); 1H NMR (300 MHz, CD3OD) δ 7.62 (d, J = 20.91 Hz, 1H), 7.29 (d, J = 36.09 Hz, 1H), 7.09 (d, J = 1.54 Hz, 1H), 6.22-6.41 (m, 1H), 3.67-4.17 (m, 6H), 2.12-2.56 (m, 2H). |
| 74 | 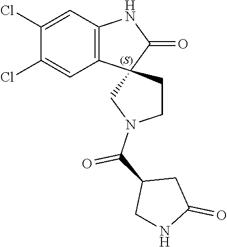 | (S)-5,6- dichloro-1′-(5- oxopyrrolidine- 3- carbonyl)spiro[in- doline-3,3′- pyrrolidin]-2- one isomer 1 | [M + H]+: 368, 370 (3:2); 1H NMR (300 MHz, CD3OD) δ 7.48 (dd, J = 11.49, 1.76 Hz, 1H), 7.11 (d, J = 2.62 Hz, 1H), 3.91-4.13 (m, 1H), 3.76-3.91 (m, 2H), 3.54-3.76 (m, 4H), 2.50-2.70 (m, 2H), 2.19-2.47 (m, 2H). |
| 75 | 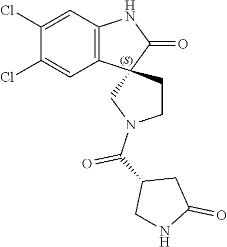 | (S)-5,6- dichloro-1′-(5- oxopyrrolidine- 3- carbonyl)spiro[in- doline-3,3′- pyrrolidin]-2- one isomer 2 | [M + H]+: 368, 370 (3:2); 1H NMR (300 MHz, CD3OD) δ 7.47 (dd, J = 24.90, 1.94 Hz, 1H), 7.11 (d, J = 3.06 Hz, 1H), 3.89-4.08 (m, 2H), 3.62-3.89 (m, 3H), 3.59 (d, J = 2.82 Hz, 2H), 2.55-2.71 (m, 2H), 2.18-2.49 (m, 2H). |
| 76 | 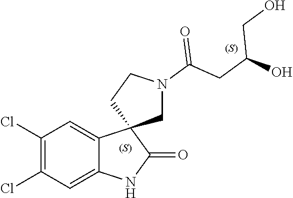 | (S)-5,6- dichloro-1′- ((S)-3,4- dihydroxybutan- oyl)spiro[indoline- 3,3′- pyrrolidin]-2- one | [M + H]+: 359, 361 (3:2); 1H NMR (300 MHz, CD3OD) δ 7.49 (dd, J = 40.69, 1.78 Hz, 1H), 7.10 (d, J = 2.00 Hz, 1H), 3.99-4.22 (m, 2H), 3.68-3.98 (m, 3H), 3.57 (dd, J = 15.21, 5.27 Hz, 2H), 2.48-2.76 (m, 2H), 2.35-2.48 (m, 1H), 2.17-2.35 (m, 1H). |
| 77 | 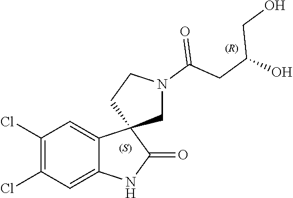 | (S)-5,6- dichloro-1′- ((R)-3,4- dihydroxybutan- oyl)spiro[indoline- 3,3′- pyrrolidin]-2- one | [M + H]+: 359, 361 (3:2); 1H NMR (300 MHz, CD3OD) δ 7.41-7.53 (m, 1H), 7.10 (d, J = 1.82 Hz, 1H), 3.94-4.2 (m, 2H), 3.84- 3.94 (m, 2H), 3.65-3.82 (m, 1H), 3.57 (dd, J = 19.96, 5.24 Hz, 2H), 2.56-2.78 (m, 1H), 2.53 (d, J = 6.44 Hz, 1H), 2.142.49 (m, 2H). |
| 78 | 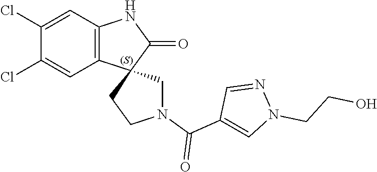 | (S)-5,6- dichloro-1′-(1- (2- hydroxyethyl)- 1H-pyrazole-4- carbonyl)spiro[in- doline-3,3′- pyrrolidin]-2- one | [M + H]+: 395, 397 (3:2); 1H NMR (400 MHz, CD3OD) δ 8.19 (d, J = 45.50 Hz, 1H), 7.96 (d, J = 37.15 Hz, 1H), 7.46 (d, J = 30.36 Hz, 1H), 7.10 (d, J = 7.05 Hz, 1H), 4.22-4.36 (m, 3H), 3.97-4.22 (m, 2H), 3.82-3.97 (m, 3H), 2.21-2.54 (m, 2H). |
| 79 | 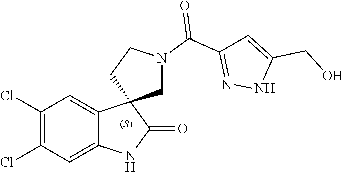 | (S)-5,6- dichloro-1′-(5- (hydroxymethyl)- 1H-pyrazole- 3- carbonyl)spiro[in- doline-3,3′- pyrrolidin]-2- one | [M + H]+: 381, 383 (3:2); 1H NMR (400 MHz, CD3OD) δ 7.46 (d, J = 38.43 Hz, 1H), 7.10 (d, J = 8.93 Hz, 1H), 6.73 (d, J = 21.96 Hz, 1H), 4.67 (d, J = 20.08 Hz, 2H), 4.37 (s, 1H), 4.24 (s, 1H), 3.83-4.13 (m, 2H), 2.21-2.50 (m, 2H). |
| 80 | 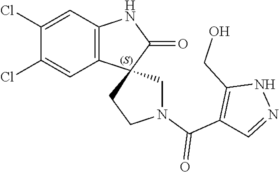 | (S)-5,6- dichloro-1′-(5- (hydroxymethyl)- 1H-pyrazole- 4- carbonyl)spiro[in- doline-3,3′- pyrrolidin]-2- one | [M + H]+: 381, 383 (3:2); 1H NMR (400 MHz, CD3OD) δ 8.04 (d, J = 70.97 Hz, 1H), 7.48 (d, J = 40.18 Hz, 1H), 7.10 (d, J = 9.24 Hz, 1H), 4.81 (s, 2H), 4.08- 4.29 (m, 1H), 3.82-4.08 (m, 3H), 2.24-2.53 (m, 2H). |
| 81 | 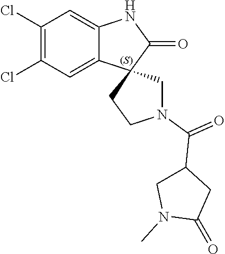 | (3S)-5,6- dichloro-1′-(1- methyl-5- oxopyrrolidine- 3- carbonyl)spiro[in- doline-3,3′- pyrrolidin]-2- one | [M + H]+: 382, 384 (3:2); 1H NMR (300 MHz, CD3OD) δ 7.43-7.55 (m, 1H), 7.10 (d, J = 3.51 Hz, 1H), 3.79-4.11 (m, 4H), 3.45-3.78 (m, 3H), 2.82-2.94 (m, 3H), 2.60-2.78 (m, 2H), 2.21-2.50 (m, 2H). |
| 82 | 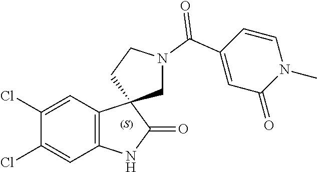 | (S)-5,6- dichloro-1′-(1- methyl-2-oxo- 1,2- dihydropyridine- 4- carbonyl)spiro[in- doline-3,3′- pyrrolidin]-2- one | [M + H]+: 392, 394 (3:2); 1H NMR (400 MHz, CD3OD) δ 7.77 (dd, J = 30.07, 6.86 Hz, 1H), 7.50 (d, J = 53.68 Hz, 1H), 7.09 (d, J = 16.40 Hz, 1H), 6.67 (dd, J = 41.91, 1.82 Hz, 1H), 6.38- 6.54 (m, 1H), 3.91-4.11 (m, 2H), 3.75-3.91 (m, 2H), 3.60 (d, J = 25.30 Hz, 3H), 2.25- 2.47 (m, 2H). |
| 83 | 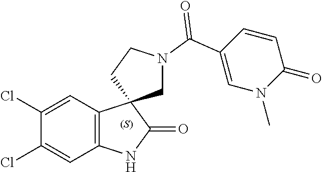 | (S)-5,6- dichloro-1′-(1- methyl-6-oxo- 1,6- dihydropyridine- 3- carbonyl)spiro[in- doline-3,3′- pyrrolidin]-2- one | [M + H]+: 392, 394 (3:2); 1H NMR (400 MHz, CD3OD) δ 8.04-8.3 (m, 1H), 7.81 (s, 1H), 7.50 (d, J = 68.21 Hz, 1H), 7.10 (s, 1H), 6.58 (s, 1H), 3.76-4.2 (m, 4H), 3.5-3.76 (m, 3H), 2.36- 2.49 (m, 1H), 2.24-2.36 (m, 1H). |
| 84 | 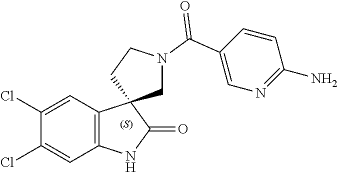 | (S)-1′-(6- aminonicotinoyl)- 5,6- dichlorospiro[in- doline-3,3′- pyrrolidin]-2- one | [M + H]+: 377, 379 (3:2); 1H NMR (300 MHz, DMSO- d6 + D2O) δ 8.19 (s, 1H), 7.28-7.90 (m, 2H), 7.08 (s, 1H), 6.45 (d, J = 8.71 Hz, 1H), 3.71-4.03 (m, 4H), 2.05-2.33 (m, 2H). |
| 85 | 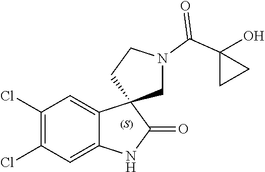 | (S)-5,6- dichloro-1′-(1- hydroxycyclopro- pane-1- carbonyl)spiro[in- doline-3,3′- pyrrolidin]-2- one | [M + H]+: 341, 343 (3:2); 1H NMR (400 MHz, CD3OD) δ 7.40 (d, J = 60.30 Hz, 1H), 7.11 (s, 1H), 4.27- 4.42 (m, 1H), 4.15 (s, 1H), 3.66-3.99 (m, 2H), 2.13-2.5 (m, 2H), 1.07-1.34 (m, 2H), 0.87-1.03 (m, 2H). |
| 86 | 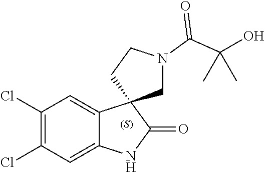 | (S)-5,6- dichloro-1′-(2- hydroxy-2- methylpropanoyl) spiro[indoline- 3,3′- pyrrolidin]-2- one | [M + H]+: 343, 345 (3:2); 1H NMR (300 MHz, CD3OD) δ 7.40 (d, J = 53.14 Hz, 1H), 7.11 (s, 1H), 4.20- 4.40 (m, 1H), 4.02-4.20 (m, 1H), 3.65-3.98 (m, 2H), 2.04-2.47 (m, 2H), 1.49 (d, J = 3.87 Hz, 3H), 1.41 (d, J = 10.45 Hz, 3H). |
| 87 | 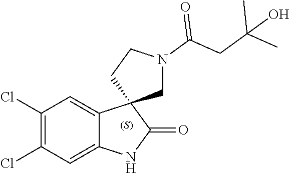 | (S)-5,6- dichloro-1′-(3- hydroxy-3- methylbutanoyl) spiro[indoline- 3,3′-pyrrolidin]- 2-one | [M + H]+: 357, 359 (3:2); 1H NMR (400 MHz, DMSO- d6) δ 10.79 (s, 1H), 7.57 (d, J = 49.72 Hz, 1H), 7.05 (d, J = 4.28 Hz, 1H), 4.84 (d, J = 30.14 Hz, 1H), 3.85-3.94 (m, 1H), 3.79 (q, J = 10.83 Hz, 1H), 3.70 (t, J = 7.20 Hz, 1H), 3.52-3.66 (m, 1H), 2.35-2.43 (m, 2H), 2.10-2.31 (m, 2H), 1.23 (d, J = 2.91 |
| | | | Hz, 3H), 1.18 (d, J = 1.16 |
| | | | Hz, 3H). |
| 88 | 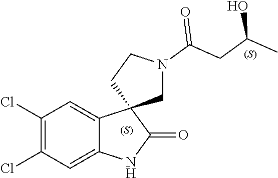 | (S)-5,6- dichloro-1′- ((S)-3- hydroxybutanoyl) spiro[indoline- 3,3′- pyrrolidin]-2- one | [M + H]+: 343, 345 (3:2); H NMR (400 MHz, CD3OD) δ 7.48 (d, J = 51.60 Hz, 1H), 7.10 (d, J = 3.23 Hz, 1H), 4.18-4.34 (m, 1H), 4.03 (t, J = 7.18 Hz, 1H), 3.87-3.96 (m, 1H), 3.76-3.87 (m, 1H), 3.74 (d, J = 2.13 Hz, 1H), 2.67 (dd, J = 14.67, 8.68 Hz, 1H), 2.38-2.55 (m, 2H), 2.16-2.38 (m, 1H), 1.27 |
| | | | (dd, J = 25.35, 6.24 Hz, 3H). |
| 89 | 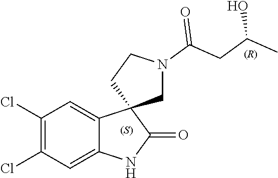 | (S)-5,6- dichloro-1′- ((R)-3- hydroxybutanoyl) spiro[indoline- 3,3′- pyrrolidin]-2- one | [M + H]+: 343, 345 (3:2); 1H NMR (400 MHz, CD3OD) δ 7.48 (d, J = 51.55 Hz, 1H), 7.10 (d, J = 3.20 Hz, 1H), 4.19-4.34 (m, 1H), 4.03 (t, J = 7.17 Hz, 1H), 3.88-3.96 (m, 1H), 3.79-3.88 (m, 1H), 3.74 (d, J = 2.18 Hz, 1H), 2.67 (dd, J = 14.68, 8.69 Hz, 1H), 2.39-2.57 (m, 2H), 2.16-2.39 (m, 1H), 1.27 |
| | | | (dd, J = 25.38, 6.26 Hz, 3H). |
| 90 | 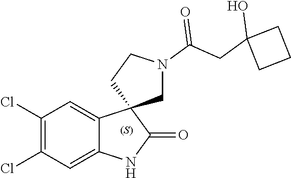 | (S)-5,6- dichloro-1′-(2- (1- hydroxycyclobu- tyl)acetyl)spiro[in- doline-3,3′- pyrrolidin]-2- one | [M + H]+: 369, 371 (3:2); 1H NMR (400 MHz, CD3OD) δ 7.48 (d, J = 49.03 Hz, 1H), 7.10 (d, J = 1.88 Hz, 1H), 4.06 (t, J = 71.9 Hz, 1H), 3.92 (s, 1H), 3.69-3.89 (m, 2H), 2.65-2.85 (m, 2H), 2.22-2.48 (m, 3H), 2.05-2.22 (m, 3H), 1.73-1.88 (m, 1H), 1.59-1.73 (m, 1H). |
| 91 | 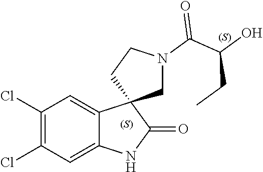 | (S)-5,6- dichloro-1′- ((S)-2- hydroxybutanoyl) spiro[indoline- 3,3′- pyrrolidin]-2- one | [M + H]+: 343, 345 (3:2); 1H NMR (300 MHz, CD3OD) δ 7.45 (d, J = 28.30 Hz, 1H), 7.10 (d, J = 2.06 Hz, 1H), 4.16-4.39 (m, 1H), 3.89-4.09 (m, 2H), 3.73-3.89 (m, 2H), 2.18-2.50 (m, 2H), 1.55-1.93 (m, 2H), 1.02 (dt, J = 17.69, 7.39 Hz, 3H). |
| 92 | 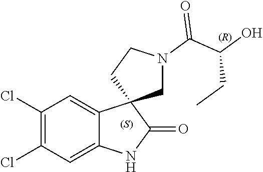 | (S)-5,6- dichloro-1′- ((R)-2- hydroxybutanoyl) spiro[indoline- 3,3′- pyrrolidin]-2- one | [M + H]+: 343, 345 (3:2); 1H NMR (400 MHz, CD3OD) δ 7.40 (d, J = 30.73 Hz, 1H), 7.11 (d, J = 2.51 Hz, 1H), 4.18-4.39 (m, 1H), 3.92-4.17 (m, 1H), 3.63-3.92 (m, 3H), 2.15-2.50 (m, 2H), 1.55-1.91 (m, 2H), 1.03 (dt, J = 29.88, 7.44 Hz, 3H). |
| 135 | 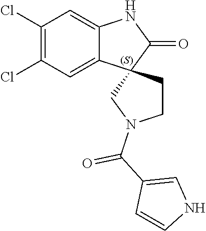 | (S)-5,6- dichloro-1′- (1H-pyrrole-3- carbonyl)spiro[in- doline-3,3′- pyrrolidin]-2- one | [M + H]⁺: 350, 352 (3:2); ¹H NMR (400 MHz, DMSO-d₆) δ 11.26 (brs, 1H), 10.77 (brs, 1H), 7.53 (d, J = 32.15 Hz, 1H), 7.28 (d, J = 33.79 Hz, 1H), 7.05 (s, 1H), 6.78 (s, 1H), 6.44 (d, J = 23.37 Hz, 1H), 4.22-3.93 (m, 2H), 3.81-3.58 (m, 2H), 2.39-2.11 (m, 2H). |
| 136 | 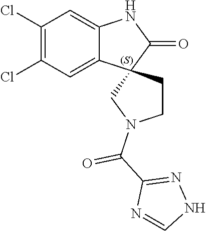 | (S)-5,6- dichloro-1′- (1H-1,2,4- triazole-3- carbonyl)spiro[in- doline-3,3′- pyrrolidin]-2- one | [M + H]⁺: 352, 354 (3:2); ¹H NMR (300 MHz, CD₃OD) δ 8.46 (d, J = 24.55 Hz, 1H), 7.49 (d, J = 22.96 Hz, 1H), 7.11 (d, J = 5.59 Hz, 1H), 4.45 (t, J = 7.18 Hz, 1H), 4.32 (q, J = 12.23 Hz, 1H), 4.20-3.85 (m, 2H), 2.55-2.23 (m, 2H). |
| 137 | 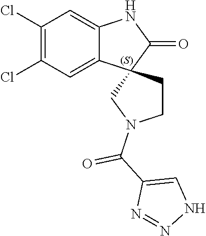 | (S)-5,6- dichloro-1′- (1H-1,2,3- triazole-4- carbonyl)spiro[in- doline-3,3′- pyrrolidin]-2- one | [M + H]⁺: 352, 354 (3:2); ¹H NMR (300 MHz, CD₃OD) δ 8.28 (s, 1H), 7.49 (d, J = 17.28 Hz, 1H), 7.11 (d, J = 5.23 Hz, 1H), 4.53-4.23 (m, 2H), 4.20-3.83 (m, 2H ), 2.57-2.23 (m, 2H). |
| 141 | 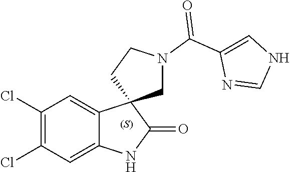 | (S)-5,6- dichloro-1′- (1H-imidazole- 4- carbonyl)spiro[in- doline-3,3′- pyrrolidin]-2- one | [M + H ]⁺: 351, 353 (3:2); ¹H NMR (400 MHz, CD₃OD) δ 7.77 (d, J = 37.07 Hz, 2H), 7.48 (d, J = 45.19 Hz, 1H), 7.10 (d, J = 8.00 Hz, 1H), 4.46-4.15 (m, 2H), 4.15-3.81 (m, 2H), 2.55-2.21 (m, 2H). |
| 147 | 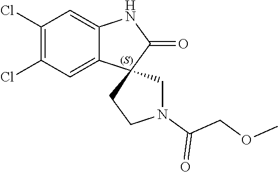 | (S)-5,6- dichloro-1′-(2- methoxyacetyl) spiro[indoline- 3,3′-pyrrolidin]- 2-one | [M + H]⁺; 329, 331 (3:2); ¹H NMR (400 MHz, CD₃OD) δ 7.46 (d, J = 26.93 Hz, 1H), 7.10 (d, J = 1.18 Hz, 1H), 4.23 (q, J = 14.80 Hz, 1H), 4.17-4.07 (m, 1H), 4.00-3.83 (m, 2H), 3.83-3.68 (m, 2H), 2.45 (d, J = 24.29 Hz, 3H), 2.47-2.16 (m, 2H). |
| 150 | 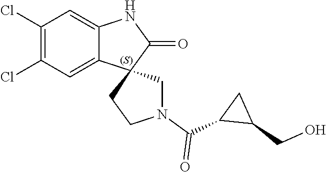 | (S)-5,6- dichloro-1′- (1R,2R)-rel-2- (hydroxymethyl) cyclopropane- 1- carbonyl)spiro[in- doline-3,3′- pyrrolidin]-2- one | [M + H]⁺: 355, 357 (3:2); ¹H NMR (300 MHz, CD₃OD) δ 7.52-7.41 (m, 1H), 7.11 (d, J = 2.93 Hz, 1H), 4.29-4.08 (m, 1H), 4.08-3.80 (m, 2H), 3.80-3.58 (m, 2H), 3.49-3.37 (m, 1H), 2.53-2.15 (m, 2H), 1.93-1.56 (m, 2H), 1.24-1.14 (m, 1H), 1.01-0.79 (m, 1H). |
| 151 | 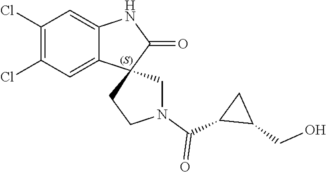 | (S)-5,6- dichloro-1′- (1R,2S)-rel-2- (hydroxymethyl) cyclopropane- 1- carbonyl)spiro[in- doline-3,3′- pyrrolidin]-2- one | [M + H]⁺: 355, 357 (3:2); ¹H NMR (300 MHz, CD₃OD) δ 7.63-7.37 (m, 1H), 7.14-7.05 (m, 1H), 4.28-4.02 (m, 1H), 4.03-3.77 (m, 2H), 3.77-3.53 (m, 2H), 3.54-3.35 (m, 1H), 2.52-1.84 (m, 3H), 1.79-1.52 (m, 1H), 1.16-0.95 (m, 2H). |
| 152 | 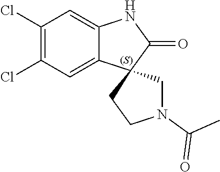 | (S)-1′-acetyl- 5,6- dichlorospiro[in- doline-3,3′- pyrrolidin]-2- one | [M + H]⁺: 299, 301 (3:2); ¹H NMR (400 MHz, CD₃OD) δ 7.46 (d, J = 22.43 Hz, 1H), 7.10 (d, J = 2.57 Hz, 1H), 4.07-3.62 (m, 4H), 2.47-2.19 (m, 2H), 2.19-2.04 (m, 3H). |
| 156 | 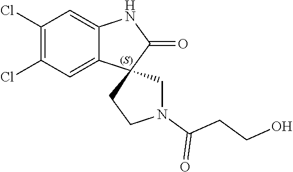 | (S)-5,6- dichloro-1′-(3- hydroxypropan- oyl)spiro[indoline- 3,3′- pyrrolidin]-2- one | [M + H]⁺: 329, 331 (3:2); ¹H NMR (400 MHz, CD₃OD) δ 7.48 (d, J = 38.68 Hz, 1H), 7.10 (d, J = 2.07 Hz, 1H), 4.09-3.98 (m, 1H), 3.97-3.80 (m, 4H), 3.75 (q, J = 12.27 Hz, 1H), 2.81-2.49 (m, 2H), 2.49-2.16 (m, 2H). |
| 157 | 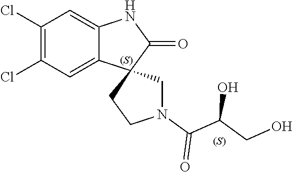 | (S)-5,6- dichloro-1′-((S)- 2,3- dihydroxypropa- noyl)spiro[indoline- 3,3′- pyrrolidin]-2- one | [M + H]⁺: 345, 347 (3:2); ¹H NMR (300 MHz, CD₃OD) δ 7.48 (d, J = 23.23 Hz, 1H), 7.10 (s, 1H), 4.43 (dt, J = 36.17, 5.63 Hz, 1H), 4.12 (t, J = 7.15 Hz, 1H), 4.05-3.89 (m, 1H), 3.89-3.65 (m, 4H), 2.51-2.15 (m, 2H). |
| 158 | 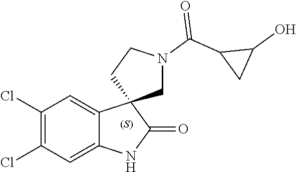 | (3S)-5,6- dichloro-1′-(2- hydroxycyclopro- pane-1- carbonyl)spiro[in- doline-3,3′- pyrrolidin]-2- one | [M + H]⁺: 341, 343 (3:2); ¹H NMR (300 MHz, DMSO-d₆) δ 10.78 (brs, 1H), 9.70 (d, J = 8.86 Hz, 1H), 7.59 (d, J = 25.41 Hz, 1H), 7.06 (d, J = 4.04 Hz, 1H), 3.87 (t, J = 7.05 Hz, 1H), 3.78-3.61 (m, 2H), 3.61-3.50 (m, 1H), 2.78-2.55 (m, 4H), 2.35-2.10 (m, 2H). |
| 159 | 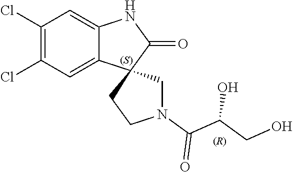 | (S)-5,6- dichloro-1′- ((R)-2,3- dihydroxypropa- noyl)spiro[indoline- 3,3′- pyrrolidin]-2- one | [M + H]⁺: 345, 347 (3:2); ¹H NMR (300 MHz, CD₃OD) δ 7.47 (d, J = 55.53 Hz, 1H), 7.11 (d, J = 2.71 Hz, 1H), 4.57-4.31 (m, 1H), 4.21-4.01 (m, 1H), 4.01-3.83 (m, 2H), 3.83-3.63 (m, 3H), 2.53-2.12 (m, 2H). |

### Example 8. Compound 93 ((3S)-5,6-dichloro-1′-[(1r,3r)-3-hydroxy-3-(hydroxymethyl)cyclobutanecarbonyl]-1H-spiro[indole-3,3′-pyrrolidin]-2-one) and Compound 94 ((3S)-5,6-dichloro-1′-[(1s,3s)-3-hydroxy-3-(hydroxymethyl)cyclobutanecarbonyl]-1H-spiro[indole-3,3′-pyrrolidin]-2-one))

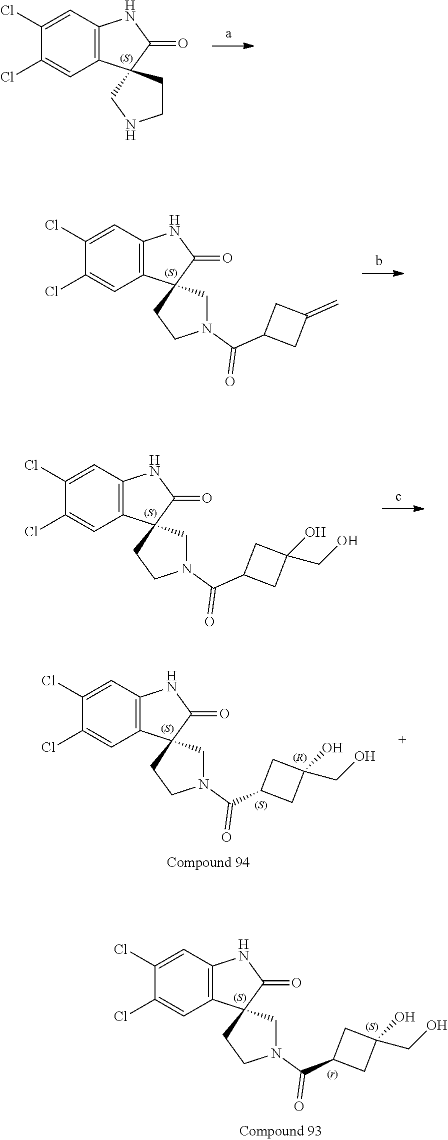

### Step a:

To a stirred solution of 3-methylidenecyclobutane-1-carboxylic acid (38.0 mg, 0.340 mmol) in DMF (1 mL) were added EDCI (89.0 mg, 0.470 mmol) and HOBT (63.0 mg, 0.470 mmol) at room temperature. To the above mixture was added TEA (94.0 mg, 0.930 mmol) and (3S)-5,6-dichloro-1H-spiro[indole-3,3′-pyrrolidin]-2-one (80.0 mg, 0.310 mmol). The reaction mixture was stirred for 1.5 h, diluted with water (20 mL) and extracted with EA (3×20 mL). The combined organic layers were washed with brine (3×20 mL) and dried over anhydrous Na₂SO₄. After filtration, the filtrate was concentrated under reduced pressure to afford (3S)-5,6-dichloro-1′-(3-methylidenecyclobutanecarbonyl)-1H-spiro[indole-3,3′-pyrrolidin]-2-one as an off-white solid (0.120 g, 62%), which was used in the next step without purification: LCMS (ESI) calc'd for C₁₇H₁₆Cl₂N₂O₂ [M+H]⁺: 351, 353 (3:2), found 351, 353 (3:2).

### Step b:

To a stirred solution of (3S)-5,6-dichloro-1′-(3-methylidenecyclobutanecarbonyl)-1H-spiro[indole-3,3′-pyrrolidin]-2-one (80.0 mg, 0.230 mmol) in THE (0.5 mL), acetone (0.5 mL) and H₂O (0.5 mL) were added NMO (0.160 g, 1.37 mmol) and K₂OsO₄·2H₂O (8.39 mg, 0.02 mmol) at room temperature. The reaction mixture was stirred for 3 h, quenched with saturated aq. Na₂SO₃ (0.5 mL) and concentrated under reduced pressure. The residue was purified by Prep-HPLC with the following conditions: Column: XBridge Prep C18 OBD Column, 19×150 mm, 5 μm; Mobile Phase A: Water (plus 10 mmol/L NH₄HCO₃), Mobile Phase B: ACN; Flow rate: 20 mL/min; Gradient: 30% B to 50% B in 4.5 min, 50% B; Wavelength: UV 254/210 nm; Retention Time: 4.35 min. The fractions containing the desired product were collected and concentrated under reduced pressure to afford (3S)-5,6-dichloro-1′-[3-hydroxy-3-(hydroxymethyl)cyclobutanecarbonyl]-1H-spiro[indole-3,3′-pyrrolidin]-2-one as an off-white solid (61.0 mg, 69%): LCMS (ESI) calc'd for C₁₇H₁₈Cl₂N₂O₄ [M+H]⁺: 385, 387 (3:2), found 385, 387 (3:2); ¹H NMR (400 MHz, DMSO-d₆) δ 10.75 (s, 1H), 7.51 (dd, J=36.2, 10.1 Hz, 1H), 7.05 (dd, J=4.1, 1.1 Hz, 1H), 4.97-4.76 (m, 1H), 4.63-4.44 (m, 1H), 3.78-3.35 (m, 4H), 3.31-3.08 (m, 3H), 2.34-2.18 (m, 2H), 2.18-1.87 (m, 3H).

### Step c:

(3S)-5,6-dichloro-1′-[3-hydroxy-3-(hydroxymethyl)cyclobutanecarbonyl]-1H-spiro[indole-3,3′-pyrrolidin]-2-one (57.0 mg, 0.150 mmol) was separated by Prep Chiral HPLC with the following conditions: Column: Lux 5 μm Cellulose-4, 2.12×25 cm, 5 μm; Mobile Phase A: Hex (plus 0.5% 2M NH3-MeOH)-HPLC, Mobile Phase B: EtOH-HPLC; Flow rate: 20 mL/min; Gradient: 20% B to 20% B in 35 min; Wavelength: 220/254 nm; Retention time 1: 6.76 min; Retention time 2: 12.28 min; Sample Solvent: MeOH:EtOH=1:1-HPLC; Injection Volume: 0.6 mL; Number Of Runs: 4. The faster eluting isomer at 6.76 min was obtained (3S)-5,6-dichloro-1′-[(1s,3s)-3-hydroxy-3-(hydroxymethyl)cyclobutanecarbonyl]-1H-spiro[indole-3,3′-pyrrolidin]-2-one as an off-white solid (12.9 mg, 22%): LCMS (ESI) calc'd for C₁₇H₁₈Cl₂N₂O₄ [M+H]⁺: 385, 387 (3:2), found 385, 387 (3:2); ¹H NMR (400 MHz, DMSO-d₆) δ 10.79-10.72 (brs, 1H), 7.50 (d, J=35.98 Hz, 1H), 7.05 (d, J=3.91 Hz, 1H), 4.93 (d, J=15.89 Hz, 1H), 4.56 (d, J=47.76 Hz, 1H), 3.64-3.80 (m, 2H), 3.53-3.64 (m, 2H), 3.37 (s, 1H), 3.29 (s, 1H), 2.60-2.88 (m, 1H), 2.19-2.35 (m, 2H), 1.95-2.19 (nm, 4H). The slower eluting isomer at 12.28 min was obtained (3S)-5,6-dichloro-1′-[(1r,3r)-3-hydroxy-3-(hydroxymethyl)cyclobutanecarbonyl]-1H-spiro[indole-3,3′-pyrrolidin]-2-one as an off-white solid (13.6 mg, 23%): LCMS (ESI) calc'd for C₁₇H₈Cl₂N₂O₄ [M+H]⁺: 385, 387 (3:2), found 385, 387 (3:2); ¹H NMR (400 Mhz, DMSO-d₆) δ 10.78-10.72 (brs, 1H), 7.52 (d, J=36.80 Hz, 1H), 7.05 (d, J=4.16 Hz, 1H), 4.82 (d, J=27.34 Hz, 1H), 4.39-4.57 (m, 1H), 3.61-3.77 (m, 2H), 3.52-3.60 (m, 2H), 3.07-3.30 (m, 3H), 2.20-2.38 (m, 3H), 2.08-2.20 (m, 1H), 1.88-2.07 (m, 2H).

The compounds in Table 1B below were prepared in an analogous fashion to that described for Compound 93, starting from Intermediate 1S and 3-methylidenecyclopentane-1-carboxylic acid, which was commercially available.

TABLE 1B

| Compound | | | |
|---|---|---|---|
| Number | Structure | Chemical Name | MS: (M + H)⁺ & ¹H MNR |
| 95 | 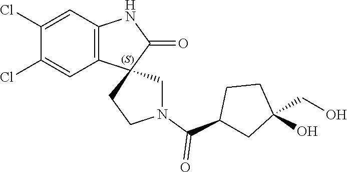 | (S)-5,6-dichloro- 1′-((1R,3S)-rel-3- hydroxy-3- (hydroxymethyl) cyclopentane-1- carbonyl)spiro [indoline-3,3′- pyrrolidin]-2-one isomer 1 | [M + H]⁺: 399, 401 (3:2); ¹H NMR (300 MHz, CD₃OD) δ 7.43 (d, J = 24.36 Hz, 1H), 7.10 (t, J = 2.70 Hz, 1H), 3.91-4.13 (m, 1H), 3.61-3.91 (m, 3H), 3.43-3.58 (m, 2H), 2.98-3.27 (m, 1H), 1.83-2.49 (m, 6H), 1.69-1.81 (m, 2H). |
| 96 | 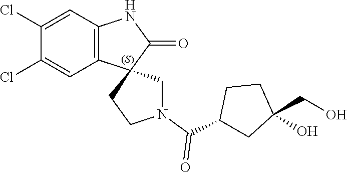 | (S)-5,6-dichloro- 1′-((1R,3S)-rel-3- hydroxy-3- (hydroxymethyl) cyclopentane-1- carbonyl)spiro [indoline-3,3′- pyrrolidin]-2-one isomer 2 | [M + H]⁺: 399, 401 (3:2); ¹H NMR (300 MHz, CD₃OD) δ 7.43 (dd, J = 14.24, 2.20 Hz, 1H), 7.11 (d, J = 2.69 Hz, 1H), 3.91-4.15 (m, 1H), 3.63-3.92 (m, 3H), 3.42- 3.58 (m, 2H), 2.99-3.25 (m, 1H), 2.19-2.48 (m, 2H), 1.94-2.19 (m, 3H), 1.64-1.94 (m, 3H). |
| 97 | 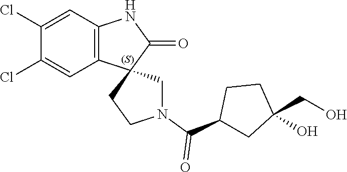 | (S)-5,6-dichloro- 1′-((1R,3R)-rel-3- hydroxy-3- (hydroxymethyl) cyclopentane-1- carbonyl)spiro [indoline-3,3′- pyrrolidin]-2-one isomer 1 | [M + H]⁺: 399, 401 (3:2); ¹H NMR (300 MHz, CD₃OD) δ 7.43 (dd, J = 16.94, 2.18 Hz, 1H), 7.10 (t, J = 3.26 Hz, 1H), 3.93-4.16 (m, 1H), 3.62-3.92 (m, 3H), 3.55 (d, J = 10.15 Hz, 2H), 3.36-3.46 (m, 0.5H), 3.17-3.30 (m, 0.5H), 2.29-2.48 (m, 2H), 2.13-2.29 (m, 1H), 1.79-2.13 (m, 4H), 1.59-1.74 (m, 1H). |
| 98 | 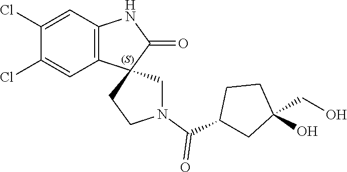 | (S)-5,6-dichloro- 1′-((1R,3R)-rel-3- hydroxy-3- (hydroxymethyl) cyclopentane-1- carbonyl)spiro [indoline-3,3′- pyrrolidin]-2-one isomer 2 | [M + H]⁺: 399, 401 (3:2); ¹H NMR (300 MHz, CD₃OD) δ 7.42 (dd, J = 21.49, 2.06 Hz, 1H), 7.03-7.19 (m, 1H), 3.92-4.15 (m, 1H), 3.61-3.92 (m, 3H), 3.54 (d, J = 7.61 Hz, 2H), 3.37-3.46 (m, 0.5H), 3.17-3.3 (m, 0.5H), 2.29- 2.49 (m, 2H), 2.02-2.29 (m, 1H), 1.58-2.02 (m, 5H). |

### Example 9. Compound 99 ((3S)-5,6-dichloro-1′-[(1R,3R)-rel-3-(hydroxymethyl)cyclopentanecarbonyl]-1H-spiro[indole-3,3′-pyrrolidin]-2-one isomer 1) and Compound 100 ((3S)-5,6-dichloro-1′-[(1R,3R)-rel-3-(hydroxymethyl)cyclopentanecarbonyl]-1H-spiro[indole-3,3′-pyrrolidin]-2-one isomer 2)

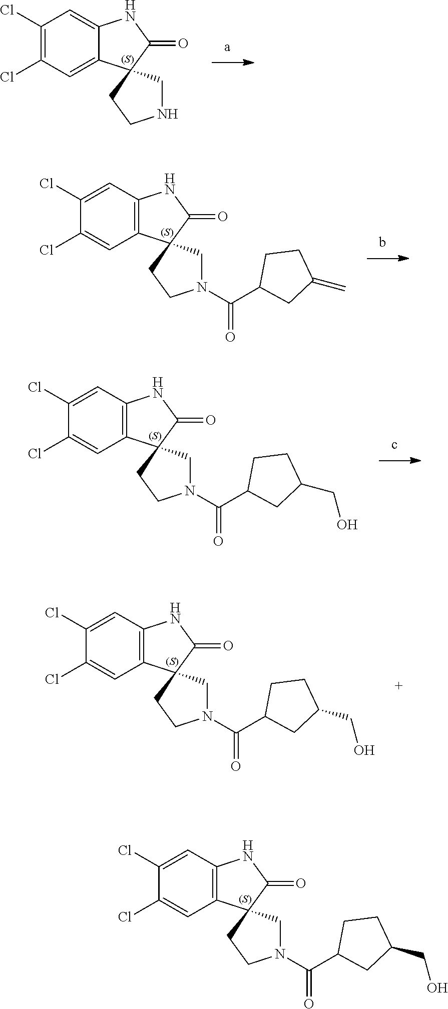

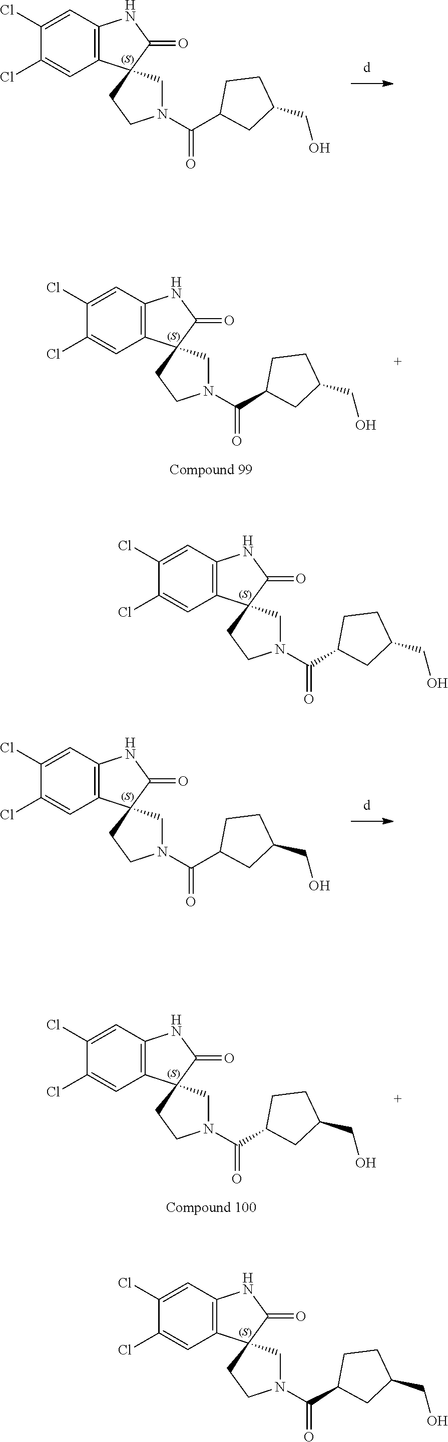

### Step a:

To a stirred solution of 3-methylidenecyclopentane-1-carboxylic acid (0.120 g, 0.930 mmol), EDCI (0.220 g, 1.17 mmol) and HOBT (0.160 g, 1.17 mmol) in DMF (3 mL) were added TEA (0.240 g, 2.33 mmol) and (3S)-5,6-dichloro-1H-spiro[indole-3,3′-pyrrolidin]-2-one (0.200 g, 0.780 mmol) at room temperature. The reaction mixture was stirred for 2 h, quenched with MeOH (0.5 mL) and purified by reverse phase chromatography, eluting with 50% ACN in water (plus 0.05% TFA) to afford (3S)-5,6-dichloro-1′-(3-methylidenecyclopentanecarbonyl)-1H-spiro[indole-3,3′-pyrrolidin]-2-one as a light yellow solid (0.200 g, 70%): LCMS (ESI) calc'd for C₁₈H₁₈Cl₂N₂O₂ [M+H]⁺: 365, 367 (3:2) found 365, 367 (3:2); ¹H NMR (300 MHz, CDCl₃) δ 8.14-8.37 (m, 1H), 7.23 (s, 1H), 7.09 (d, J=8.62 Hz, 1H), 4.85-4.99 (m, 2H), 3.82-4.14 (m, 3H), 3.65-3.82 (m, 1H), 2.78-3.11 (m, 1H), 2.46-2.78 (m, 4H), 2.19-2.46 (m, 2H), 1.88-2.19 (m, 2H).

### Step b:

To a stirred solution of (3S)-5,6-dichloro-1′-(3-methylidenecyclopentanecarbonyl)-1H-spiro[indole-3,3′-pyrrolidin]-2-one (0.150 g, 0.410 mmol) in THE (5 mL) was added BH₃-Me₂S (94 uL, 1.23 mmol, 10 M) dropwise at 0° C. under nitrogen atmosphere. The reaction solution was stirred at 0° C. for 2 h under nitrogen atmosphere. NaOH (41.0 mg, 1.03 mmol) and H₂O₂ (35.0 mg, 1.03 mmol, 30%) were then added to the reaction mixture, which was then stirred for 30 min, diluted with water (20 mL) and extracted with EA (3×20 mL). The combined organic layers were washed with brine (2×20 mL) and dried over anhydrous Na₂SO₄. After filtration, the filtrate was concentrated under reduced pressure. The residue was purified by Prep-HPLC with the following conditions: Column: XBridge Prep C18 OBD Column, 19×150 mm, 5 μm; Mobile Phase A: Water (plus 10 mmol/L NH₄HCO₃), Mobile Phase B: ACN; Flow rate: 20 mL/min; Gradient: 30% B to 50% B in 4.5 min, 50% B; Wavelength: 210 nm; Retention time: 4.35 min. The fractions containing the desired product were collected and concentrated under reduced pressure to afford (3S)-5,6-dichloro-1′-[3-(hydroxymethyl)cyclopentanecarbonyl]-1H-spiro[indole-3,3′-pyrrolidin]-2-one as an off-white solid (50.0 mg, 31%): LCMS (ESI) calc'd for C₁₈H₂₀Cl₂N₂O₃ [M+H]⁺: 383, 385 (3:2) found 383, 385 (3:2); ¹H NMR (400 MHz, CD₃OD) δ 7.36-7.47 (m, 1H), 7.10 (d, J=4.35 Hz, 1H), 3.94-4.12 (m, 1H), 3.62-3.93 (m, 3H), 3.40-3.58 (m, 2H), 2.88-3.18 (m, 1H), 2.10-2.47 (m, 3H), 1.28-2.09 (m, 6H).

### Step c:

(3S)-5,6-dichloro-1′-[3-(hydroxymethyl)cyclopentanecarbonyl]-1H-spiro[indole-3,3′-pyrrolidin]-2-one (50.0 mg, 0.130 mmol) was separated by Prep Chiral HPLC with the following conditions: Column: CHIRALPAK IF, 2×25 cm, 5 μm; Mobile Phase A: MtBE (plus 0.5% IPA)-HPLC, Mobile Phase B: EtOH-HPLC; Flow rate: 20 mL/min; Gradient: 10% B to 10% B in 30 min; Wavelength: UV 220/254 nm; Retention time 1: 16.34 min; Retention time 2: 24.17 min; Sample Solvent: EtOH-HPLC; Injection Volume: 0.75 mL; Number Of Runs: 4. Two peaks were isolated, each containing two isomers, the faster eluting peak 1 at 16.34 min was obtained as an off-white solid (18.0 mg, 36%); The slower-eluting peak 2 at 24.17 min was obtained as an off-white solid (18.0 mg, 36%).

### Step d:

Peak 1 (18.0 mg, 0.047 mmol) was separated by Prep Chiral-HPLC with the following conditions: Column: CHIRALPAK IG, 2×25 cm, 5 μm; Mobile Phase A: Hex (plus 0.5% 2 MNH3-MeOH)-HPLC, Mobile Phase B: EtOH-HPLC; Flow rate: 20 mL/min; Gradient: 50% B to 50% B in 21 min; Wavelength: UV 220/254 nm; Retention time 1: 9.31 min; Retention time 2: 16.17 min; Sample Solvent: EtOH-HPLC; Injection Volume: 1 mL; Number Of Runs: 2. The faster-eluting isomer at 9.31 min was obtained (3S)-5,6-dichloro-1′-[(1R,3R)-rel-3-(hydroxymethyl)cyclopentanecarbonyl]-1H-spiro[indole-3,3′-pyrrolidin]-2-one isomer 1 as an off-white solid (4.40 mg, 24%): LCMS (ESI) calc'd for C₁₈H₂₀Cl₂N₂O₃ [M+H]⁺: 383, 385 (3:2) found 383, 385 (3:2); ¹H NMR (400 MHz, CD₃OD) δ 7.42 (d, J=28.8 Hz, 1H), 7.10 (d, J=4.0 Hz, 1H), 4.11-3.95 (m, 1H), 3.93-3.65 (m, 3H), 3.54-3.40 (m, 2H), 3.15-2.96 (m, 1H), 2.46-2.18 (m, 3H), 2.08-1.63 (m, 4H), 1.46-1.30 (m, 2H). The slower-eluting isomer at 16.17 min was obtained (3S)-5,6-dichloro-1′-[(1R,3S)-rel-3-(hydroxymethyl)cyclopentanecarbonyl]-1H-spiro[indole-3,3′-pyrrolidin]-2-one isomer 1 as an off-white solid (5.00 mg, 27%): LCMS (ESI) calc'd for C₁₈H₂₀Cl₂N₂O₃ [M+H]⁺: 383, 385 (3:2) found 383, 385 (3:2); ¹H NMR (400 MHz, CD₃OD) δ 7.42 (d, J=30.4 Hz, 1H), 7.10 (d, J=4.6 Hz, 1H), 4.11-3.96 (m, 1H), 3.93-3.66 (m, 3H), 3.60-3.46 (m, 2H), 3.16-2.96 (m, 1H), 2.47-2.28 (m, 2H), 2.26-2.10 (m, 2H), 2.09-1.94 (m, 1H), 1.92-1.77 (m, 2H), 1.61-1.45 (m, 2H).

### Step e:

Peak 2 (18.0 mg, 0.05 mmol) was separated by Prep Chiral HPLC with the following conditions: Column: CHIRALPAK IG, 2×25 cm, 5 μm; Mobile Phase A: Hex (plus 0.5% 2 M NH₃-MeOH)-HPLC, Mobile Phase B: EtOH-HPLC; Flow rate: 20 mL/min; Gradient: 50% B to 50% B in 36 min; Wavelength: UV 220/254 nm; Retention time 1: 12.42 min; Retention time 2: 24.05 min; Sample Solvent: EtOH-HPLC; Injection Volume: 0.5 mL; Number Of Runs: 2. The faster-eluting isomer at 12.42 min was obtained (3S)-5,6-dichloro-1′-[(1R,3R)-rel-3-(hydroxymethyl)cyclopentanecarbonyl]-1H-spiro[indole-3,3′-pyrrolidin]-2-one isomer 2 as a light blue solid (4.60 mg, 25%): LCMS (ESI) calc'd for C₁₈H₂₀Cl₂N₂O₃ [M+H]⁺: 383, 385 (3:2) found 383, 385 (3:2); ¹H NMR (400 MHz, CD₃OD) δ 7.42 (d, J=25.8 Hz, 1H), 7.10 (d, J=4.6 Hz, 1H), 4.12-3.94 (m, 1H), 3.93-3.65 (m, 3H), 3.52-3.38 (m, 2H), 3.15-2.94 (m, 1H), 2.49-2.17 (m, 3H), 2.13-1.58 (m, 5H), 1.49-1.25 (m, 1H). The slower-eluting isomer at 24.05 min was obtained (3S)-5,6-dichloro-1′-[(1R,3S)-rel-3-(hydroxymethyl)cyclopentanecarbonyl]-1H-spiro[indole-3,3′-pyrrolidin]-2-one isomer 2 as a light blue solid (4.80 mg, 26): LCMS (ESI) calc'd for C₁₈H₂₀Cl₂N₂O₃ [M+H]⁺: 383, 385 (3:2) found 383, 385 (3:2); ¹H NMR (400 MHz, CD₃OD) δ 7.42 (d, J=24.4 Hz, 1H), 7.10 (d, J=3.9 Hz, 1H), 4.11-3.95 (m, 1H), 3.91-3.66 (m, 3H), 3.59-3.48 (m, 2H), 3.15-2.96 (m, 1H), 2.45-2.31 (m, 3H), 2.26-2.10 (m, 2H), 2.06-1.93 (m, 1H), 1.92-1.76 (m, 2H), 1.49-1.25 (m, 1H).

### Example 10. Compound 101 ((3S)-5,6-dichloro-1′-[(1S,3R,4S)-3,4-dihydroxycyclopentanecarbonyl]-1H-spiro[indole-3,3′-pyrrolidin]-2-one) and Compound 102 ((3S)-5,6-dichloro-1′-[(1R,3R,43)-3,4-dihydroxycyclopentanecarbonyl]-1H-spiro[indole-3,3′-pyrrolidin]-2-one)

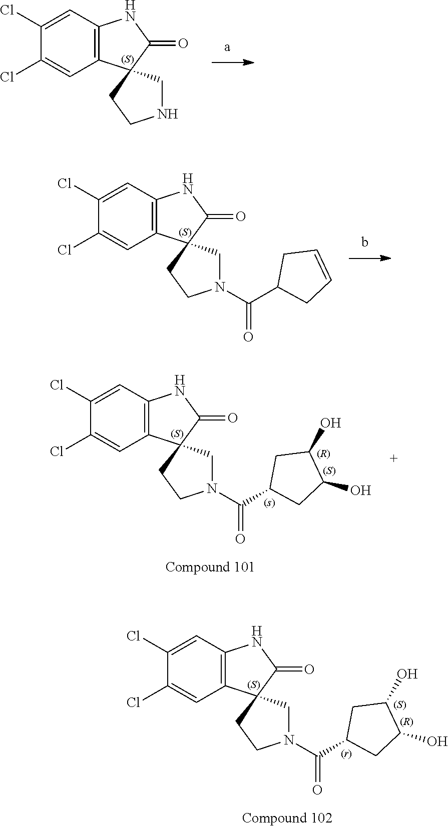

### Step a:

To a stirred solution of cyclopent-3-ene-1-carboxylic acid (52.0 mg, 0.470 mmol), EDCI (0.110 g, 0.580 mmol) and HOBT (79.0 mg, 0.580 mmol) in DMF (2 mL) were added TEA (79.0 mg, 0.780 mmol) and (3S)-5,6-dichloro-1H-spiro[indole-3,3′-pyrrolidin]-2-one (0.100 g, 0.390 mmol) at room temperature. The reaction mixture was stirred for 2 h, quenched with MeOH (0.5 mL) and purified by reverse phase chromatography, eluting with 50% ACN in water (plus 0.05% TFA) to afford (3S)-5,6-dichloro-1′-(cyclopent-3-ene-1-carbonyl)-1H-spiro[indole-3,3′-pyrrolidin]-2-one as a light yellow oil (0.110 g, 80%): LCMS (ESI) calc'd for C₁₇H₁₆Cl₂N₂O₂ [M+H]⁺: 351, 353 (3:2) found 351, 353 (3:2); ¹H NMR (400 MHz, DMSO-d₆) δ 10.77 (d, J=10.54 Hz, 1H), 7.56 (d, J=34.26 Hz, 1H), 7.06 (d, J=7.42 Hz, 1H), 5.54-5.77 (m, 2H), 3.83-3.96 (m, 1H), 3.78 (s, 1H), 3.67-3.74 (m, 1H), 3.58-3.67 (m, 2H), 3.14-3.41 (m, 1H), 2.53-2.69 (m, 3H), 2.12-2.32 (m, 2H).

### Step b:

To a stirred solution of (3S)-5,6-dichloro-1′-(cyclopent-3-ene-1-carbonyl)-1H-spiro[indole-3,3′-pyrrolidin]-2-one (0.110 g, 0.310 mmol) and NMO (0.110 g, 0.940 mmol) in THE (0.5 mL), acetone (0.5 mL) and H₂O (0.5 mL) was added K₂OsO₄·2H₂O (12.0 mg, 0.03 mmol) at room temperature. The reaction mixture was stirred for 1 h, quenched with saturated aq. Na₂S₂O₃ (10 mL) and extracted with EA (3×20 mL). The combined organic layers were washed with brine (2×20 mL) and dried over anhydrous Na₂SO₄. After filtration, the filtrate was concentrated under reduced pressure. The residue was purified by Prep-HPLC with the following conditions: Column: XBridge Prep C18 OBD Column, 19×150 mm, 5 μm; Mobile Phase A: Water (plus 10 mmol/L NH₄HCO₃), Mobile Phase B: ACN; Flow rate: 20 mL/min; Gradient: 40% B to 60% B in 4.5 min, 60% B; Wavelength: 254/210 nm; Retention time 1: 4.35 min, Retention time 2: 5.01 min. The faster-eluting isomer at 4.35 min was obtained (3S)-5,6-dichloro-1′-[(1S,3R,4S)-3,4-dihydroxycyclopentanecarbonyl]-1H-spiro[indole-3,3′-pyrrolidin]-2-one as an off-white solid (47.1 mg, 39%): LCMS (ESI) calc'd for C₁₇H₁₈Cl₂N₂O₄ [M+H]⁺: 385, 387 (3:2) found 385, 387 (3:2); ¹H NMR (400 MHz, DMSO-d₆) δ 10.69-10.60 (brs, 1H), 7.55 (d, J=33.7 Hz, 1H), 7.05 (d, J=5.7 Hz, 1H), 4.48-4.31 (m, 2H), 3.97-3.79 (m, 3H), 3.76-3.50 (m, 3H), 3.26-3.03 (m, 1H), 2.26 (t, J=7.1 Hz, 1H), 2.19-2.11 (m, 1H), 1.95-1.63 (m, 4H). The slower-eluting isomer at 5.01 min was obtained (3S)-5,6-dichloro-1′-[(1R,3R,4S)-3,4-dihydroxycyclopentanecarbonyl]-1H-spiro[indole-3,3′-pyrrolidin]-2-one as an off-white solid (2.50 mg, 2%): LCMS (ESI) calc'd for C₁₇H₁₈Cl₂N₂O₄ [M+H]⁺: 385, 387 (3:2) found 385, 387 (3:2); ¹H NMR (400 MHz, DMSO-d₆) δ 7.53 (d, J=32.7 Hz, 1H), 7.05 (d, J=5.9 Hz, 1H), 4.48-4.31 (m, 2H), 3.97-3.79 (m, 2H), 3.76-3.53 (m, 4H), 2.91-2.72 (m, 1H), 2.26 (t, J=7.1 Hz, 1H), 2.19-2.07 (m, 1H), 2.03-1.87 (m, 2H), 1.78-1.65 (m, 2H).

### Example 11. Compound 103 ((3S)-5,6-dichloro-1′-[(1R,(3R,4R)-rel)-3,4-dihydroxycyclohexanecarbonyl]-1H-spiro[indole-3,3′-pyrrolidin]-2-one isomer 1) and Compound 104 ((3S)-5,6-dichloro-1′-[(1R,(3R,4R)-rel)-3,4-dihydroxycyclohexanecarbonyl]-1H-spiro[indole-3,3′-pyrrolidin]-2-one isomer 2)

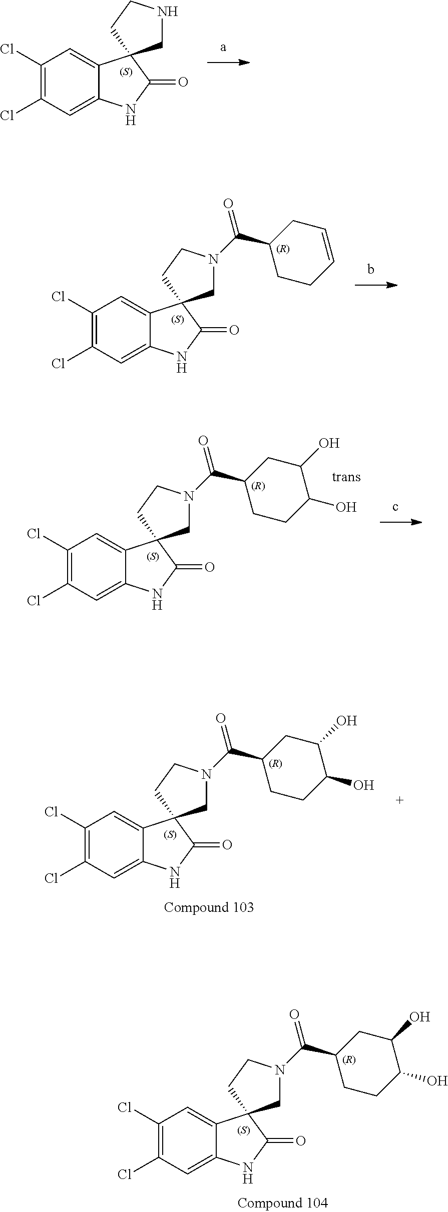

### Step a:

To a stirred solution of (1R)-cyclohex-3-ene-1-carboxylic acid (59.0 mg, 0.470 mmol), EDCI (0.110 g, 0.580 mmol) and HOBT (79.0 mg, 0.580 mmol) in DMF (2 mL) were added TEA (79.0 mg, 0.780 mmol) and (3S)-5,6-dichloro-1H-spiro[indole-3,3′-pyrrolidin]-2-one (0.100 g, 0.390 mmol) at room temperature. The reaction mixture was stirred for 1 h, quenched with MeOH (1 mL) and purified by reverse phase chromatography, eluting with 50% ACN in water (plus 0.05% TFA) to afford (3S)-5,6-dichloro-1′-[(1R)-cyclohex-3-ene-1-carbonyl]-1H-spiro[indole-3,3′-pyrrolidin]-2-one as an off-white solid (0.120 g, 84%): LCMS (ESI) calc'd for C₁₈H₁₈Cl₂N₂O₂ [M+H]⁺: 365, 367 (3:2) found 365, 367 (3:2); ¹H NMR (400 MHz, DMSO-d₆) δ 10.77 (d, J=8.2 Hz, 1H), 7.55 (d, J=45.7 Hz, 1H), 7.05 (d, J=6.7 Hz, 1H), 5.78-5.58 (m, 2H), 3.95-3.78 (m, 2H), 3.71-3.54 (m, 2H), 2.76-2.56 (m, 1H), 2.36-1.94 (m, 6H), 1.80 (dd, J=40.4, 13.0 Hz, 1H), 1.56-1.39 (m, 1H).

### Step b:

To a stirred solution of (3S)-5,6-dichloro-1′-[(1R)-cyclohex-3-ene-1-carbonyl]-1H-spiro[indole-3,3′-pyrrolidin]-2-one (0.150 g, 0.410 mmol) and H₂O₂ (0.5 mL, 6.44 mmol, 30%) in ACN (1 mL) and H₂O (1 mL,) was added HCOOH (0.5 mL) at room temperature. The reaction mixture was stirred at 50° C. for 4 h. Aq. NaOH (2 mL, 10M) was added dropwise to the mixture, which was then stirred at 40° C. for 4 h and concentrated under reduced pressure. The residue was purified by reverse phase chromatography, eluting with 30% ACN in water (plus 10 mmol/L NH₄HCO₃) to afford (3S)-5,6-dichloro-1′-[(1R)-(trans)-3,4-dihydroxycyclohexanecarbonyl]-1H-spiro[indole-3,3′-pyrrolidin]-2-one as an off-white solid (80.0 mg, 48%), which was used in the next step without purification: LCMS (ESI) calc'd for C₁₈H₂₀Cl₂N₂O₄ [M+H]⁺: 399, 401 (3:2) found 399, 401 (3:2).

### Step c:

(3S)-5,6-dichloro-1′-[(1R)-(trans)-3,4-dihydroxycyclohexanecarbonyl]-1H-spiro[indole-3,3′-pyrrolidin]-2-one (80.0 mg, 0.200 mmol) was separated by Prep Chiral HPLC with the following conditions: Column: CHIRAL ART Cellulose-SB, 2×25 cm, 5 μm; Mobile Phase A: Hex (plus 0.5% 2M NH3-MeOH)-HPLC, Mobile Phase B: EtOH-HPLC; Flow rate: 20 mL/min; Gradient: 30% B to 30% B in 24 min; Wavelength: UV 254/220 nm; Retention time 1: 9.18 min; Retention time 2: 20.67 min; Sample Solvent: EtOH-HPLC; Injection Volume: 1.2 mL; Number Of Runs: 3. The faster-eluting isomer at 9.18 min was obtained (3S)-5,6-dichloro-1′-[(1R,(3R,4R)-rel)-3,4-dihydroxycyclohexanecarbonyl]-1H-spiro[indole-3,3′-pyrrolidin]-2-one isomer 1 as an off-white solid (26.2 mg, 32%): LCMS (ESI) calc'd for C₁₈H₂₀Cl₂N₂O₄ [M+H]⁺: 399, 401 (3:2) found 399, 401 (3:2); ¹H NMR (400 MHz, CD₃OD) δ 7.42 (d, J=20.1 Hz, 1H), 7.10 (d, J=3.9 Hz, 1H), 4.12-3.97 (m, 1H), 3.98-3.84 (m, 2H), 3.84-3.75 (m, 1H), 3.74-3.56 (m, 2H), 3.05-2.84 (m, 1H), 2.48-2.31 (m, 2H), 2.28-2.01 (m, 2H), 1.98-1.80 (m, 2H), 1.76-1.61 (m, 2H). The slower-eluting isomer at 20.67 mi was obtained (3S)-5,6-dichloro-1′-[(1R, (3R,4R)-rel)-3,4-dihydroxycyclohexanecarbonyl]-1H-spiro[indole-3,3′-pyrrolidin]-2-one isomer 2 as an off-white solid (31.5 mg, 39%): LCMS (ESI) calc'd for C₁₈H₂₀Cl₂N₂O₄ [M+H]⁺: 399, 401 (3:2) found 399, 401 (3:2); ¹H NR (400 MHz, CD₃D) δ 7.43 (d, J 32.8 Hz, 1H), 7.10 (d, J=5.2 Hz, 1H), 4.09-3.98 (m, 1H), 3.98-3.75 (m, 3H), 3.65 (d, J=12.3 Hz, 1H), 3.53-3.37 (m, 1H), 2.79-2.59 (m, 1H), 2.44-2.28 (3, 2H), 2.09-1.90 (m, 2H), 1.82-1.27 (m, 4H).

The compounds in Table 1C below were prepared in an analogous fashion to that described for Compound 103, starting from Intermediate 1 S and the corresponding (1S)-cyclopent-3-ene-1-carboxylic acid or (1S)-cyclohex-3-ene-1-carboxylic acid, which were commercially available.

TABLE 1C

| Compound | | | |
|---|---|---|---|
| Number | Structure | Chemical Name | MS: (M + H)⁺ & ¹H MNR |
| 105 | 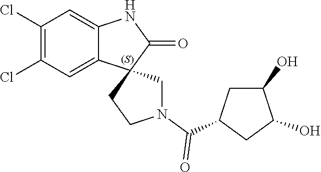 | (S)-5,6-dichloro- 1′-((3R,4R)-rel- 3,4- dihydroxycyclopentane- 1-carbonyl)spiro [indoline-3,3′- pyrrolidin]-2-one isomer 1 | [M + H]⁺: 385, 387 (3:2); ¹H NMR (400 MHz, CD₃OD) δ 7.44 (d, J = 32.77 Hz, 1H), 7.10 (d, J = 5.25 Hz, 1H), 3.96-4.1 (m, 3H), 3.86-3.97 (m, 1H), 3.63-3.85 (m, 2H), 3.34-3.39 (m, 0.5H), 3.12- 3.25 (m, 0.5H), 2.10-2.47 (m, 4H), 1.72-1.98 (m, 2H). |
| 106 | 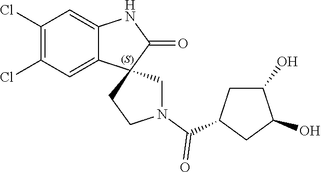 | (S)-5,6-dichloro- 1′-((3R,4R)-rel- 3,4- dihydroxycyclope ntane-1- carbonyl)spiro [indoline-3,3′- pyrrolidin]-2-one isomer 2 | [M + H]⁺: 385, 387 (3:2); ¹H NMR (400 MHz, CD₃OD) δ 7.44 (d, J = 16.44 Hz, 1H), 7.10 (d, J = 4.25 Hz, 1H), 3.89-4.13 (m, 3H), 3.63-3.89 (m, 3H), 3.35-3.4 (m, 0.5H), 3.15-3.25 (m, 0.5H), 2.11- 2.47 (m, 4H), 1.74 - 2.02 (m, 2H). |
| 107 | 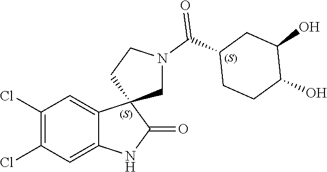 | (S)-5,6-dichloro- 1′-((1S,(3R,4R)- rel)-3,4- dihydroxycyclohexane- 1-carbonyl)spiro [indoline-3,3′- pyrrolidin]-2-one isomer 1 | [M + H]⁺: 399, 401 (3:2); ¹H NMR (400 MHz, CD₃OD) δ 7.43 (d, J = 23.52 Hz, 1H), 7.10 (d, J = 5.80 Hz, 1H), 3.94-4.13 (m, 1H), 3.58-3.93 (m, 3H), 3.36-3.47 (m, 2H), 2.54-2.80 (m, 1H), 2.17-2.48 (m, 2H), 1.75-2.13 (m, 3H), 1.28- 1.67 (m, 3H). |
| 108 | 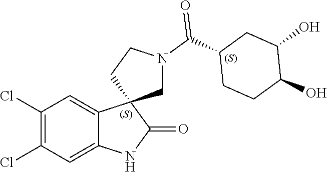 | (S)-5,6-dichloro- 1′-((1S,(3R,4R)- rel)-3,4- dihydroxycyclohexane- 1- carbonyl)spiro [indoline-3,3′- pyrrolidin]-2-one isomer 2 | [M + H]⁺: 399, 401 (3:2); ¹H NMR (400 MHz, CD₃OD) δ 7.41 2H), 2.77-3.09 (m, 1H), 2.13-2.47 (d, J = 35.82 Hz, 1H), 7.10 (d, J = (m, 2H), 2.01-2.13 (m, 1H), 1.79- 7.50 Hz, 1H), 3.93-4.14 (m, 1H), 3.76-3.93 (m, 3H), 3.62-3.73 (m, 1.99 (m, 2H), 1.49-1.79 (m, 3H). |

### Example 12. Compound 109 ((3S)-5,6-dichloro-1′-[(3S,5S)-5-(methoxymethyl)pyrrolidine-3-carbonyl]-1H-spiro[indole-3,3′-pyrrolidin]-2-one) and Compound 110 ((3S)-5,6-dichloro-1′-[(3R,5S)-5-(methoxymethyl)pyrrolidine-3-carbonyl]-1H-spiro[indole-3,3′-pyrrolidin]-2-one

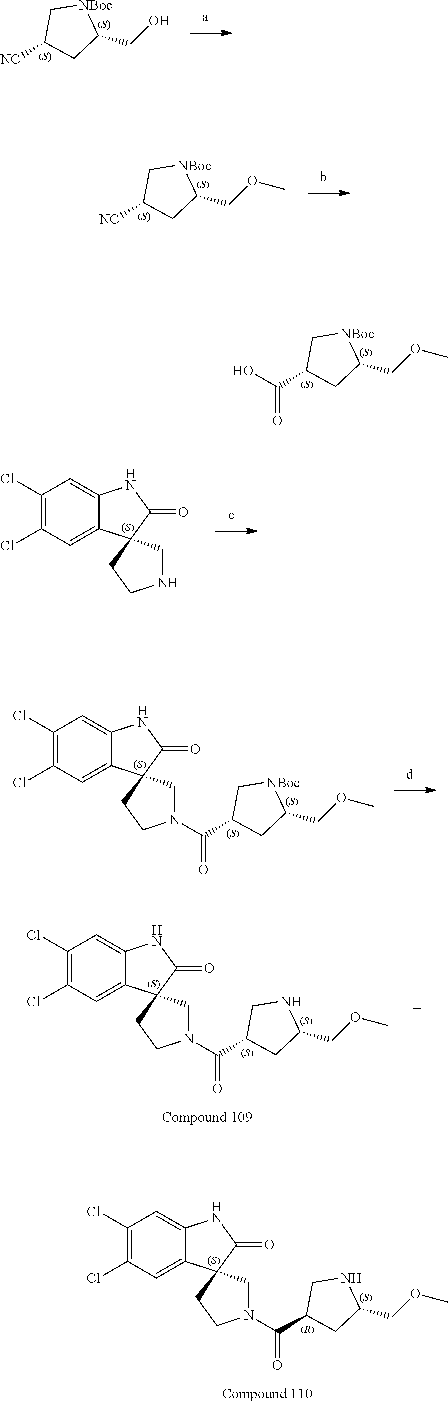

### Step a:

To a stirred solution of tert-butyl (2S,4S)-4-cyano-2-(hydroxymethyl)pyrrolidine-1-carboxylate (0.500 g, 2.21 mmol) in THE (5 mL) was added NaH (0.180 g, 4.38 mmol, 60% in oil) in portions at 0° C. under nitrogen atmosphere. After stirring for 15 min, CH₃I (0.630 g, 4.42 mmol) was added and the reaction mixture was then stirred at room temperature for 1 h, quenched with water (20 mL) at 0° C. and extracted with EA (2×30 mL). The combined organic layers were washed with brine (2×20 mL) and dried over anhydrous Na₂SO₄. After filtration, the filtrate was concentrated under reduced pressure. The residue was purified by silica gel column chromatography, eluting with PE/EA (1/4) to afford tert-butyl (2S,4S)-4-cyano-2-(methoxymethyl)pyrrolidine-1-carboxylate as an light yellow solid (0.260 g, 48%): LCMS (ESI) calc'd for C₁₂H₂₀N₂O₃ [M+H−56]⁺: 185 found 185; ¹H NMR (400 MHz, DMSO-d₆) δ 3.83-3.93 (m, 1H), 3.71-3.83 (m, 1H), 3.33-3.54 (m, 3H), 3.26-3.33 (m, 4H), 2.28-2.46 (m, 1H), 1.97-2.10 (m, 1H), 1.41 (d, J=2.41 Hz, 9H).

### Step b:

To a stirred solution of tert-butyl (2S,4S)-4-cyano-2-(methoxymethyl)pyrrolidine-1-carboxylate (0.250 g, 1.04 mmol) in MeOH (1 mL) was added a solution of NaOH (83.0 mg, 2.08 mmol) in H₂O (1 mL) at room temperature. The reaction mixture was stirred at 80° C. for 2 h, cooled to room temperature and diluted with water (20 mL). The mixture was acidified with saturated aq. citric acid to pH 6 and extracted with EA (2×20 mL). The combined organic layers were washed with brine (2×20 mL) and dried over anhydrous Na₂SO₄. After filtration, the filtrate was concentrated under reduced pressure to afford (3S,5S)-1-(tert-butoxycarbonyl)-5-(methoxymethyl)pyrrolidine-3-carboxylic acid as a yellow oil (0.230 g, 83%), which was used directly in the next step without purification: LCMS (ESI) calc'd for C₁₂H₂₁NO₅ [M+H]⁺: 260 found 260.

### Step c:

To a stirred solution of (3S,5S)-1-(tert-butoxycarbonyl)-5-(methoxymethyl)pyrrolidine-3-carboxylic acid (97.0 mg, 0.370 mmol) in DMF (1.50 mL) were added HOBT (50.0 mg, 0.370 mmol), EDCI (71.0 mg, 0.370 mmol), TEA (94.0 mg, 0.930 mmol) and (3S)-5,6-dichloro-1H-spiro[indole-3,3-pyrrolidin]-2-one (80.0 mg, 0.310 mmol) at room temperature. The reaction mixture was stirred overnight, quenched with MeOH (0.5 mL) and purified by reverse phase chromatography, eluting with 56% ACN in water (plus 0.05% TFA) to afford tert-butyl (2S,4S)-4-[[(3S)-5,6-dichloro-2-oxo-1H-spiro[indole-3,3-pyrrolidin]-1-yl]carbonyl]-2-(methoxymethyl)pyrrolidine-1-carboxylate as a yellow solid (0.100 g, 64%): LCMS (ESI) calc'd for C₂₃H₂₉Cl₂N₃O₅ [M+H]⁺: 498, 500 (3:2) found 498, 500 (3:2); ¹H NMR (300 MHz, CDCl₃) δ 7.96-8.21 (m, 1H), 7.16-7.27 (m, 1H), 7.08 (d, J=11.39 Hz, 1H), 4.05-4.19 (m, 2H), 3.80-4.05 (m, 3H), 3.56-3.80 (m, 3H), 3.29-3.51 (m, 5H), 2.41-2.57 (m, 1H), 2.17-2.41 (m, 2H), 1.98-2.17 (m, 1H), 1.46-1.54 (m, 9H).

### Step d:

To a stirred solution of tert-butyl (2S,4S)-4-[[(3S)-5,6-dichloro-2-oxo-1H-spiro[indole-3,3-pyrrolidin]-1-yl]carbonyl]-2-(methoxymethyl)pyrrolidine-1-carboxylate (0.100 g, 0.200 mmol) in DCM (1 mL) was added TFA (1 mL) at room temperature. The reaction mixture was stirred for 1 h and concentrated under reduced pressure. The residue was purified by reverse phase chromatography, eluting with 60% ACN in water (plus 0.05% TFA) to afford the product (60.0 mg) as an off-white solid. The product was separated by Prep-Chiral-HPLC with the following conditions: Column: (R, R)-WHELK-O1-Kromasil, 2.11×25 cm, 5 μm; Mobile Phase A: Hex (plus 0.5% 2 MNH3-MeOH)-HPLC, Mobile Phase B: EtOH-HPLC; Flow rate: 20 mL/min; Gradient: 30% B to 30% B in 35 min; Wavelength: UV 220/254 nm; Retention Time 1: 21.14 min; Retention Time 2: 25.96 min; Sample Solvent: EtOH-HPLC; Injection Volume: 1 mL; Number Of Runs: 3. The faster-eluting isomer at 21.14 min was obtained (3S)-5,6-dichloro-1′-[(3S,5S)-5-(methoxymethyl)pyrrolidine-3-carbonyl]-1H-spiro[indole-3,3′-pyrrolidin]-2-one as an off-white solid (23.8 mg, 29%): LCMS (ESI) calc'd for C₁₈H₂₁Cl₂N₃O₃ [M+H]⁺: 398, 400 (3:2) found 389, 400 (3:2); ¹H NMR (300 MHz, CD₃OD) δ 7.45 (d, J=18.13 Hz, 1H), 7.10 (d, J=3.60 Hz, 1H), 3.91-4.13 (m, 1H), 3.61-3.91 (m, 3H), 3.40-3.52 (m, 2H), 3.34-3.40 (m, 4H), 3.00-3.30 (m, 3H), 2.02-2.5 (m, 3H), 1.73-1.98 (m, 1H). The slower-eluting enantiomer at 25.96 min was obtained (3S)-5,6-dichloro-1′-[(3R,5S)-5-(methoxymethyl)pyrrolidine-3-carbonyl]-1H-spiro[indole-3,3′-pyrrolidin]-2-one as an off-white solid (2.40 mg, 3%): LCMS (ESI) calc'd for C₁₈H₂₁Cl₂N₃O₃ [M+H]⁺: 398, 400 (3:2) found 389, 400 (3:2); ¹H NMR (300 MHz, CD₃OD) δ 7.38-7.53 (m, 1H), 7.10 (d, J=2.90 Hz, 1H), 3.96-4.12 (m, 1H), 3.60-3.96 (m, 3H), 3.44-3.54 (m, 2H), 3.39 (d, J=3.23 Hz, 3H), 2.99-3.27 (m, 4H), 2.08-2.47 (m, 3H), 1.62-1.88 (m, 1H).

The compounds in Table 1D below were prepared in an analogous fashion to that described for Compound 109, starting from Intermediate 1S and (3R,5R)-1-(tert-butoxycarbonyl)-5-(methoxymethyl)pyrrolidine-3-carboxylic acid, which was in turn prepared analogously to (3S,5S)-1-(tert-butoxycarbonyl)-5-(methoxymethyl)pyrrolidine-3-carboxylic acid, starting from tert-butyl (2R,4R)-4-cyano-2-(hydroxymethyl)pyrrolidine-1-carboxylate.

TABLE 1D

| Compound | | | |
|---|---|---|---|
| Number | Structure | Chemical Name | MS: (M + H)⁺ & ¹H MNR |
| 111 | 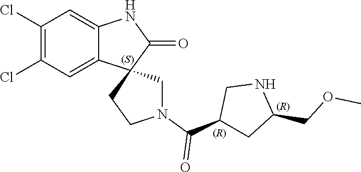 | (S)-5,6-dichloro- 1′-((3R,5R)-5- (methoxymethyl) pyrrolidine-3- carbonyl)spiro [indoline-3,3′- pyrrolidin]-2-one | [M + H]⁺: 398, 400 (3:2); ¹H NMR (300 MHz, CD₃OD) δ 7.45 (d, J = 18.27 Hz, 1H), 7.10 (d, J = 3.64 Hz, 1H), 3.60-4.11 (m, 4H), 3.43-3.57 (m, 3H), 3.35-3.43 (m, 4H), 3.08-3.24 (m, 2H), 1.80-2.44 (m, 4H). |
| 112 | 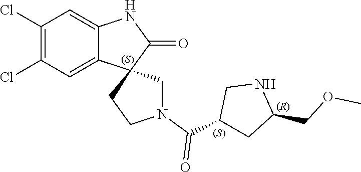 | (S)-5,6-dichloro- 1′-((3S,5R)-5- (methoxymethyl) pyrrolidine-3- carbonyl)spiro [indoline-3,3′- pyrrolidin]-2-one | [M + H]⁺: 398, 400 (3:2); ¹H 7.36-7.5 (m, 1H), 7.10 (d, J = 4H), 3.34-3.53 (m, 7H), 3.00- NMR (300 MHz, CD₃OD) δ 4.15 Hz, 1H), 3.59-4.12 (m, 3.28 (m, 2H), 2.06-2.50 (m, 3H), 1.61-1.89 (m, 1H). |

### Example 13. Compound 113 ((3S)-5,6-dichloro-1-[(3S,53)-5-(hydroxymethyl)pyrrolidine-3-carbonyl]-1H-spiro[indole-3,3-pyrrolidin]-2-one) and Compound 114 ((3S)-5,6-dichloro-1′-[(3R,5S)-5-(hydroxymethyl)pyrrolidine-3-carbonyl]-1H-spiro[indole-3,3′-pyrrolidin]-2-one)

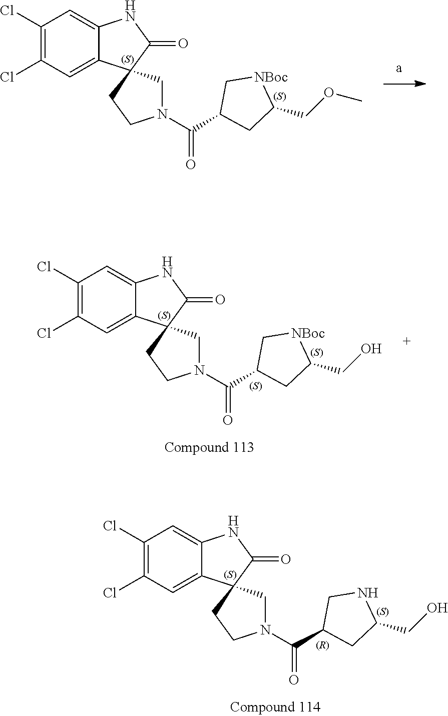

### Step a:

To a stirred solution of tert-butyl (2S)-4-[[(3S)-5,6-dichloro-2-oxo-1H-spiro[indole-3,3-pyrrolidin]-1-yl]carbonyl]-2-(methoxymethyl)pyrrolidine-1-carboxylate (0.100 g, 0.200 mmol) in DCM (2 mL) was added BBr₃ (0.100 g, 0.40 mmol) at 0° C. under nitrogen atmosphere. The reaction mixture was stirred at room temperature for 1 h, quenched with MeOH (2 mL) and concentrated under reduced pressure. The residue was purified by reverse phase chromatography, eluting with 56% ACN in water (plus 0.05% TFA) to afford the product as an off-white solid (50.0 mg). The product (50.0 mg) was separated by Prep Chiral HPLC with the following conditions: Column: Lux 5 μm Cellulose-2, 2.12×25 cm, 5 μm; Mobile Phase A: Hex (plus 0.5% IPA)-HPLC, Mobile Phase B: IPA-HPLC; Flow rate: 18 mL/min; Gradient: 50% B to 50% B in 35 min; Wavelength: UV 220/254 nm; Retention Time 1: 15.26 min; Retention Time 2: 27.23 min; Injection Volume: 1 mL; Number Of Runs: 4. The faster-eluting isomer at 15.26 min was obtained (3S)-5,6-dichloro-1-[(3S,5S)-5-(hydroxymethyl)pyrrolidine-3-carbonyl]-1H-spiro[indole-3,3-pyrrolidin]-2-one as an off-white solid (17.0 mg, 22%): LCMS (ESI) calc'd for C₁₇H₁₉Cl₂N₃O₃ [M+H]⁺: 384, 386 (3:2) found 384, 386 (3:2); ¹H NMR (300 MHz, CD₃OD) δ 7.49 (d, J=18.79 Hz, 1H), 7.11 (d, J=3.62 Hz, 1H), 3.90-4.12 (m, 2H), 3.74-3.90 (m, 3H), 3.56-3.74 (m, 3H), 3.39-3.56 (m, 2H), 1.99-2.50 (m, 4H). The slower-eluting isomer at 27.23 min was obtained (3S)-5,6-dichloro-1-[(3R,5S)-5-(hydroxymethyl)pyrrolidine-3-carbonyl]-1H-spiro[indole-3,3-pyrrolidin]-2-one as an off-white solid (4.40 mg, 5%): LCMS (ESI) calc'd for C₁₇H₁₉Cl₂N₃O₃ [M+H]⁺: 384, 386 (3:2) found 384,386 (3:2); ¹H NMR (300 MHz, CD₃OD) δ 7.45 (d, J=14.37 Hz, 1H), 7.10 (d, J=3.44 Hz, 1H), 3.77-4.12 (m, 4H), 3.60-3.70 (m, 3H), 2.98-3.31 (m, 3H), 2.17-2.48 (m, 3H), 1.68-1.93 (m, 1H).

The compound in Table 1E below was prepared in an analogous fashion to that described for Compound 113, starting from tert-butyl (2R)-4-[[(3S)-5,6-dichloro-2-oxo-1H-spiro[indole-3,3-pyrrolidin]-1-yl]carbonyl]-2-(methoxymethyl)pyrrolidine-1-carboxylate.

TABLE 1E

| Compound | | | MS: (M + H)⁺ & ¹H |
|---|---|---|---|
| No | Structure | Chemical Name | MNR |
| 115 | 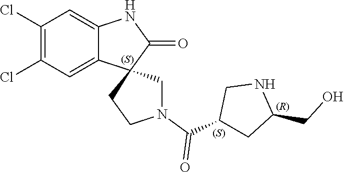 | (S)-5,6-dichloro-1′-((3S,5R)- 5- (hydroxymethyl)pyrrolidine- 3-carbonyl)spiro[indoline- 3,3′-pyrrolidin]-2-one | [M + H]⁺: 384, 386 (3:2); ¹H NMR (400 MHz, CD₃OD) δ 7.45 (d, J = 28.38 Hz, 1H), 7.10 (d, J = 4.87 Hz, 1H), 3.75- 4.14 (m, 4H), 3.59-3.71 (m, 3H), 2.98-3.30 (m, 4H), 2.13-2.50 (m, 2H), 1.68-1.83 (m, 1H). |
| 116 | 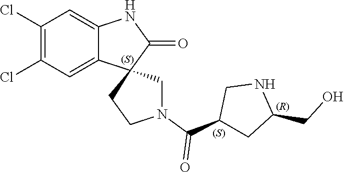 | (S)-5,6-dichloro-1′- ((3R,5R)-5- (hydroxymethyl)pyrrolidine- 3-carbonyl)spiro[indoline- 3,3′-pyrrolidin]-2-one | [M + H]⁺: 384, 386 (3:2); ¹H NMR (400 MHz, CD₃OD) δ 7.45 (d, J = 23.43 Hz, 1H), 7.10 (d, J = 4.49 Hz, 1H), 3.78- 4.10 (m, 4H), 3.53-3.69 (m, 3H), 3.35-3.43 (m, 1H), 3.02-3.23 (m, 2H), 2.31-2.47 (m, 2H), 2.06-2.28 (m, 1H), 1.82-1.99 (m, 1H) |

The compound in Table 1F below was prepared in an analogous fashion to that described for Compound 1, starting from glycolic acid and the appropriately substituted 1H-spiro[indole-3,3′-pyrrolidin]-2-one intermediate, which in turn was prepared analogously to Intermediates 1S and 1R.

TABLE 1F

| Compound | | | MS: (M + H)⁺ & ¹H |
|---|---|---|---|
| No | Structure | Chemical Name | MNR |
| 117 | 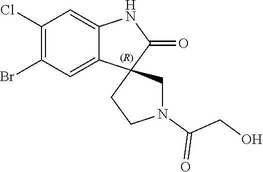 | (R)-5-bromo-6-chloro-1′-(2- hydroxyacetyl)spiro[indoline- 3,3′-pyrrolidin]-2-one | [M + H]⁺: 359, 361 (3:2); ¹H NMR (400 MHz, CD₃OD) δ 7.59 (d, J = 24.94 Hz, 1H), 7.12 (s, 1H), 4.23-4.37 (m, 1H), 4.18 (s, 1H), 3.78-3.98 (m, 2H), 3.73 (dd, J = 13.81, 11.73 Hz, 2H), 2.16- 2.48 (m, 2H). |
| 118 | 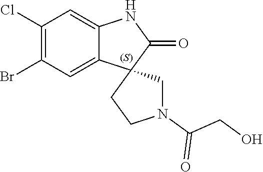 | (S)-5-bromo-6-chloro-1′-(2- hydroxyacetyl)spiro[indoline- 3,3′-pyrrolidin]-2-one | [M + H]⁺: 359, 361 (3:2); ¹H NMR (400 MHz, CD₃OD) δ 7.59 (d, J = 24.99 Hz, 1H), 7.12 (s, 1H), 4.22-4.38 (m, 1H), 4.18 (s, 1H), 3.66-3.99 (m, 4H), 2.14-2.48 (m, 2H). |
| 119 | 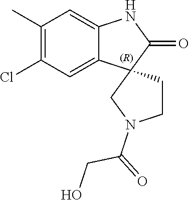 | (R)-5-chloro-1′-(2- hydroxyacetyl)-6- methylspiro[indoline-3,3′- pyrrolidin]-2-one | [M + H]⁺: 295, 297 (3:2); ¹H NMR (400 MHz, CD₃OD) δ 7.27 (d, J = 22.35 Hz, 1H), 6.84-6.94 (m, 1H), 4.22-4.38 (m, 1H), 4.18 (s, 1H), 3.61-3.98 (m, 4H), 2.14-2.46 (m, 5H). |
| 120 | 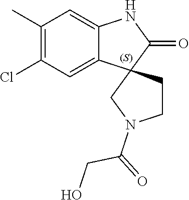 | (S)-5-chloro-1′-(2- hydroxyacetyl)-6- methylspiro[indoline-3,3′- pyrrolidin]-2-one | [M + H]:295, 297 (3:2); ¹H NMR (400 MHz, CD₃OD) δ 7.27 (d, J = 22.48 Hz, 1H), 6.89 (s, 1H), 4.22-4.39 (m, 1H), 4.18 (s, 1H), 3.63-4.01 (m, 4H), 2.16-2.49 (m, 5H). |

### Example 14. Compound 121 ((3S)-5,6,7-trichloro-1′-(2-hydroxyacetyl)-1H-spiro[indole-3,3′-pyrrolidin]-2-one) and Compound 122 ((3R)-5,6,7-trichloro-1′-(2-hydroxyacetyl)-1H-spiro[indole-3,3′-pyrrolidin]-2-one)

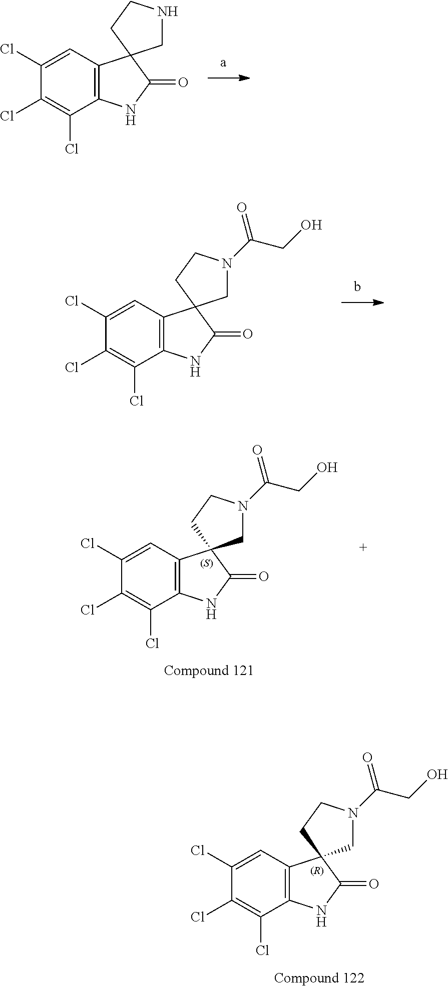

### Step a:

To a stirred solution of glycolic acid (45.0 mg, 0.590 mmol), HOBT (91.0 mg, 0.670 mmol) and EDCI (0.130 g, 0.680 mmol) in DMF (2 mL) were added TEA (0.180 g, 1.80 mmol) and 4,5,6-trichloro-1H-spiro[indole-3,3′-pyrrolidin]-2-one (Intermediate 2, 80.0 mg, 0.270 mmol) at room temperature. The reaction mixture was stirred for 2 h, quenched with MeOH (0.5 mL) and purified by reverse phase chromatography, eluting with 55% ACN in water (plus 10 mM NH₄HCO₃) to afford 5,6,7-trichloro-1′-(2-hydroxyacetyl)-1H-spiro[indole-3,3′-pyrrolidin]-2-one as an off-white solid (47.4 mg, 30%): LCMS (ESI) calc'd for C₁₃H₁₁C₁₃N₂O₃ [M+H]⁺: 349, 351, 353 (3:3:1), found 349, 351, 353 (3:3:1); ¹H NMR (400 MHz, DMSO-d₆) δ 11.29 (s, 1H), 7.60 (d, J=41.32 Hz, 1H), 4.72-4.67 (brs, 1H), 4.03-4.25 (m, 1H), 3.99 (s, 1H), 3.67-3.8 (m, 3H), 3.61-3.67 (m, 1H), 2.16-2.35 (m, 2H).

### Step b:

5,6,7-trichloro-1′-(2-hydroxyacetyl)-1H-spiro[indole-3,3′-pyrrolidin]-2-one (40.0 mg, 0.110 mmol) was separated by Prep Chiral HPLC with the following condition: Column: CHIRALPAK IE, 2×25 cm, 5 μm; Mobile Phase A: Hex (plus 0.5% IPA), Mobile Phase B: EtOH; Flow rate: 20 mL/min; Gradient: 50% B to 50% B in 30.5 min; Detector: UV 220/254 nm; Retention time 1: 16.04 min; Retention time 2: 25.32 min. The faster-eluting enantiomer at 16.04 min was obtained (3S)-5,6,7-trichloro-1′-(2-hydroxyacetyl)-1H-spiro[indole-3,3′-pyrrolidin]-2-one as an off-white solid (13.9 mg, 34%): LCMS (ESI) calc'd for C₁₃H₁₁C₁₃N₂O₃ [M+H]⁺: 349, 351, 353 (3:3:1), found 349, 351, 353 (3:3:1); ¹H NMR (400 MHz, DMSO-d₆) δ 11.25 (s, 1H), 7.60 (d, J=41.3 Hz, 1H), 4.72-4.67 (brs, 1H), 4.03-4.22 (m, 1H), 3.99 (s, 1H), 3.66-3.8 (m, 3H), 3.64 (d, J=2.8 Hz, 1H), 2.24-2.36 (m, 1H), 2.16-2.24 (m, 1H). The slower-eluting enantiomer at 25.32 min was obtained (3R)-5,6,7-trichloro-1′-(2-hydroxyacetyl)-1H-spiro[indole-3,3′-pyrrolidin]-2-one as an off-white solid (12.9 mg, 32%): LCMS (ESI) calc'd for C₁₃H₁₁C₁₃N₂O₃ [M+H]⁺: 349, 351, 353 (3:3:1), found 349, 351, 353 (3:3:1); ¹H NMR (400 MHz, DMSO-d₆) δ 11.21 (s, 1H), 7.60 (d, J=41.3 Hz, 1H), 4.72-4.67 (brs, 1H), 4.03-4.22 (m, 1H), 3.99 (s, 1H), 3.66-3.8 (m, 3H), 3.64 (d, J=2.8 Hz, 1H), 2.24-2.36 (m, 1H), 2.16-2.24 (m, 1H).

### Example 15. Compound 129 ((3S)-5,6-dichloro-1′-(2-hydroxyacetyl)-7-methyl-1H-spiro[indole-3,3′-pyrrolidin]-2-one)

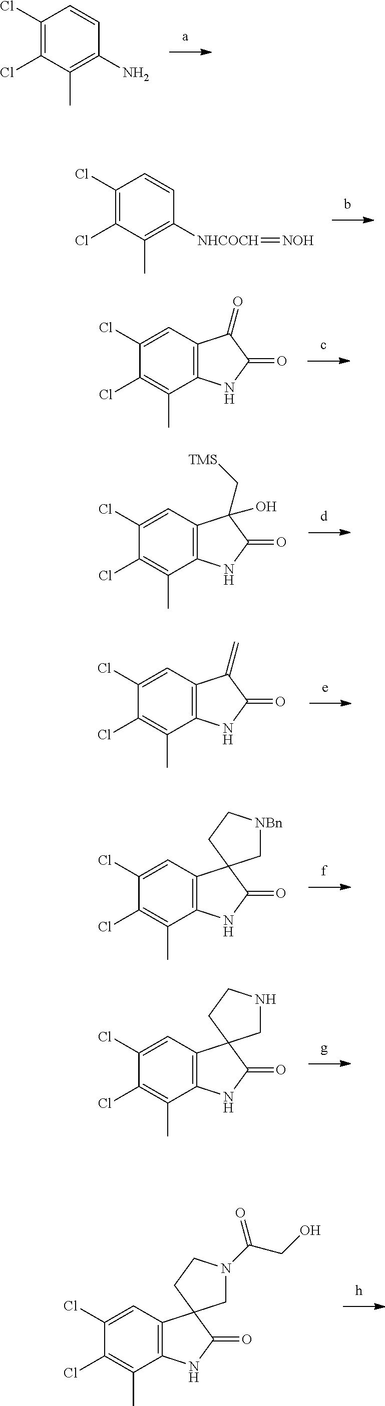

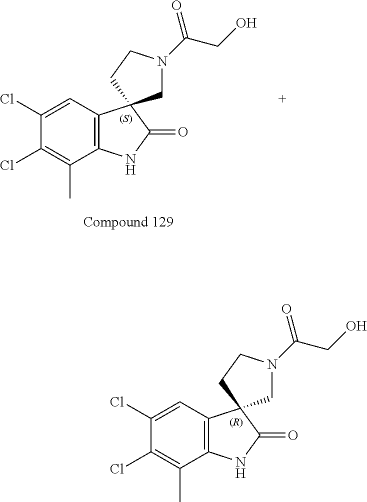

### Step a:

To a stirred solution of 3,4-dichloro-2-methylaniline (1.70 g, 9.66 mmol) and Na₂SO₄ (8.23 g, 57.9 mmol) in H₂O (40.0 mL) were added hydroxylamine hydrochloride (2.01 g, 29.0 mmol) and chloral hydrate (1.92 g, 11.6 mmol) in portions at room temperature. Conc. HCl (0.48 mL, 12 N) was then added dropwise over 3 min. The reaction mixture was stirred at 70° C. for 5 h. After cooling to room temperature, the precipitate was collected by filtration and washed with water (3×10 mL) to afford N-(3,4-dichloro-2-methylphenyl)-2-(N-hydroxyimino)acetamide as a yellow solid (1.70 g, 71%): LCMS (ESI) calc'd for C₉H₈Cl₂N₂O₂ [M−H]⁻: 245, 247 (3:2), found 245, 247 (3:2); ¹H NMR (300 MHz, CDCl₃) δ 8.24 (s, 2H), 7.80 (d, J=8.8 Hz, 1H), 7.63 (s, 1H), 7.36 (d, J=8.8 Hz, 1H), 2.40 (s, 3H).

### Step b:

N-(3,4-dichloro-2-methylphenyl)-2-(N-hydroxyimino)acetamide (1.70 g, 6.88 mmol) was added to conc. H₂SO₄ (15 mL) in portions at 80° C. The reaction mixture was stirred for 2 h. After cooling to room temperature, the reaction was poured into ice water (60 mL). The precipitate was filtered off and washed with water (3×10 mL) to afford 5,6-dichloro-7-methyl-1H-indole-2,3-dione as a light brown solid (1.30 g, 82%): LCMS (ESI) calc'd for C₉H₅Cl₂NO₂ [M−H]⁻: 228, 230 (3:2), found 228, 230 (3:2); ¹H NMR (300 MHz, DMSO-d₆) δ 11.27 (s, 1H), 7.64 (s, 1H), 2.28 (s, 3H).

### Step c:

To a solution of 5,6-dichloro-7-methyl-1H-indole-2,3-dione (1.30 g, 5.65 mmol) in THE (30 mL) was added (trimethylsilyl)methyl magnesium chloride in THE (18 mL, 159 mmol) at −78° C. under nitrogen atmosphere. The reaction mixture was stirred for 2 h, quenched with saturated aq. NH₄Cl (25 mL) and extracted with EA (3×20 mL). The combined organic layers were washed with brine (2×20 mL) and dried over anhydrous Na₂SO₄. After filtration, the filtrate was concentrated under reduced pressure. The residue was purified by silica gel chromatography, eluting with PE/EA (3/1) to afford 5,6-dichloro-3-hydroxy-7-methyl-3-[(trimethylsilyl)methyl]-1H-indol-2-one as a yellow solid (0.800 g, 44%): LCMS (ESI) calc'd for C₁₃H₁₇Cl₂NO₂Si [M−H]⁻: 316, 318 (3:2), found 316, 318 (3:2); ¹H NMR (400 MHz, CDCl₃) δ 9.17 (s, 1H), 7.33 (s, 1H), 3.15 (s, 1H), 2.37 (s, 3H), 1.52 (s, 2H), −0.16 (s, 9H).

### Step d:

To a solution of 5,6-dichloro-3-hydroxy-7-methyl-3-[(trimethylsilyl)methyl]-1H-indol-2-one (0.800 g, 2.51 mmol) in DCM (2 mL) was added BF₃·Et₂O (3.50 g, 24.7 mmol) at −78° C. under nitrogen atmosphere. The reaction mixture was stirred at room temperature for 2 h and filtered. The filter cake was washed with DCM (5 mL) to afford 5,6-dichloro-7-methyl-3-methylidene-1H-indol-2-one as a yellow solid (0.470 g, 82%): LCMS (ESI) calc'd for C₁₀H₇Cl₂NO [M+H]⁺: 228, 230 (3:2), found 228, 230 (3:2); ¹H NMR (400 MHz, DMSO-d₆) δ 10.90 (s, 1H), 7.83 (s, 1H), 6.48 (s, 1H), 6.28 (s, 1H), 2.29 (s, 3H).

### Step e:

To a solution of 5,6-dichloro-7-methyl-3-methylidene-1H-indol-2-one (0.470 g, 2.06 mmol) in THE (8 mL) were added TFA (0.258 g, 2.27 mmol) and benzyl(methoxymethyl)[(trimethylsilyl)methyl]amine (0.538 g, 2.27 mmol) at −78° C. under nitrogen atmosphere. The reaction mixture was stirred at room temperature for 2 h, basified to pH 8 with saturated aq. NaHCO₃ and extracted with EA (3×20 mL). The combined organic layers were washed with brine (2×15 mL) and dried over anhydrous Na₂SO₄. After filtration, the filtrate was concentrated under reduced pressure. The residue was purified by silica gel chromatography, eluting with PE/EA (1/1) to afford 1′-benzyl-5,6-dichloro-7-methyl-1H-spiro[indole-3,3′-pyrrolidin]-2-one as a light yellow solid (0.500 g, 67%): LCMS (ESI) calc'd for C₁₉H₁₈Cl₂N₂O [M+H]⁺: 361, 363 (3:2), found 361, 363 (3:2); ¹H NMR (300 MHz, CDCl₃) δ 8.68 (s, 1H), 7.51 (s, 1H), 7.44-7.29 (m, 5H), 3.78 (s, 2H), 3.25-3.11 (m, 1H), 3.00-2.62 (m, 3H), 2.49-2.39 (m, 1H), 2.36 (s, 3H), 2.16-2.00 (m, 1H).

### Step f:

To a solution of 1′-benzyl-5,6-dichloro-7-methyl-1H-spiro[indole-3,3′-pyrrolidin]-2-one (0.500 g, 1.38 mmol) in DCE (5 mL) was added chloroethyl chloroformate (1.00 g, 6.99 mmol) in one portion at room temperature. The reaction mixture was stirred at 60° C. for 2 h and concentrated under reduced pressure. The residue was dissolved in MeOH (5 mL), stirred at 60° C. for 1 h and evaporated. The residue was purified by reverse phase chromatography, eluting with 45% ACN in water (plus 20 mM NH₄HCO₃) to afford 5,6-dichloro-7-methyl-1H-spiro[indole-3,3′-pyrrolidin]-2-one as a light yellow solid (0.220 g, 59%): LCMS (ESI) calc'd for C₁₂H₁₂Cl₂N₂O [M+H]⁺: 271, 273 (3:2), found 271, 273 (3:2); ¹H NMR (300 MHz, CD₃OD) δ 7.50 (s, 1H), 3.80-3.59 (m, 3H), 3.56-3.49 (m, 1H), 2.53-2.30 (m, 5H).

### Step g:

To a solution of glycolic acid (67.0 mg, 0.890 mmol), EDCI (0.212 g, 1.11 mmol) and HOBT (0.149 g, 1.11 mmol) in DMF (1 mL) were added 5,6-dichloro-7-methyl-1H-spiro[indole-3,3′-pyrrolidin]-2-one (0.200 g, 0.738 mmol) and TEA (0.224 g, 2.21 mmol) at room temperature. The reaction mixture was stirred for 2 h, diluted with water (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by silica gel column chromatography, eluting with PE/EA (1/1) to afford 5,6-dichloro-1′-(2-hydroxyacetyl)-7-methyl-1H-spiro[indole-3,3′-pyrrolidin]-2-one as an off-white solid (70.0 mg, 29%): LCMS (ESI) calc'd for C₁₄H₁₄Cl₂N₂O₃ [M+H]⁺: 329, 331 (3:2), found 329, 331 (3:2); ¹H NMR (300 MHz, CD₃OD) δ 7.32 (d, J=18.3 Hz, 1H), 4.33-4.17 (m, 2H), 3.92-3.68 (m, 4H), 2.46-2.25 (m, 5H).

### Step h:

5,6-Dichloro-1′-(2-hydroxyacetyl)-7-methyl-1H-spiro[indole-3,3′-pyrrolidin]-2-one (30.0 mg, 0.0911 mmol) was separated by Prep Chiral HPLC with the following conditions: Column: (R, R)-WHELK-O1-Kromasil, 2.12×25 cm, 5 μm; Mobile Phase A: Hex (plus 0.5% IPA), Mobile Phase B: EtOH; Flow rate: 20 mL/min; Gradient: 35% B to 35% B in 22 min; Detector: UV 220/254 nm; Retention time 1: 15.22 min; Retention time 2: 19.77 min; Sample Solvent: EtOH. The faster-eluting enantiomer at 15.22 min was obtained (3S)-5,6-dichloro-1′-(2-hydroxyacetyl)-7-methyl-1H-spiro[indole-3,3′-pyrrolidin]-2-one as an off-white solid (10.0 mg, 33%): LCMS (ESI) calc'd for C₁₄H₁₄Cl₂N₂O₃ [M+H]⁺: 329, 331 (3:2), found 329, 331 (3:2); ¹H NMR (300 MHz, DMSO-d₆) δ10.91 (s, 1H), 7.43 (d, J=29.4 Hz, 1H), 4.73-4.61 (m, 1H), 4.24-3.95 (m, 2H), 3.73 (q, J=7.7 Hz, 2H), 3.63 (d, J=14.1 Hz, 2H), 2.31 (s, 3H), 2.27-2.19 (m, 1H), 2.18-2.07 (m, 1H). The slower-eluting enantiomer at 19.77 min was obtained (3R)-5,6-dichloro-1′-(2-hydroxyacetyl)-7-methyl-1H-spiro[indole-3,3′-pyrrolidin]-2-one as an off-white solid (12.2 mg, 41%): LCMS (ESI) calc'd for C₁₄H₁₄Cl₂N₂O₃ [M+H]⁺: 329, 331 (3:2), found 329, 331 (3:2); ¹H NMR (300 MHz, DMSO-d₆) δ 10.91 (s, 1H), 7.43 (d, J=29.4 Hz, 1H), 4.73-4.61 (m, 1H), 4.24-3.95 (m, 2H), 3.73 (q, J=7.7 Hz, 2H), 3.63 (d, J=14.1 Hz, 2H), 2.31 (s, 3H), 2.27-2.19 (m, 1H), 2.18-2.07 (m, 1H).

The compounds in Table 1G below were prepared in an analogous fashion to that described for Compound 129, starting either from the corresponding anilines, or from the corresponding 1H-indole-2,3-diones which were available from commercial sources.

TABLE 1G

| Compound | | | |
|---|---|---|---|
| No. | Structure | Chemical Name | MS: (M + H)⁺ & ¹H MNR |
| 127 | 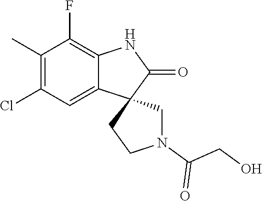 [SMILES: Cc1c(Cl)cc2c(c1F)NC(=O)[C@@]21CCN(C(=O)CO)C1] | (S)-5-chloro-7- fluoro-1′-(2- hydroxyacetyl)-6- methylspiro [indoline-3,3′- pyrrolidin]-2-one | [M + H]⁺: 313, 315 (3:2); ¹H NMR (400 MHz, CD₃OD) δ 7.15 (dd, J = 24.86, 1.07 Hz, 1H), 4.34-4.19 (m, 1H), 4.15 (s, 1H), 3.98-3.65 (m, 4H), 2.52-2.09 (m, 5H). |
| 128 | 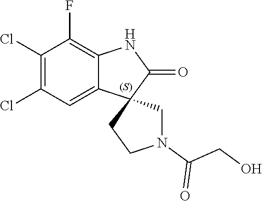 | (S)-5,6-dichloro- 7-fluoro-1′-(2- hydroxyacetyl) spiro[indoline-3,3′- pyrrolidin]-2-one | [M + H]⁺: 333, 335 (3:2); ¹H NMR (300 MHz, CD₃OD) δ 7.33 (dd, J = 21.35, 1.48 Hz, 1H), 4.35-4.20 (m, 1H), 4.15 (s, 1H), 3.98-3.65 (m, 4H), 2.51-2.15 (m, 2H). |
| 140 | 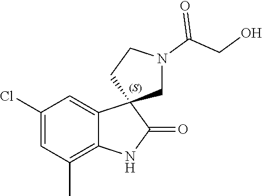 | (S)-5-chloro-1′- (2- hydroxyacetyl)-7- methylspiro [indoline-3,3′- pyrrolidin]-2-one | [M + H]⁺: 295, 297 (3:1); ¹H NMR (400 MHz, CD₃OD) δ 7.17-7.07 (m, 2H), 4.37-4.24 (m, 1H), 4.18 (s, 1H), 4.00-3.73 (m, 3H), 3.69 (dd, J = 11.48, 4.80 Hz, 1H), 2.46-2.29 (m, 1.5H), 2.28 (d, J = 0.93 Hz, 3H), 2.23-2.15 (m, 0.5 H). |

### Example 16. Compound 123 (5,6-dichloro-1′-(2-hydroxyacetyl)-5′-methyl-1H-spiro[indole-3,3′-pyrrolidin]-2-one isomer 1), Compound 124 (5,6-dichloro-1′-(2-hydroxyacetyl)-5′-methyl-1H-spiro[indole-3,3′-pyrrolidin]-2-one isomer 2), Compound 125 (5,6-dichloro-1′-(2-hydroxyacetyl)-5′-methyl-1H-spiro[indole-3,3′-pyrrolidin]-2-one isomer 3), and Compound 126 (5,6-dichloro-1′-(2-hydroxyacetyl)-5′-methyl-1H-spiro[indole-3,3′-pyrrolidin]-2-one isomer 4)

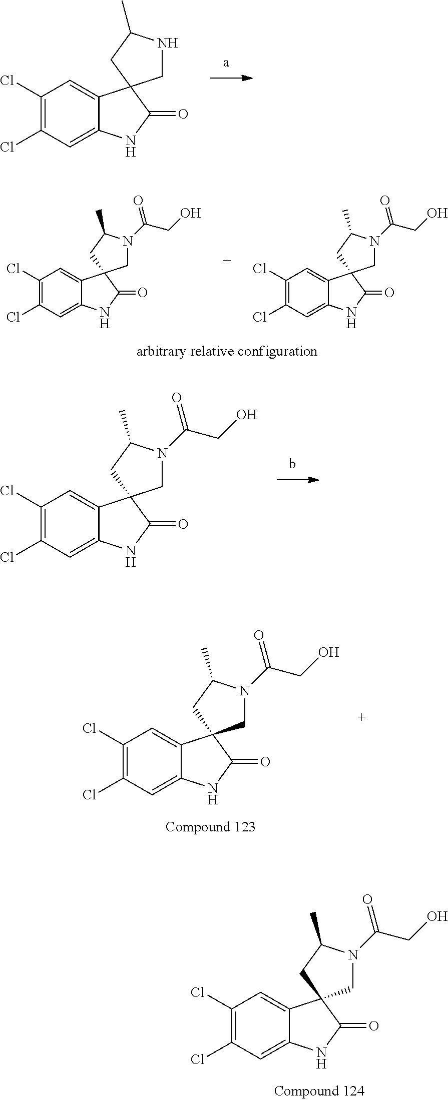 [SMILES: CC1CC2(CN1)C(=O)Nc1cc(Cl)c(Cl)cc12 ; C[C@@H]1C[C@]2(CN1C(=O)CO)C(=O)Nc1cc(Cl)c(Cl)cc12 ; C[C@H]1C[C@]2(CN1C(=O)CO)C(=O)Nc1cc(Cl)c(Cl)cc12 ; C[C@H]1C[C@]2(CN1C(=O)CO)C(=O)Nc1cc(Cl)c(Cl)cc12 ; C[C@H]1C[C@]2(CN1C(=O)CO)C(=O)Nc1cc(Cl)c(Cl)cc12 ; C[C@@H]1C[C@@]2(CN1C(=O)CO)C(=O)Nc1cc(Cl)c(Cl)cc12]

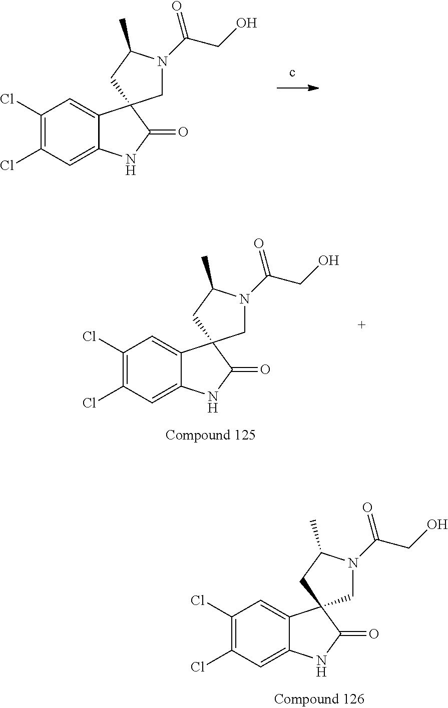 [SMILES: C[C@@H]1C[C@]2(CN1C(=O)CO)C(=O)Nc1cc(Cl)c(Cl)cc12 ; C[C@@H]1C[C@]2(CN1C(=O)CO)C(=O)Nc1cc(Cl)c(Cl)cc12 ; C[C@H]1C[C@@]2(CN1C(=O)CO)C(=O)Nc1cc(Cl)c(Cl)cc12]

### Step a:

To a stirred mixture of glycolic acid (44.0 mg, 0.580 mmol) amd HOBT (79.0 mg, 0.580 mmol) in DMF (2 mL) were added 5,6-dichloro-5′-methyl-1H-spiro[indole-3,3′-pyrrolidin]-2-one (Intermediate 3, 0.150 g, 0.390 mmol) and TEA (0.120 g, 1.17 mmol) at room temperature. The reaction mixture was stirred at 40° C. for 1 h, quenched with MeOH (0.5 mL) and purified by Prep-HPLC with the following conditions: Column: SunFire Prep C18 OBD Column, 19×150 mm, 5 μm 10 nm; Mobile Phase A: Water (plus 0.05% TFA), Mobile Phase B: ACN; Flow rate: 20 mL/min; Gradient: 30% B to 40% B in 5.5 min, 40% B; Wavelength: UV 254/210 nm; Retention Time 1: 3.75 min; Retention Time 2: 5.00 min. The fraction at 3.75 min was obtained 5,6-dichloro-1′-(2-hydroxyacetyl)-5′-methyl-1H-spiro[indole-3,3′-pyrrolidin]-2-one diastereoisomer A as a colorless oil (45.0 mg, 27%): LCMS (ESI) calc'd for C₁₄H₁₄Cl₂N₂O₃ [M+H]⁺: 329, 331 (3:2) found 329, 331 (3:2); ¹H NMR (400 MHz, CD₃OD) δ 7.12 (s, 2H), 4.61-4.23 (m, 1H), 4.23-4.03 (m, 2H), 3.81-3.53 (m, 2H), 2.53-2.02 (m, 2H), 1.48 (dd, J=6.3, 2.5 Hz, 3H). The fraction at 5.00 min was obtained 5,6-dichloro-1′-(2-hydroxyacetyl)-5′-methyl-1H-spiro[indole-3,3′-pyrrolidin]-2-one diastereoisomer B as a colorless oil (45.0 mg, 27%): LCMS (ESI) calc'd for C₁₄H₁₄Cl₂N₂O₃ [M+H]⁺: 329, 331 (3:2) found 329, 331 (3:2); ¹H NMR (400 MHz, CD₃OD) δ 7.60 (d, J=7.4 Hz, 1H), 7.07 (s, 1H), 4.60-4.35 (m, 1H), 4.32-4.04 (m, 2H), 3.88-3.53 (m, 2H), 2.68-2.44 (m, 1H), 2.19-1.95 (m, 1H), 1.47 (d, J=6.2 Hz, 3H).

### Step b:

5,6-dichloro-1′-(2-hydroxyacetyl)-5′-methyl-1H-spiro[indole-3,3′-pyrrolidin]-2-one diastereoisomer A (45.0 mg, 0.140 mmol) was separated by Prep-Chiral HPLC with the following conditions: Column: CHIRAL ART Amylose-SA, 2×25 cm, 5 μm; Mobile Phase A: Hex (plus 0.5% 2M NH3-MeOH)-HPLC, Mobile Phase B: EtOH-HPLC; Flow rate: 20 mL/min; Gradient: 50% B to 50% B in 16 min; Wavelength: UV 220/254 nm; Retention Time 1: 6.95 min; Retention Time 2: 12.33 min; Sample Solvent: EtOH-HPLC; Injection Volume: 1.5 mL; Number Of Runs: 4. The faster eluting isomer at 6.95 min was obtained 5,6-dichloro-1′-(2-hydroxyacetyl)-5′-methyl-1H-spiro[indole-3,3′-pyrrolidin]-2-one isomer 1 as an off-white solid (12.6 mg, 28%): LCMS (ESI) calc'd for C₁₄H₁₄Cl₂N₂O₃ [M+H]⁺: 329, 331 (3:2) found 329, 331 (3:2); ¹H NMR (300 MHz, CD₃OD) δ 7.59 (s, 1H), 7.06 (s, 1H), 4.64-4.35 (m, 1H), 4.36-4.00 (m, 2H), 3.85-3.55 (m, 2H), 2.68-2.36 (m, 1H), 2.21-1.97 (m, 1H), 1.47 (d, J=6.3 Hz, 3H). The slower eluting isomer at 12.33 min was obtained 5,6-dichloro-1′-(2-hydroxyacetyl)-5′-methyl-1H-spiro[indole-3,3′-pyrrolidin]-2-one isomer 2 as an off-white solid (17.3 mg, 38%): LCMS (ESI) calc'd for C₁₄H₁₄Cl₂N₂O₃ [M+H]⁺: 329, 331 (3:2) found 329, 331 (3:2); ¹H NMR (300 MHz, CD₃OD) δ 7.59 (s, 1H), 7.06 (s, 1H), 4.60-4.33 (m, 1H), 4.31-4.01 (m, 2H), 3.89-3.48 (m, 2H), 2.66-2.35 (m, 1H), 2.24-1.97 (m, 1H), 1.47 (d, J=6.3 Hz, 3H).

### Step c:

5,6-dichloro-1′-(2-hydroxyacetyl)-5′-methyl-1H-spiro[indole-3,3′-pyrrolidin]-2-one diastereoisomer B (45.0 mg, 0.140 mmol) was separated by Prep Chiral HPLC with the following conditions: Column: CHIRAL ART Amylose-SA, 2×25 cm, 5 μm; Mobile Phase A: Hex (plus 0.5% 2 MNH3-MeOH)-HPLC, Mobile Phase B: EtOH-HPLC; Flow rate: 20 mL/min; Gradient: 50% B to 50% B in 10 min; Wavelength: UV 220/254 nm; Retention Time 1: 5.29 min; Retention Time 2: 8.44 min; Sample Solvent: EtOH-HPLC; Injection Volume: 1.5 mL; Number Of Runs: 3. The faster eluting isomer at 5.29 min was obtained 5,6-dichloro-1′-(2-hydroxyacetyl)-5′-methyl-1H-spiro[indole-3,3′-pyrrolidin]-2-one isomer 3 as an off-white solid (13.5 mg, 30%): LCMS (ESI) calc'd for C₁₄H₁₄Cl₂N₂O₃ [M+H]⁺: 329, 331 (3:2) found 329, 331 (3:2); ¹H NMR (300 MHz, CD₃OD) δ 7.12 (s, 2H), 4.60-4.41 (m, 1H), 4.34-4.01 (m, 2H), 3.80-3.55 (m, 2H), 2.54-1.98 (m, 2H), 1.48 (d, J=6.1 Hz, 3H). The slower eluting isomer at 8.44 min was obtained 5,6-dichloro-1′-(2-hydroxyacetyl)-5′-methyl-1H-spiro[indole-3,3′-pyrrolidin]-2-one isomer 4 as an off-white solid (15.7 mg, 35%): LCMS (ESI) calc'd for C₁₄H₁₄Cl₂N₂O₃ [M+H]⁺: 329, 331 (3:2) found 329, 331 (3:2); ¹H NMR (300 MHz, CD₃OD) δ 7.12 (s, 2H), 4.61-4.39 (m, 1H), 4.34-4.01 (m, 2H), 3.79-3.60 (m, 2H), 2.55-2.27 (m, 1H), 2.27-2.03 (m, 1H), 1.48 (d, J=6.2 Hz, 3H).

### Example 17. Compound 133 (5,6-dichloro-1′-(2-hydroxyacetyl)-2′-methylspiro[indoline-3,3′-pyrrolidin]-2-one diastereoisomer 1) and Compound 134 (5,6-dichloro-1′-(2-hydroxyacetyl)-2′-methylspiro[indoline-3,3′-pyrrolidin]-2-one diastereoisomer 2)

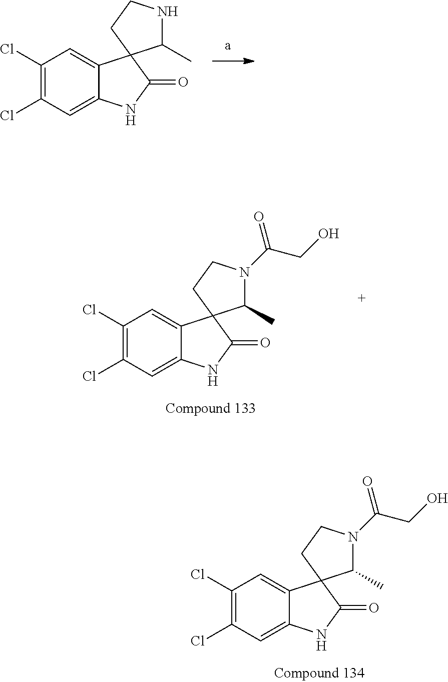 [SMILES: CC1NCCC12C(=O)Nc1cc(Cl)c(Cl)cc12 ; C[C@@H]1N(C(=O)CO)CCC12C(=O)Nc1cc(Cl)c(Cl)cc12 ; C[C@H]1N(C(=O)CO)CCC12C(=O)Nc1cc(Cl)c(Cl)cc12]

### Step a:

To a stirred solution of glycolic acid (54.7 mg, 0.719 mmol) and HOBT (97.2 mg, 0.719 mmol), EDCI (0.137 g, 0.715 mmol) in DMF (0.5 mL) were added TEA (0.145 g, 1.44 mmol) and 5,6-dichloro-2′-methyl-1H-spiro[indole-3,3′-pyrrolidin]-2-one (0.130 g, 0.479 mmol) at room temperature. The mixture was stirred for 2 h, diluted with water (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by Prep-HPLC with the following conditions: Column: X Bridge Prep C18 OBD Column, 19×150 mm, 5 μm; Mobile Phase A: Water (plus 10 mM NH₄HCO₃), Mobile Phase B: ACN; Flow rate: 20 mL/min; Gradient: 30% B to 40% B in 5.5 min, 40% B; Detector: UV 254/220 nm; Retention time 1: 4.80 min, Retention time 2: 5.30 min. The faster-eluting diastereoisomer at 4.80 min was obtained 5,6-dichloro-1′-(2-hydroxyacetyl)-2′-methyl-1H-spiro[indole-3,3′-pyrrolidin]-2-one diastereoisomer 1 as an off-white solid (44.6 mg, 28%): LCMS (ESI) calc'd for C₁₄H₁₄Cl₂N₂O₃ [M+H]⁺: 329, 331 (3:2) found 329, 331 (3:2); ¹H NMR (400 MHz, CD₃OD) δ 7.48-7.36 (m, 1H), 7.13-7.05 (m, 1H), 4.37-4.16 (m, 3H), 4.07-3.88 (m, 1H), 3.88-3.76 (m, 1H), 2.58-2.37 (m, 1H), 2.35-2.22 (m, 1H), 1.37-1.25 (m, 3H). The slower-eluting diastereoisomer at 5.30 min was obtained 5,6-dichloro-1′-(2-hydroxyacetyl)-2′-methyl-1H-spiro[indole-3,3′-pyrrolidin]-2-one diastereoisomer 2 as an off-white solid (3.5 mg, 2%): LCMS (ESI) calc'd for C₁₄H₁₄Cl₂N₂O₃ [M+H]⁺: 329, 331 (3:2) found 329, 331 (3:2); ¹H NMR (400 MHz, CD₃OD) δ 7.54 (s, 1H), 7.10 (s, 1H), 4.33-4.13 (m, 3H), 4.01-3.69 (m, 2H), 2.61-2.39 (m, 1H), 2.39-2.15 (m, 1H), 1.42-1.22 (m, 3H).

### Example 18. Compound 130 (5,6-dichloro-1′-(2-hydroxyacetyl)-4′-methylspiro[indoline-3,3′-pyrrolidin]-2-one diastereoisomer 1) and Compound 131 (5,6-dichloro-1′-(2-hydroxyacetyl)-4′-methylspiro[indoline-3,3′-pyrrolidin]-2-one diastereoisomer 2)

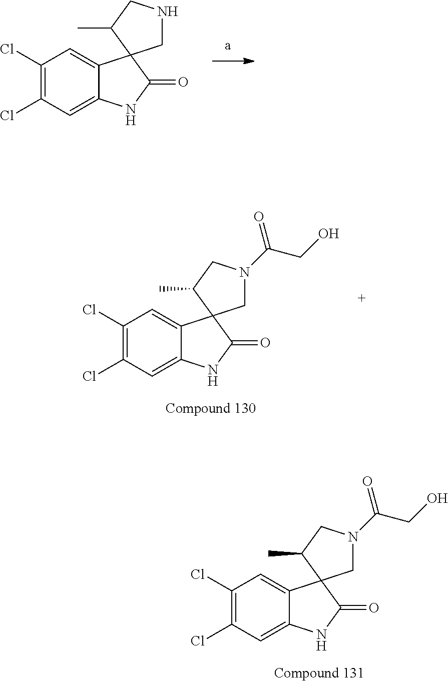 [SMILES: CC1CNCC12C(=O)Nc1cc(Cl)c(Cl)cc12 ; C[C@@H]1CN(C(=O)CO)CC12C(=O)Nc1cc(Cl)c(Cl)cc12 ; C[C@H]1CN(C(=O)CO)CC12C(=O)Nc1cc(Cl)c(Cl)cc12]

### Step a:

To a stirred solution of glycolic acid (25.2 mg, 0.331 mmol), HOBT (44.9 mg, 0.332 mmol) and EDCI (63.6 mg, 0.332 mmol) in DMF (1 mL) were added TEA (67.2 mg, 0.663 mmol) and 5,6-dichloro-4′-methyl-1H-spiro[indole-3,3′-pyrrolidin]-2-one (60.0 mg, 0.221 mmol) at room temperature. The reaction mixture was stirred for 2 h, diluted with water (30 mL) and extracted with EA (3×20 mL). The combined organic layers were washed with brine (3×20 mL) and dried over anhydrous Na₂SO₄. After filtration, the filtrate was concentrated under reduced pressure. The residue was purified by Prep-HPLC with the following conditions: Column: X Bridge Prep C18 OBD Column, 19×100 mm, 5 μm; Mobile Phase A: Water (plus 10 mM NH₄HCO₃), Mobile Phase B: ACN; Flow rate: 20 mL/min; Gradient: 25% B to 50% B in 4.5 min, 50% B, Detector: UV 254/220 nm; Retention time 1: 4.35 min, Retention time 2: 4.60 min. The faster-eluting diastereoisomer at 4.35 min was obtained 5,6-dichloro-1′-(2-hydroxyacetyl)-4′-methyl-1H-spiro[indole-3,3′-pyrrolidin]-2-one diastereoisomer 1 as an off-white solid (2.60 mg, 4%): LCMS (ESI) calc'd for C₁₄H₁₄Cl₂N₂O₃ [M+H]⁺: 329, 331 (3:2) found 329, 331 (3:2); ¹H NMR (300 MHz, CD₃OD) δ 7.27 (d, J=38.03 Hz, 1H), 7.14 (s, 1H), 4.42-4.21 (m, 1H), 4.21-3.90 (m, 2H), 3.87-3.69 (m, 2H), 3.53-3.36 (m, 1H), 2.97-2.71 (m, 1H), 0.75 (d, J=6.78 Hz, 3H). The slower-eluting diastereoisomer at 4.60 min was obtained 5,6-dichloro-1′-(2-hydroxyacetyl)-4′-methyl-1H-spiro[indole-3,3′-pyrrolidin]-2-one diastereoisomer 2 as an off-white solid (4.40 mg, 6%): LCMS (ESI) calc'd for C₁₄H₁₄Cl₂N₂O₃ [M+H]⁺: 329, 331 (3:2) found 329, 331 (3:2): ¹H NMR (300 MHz, CD₃OD) δ 7.55 (d, J=3.10 Hz, 1H), 7.08 (d, J=1.56 Hz, 1H), 4.32-4.14 (m, 2H), 4.03-3.91 (m, 1H), 3.88-3.76 (m, 1H), 3.72 (d, J=12.42 Hz, 1H), 3.61-3.50 (m, 1H), 2.93-2.66 (m, 1H), 0.88 (dd, J=6.78, 2.30 Hz, 3H).

### Example 19. Compound 144 ((3S)-1′-(5-aminopyrazin-2-yl)-5,6-dichloro-1H-spiro[indole-3,3′-pyrrolidin]-2-one)

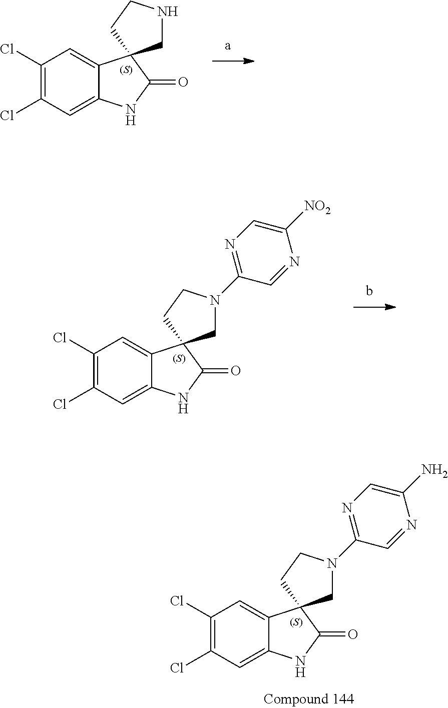

### Step a:

To a stirred solution of (3S)-5,6-dichloro-1H-spiro[indole-3,3′-pyrrolidin]-2-one (60.0 mg, 0.233 mmol) and 2-chloro-5-nitropyrazine (45.0 mg, 0.282 mmol) in DMA (1.00 mL) was added Cs₂CO₃ (0.152 g, 0.467 mmol) at room temperature. The reaction mixture was stirred for 1 h and directly purified by reverse phase chromatography, eluting with 50% ACN in water (plus 10 mM NH₄HCO₃) to afford (3S)-5,6-dichloro-1′-(5-nitropyrazin-2-yl)-1H-spiro[indole-3,3′-pyrrolidin]-2-one as a yellow solid (60.0 mg, 68%): LCMS (ESI) calc'd for C₁₅H₁₁Cl₂N₅O₃ [M−H]⁻: 378, 380 (3:2), found 378, 380 (3:2); ¹H NMR (400 MHz, DMSO-d₆) δ 10.87 (s, 1H), 9.08 (s, 1H), 8.02 (d, J=48.2 Hz, 1H), 7.70 (s, 1H), 7.09 (s, 1H), 4.03-3.93 (m, 4H), 2.41 (s, 2H).

### Step b:

To a stirred solution of (3S)-5,6-dichloro-1′-(5-nitropyrazin-2-yl)-1H-spiro[indole-3,3′-pyrrolidin]-2-one (60.0 mg, 0.159 mmol) and CaCl₂) (79.0 mg, 0.712 mmol) in EtOH (4 mL) and H₂O (1 mL) was added Fe (0.264 g, 4.73 mmol) at room temperature. The resulting mixture was stirred for 2 h at 80° C. and filtered. The filter cake was washed with EtOH (3×10 mL) and the filtrate concentrated under reduced pressure. The residue was purified by Prep-HPLC with the following conditions: Column: SunFire Prep C18 OBD Column, 19×150 mm, 5 μm; Mobile Phase A: Water (plus 10 mM NH₄OAc), Mobile Phase B: ACN; Flow rate: 20 mL/min; Gradient: 30% B to 50% B in 4.5 min, 50% B; Detector: UV 254/210 nm; Retention time: 4.35 min; The fractions containing the desired product were collected and concentrated under reduced pressure to afford (3S)-1′-(5-aminopyrazin-2-yl)-5,6-dichloro-1H-spiro[indole-3,3′-pyrrolidin]-2-one as a yellow solid (2.10 mg, 4%): LCMS (ESI) calc'd for C₁₅H₁₃Cl₂N₅O [M+H]⁺: 350, 352 (3:2) found 350, 352 (3:2): ¹H NMR (400 MHz, CD₃OD) δ 7.69 (d, J=1.7 Hz, 1H), 7.51 (d, J=1.7 Hz, 1H), 7.37 (s, 1H), 7.11 (s, 1H), 3.89-3.63 (m, 4H), 2.55-2.45 (m, 1H), 2.35-2.24 (m, 1H).

The compounds in Table 1H below were prepared in an analogous fashion to that described for Compound 144, starting from (3S)-5,6-dichloro-1H-spiro[indole-3,3′-pyrrolidin]-2-one and the corresponding heteroaryl chlorides, which were available from commercial sources.

TABLE 1H

| Compound | | | |
|---|---|---|---|
| No. | Structure | Chemical Name | MS: (M + H)⁺ & ¹H MNR |
| 139 | 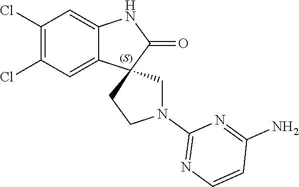 | (S)-1′-(4- aminopyrimidin-2- yl)-5,6- dichlorospiro [indoline- 3,3′-pyrrolidin]-2- one | [M + H]⁺: 350, 352 (3:2); ¹H NMR (300 MHz, CD₃OD) δ 7.68 (d, J = 7.24 Hz, 1H), 7.55 (s, 1H), 7.13 (s, 1H), 6.18 (d, J = 7.23 Hz, 1H), 4.12-3.75 (m, 4H), 2.59-2.38 (m, 2H). |
| 145 | 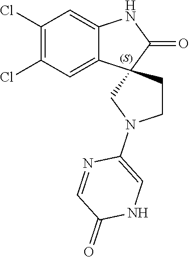 | (S)-5,6-dichloro-1′- (5-oxo-4,5- dihydropyrazin-2- yl)spiro[indoline- 3,3′-pyrrolidin]-2-one | [M + H]⁺: 351, 353 (3:2); ¹H NMR (400 MHz, DMSO-d₆) δ 11.53 (s, 1H), 10.77 (s, 1H), 7.52 (s, 1H), 7.06 (s, 1H), 6.78 (d, J = 4.27 Hz, 1H), 6.65-6.59 (m, 1H), 4.11- 3.93 (m, 4H), 2.29-2.14 (m, 2H). |
| 148 | 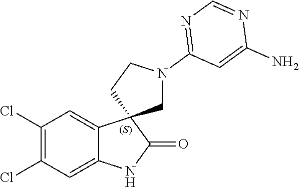 | (S)-1′-(6- aminopyrimidin-4- yl)-5,6- dichlorospiro [indoline- 3,3′-pyrrolidin]-2- one | [M + H]⁺: 350, 352 (3:2); ¹H NMR (400 MHz, DMSO-d₆) δ 10.78 (s, 1H), 7.94 (s, 1H), 7.51 (s, 1H), 7.06 (s, 1H), 6.21 (s, 2H), 5.39 (s, 1H), 3.79-3.53 (m, 4H), 2.35-2.20 (m, 2H). |
| 149 | 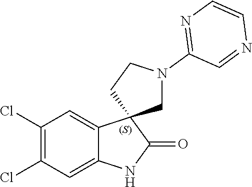 | (S)-5,6-dichloro-1′- (pyrazin-2- yl)spiro[indoline- 3,3′-pyrrolidin]-2-one | [M + H]⁺: 335, 337 (3:2); ¹H NMR (400 MHz, DMSO-d₆) δ 10.81 (brs, 1H), 8.08 (dd, J = 2.70, 1.48 Hz, 1H), 8.04 (d, J = 1.56 Hz, 1H), 7.83 (d, J = 2.72 Hz, 1H), 7.59 (s, 1H), 7.07 (s, 1H), 3.88-3.79 (m, 2H), 3.75 (s, 2H), 2.38-2.31 (m, 2H). |

### Example 20. Compound 153 ((3S)-5,6-dichloro-1′-(1H-pyrazol-3-yl)-1H-spiro[indole-3,3′-pyrrolidin]-2-one)

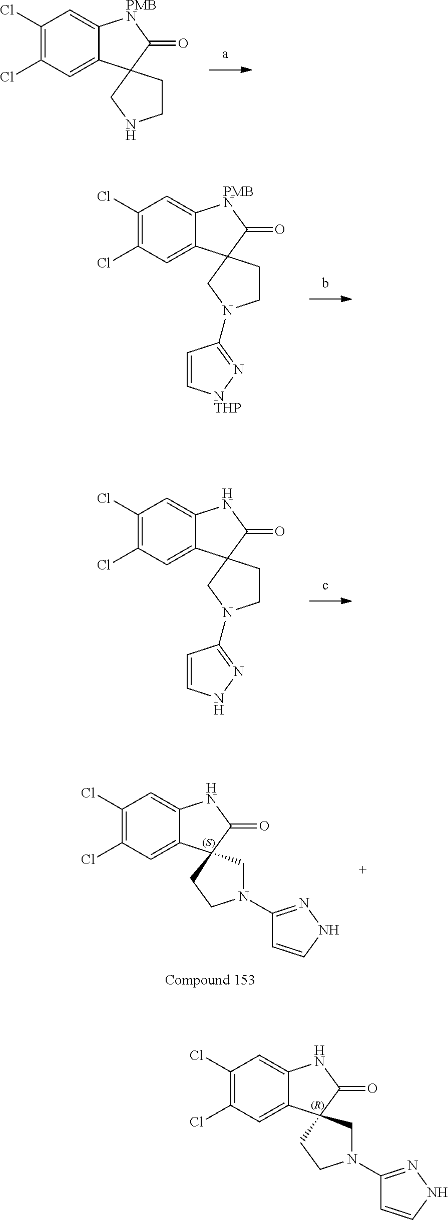

### Step a:

To a stirred mixture of 5,6-dichloro-1-[(4-methoxyphenyl)methyl]spiro[indole-3,3-pyrrolidin]-2-one (0.150 g, 0.398 mmol) and 3-bromo-1-(tetrahydropyran-2-yl)pyrazole (0.184 g, 0.796 mmol) in dioxane (2 mL) were added EPhos Pd G4 (36.5 mg, 0.0400 mmol), EPhos (21.3 mg, 0.0400 mmol) and Cs₂CO₃ (0.259 g, 0.795 mmol) at room temperature under nitrogen atmosphere. The reaction mixture was stirred at 110° C. for 2 days, quenched with water (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 55% ACN in water (plus 0.05% TFA) to afford 5,6-dichloro-1-[(4-methoxyphenyl)methyl]-1′-[1-(tetrahydropyran-2-yl)pyrazol-3-yl]spiro[indole-3,3′-pyrrolidin]-2-one as a brown solid (0.110 g, 42%): LCMS (ESI) calc'd for C₂₇H₂₈Cl₂N₄O₃ [M+H]⁺: 527, 529 (3:2) found 527, 529 (3:2); ¹H NMR (300 MHz, CDCl₃) δ 7.46 (d, J=2.51 Hz, 1H), 7.35 (d, J=1.07 Hz, 1H), 7.24-7.18 (m, 2H), 6.93-6.87 (m, 2H), 6.84 (s, 1H), 5.67 (d, J=2.54 Hz, 1H), 5.32 (s, 1H), 5.30-5.22 (m, 1H), 4.96-4.77 (m, 2H), 4.14-4.04 (m, 1H), 4.04-3.97 (m, 1H), 3.85-3.69 (m, 5H), 3.59 (d, J=9.55 Hz, 1H), 2.66-2.49 (m, 2H), 2.22-1.97 (m, 4H), 1.80-1.52 (m, 2H).

### Step b:

To a stirred solution of 5,6-dichloro-1-[(4-methoxyphenyl)methyl]-1-[1-(tetrahydropyran-2-yl)pyrazol-3-yl]spiro[indole-3,3-pyrrolidin]-2-one (0.110 g, 0.208 mmol) in DCM (1 mL) and TFA (1 mL) was added trifluoromethanesulfonic acid (0.313 g, 2.09 mmol) dropwise at room temperature. The reaction mixture was stirred for 2 h and concentrated under reduced pressure. The residue was purified by Prep-HPLC with the following conditions: Column: XBridge Prep C18 OBD Column, 19×150 mm, 5 μm; Mobile Phase A: Water (10 mM NH₄HCO₃), Mobile Phase B: ACN; Flow rate: 20 mL/min; Gradient: 30% B to 50% B in 4.5 min; Detector: UV 254/210 nm; Retention Time: 4.30 min. The fractions containing the desired product were collected and concentrated under reduced pressure to afford 5,6-dichloro-1-(1H-pyrazol-3-yl)-1H-spiro[indole-3,3-pyrrolidin]-2-one as an off-white solid (60.0 mg, 89%): LCMS (ESI) calc'd for C₁₄H₁₂Cl₂N₄O [M+H]⁺: 323, 325 (3:2) found 323, 325 (3:2); ¹H NMR (400 MHz, CD₃OD) δ 7.47 (s, 1H), 7.35 (s, 1H), 7.10 (s, 1H), 5.70 (s, 1H), 3.81-3.70 (m, 1H), 3.70-3.61 (m, 1H), 3.61-3.52 (m, 2H), 2.57-2.44 (m, 1H), 2.27-2.15 (m, 1H).

### Step c:

The 5,6-dichloro-1′-(1H-pyrazol-3-yl)-1H-spiro[indole-3,3′-pyrrolidin]-2-one (28.0 mg, 0.0866 mmol) was purified by Prep-CHIRAL-HPLC with the following conditions: Column: CHIRAL ART Cellulose-SC, 2×25 cm, 5 μm; Mobile Phase A: Hex (plus 0.3% IPA)-HPLC, Mobile Phase B: EtOH-HPLC; Flow rate: 20 mL/min; Gradient: 15% B to 15% B in 15 min; Wavelength: UV 220/254 nm; Retention Time 1: 10.80 min; Retention Time 2: 13.84 min; Sample Solvent: EtOH:DCM=1:1; Injection Volume: 1 mL; Number Of Runs: 2. The faster-eluting enantiomer at 10.80 min was obtained (3S)-5,6-dichloro-1′-(1H-pyrazol-3-yl)-1H-spiro[indole-3,3′-pyrrolidin]-2-one as an off-white solid (11.0 mg, 39%): LCMS (ESI) calc'd for C₁₄H₁₂Cl₂N₄O [M+H]⁺: 323, 325 (3:2) found 323, 325 (3:2); ¹H NMR (300 MHz, CD₃OD) δ 7.47 (d, J=2.41 Hz, 1H), 7.35 (s, 1H), 7.09 (s, 1H), 5.70 (d, J=2.40 Hz, 1H), 3.81-3.70 (m, 1H), 3.69-3.59 (m, 1H), 3.56 (d, J=3.06 Hz, 2H), 2.56-2.42 (m, 1H), 2.28-2.14 (m, 1H). The slower-eluting enantiomer at 13.84 min was obtained (3R)-5,6-dichloro-1′-(1H-pyrazol-3-yl)-1H-spiro[indole-3,3′-pyrrolidin]-2-one as an off-white solid (10.0 mg, 36%): LCMS (ESI) calc'd for C₁₄H₁₂Cl₂N₄O [M+H]⁺: 323, 325 (3:2) found 323, 325 (3:2); ¹H NMR (300 MHz, CD₃OD) δ 7.47 (d, J=2.40 Hz, 1H), 7.35 (s, 1H), 7.09 (s, 1H), 5.69 (d, J=2.41 Hz, 1H), 3.81-3.70 (m, 1H), 3.70-3.58 (m, 1H), 3.56 (d, J=3.10 Hz, 2H), 2.56-2.42 (m, 1H), 2.27-2.17 (m, 1H).

The compound in Table 11 below was prepared in an analogous fashion to that described for Compound 153, starting from 5,6-dichloro-1-[(4-methoxyphenyl)methyl]spiro[indole-3,3-pyrrolidin]-2-one and the corresponding heteroaryl bromide, which was available from commercial sources.

| Compound | | | |
|---|---|---|---|
| No. | Structure | Chemical Name | MS: (M + H)⁺ & ¹H MNR |
| 146 | 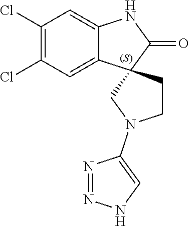 | (S)-5,6-dichloro-1′- (1H-1,2,3-triazol-4- yl)spiro[indoline- 3,3′-pyrrolidin]-2- one | [M + H]⁺: 324, 326 (3:2); ¹H NMR (300 MHz, CD₃OD) δ 7.38 (s, 1H), 7.20 (s, 1H), 7.10 (s, 1H), 3.84-3.62 (m, 2H), 3.60 (s, 2H), 2.57-2.44 (m, 1H), 2.34- 2.20 (m, 1H). |

### Example 21. Compound 132 ((S)-1′-(5-amino-1,3,4-oxadiazol-2-yl)-5,6-dichlorospiro[indoline-3,3′-pyrrolidin]-2-one)

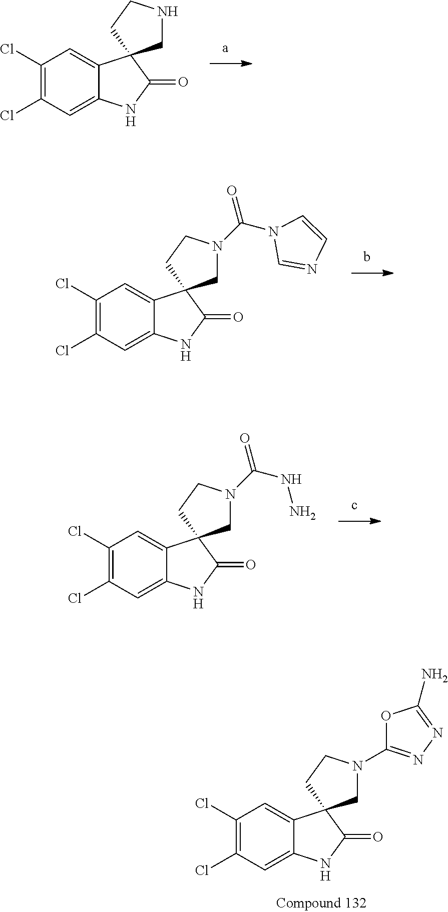 [SMILES: O=C1Nc2cc(Cl)c(Cl)cc2[C@]12CCNC2 ; O=C(N1CC[C@]2(C1)C(=O)Nc1cc(Cl)c(Cl)cc12)n1ccnc1 ; NNC(=O)N1CC[C@]2(C1)C(=O)Nc1cc(Cl)c(Cl)cc12 ; Nc1nnc(N2CC[C@]3(C2)C(=O)Nc2cc(Cl)c(Cl)cc23)o1]

### Step a:

To a stirred solution of (3S)-5,6-dichloro-1H-spiro[indole-3,3′-pyrrolidin]-2-one (0.100 g, 0.389 mmol) in THF (2 mL) was added CDI (0.189 g, 1.34 mmol) in portions at room temperature under nitrogen atmosphere. The reaction mixture was stirred for 6 h, quenched with water (20 mL) and extracted with EA (3×20 mL). The combined organic layers were washed with brine (3×20 mL) and dried over anhydrous Na₂SO₄. After filtration, the filtrate was concentrated under reduced pressure to afford (3S)-5,6-dichloro-1′-(imidazole-1-carbonyl)-1H-spiro[indole-3,3′-pyrrolidin]-2-one as a light brown oil (0.120 g, 88%), which was used directly in the next step without purification: LCMS (ESI) calc'd for C₁₅H₁₂Cl₂N₄O₂ [M+H]⁺: 351, 353 (3:2) found 351, 353 (3:2).

### Step b:

To a stirred solution of (3S)-5,6-dichloro-1′-(imidazole-1-carbonyl)-1H-spiro[indole-3,3′-pyrrolidin]-2-one (0.120 g, 0.342 mmol) in THE (2 mL) was added hydrazine hydrate (28.5 mg, 0.558 mmol, 98%) at room temperature under nitrogen atmosphere. The reaction mixture was stirred for 4 h and concentrated under reduced pressure. The residue was purified by reverse phase chromatography, eluting with 50% ACN in water (plus 0.05% TFA) to afford (3S)-5,6-dichloro-2-oxo-1H-spiro[indole-3,3′-pyrrolidine]-1′-carbohydrazide as a light yellow oil (0.100 g, 83%): LCMS (ESI) calc'd C₁₂H₁₂Cl₂N₄O₂ for [M+H]⁺: 315, 317 (3:2) found 315, 317 (3:2); ¹H NMR (400 MHz, DMSO-d₆) δ 10.82 (s, 1H), 9.17 (s, 1H), 7.55 (s, 1H), 7.07 (s, 1H), 3.77-3.55 (m, 4H), 2.26 (t, J=7.1 Hz, 2H).

### Step c:

To a stirred solution of (3S)-5,6-dichloro-2-oxo-1H-spiro[indole-3,3′-pyrrolidine]-1′-carbohydrazide (0.100 g, 0.317 mmol) in EtOH (2 mL) was added BrCN (67.2 mg, 0.634 mmol) at room temperature under nitrogen atmosphere. The reaction mixture was stirred for 16 h and concentrated under reduced pressure. The residue was purified by Prep-HPLC with the following conditions: Column: SunFire Prep C18 OBD Column, 19×150 mm, 5 μm; Mobile Phase A: water (plus 0.05% TFA), Mobile Phase B: ACN; Flow rate: 20 mL/min; Gradient: 25% B to 50% B in 4.5 min, 50% B; Wavelength: UV 254/210 nm; Retention Time: 4.35 min. The fractions containing the desired product were collected and concentrated under reduced pressure to afford (S)-1′-(5-amino-1,3,4-oxadiazol-2-yl)-5,6-dichlorospiro[indoline-3,3′-pyrrolidin]-2-one as an off-white solid (21.7 mg, 20%): LCMS (ESI) calc'd for C₁₃H₁₁Cl₂N₅O₂ [M+H]⁺: 340, 342 (3:2) found 340, 342 (3:2); ¹H NMR (400 MHz, CD₃OD) δ 7.47 (s, 1H), 7.11 (s, 1H), 3.93-3.75 (m, 2H), 3.77 (d, J=10.4 Hz, 1H), 3.68 (d, J=10.3 Hz, 1H), 2.49-2.47 (m, 1H), 2.46-2.44 (m, 1H).

### Example 22. Compound 138 ((3S)-5,6-dichloro-1′-(2-hydroxyethanesulfonyl)-1H-spiro[indole-3,3′-pyrrolidin]-2-one)

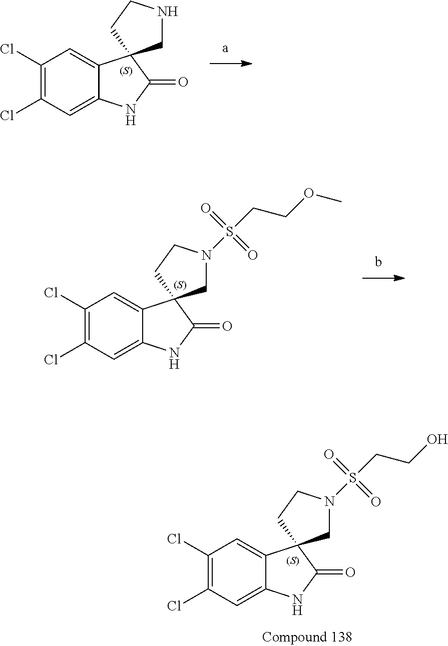

### Step a:

To a stirred mixture of (3S)-5,6-dichloro-1H-spiro[indole-3,3′-pyrrolidin]-2-one (40.0 mg, 0.155 mmol) in DCM (1.00 mL) was added TEA (31.0 mg, 0.208 mmol) and 2-methoxyethanesulfonyl chloride (25.0 mg, 0.158 mmol) dropwise at 0° C. The reaction mixture was stirred at room temperature for 2 h, diluted with water (20 mL) and extracted with DCM (3×20 mL). The combined organic layers were washed with brine (3×20 mL) and dried over anhydrous Na₂SO₄. After filtration, the filtrate was concentrated under reduced pressure to afford (3S)-5,6-dichloro-1′-(2-methoxyethanesulfonyl)-1H-spiro[indole-3,3′-pyrrolidin]-2-one as a yellow solid (51.4 mg, 87%), which was used directly in the next step without purification: LCMS (ESI) calc'd for C₁₄H₁₆Cl₂N₂O₄S [M+H]⁺: 379, 381 (3:2) found 379, 381 (3:2).

### Step b:

To a stirred solution of (3S)-5,6-dichloro-1′-(2-methoxyethanesulfonyl)-1H-spiro[indole-3,3′-pyrrolidin]-2-one (51.0 mg, 0.134 mmol) in DCM (1.00 mL) was added BBr₃ (68.0 mg, 0.271 mmol) at room temperature. The reaction mixture was stirred for 2 h, quenched with MeOH (5 mL) at 0° C. and concentrated under reduced pressure. The residue was purified by Prep-HPLC with the following conditions: Column: XBridge Shield RP18 OBD Column, 30×150 mm, 5 μm; Mobile Phase A: Water (plus 10 mM NH₄HCO₃), Mobile Phase B: ACN; Flow rate: 25 mL/min; Gradient: 22% B to 50% B in 5.2 min; Detector: UV 210 nm; Retention time: 5.14 min; The fractions containing the desired product were collected and concentrated under reduced pressure to afford (3S)-5,6-dichloro-1′-(2-hydroxyethanesulfonyl)-1H-spiro[indole-3,3′-pyrrolidin]-2-one as an off-white solid (22.7 mg, 46%): LCMS (ESI) calc'd for C₁₃H₁₄Cl₂N₂O₄S [M+H]⁺: 365, 367 (3:2) found 365, 367 (3:2); ¹H NMR (400 MHz, DMSO-d₆) δ 10.80 (s, 1H), 7.63 (s, 1H), 7.05 (s, 1H), 5.09 (t, J=5.4 Hz, 1H), 3.82 (q, J=6.1 Hz, 2H), 3.73-3.60 (m, 2H), 3.55 (q, J=10.1 Hz, 2H), 3.37 (d, J=6.3 Hz, 2H), 2.30-2.15 (m, 2H).

The compounds in Table 1J below were prepared in an analogous fashion to that described for Compound 138, starting from (3S)-5,6-dichloro-1H-spiro[indole-3,3′-pyrrolidin]-2-one and the corresponding sulfonyl chlorides, which were available from commercial sources.

TABLE 1J

| Compound | | | |
|---|---|---|---|
| No. | Structure | Chemical Name | MS: (M + H)⁺ & ¹H MNR |
| 142 | 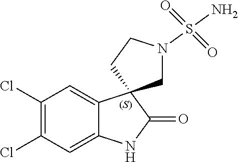 | (S)-5,6-dichloro-2- oxospiro[indoline- 3,3′-pyrrolidine]-1′- sulfonamide | [M + H]⁺: 336, 338 (3:2); ¹H NMR (300 MHz, DMSO-d₆) δ 10.81 (s, 1H), 7.55 (s, 1H), 7.06 (s, 1H), 7.01 (s, 2H), 3.56-3.47 (m, 2H), 3.42-3.28 (m, 2H), 2.30-2.18 (m, 1H), 2.13-2.02 (m, 1H). |
| 143 | 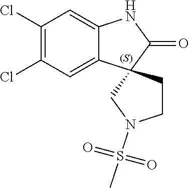 | (S)-5,6-dichloro-2- (methylsulfonyl) spiro[indoline-3,3′- pyrrolidin]-2-one | [M + H]⁺: 336, 338 (3:2); ¹H NMR (300 MHz, CD₃OD) δ 7.56 (s, 1H), 7.09 (s, 1H), 3.85-3.70 (m, 2H), 3.70-3.56 (m, 2H), 3.03 (s, 3H), 2.47- 2.26 (m, 2H). |

### Example 23. Compound 154 ((3S)-5,6-dichloro-2-oxo-1H-spiro[indole-3,3′-pyrrolidine]-1′-carboxamide) and Compound 155 ((3R)-5,6-dichloro-2-oxo-1H-spiro[indole-3,3′-pyrrolidine]-1′-carboxamide)

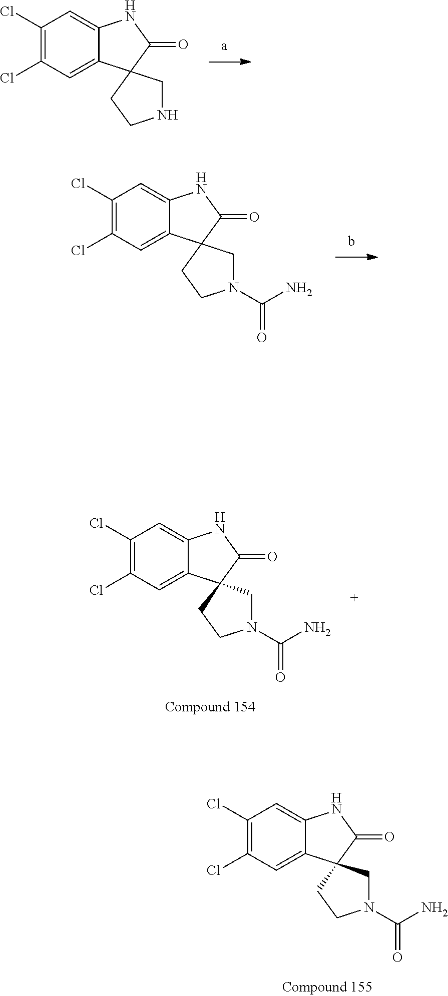 [SMILES: O=C1Nc2cc(Cl)c(Cl)cc2C12CCNC2 ; NC(=O)N1CCC2(C1)C(=O)Nc1cc(Cl)c(Cl)cc12 ; NC(=O)N1CC[C@]2(C1)C(=O)Nc1cc(Cl)c(Cl)cc12 ; NC(=O)N1CC[C@@]2(C1)C(=O)Nc1cc(Cl)c(Cl)cc12]

### Step a:

To a stirred solution of 5,6-dichloro-1H-spiro[indole-3,3-pyrrolidin]-2-one (0.100 g, 0.389 mmol) and TEA (0.118 g, 0.791 mmol) in DCM (2.00 mL) was added isocyanatotrimethylsilane (89.6 mg, 0.778 mmol) at 0° C. The reaction mixture was stirred for 2 h and concentrated under reduced pressure. The residue was purified by Prep-HPLC with the following conditions: Column: XBridge Prep Phenyl OBD Column, 19×150 mm 5 μm 13 nm; Mobile Phase A: Water (plus 10 mM NH₄HCO₃), Mobile Phase B: ACN; Flow rate: 20 mL/min; Gradient: 30% B to 50% B in 4.3 min; Detector: UV 254/210 nm; Retention time: 4.20 min. The fractions containing the desired product were collected and concentrated under reduced pressure to afford 5,6-dichloro-2-oxo-1H-spiro[indole-3,3-pyrrolidine]-1-carboxamide as an off-white solid (65.0 mg, 56%): LCMS (ESI) calc'd for C₁₂H₁₁Cl₂N₃O₂ [M+H]⁺: 300, 302 (3:2) found 300, 302 (3:2); ¹H NMR (300 MHz, DMSO-d₆) δ 10.76 (s, 1H), 7.47 (s, 1H), 7.06 (s, 1H), 5.89 (s, 2H), 3.68-3.53 (m, 2H), 3.49 (s, 2H), 2.29-2.06 (m, 2H).

### Step b:

The 5,6-dichloro-2-oxo-1H-spiro[indole-3,3′-pyrrolidine]-1′-carboxamide (58.0 mg, 0.193 mmol) was purified by Prep-Chiral HPLC with the following conditions: Column: CHIRAL ART Amylose-SA, 2×25 cm, 5 μm; Mobile Phase A: Hex (plus 0.3% IPA)-HPLC, Mobile Phase B: EtOH-HPLC; Flow rate: 20 mL/min; Gradient: 50% B to 50% B in 7 min; Wavelength: UV 220/254 nm; Retention Time 1: 5.10 min; Retention Time 2: 6.45 min; Sample Solvent: EtOH:DCM=1:1; Injection Volume: 0.3 mL; Number Of Runs: 17. The faster-eluting enantiomer at 5.10 min was obtained (3S)-5,6-dichloro-2-oxo-1H-spiro[indole-3,3′-pyrrolidine]-1′-carboxamide as an off-white solid (20.1 mg, 35%): LCMS (ESI) calc'd for C₁₂H₁₁Cl₂N₃O₂ [M+H]⁺: 300, 302 (3:2) found 300, 302 (3:2); ¹H NMR (300 MHz, CD₃OD) δ 7.40 (s, 1H), 7.10 (s, 1H), 3.87-3.64 (m, 3H), 3.64-3.53 (m, 1H), 2.47-2.34 (m, 1H), 2.31-2.16 (m, 1H). The slower-eluting enantiomer at 6.45 min was obtained (3R)-5,6-dichloro-2-oxo-1H-spiro[indole-3,3′-pyrrolidine]-1′-carboxamide as an off-white solid (22.0 mg, 38%): LCMS (ESI) calc'd for C₁₂H₁₁Cl₂N₃O₂ [M+H]⁺: 300, 302 (3:2) found 300, 302 (3:2); ¹H NMR (300 MHz, CD₃OD) δ 7.40 (s, 1H), 7.10 (s, 1H), 3.87-3.65 (m, 3H), 3.64-3.54 (m, 1H), 2.47-2.34 (m, 1H), 2.31-2.18 (m, 1H).

### Example 24. Evaluation of Kv1.3 potassium channel blocker activities

This assay is used to evaluate the disclosed compounds' activities as Kv1.3 potassium channel blockers.

### Cell Culture

CHO-K1 cells stably expressing Kv1.3 were grown in DMEM containing 10% heat-inactivated FBS, 1 mM sodium pyruvate, 2 mM L-glutamine, and G418 (500 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The currents were evoked by applying 100 ms depolarizing pulses from −90 mV (holding potential) to +40 mV were applied with 0.1 Hz frequency. Control (compound-free) and compound pulse trains for each compound concentration applied contained 20 pulses. 10-second breaks were used between pulse trains (see Table A below).

TABLE A
Voltage Protocol

| Control | 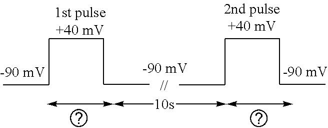 | // . . . // | 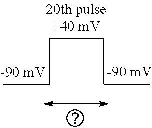 | | |
|---|---|---|---|---|---|
| | | | | / / 10 s | Compound application 1st concentration |
| Compound 1st concentration | 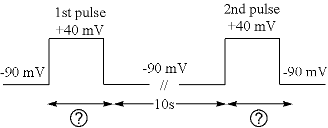 | // . . . // | 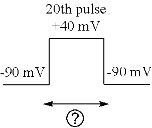 | | |
| | | | | / / 10 s | Compound application 2nd concentration |
| Compound 2nd concentration | 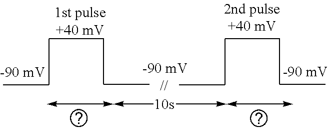 | // . . . // | 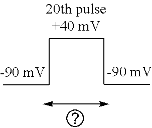 | | |
| ▪ ▪ ▪ Compound Nth Concentration | 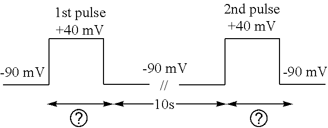 | // . . . // | 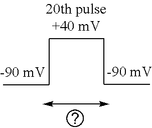 | ▪ ▪ ▪ | compound application Nth concentration |

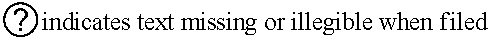

### Patch Clamp Recordings and Compound Application

Whole-cell current recordings and compound application were enabled by means of an automated patch clamp platform Patchliner (Nanion Technologies GmbH). EPC 10 patch clamp amplifier (HEKA Elektronik Dr. Schulze GmbH) along with Patchmaster software (HEKA Elektronik Dr. Schulze GmbH) was used for data acquisition. Data were sampled at 10 kHz without filtering. Passive leak currents were subtracted online using a P/4 procedure (HEKA Elektronik Dr. Schulze GmbH). Increasing compound concentrations were applied consecutively to the same cell without washouts in between. Total compound incubation time before the next pulse train was not longer than 10 seconds. Peak current inhibition was observed during compound equilibration.

### Data Analysis

AUC and peak values were obtained with Patchmaster (HEKA Elektronik Dr. Schulze GmbH). To determine IC₅₀, the last single pulse in the pulse train corresponding to a given compound concentration was used. Obtained AUC and peak values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ value is the concentration at which current inhibition is half-maximal, [compound] is the applied compound concentration, A is the fraction of current that is not blocked and nH is the Hill coefficient.

### Example 25. Evaluation of hERG Activities

This assay is used to evaluate the disclosed compounds' inhibition activities against the hERG channel.

hERG Electrophysiology

This assay is used to evaluate the disclosed compounds' inhibition activities against the hERG channel.

### Cell Culture

CHO-K1 cells stably expressing hERG were grown in Ham's F-12 Medium with glutamine containing 10% heat-inactivated FBS, 1% penicillin/streptomycin, hygromycin (100 μg/ml) and G418 (100 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM Glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The voltage protocol (see Table B) was designed to simulate voltage changes during a cardiac action potential with a 300 ms depolarization to +20 mV (analogous to the plateau phase of the cardiac action potential), a repolarization for 300 ms to −50 mV (inducing a tail current) and a final step to the holding potential of −80 mV. The pulse frequency was 0.3 Hz. Control (compound-free) and compound pulse trains for each compound concentration applied contained 70 pulses.

hERG voltage protocol

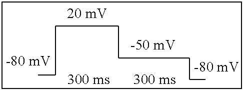

### Patch Clamp Recordings and Compound Application

Whole-cell current recordings and compound application were enabled by means of an automated patch clamp platform Patchliner (Nanion). EPC 10 patch clamp amplifier (HEKA) along with Patchmaster software (HEKA Elektronik Dr. Schulze GmbH) was used for data acquisition. Data were sampled at 10 kHz without filtering. Increasing compound concentrations were applied consecutively to the same cell without washouts in between.

### Data Analysis

AUC and PEAK values were obtained with Patchmaster (HEKA Elektronik Dr. Schulze GmbH). To determine IC₅₀ the last single pulse in the pulse train corresponding to a given compound concentration was used. Obtained AUC and PEAK values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ is the concentration at which current inhibition is half-maximal, [compound] is the applied compound concentration, A is the fraction of current that is not blocked and nH is the Hill coefficient.

Table 1 provides a summary of the inhibition activities of certain selected compounds of the instant invention against Kv1.3 potassium channel and hERG channel.

TABLE 1
IC₅₀ (μM) values of certain exemplified compounds against
Kv1.3 potassium channel and hERG channel

| Compound | | Kv1.3 | hERG |
|---|---|---|---|
| Number | Structure | IC₅₀ (μM) | IC₅₀ (μM) |
| 1 | 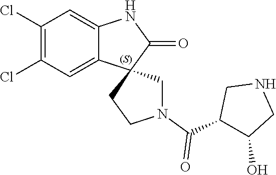 | <1 | * |
| 2 | 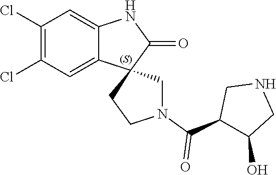 | <0.3 | >100 |
| 3 | 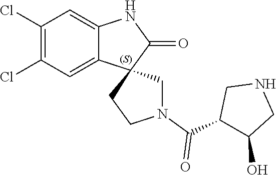 | <0.1 | >100 |
| 4 | 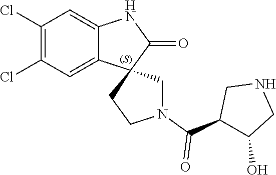 | <1 | * |
| 5 | 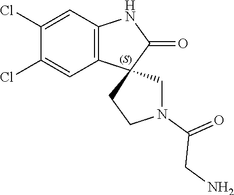 | <1 | >30 |
| 6 | 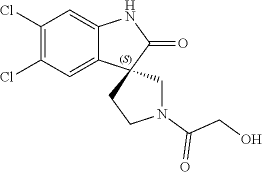 | <1 | >100 |
| 7 | 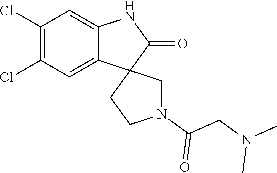 [SMILES: CN(C)CC(=O)N1CCC2(C1)C(=O)Nc1cc(Cl)c(Cl)cc12] | <3 | * |
| 8 | 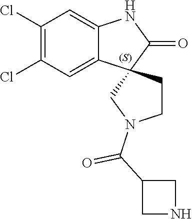 | <1 | >100 |
| 9 | 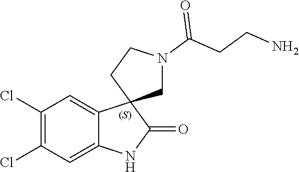 | <1 | >100 |
| 10 | 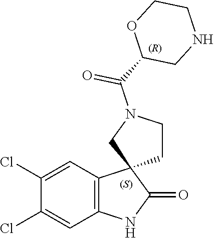 | <1 | * |
| 11 | 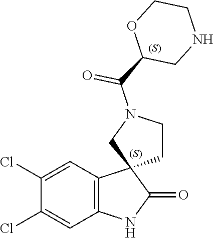 | <3 | * |
| 12 | 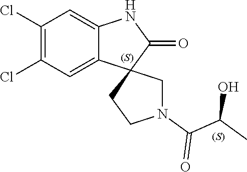 | <3 | * |
| 13 | 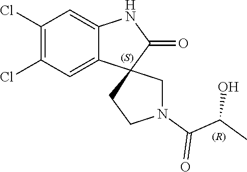 | <1 | >100 |
| 14 | 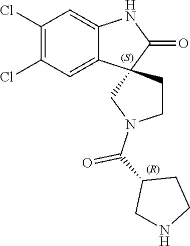 | <0.3 | >100 |
| 15 | 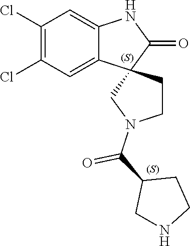 | <1 | >100 |
| 16 | 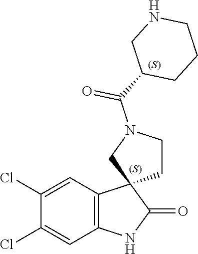 | <1 | * |
| 17 | 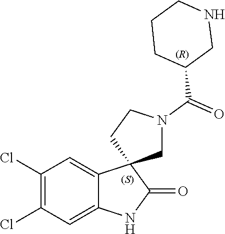 | <0.3 | >100 |
| 18 | 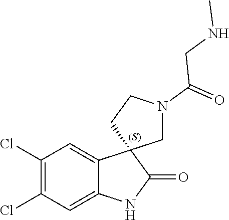 | <3 | * |
| 19 | 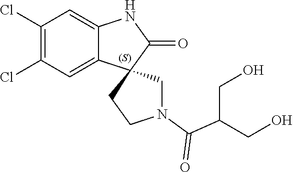 | <3 | * |
| 20 | 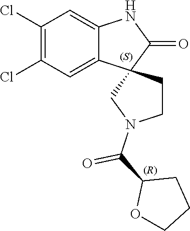 | <10 | * |
| 21 | 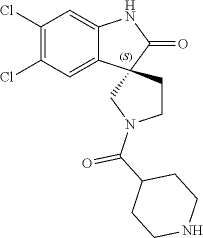 | <1 | >100 |
| 22 | 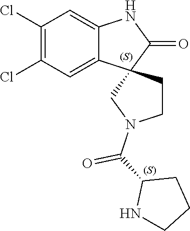 | <1 | * |
| 23 | 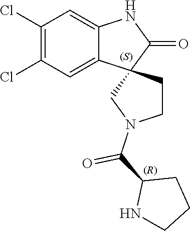 | <3 | * |
| 24 | 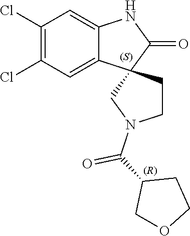 | <3 | * |
| 25 | 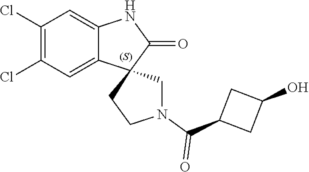 | <3 | * |
| 26 | 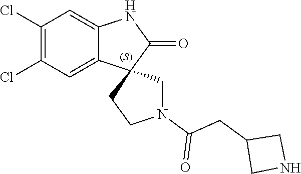 | <1 | * |
| 27 | 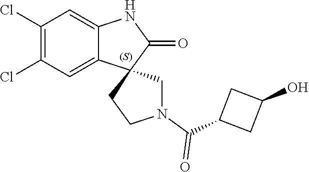 | <3 | * |
| 28 | 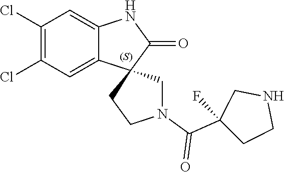 | <1 | * |
| 29 | 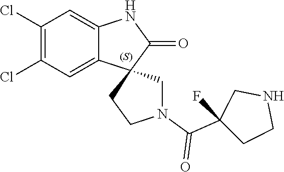 | <0.3 | >3 |
| 30 | 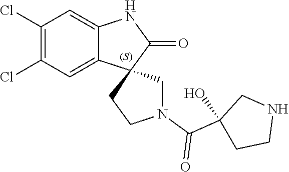 | <1 | * |
| 31 | 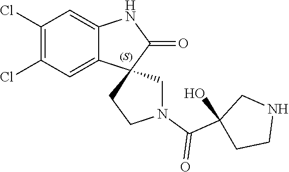 | <3 | * |
| 32 | 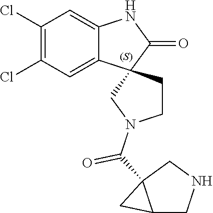 | <1 | * |
| 33 | 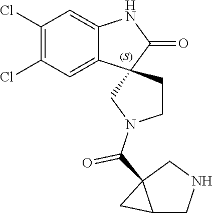 | <1 | * |
| 34 | 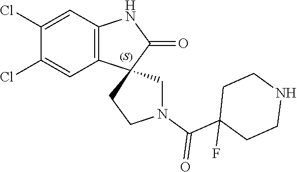 | <1 | * |
| 35 | 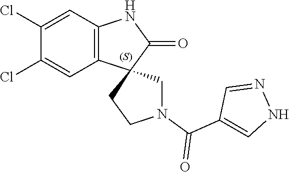 | <1 | >100 |
| 36 | 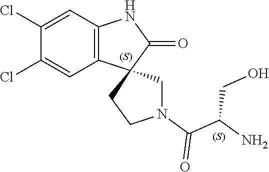 | <1 | * |
| 37 | 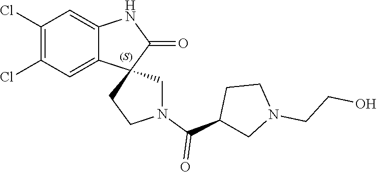 | <0.3 | >100 |
| 38 | 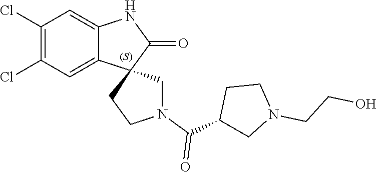 | <0.3 | >100 |
| 39 | 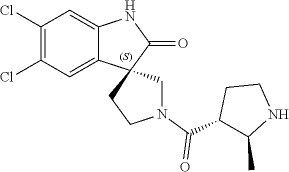 | <0.3 | >100 |
| 40 | 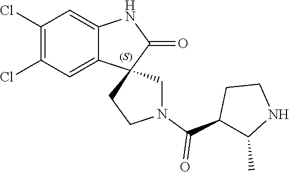 | <0.3 | >100 |
| 41 | 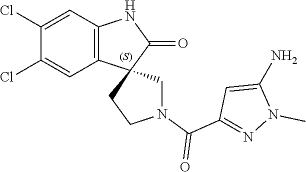 | <10 | * |
| 42 | 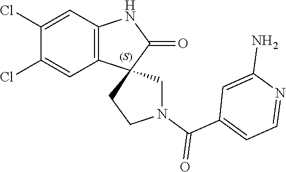 | <0.3 | >10 |
| 43 | 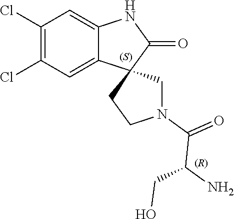 | <1 | * |
| 44 | 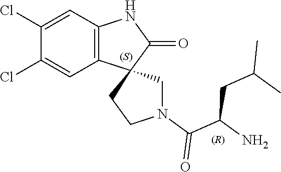 | <3 | * |
| 45 | 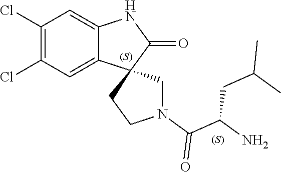 | <3 | * |
| 46 | 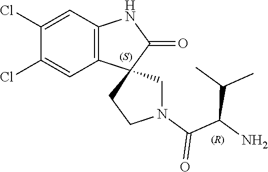 | <3 | * |
| 47 | 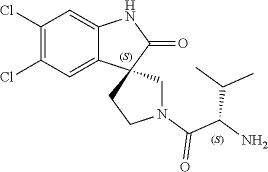 | <3 | * |
| 48 | 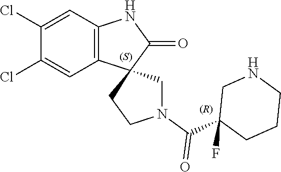 | <1 | * |
| 49 | 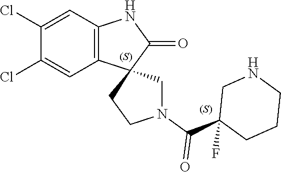 | <0.3 | >3 |
| 50 | 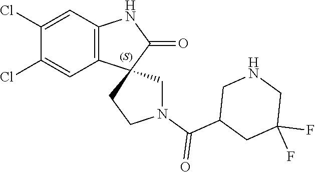 | <1 | * |
| 51 | 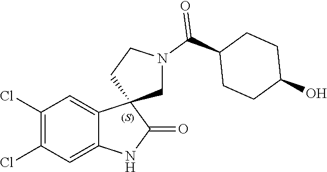 | <3 | * |
| 52 | 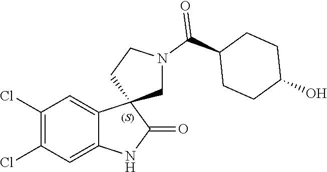 | <1 | >30 |
| 53 | 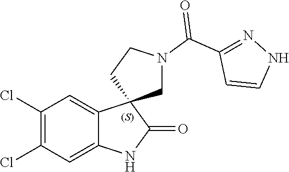 | <3 | >10 |
| 54 | 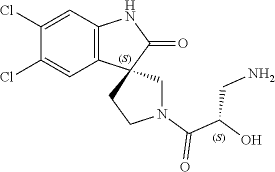 | <3 | * |
| 55 | 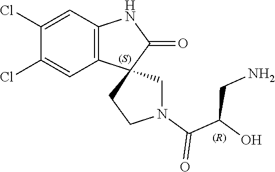 | <1 | * |
| 56 | 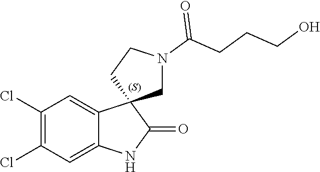 | <3 | >100 |
| 57 | 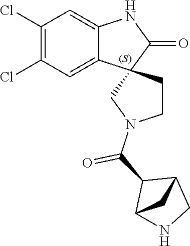 | <3 | * |
| 58 | 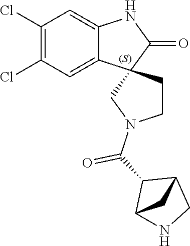 | <1 | * |
| 59 | 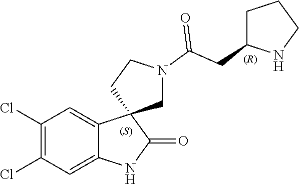 | <3 | * |
| 60 | 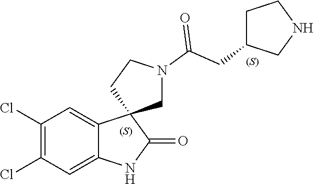 | <0.3 | >100 |
| 61 | 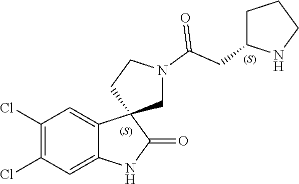 | <1 | * |
| 62 | 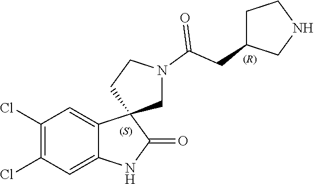 | <0.3 | >100 |
| 63 | 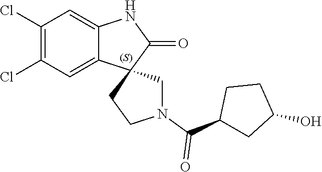 | <1 | >30 |
| 64 | 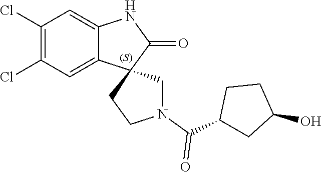 | <3 | * |
| 65 | 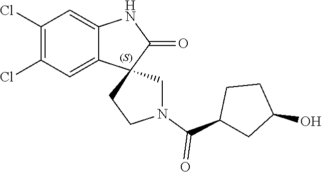 | <3 | * |
| 66 | 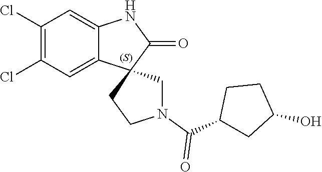 | <3 | * |
| 67 | 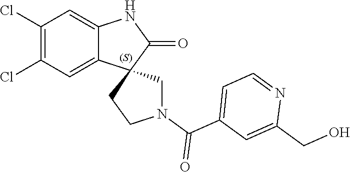 | <1 | >100 |
| 68 | 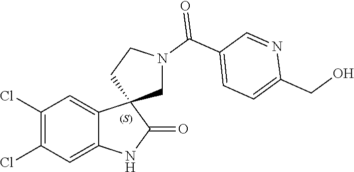 | <3 | * |
| 69 | 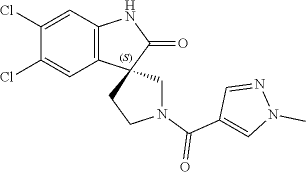 | <10 | * |
| 70 | 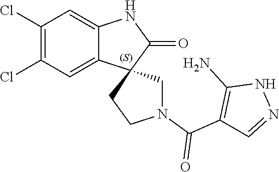 | <3 | * |
| 71 | 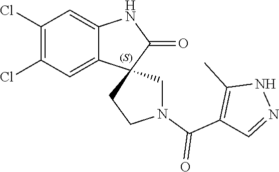 | <1 | >30 |
| 72 | 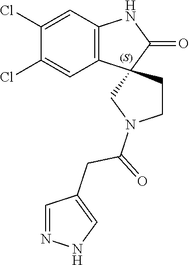 | <3 | * |
| 73 | 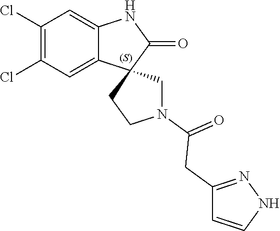 | <3 | * |
| 74 | 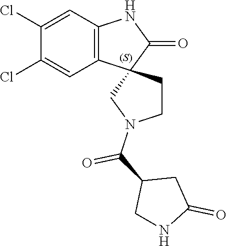 | <1 | * |
| 75 | 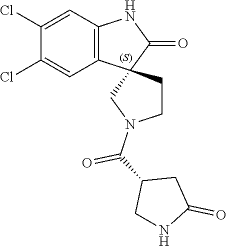 | <3 | * |
| 76 | 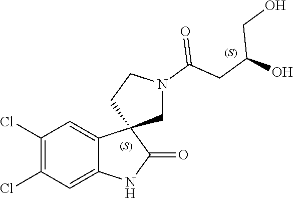 | <3 | * |
| 77 | 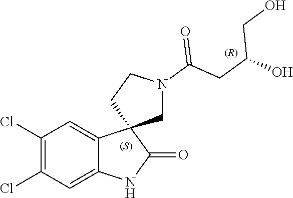 | <10 | * |
| 78 | 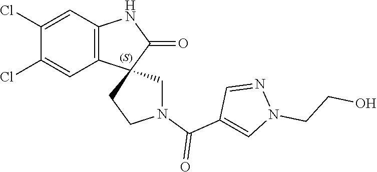 | <10 | * |
| 79 | 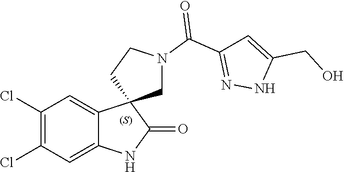 | <3 | * |
| 80 | 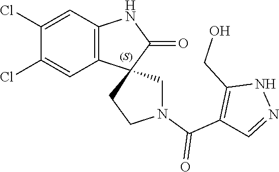 | <1 | * |
| 81 | 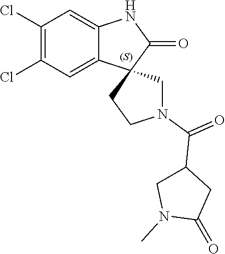 | <3 | * |
| 82 | 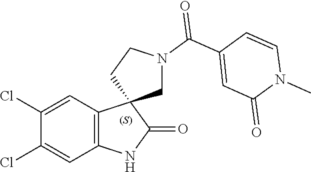 | <3 | * |
| 83 | 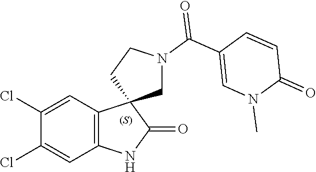 | <10 | * |
| 84 | 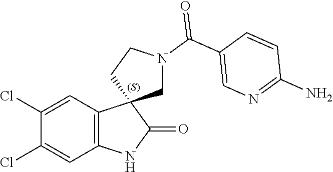 | <1 | * |
| 85 | 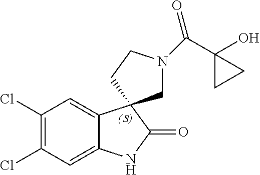 | <10 | * |
| 86 | 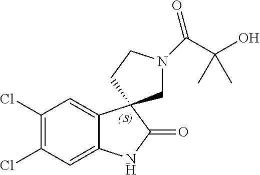 | <3 | * |
| 87 | 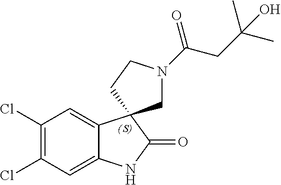 | <10 | * |
| 88 | 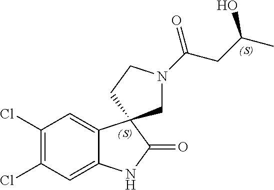 | <3 | * |
| 89 | 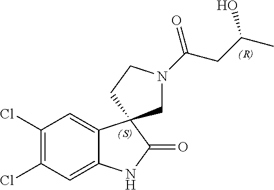 | <3 | * |
| 90 | 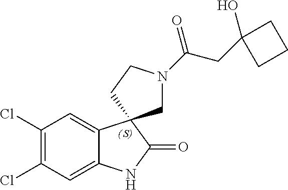 | <10 | * |
| 91 | 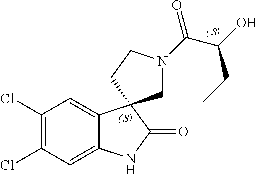 | <3 | * |
| 92 | 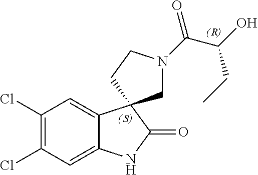 | <10 | * |
| 93 | 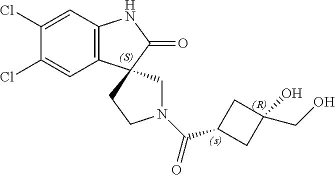 | <1 | >100 |
| 94 | 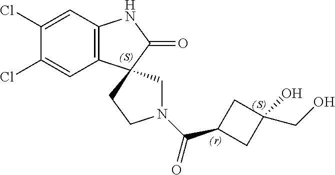 | <1 | >100 |
| 95 | 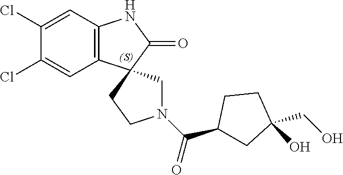 | <1 | >100 |
| 96 | 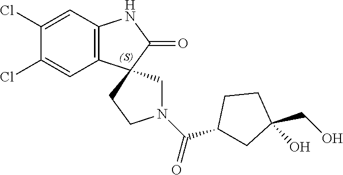 | <3 | * |
| 97 | 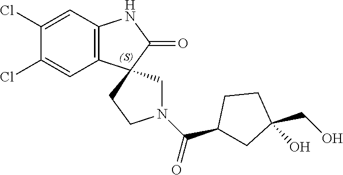 | <1 | >100 |
| 98 | 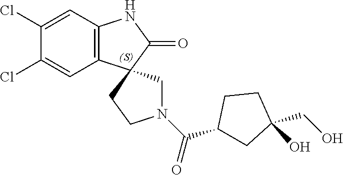 | <3 | * |
| 99 | 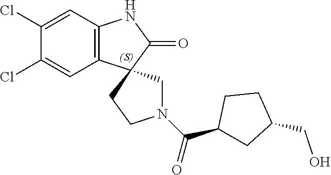 | <3 | * |
| 100 | 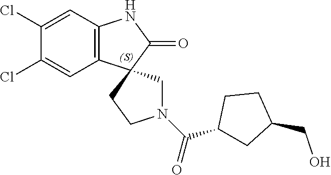 | <3 | * |
| 101 | 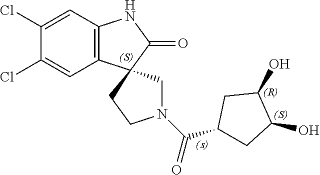 | <0.3 | >100 |
| 102 | 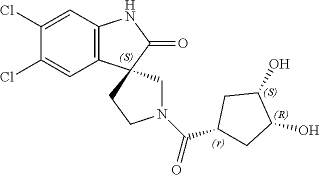 | <1 | >100 |
| 103 | 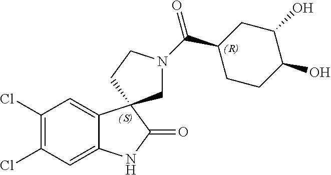 | <1 | * |
| 104 | 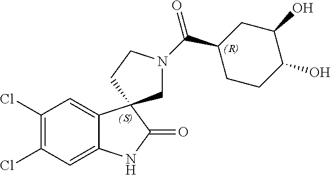 | <0.3 | >100 |
| 105 | 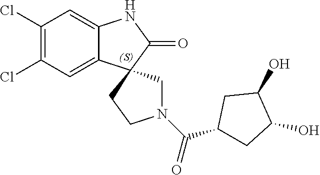 | <0.3 | >100 |
| 106 | 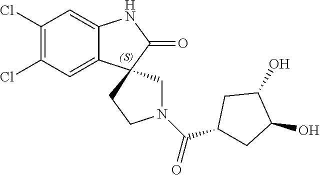 | <1 | * |
| 107 | 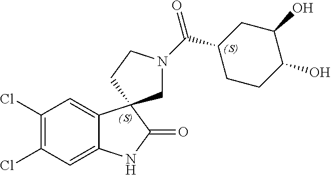 | <1 | * |
| 108 | 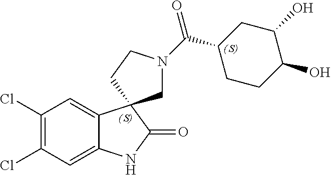 | <3 | * |
| 109 | 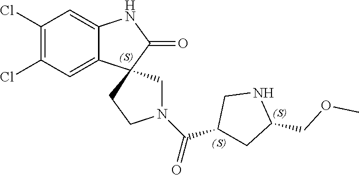 | <1 | >30 |
| 110 | 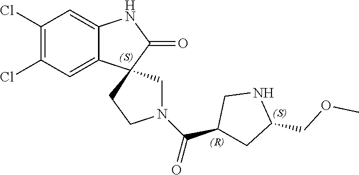 | <1 | * |
| 111 | 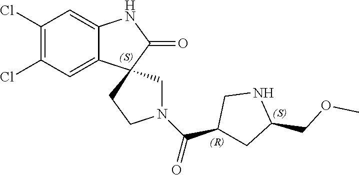 | <1 | * |
| 112 | 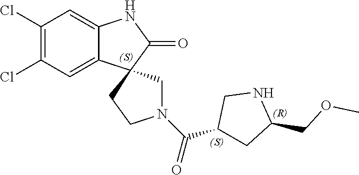 | <0.3 | >100 |
| 113 | 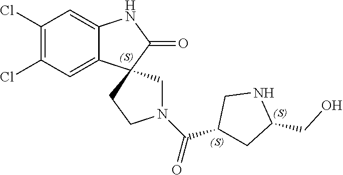 | <0.1 | >100 |
| 114 | 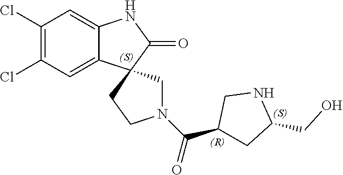 | <0.3 | >100 |
| 115 | 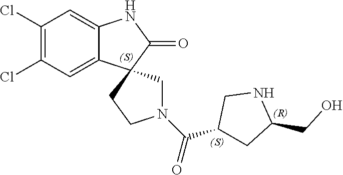 | <0.1 | >100 |
| 116 | 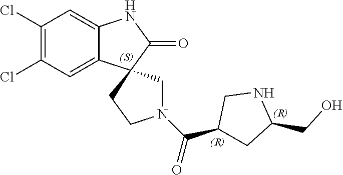 | <0.1 | >100 |
| 117 | 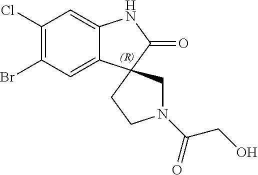 | <10 | * |
| 118 | 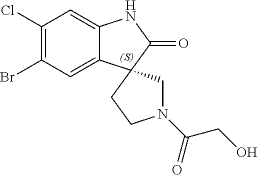 | <1 | * |
| 119 | 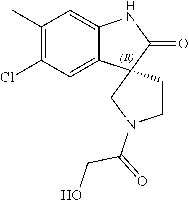 | <10 | * |
| 120 | 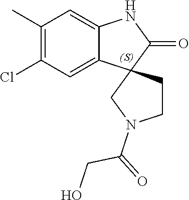 | <3 | >100 |
| 121 | 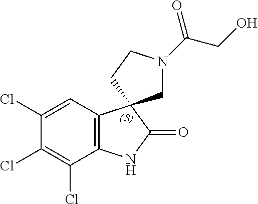 | <0.3 | >10 |
| 122 | 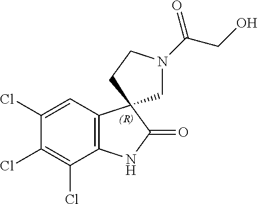 | <3 | * |
| 123 | 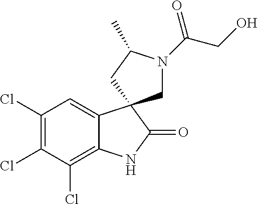 [SMILES: C[C@H]1C[C@]2(CN1C(=O)CO)C(=O)Nc1c2cc(Cl)c(Cl)c1Cl] | <30 | * |
| 124 | 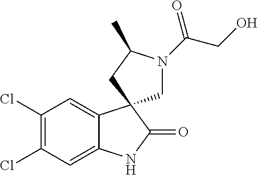 [SMILES: C[C@@H]1C[C@@]2(CN1C(=O)CO)C(=O)Nc1cc(Cl)c(Cl)cc12] | <10 | * |
| 125 | 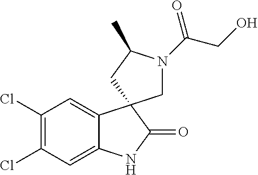 [SMILES: C[C@@H]1C[C@]2(CN1C(=O)CO)C(=O)Nc1cc(Cl)c(Cl)cc12] | <1 | * |
| 126 | 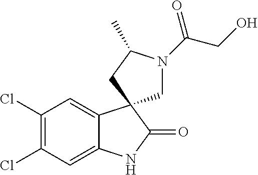 [SMILES: C[C@H]1C[C@@]2(CN1C(=O)CO)C(=O)Nc1cc(Cl)c(Cl)cc12] | <10 | * |
| 127 | 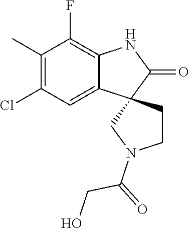 [SMILES: Cc1c(Cl)cc2c(c1F)NC(=O)[C@@]21CCN(C(=O)CO)C1] | <1 | * |
| 128 | 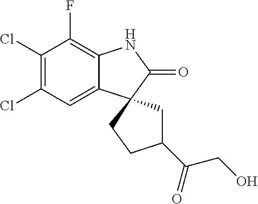 | <0.3 | >100 |
| 129 | 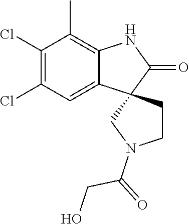 [SMILES: Cc1c(Cl)c(Cl)cc2c1NC(=O)[C@@]21CCN(C(=O)CO)C1] | <0.3 | >100 |
| 130 | 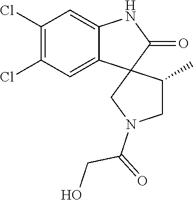 [SMILES: C[C@@H]1CN(C(=O)CO)CC12C(=O)Nc1cc(Cl)c(Cl)cc12] | <30 | * |
| 131 | 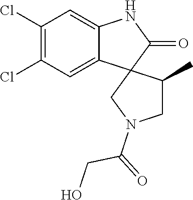 [SMILES: C[C@H]1CN(C(=O)CO)CC12C(=O)Nc1cc(Cl)c(Cl)cc12] | <1 | * |
| 132 | 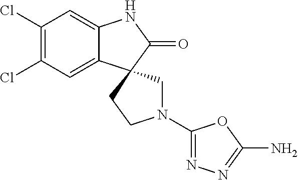 [SMILES: Nc1nnc(N2CC[C@]3(C2)C(=O)Nc2cc(Cl)c(Cl)cc23)o1] | <3 | * |
| 133 | 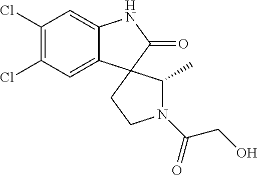 [SMILES: C[C@@H]1N(C(=O)CO)CCC12C(=O)Nc1cc(Cl)c(Cl)cc12] | <3 | * |
| 134 | 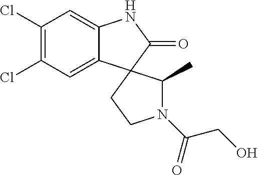 [SMILES: C[C@H]1N(C(=O)CO)CCC12C(=O)Nc1cc(Cl)c(Cl)cc12] | <10 | * |
| 135 | 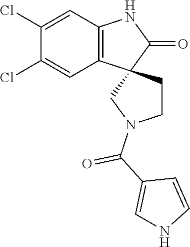 [SMILES: O=C(c1cc[nH]c1)N1CC[C@]2(C1)C(=O)Nc1cc(Cl)c(Cl)cc12] | <1 | * |
| 136 | 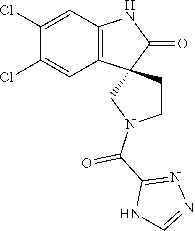 [SMILES: O=C(c1nnc[nH]1)N1CC[C@]2(C1)C(=O)Nc1cc(Cl)c(Cl)cc12] | <1 | * |
| 137 | 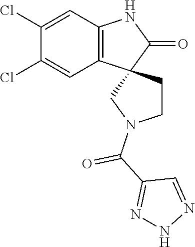 [SMILES: O=C(c1cn[nH]n1)N1CC[C@]2(C1)C(=O)Nc1cc(Cl)c(Cl)cc12] | <1 | * |
| 138 | 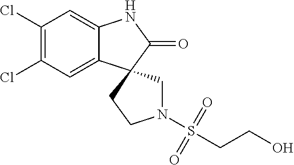 [SMILES: O=C1Nc2cc(Cl)c(Cl)cc2[C@]12CCN(S(=O)(=O)CCO)C2] | <1 | * |
| 139 | 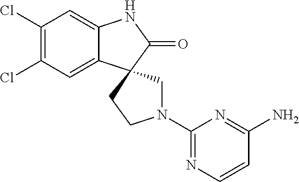 [SMILES: Nc1ccnc(N2CC[C@]3(C2)C(=O)Nc2cc(Cl)c(Cl)cc23)n1] | <1 | * |
| 140 | 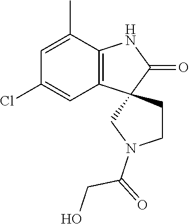 [SMILES: Cc1cc(Cl)cc2c1NC(=O)[C@@]21CCN(C(=O)CO)C1] | <1 | * |
| 141 | 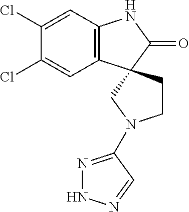 [SMILES: O=C1Nc2cc(Cl)c(Cl)cc2[C@]12CCN(c1cn[nH]n1)C2] | <1 | * |
| 142 | 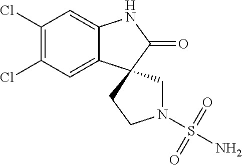 [SMILES: NS(=O)(=O)N1CC[C@]2(C1)C(=O)Nc1cc(Cl)c(Cl)cc12] | <3 | * |
| 143 | 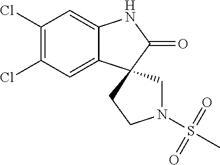 [SMILES: CS(=O)(=O)N1CC[C@]2(C1)C(=O)Nc1cc(Cl)c(Cl)cc12] | <1 | * |
| 144 | 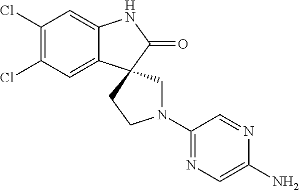 [SMILES: Nc1cnc(N2CC[C@]3(C2)C(=O)Nc2cc(Cl)c(Cl)cc23)cn1] | <1 | * |
| 145 | 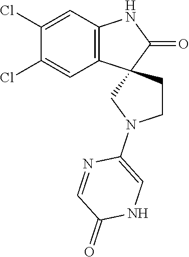 [SMILES: O=C1Nc2cc(Cl)c(Cl)cc2[C@]12CCN(c1c[nH]c(=O)cn1)C2] | <1 | * |
| 146 | 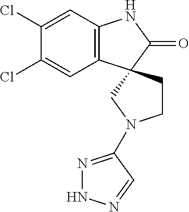 [SMILES: O=C1Nc2cc(Cl)c(Cl)cc2[C@]12CCN(c1cn[nH]n1)C2] | <3 | * |
| 147 | 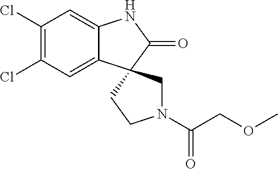 [SMILES: COCC(=O)N1CC[C@@]2(C1)C(=O)Nc1cc(Cl)c(Cl)cc12] | <1 | * |
| 148 | 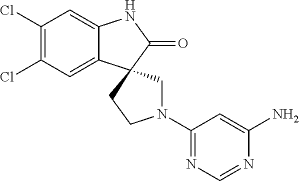 [SMILES: Nc1cc(N2CC[C@]3(C2)C(=O)Nc2cc(Cl)c(Cl)cc23)ncn1] | <3 | * |
| 149 | 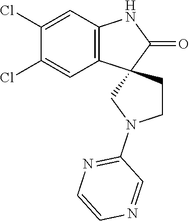 [SMILES: O=C1Nc2cc(Cl)c(Cl)cc2[C@]12CCN(c1cnccn1)C2] | <3 | * |
| 150 | 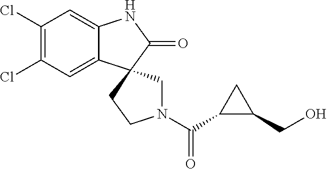 [SMILES: O=C([C@@H]1C[C@H]1CO)N1CC[C@]2(C1)C(=O)Nc1cc(Cl)c(Cl)cc12] | <1 | * |
| 151 | 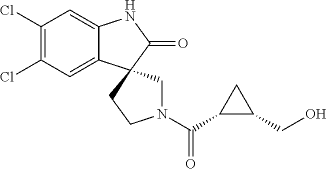 [SMILES: O=C([C@@H]1C[C@@H]1CO)N1CC[C@]2(C1)C(=O)Nc1cc(Cl)c(Cl)cc12] | <1 | * |
| 152 | 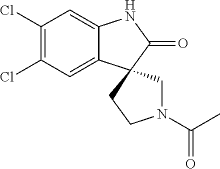 [SMILES: CC(=O)N1CC[C@]2(C1)C(=O)Nc1cc(Cl)c(Cl)cc12] | <1 | * |
| 153 | 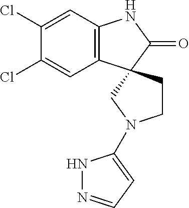 [SMILES: O=C1Nc2cc(Cl)c(Cl)cc2[C@]12CCN(c1ccn[nH]1)C2] | <3 | * |
| 154 | 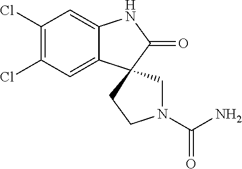 [SMILES: NC(=O)N1CC[C@]2(C1)C(=O)Nc1cc(Cl)c(Cl)cc12] | <1 | * |
| 155 | 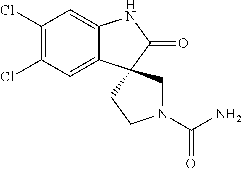 [SMILES: NC(=O)N1CC[C@@]2(C1)C(=O)Nc1cc(Cl)c(Cl)cc12] | <3 | * |
| 156 | 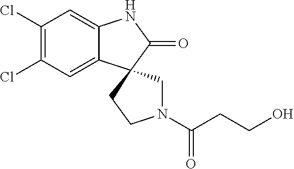 [SMILES: O=C(CCO)N1CC[C@]2(C1)C(=O)Nc1cc(Cl)c(Cl)cc12] | <1 | * |
| 157 | 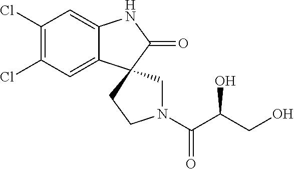 [SMILES: O=C([C@@H](O)CO)N1CC[C@]2(C1)C(=O)Nc1cc(Cl)c(Cl)cc12] | <1 | * |
| 158 | 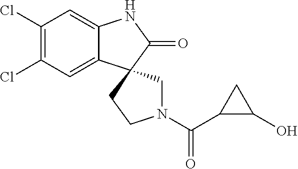 [SMILES: O=C(C1CC1O)N1CC[C@]2(C1)C(=O)Nc1cc(Cl)c(Cl)cc12] | <3 | * |
| 159 | 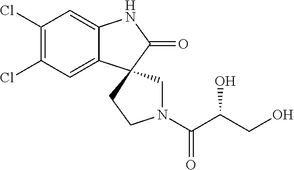 [SMILES: O=C([C@H](O)CO)N1CC[C@]2(C1)C(=O)Nc1cc(Cl)c(Cl)cc12] | <3 | * |

*Not Tested.

1. A compound of Formula I or a pharmaceutically acceptable salt thereof: 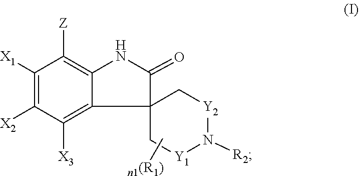
wherein:
the structural moiety 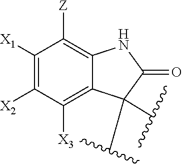
has the structure of 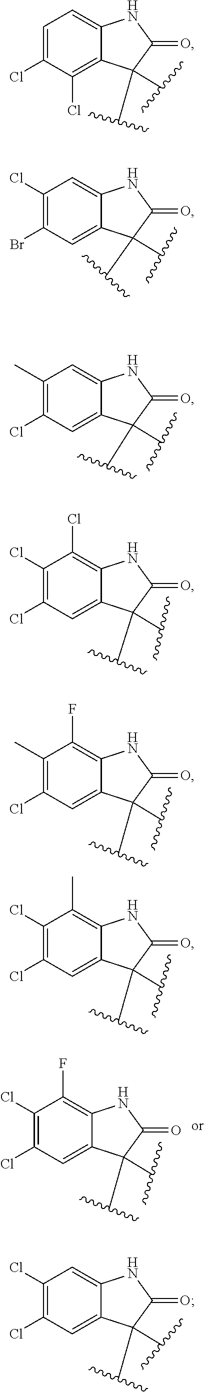
or alternatively X₁ and X₂ and the carbon atoms they are connected to taken together form a 5- or 6-membered aryl; wherein X₃ is H, halogen, CN, alkyl, cycloalkyl, halogenated alkyl, halogenated cycloalkyl, OH, SH, alkoxy, halogenated alkoxy, alkylthio, or halogenated alkylthio and Z is H, alkyl, halogenated alkyl, heteroalkyl, cycloalkyl, heterocycle, aryl, heteroaryl, halogen, CN, CF₃, OCF₃, ORₐ, NRₐR_{b}, or NRₐ(C═O)R_{b};
or alternatively X₂ and X₃ and the carbon atoms they are connected to taken together form a 5- or 6-membered aryl; wherein X₁ is H, halogen, CN, alkyl, cycloalkyl, halogenated alkyl, halogenated cycloalkyl, OH, SH, alkoxy, halogenated alkoxy, alkylthio, or halogenated alkylthio and Z is H, alkyl, halogenated alkyl, heteroalkyl, cycloalkyl, heterocycle, aryl, heteroaryl, halogen, CN, CF₃, OCF₃, ORₐ, NRₐR_{b}, or NRₐ(C═O)R_{b};
Y₁ is absent or C(R₁)₂;
Y₂ is absent, C(R₁)₂, C(R₁)₂(C═O), C(R₁)₂C(R₁)₂ or C(R₁)₂C(R₁)₂(C═O);
each occurrence of R₁ is independently H, halogen, alkyl, cycloalkyl, saturated heterocycle, aryl, heteroaryl, (CR₄R₅)ₙ₃OR_{c}, or (CR₄R₅)ₙ₃NR_{c}R_{d};
R₂ is alkyl, heteroalkyl, cycloalkyl, heterocycle, bicycloalkyl, spiroalkyl, heterobicycloalkyl, heterospiroalkyl, alkylaryl, alkylheteroaryl, aryl, heteroaryl, (CR₄R₅)ₙ₂(C═O)R₃, (CR₄R₅)ₙ₂(C═O)N(R₄)R₃, SO₂R_{c}, or SO₂NR_{c}R_{d};
each occurrence of R₃ is independently H, alkyl, cycloalkyl, heterocycle, bicycloalkyl, spiroalkyl, heterobicycloalkyl, heterospiroalkyl, alkylaryl, alkylheteroaryl, aryl, or heteroaryl;
each occurrence of R₄ and R₅ is independently H, alkyl, cycloalkyl, heterocycle, aryl, or heteroaryl;
each occurrence of Rₐ and R_{b} is independently H, alkyl, cycloalkyl, heterocycle, aryl, or heteroaryl;
or alternatively Rₐ and R_{b} together with the nitrogen atom that they are connected to form a 3-7-membered heterocycle;
each occurrence of R_{c} and R_{d} is independently H, alkyl, cycloalkyl, heterocycle, aryl, or heteroaryl;
or alternatively R_{c} and R_{d} together with the nitrogen atom that they are connected to form a 3-7-membered heterocycle;
each heterocycle comprises 1-3 heteroatoms each independently selected from the group consisting of N, O and S;
each of alkyl, cycloalkyl, heteroalkyl, heterocycle, aryl, and heteroaryl in X₁, X₃, Z, —R₁, R₂, or R₃, where applicable, are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)R_{d}, where valence permits;
n₁ is an integer from 0-4;
n₂ is an integer from 0-4; and
n₃ is an integer from 0-4.

2.-66. (canceled)

67. A compound, or a pharmaceutically acceptable salt thereof, wherein the compound has Formula Ic: 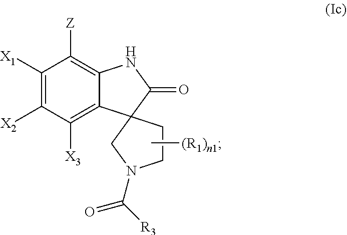
wherein:
X₁ and X₂ are Cl;
X₃ is H;
Z is H, halogen, alkyl, halogenated alkyl, CN, ORₐ, or NRₐR_{b};
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d};
R₃ is alkyl, cycloalkyl, heterocycle, aryl, heteroaryl, bicycloalkyl, spiroalkyl, heterobicycloalkyl, or heterospiroalkyl; and wherein the alkyl, cycloalkyl, heterocycle, aryl, or heteroaryl is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits;
each occurrence of R₄ and R₅ is independently H, alkyl, cycloalkyl, heterocycle, aryl, or heteroaryl;
each occurrence of Rₐ and R_{b} is independently H, alkyl, cycloalkyl, heterocycle, aryl, or heteroaryl;
or alternatively Rₐ and R_{b} together with the nitrogen atom that they are connected to form a 3-7-membered heterocycle:
each occurrence of R_{c} and R_{d} is independently H, alkyl, cycloalkyl, heterocycle, aryl, or heteroaryl;
or alternatively R_{c} and R_{d} together with the nitrogen atom that they are connected to form a 3-7-membered heterocycle;
each heterocycle comprises 1-3 heteroatoms each independently selected from the group consisting of N, O and S;
each of alkyl, cycloalkyl, heteroalkyl, heterocycle, aryl, and heteroaryl in Z, R₁, or R₃, where applicable, are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)R_{d}, where valence permits;
n₁ is an integer from 0-4; and
n₃ is an integer from 0-4.

68. (canceled)

69. The compound of claim 67, or a pharmaceutically acceptable salt thereof, wherein Z is H, F, Cl, Br, CH₃, or CF₃.

70. (canceled)

71. The compound of claim 69, or a pharmaceutically acceptable salt thereof, wherein at least one occurrence of R₁ is H, alkyl or cycloalkyl.

72. The compound of claim 71, or a pharmaceutically acceptable salt thereof, wherein n₁ is 0 or 1.

73. The compound of claim 71, or a pharmaceutically acceptable salt thereof, wherein R₃ is alkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

74. (canceled)

75. The compound of claim 71, or a pharmaceutically acceptable salt thereof, wherein R₃ is heterocycle that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

76.-77. (canceled)

78. A compound, or a pharmaceutically acceptable salt thereof, wherein the compound has Formula Id: 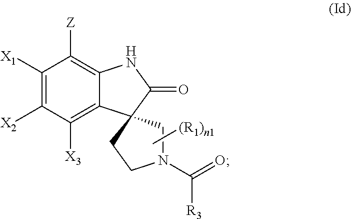
wherein:
X₁ and X₂ are Cl;
X₃ is H;
Z is H, halogen, alkyl, halogenated alkyl, CN, ORₐ, or NRₐR_{b};
each occurrence of R₁ is independently H, alkyl, cycloalkyl, halogen, saturated heterocycle, aryl, heteroaryl, (CR₄R₅)ₙ₃OR_{c}, or (CR₄R₅)ₙ₃NR_{c}R_{d};
R₃ is alkyl, cycloalkyl, heterocycle, aryl, heteroaryl, bicycloalkyl, spiroalkyl, heterobicycloalkyl, or heterospiroalkyl; and wherein the alkyl, cycloalkyl, heterocycle, aryl, or heteroaryl is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits;
each occurrence of R₄ and R₅ is independently H, alkyl, cycloalkyl, heterocycle, aryl, or heteroaryl;
each occurrence of Rₐ and R_{b} is independently H, alkyl, cycloalkyl, heterocycle, aryl, or heteroaryl;
or alternatively Rₐ and R_{b} together with the nitrogen atom that they are connected to form a 3-7-membered heterocycle;
each occurrence of R_{c} and R_{d} is independently H, alkyl, cycloalkyl, heterocycle, aryl, or heteroaryl;
or alternatively R_{c} and R_{d} together with the nitrogen atom that they are connected to form a 3-7-membered heterocycle;
each heterocycle comprises 1-3 heteroatoms each independently selected from the group consisting of N, O and S;
each of alkyl, cycloalkyl, heteroalkyl, heterocycle, aryl, and heteroaryl in Z, R₁, or R₃, where applicable, are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃R_{c}, (CR₄R₅)ₙ₃NR_{c}R_{d}, and (CR₄R₅)ₙ₃NR_{c}(C═O)R_{d}, where valence permits;
n₁ is an integer from 0-4; and
n₃ is an integer from 0-4.

79. (canceled)

80. The compound of claim 78, or a pharmaceutically acceptable salt thereof, wherein Z is H, F, Cl, Br, CH₃, or CF₃.

81. (canceled)

82. The compound of claim 80, or a pharmaceutically acceptable salt thereof, wherein at least one occurrence of R₁ is H, alkyl or cycloalkyl.

83. The compound of claim 82, or a pharmaceutically acceptable salt thereof, wherein n₁ is 0 or 1.

84. The compound of claim 82, or a pharmaceutically acceptable salt thereof, wherein R₃ is alkyl that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

85. (canceled)

86. The compound of claim 82, or a pharmaceutically acceptable salt thereof, wherein R₃ is heterocycle that is optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, and (CR₄R₅)ₙ₃NR_{c}R_{d}, where valence permits.

87.-88. (canceled)

89. The compound of claim 1, wherein R₂ is

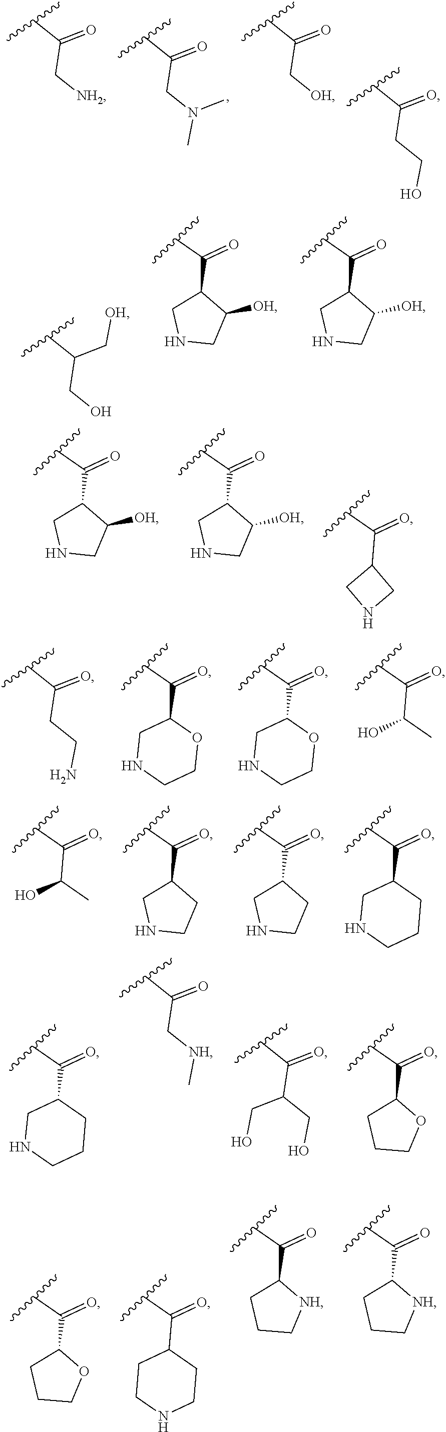

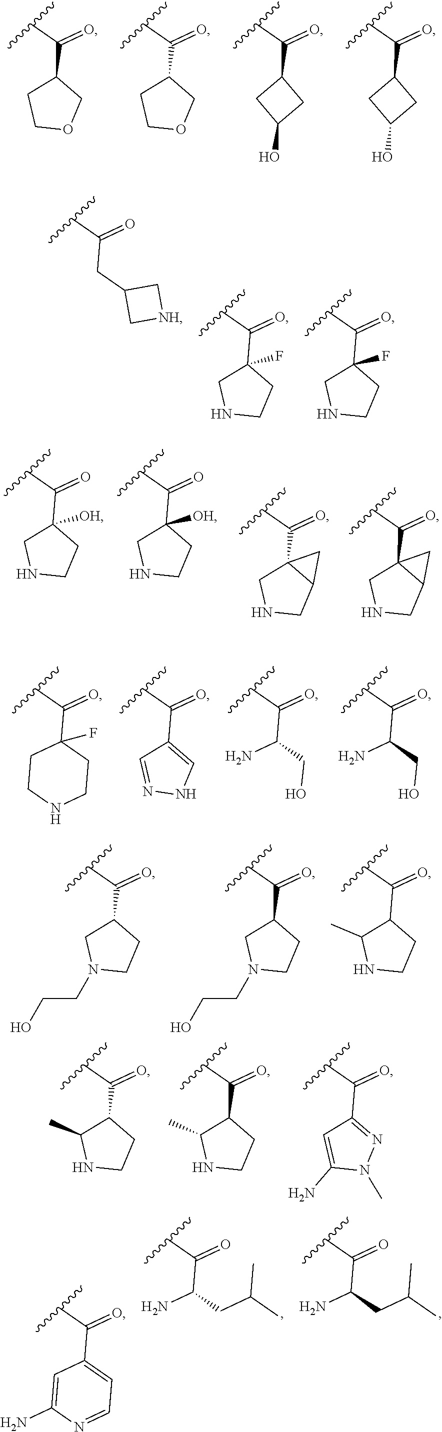

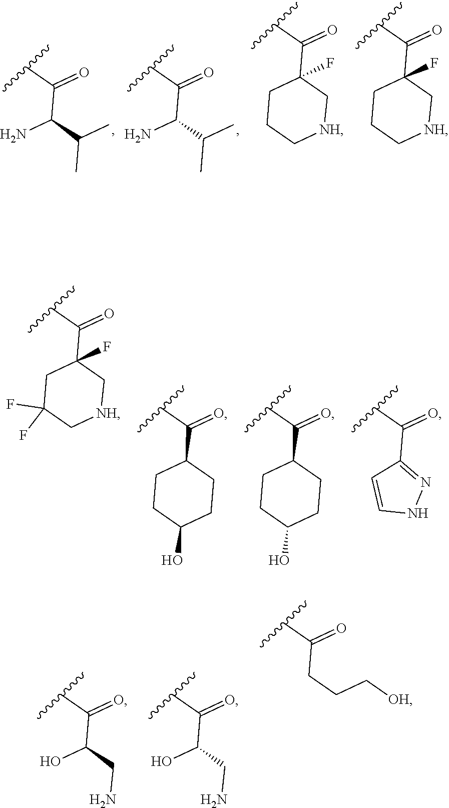

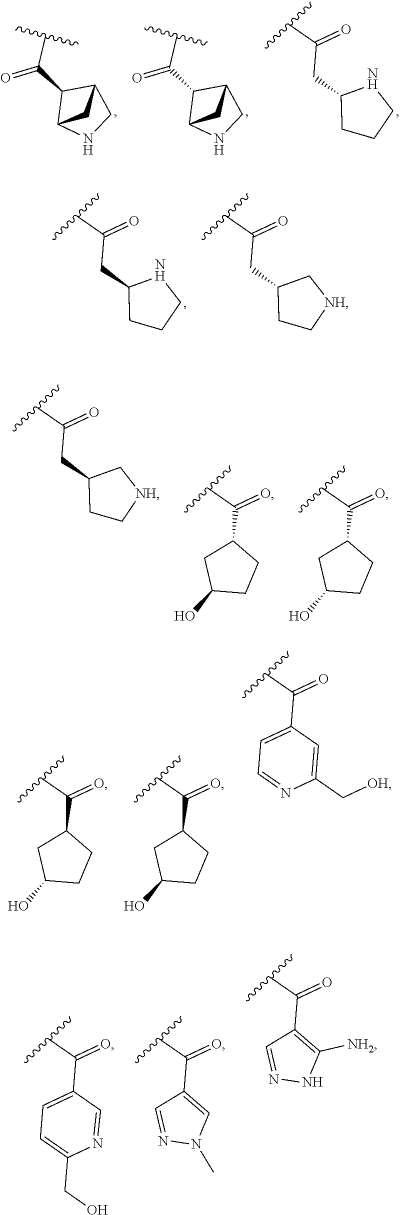

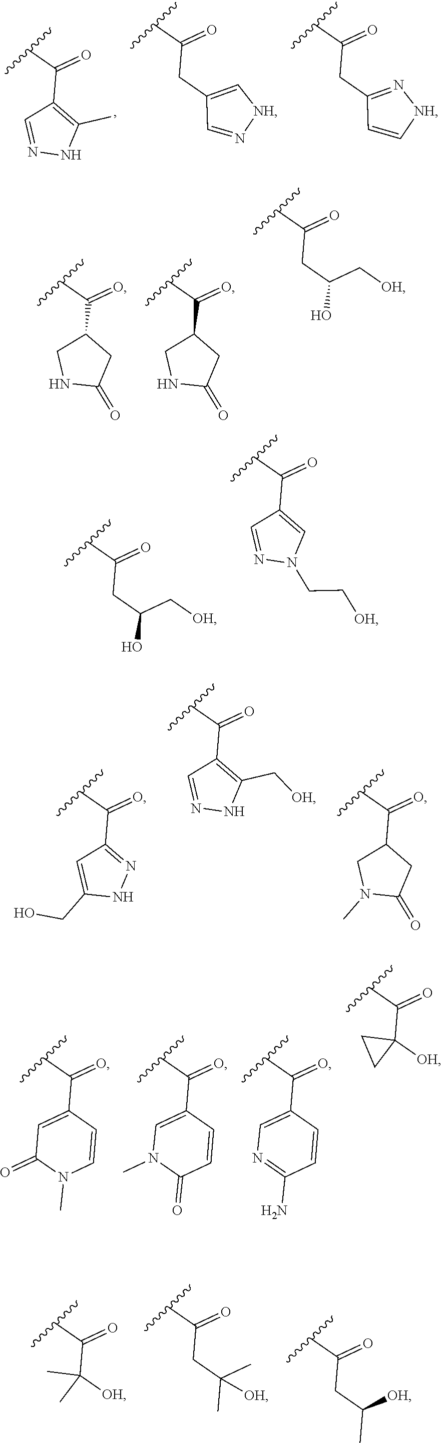

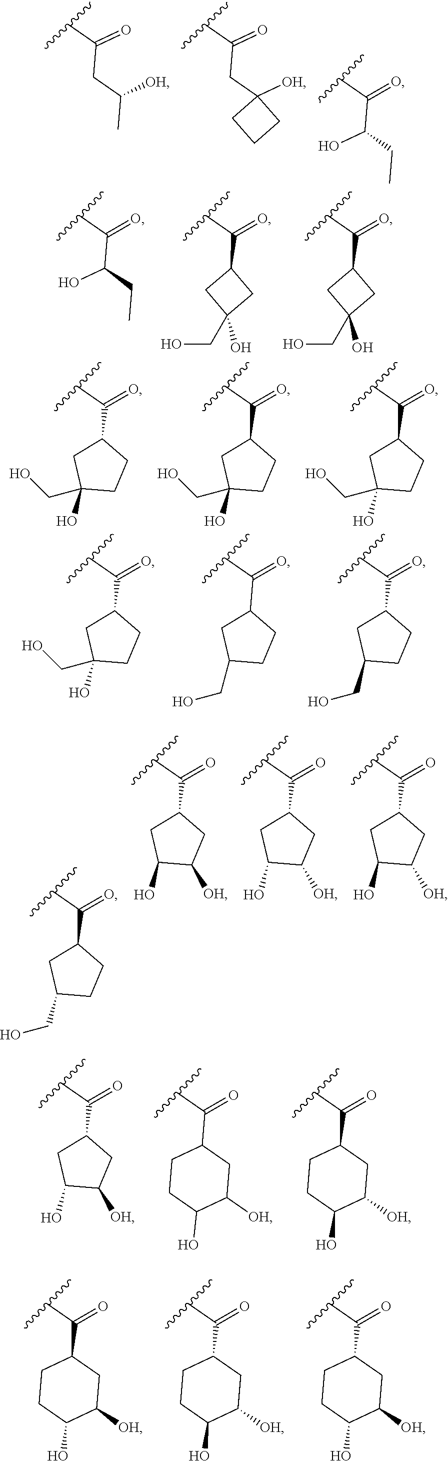

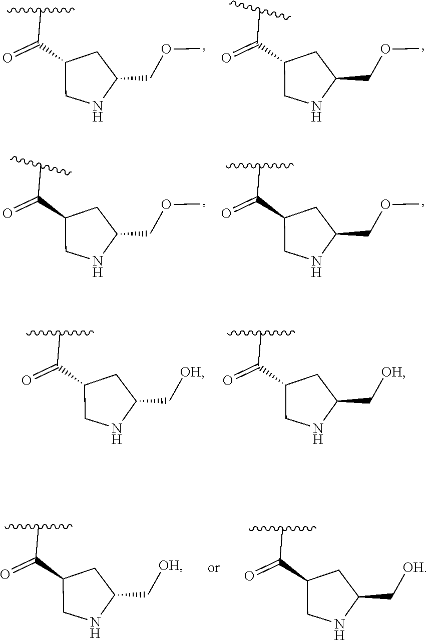

90.-91. (canceled)

92. A compound selected from the group consisting of:

| Compound | |
|---|---|
| Number | Structure |
| 1 | 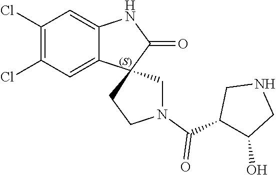 |
| 2 | 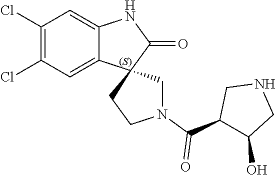 |
| 3 | 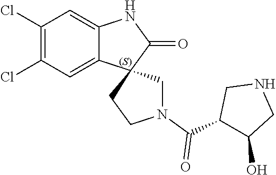 |
| 4 | 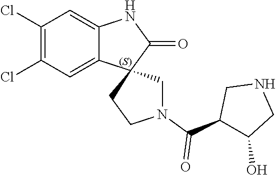 |
| 5 | 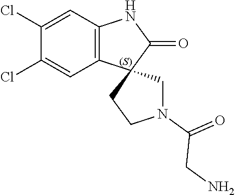 |
| 6 | 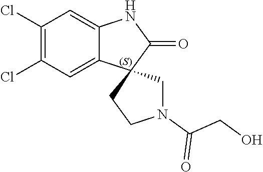 |
| 7 | 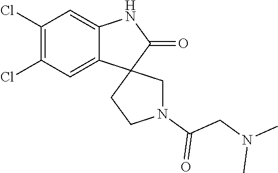 [SMILES: CN(C)CC(=O)N1CCC2(C1)C(=O)Nc1cc(Cl)c(Cl)cc12] |
| 8 | 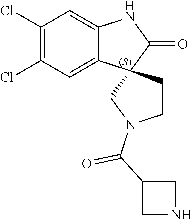 |
| 9 | 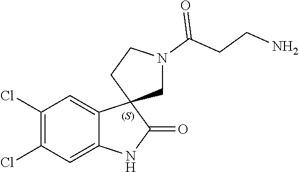 |
| 10 | 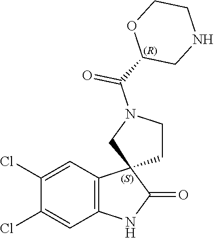 |
| 11 | 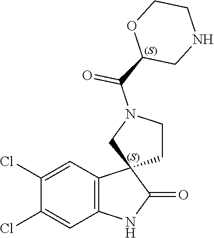 |
| 12 | 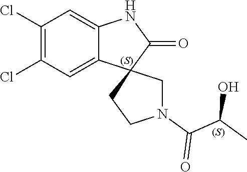 |
| 13 | 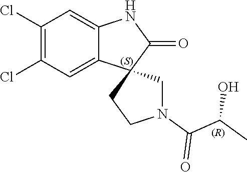 |
| 14 | 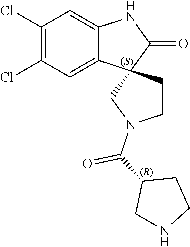 |
| 15 | 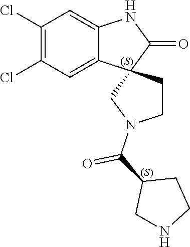 |
| 16 | 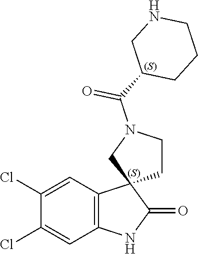 |
| 17 | 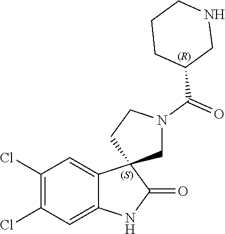 |
| 18 | 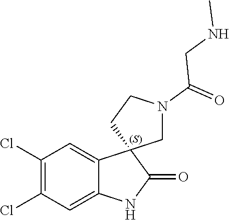 |
| 19 | 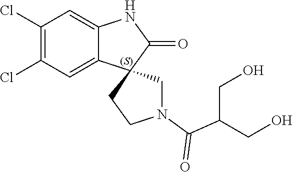 |
| 20 | 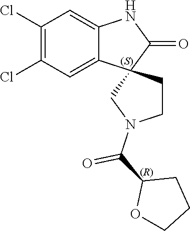 |
| 21 | 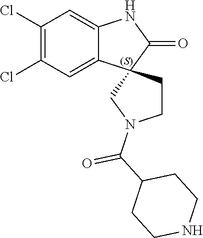 |
| 22 | 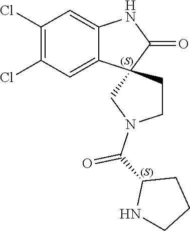 |
| 23 | 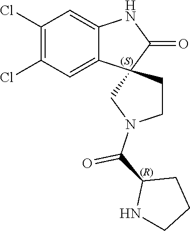 |
| 24 | 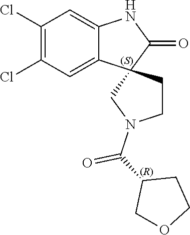 |
| 25 | 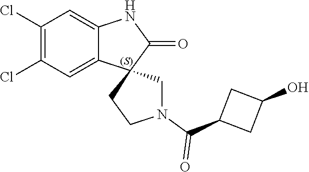 |
| 26 | 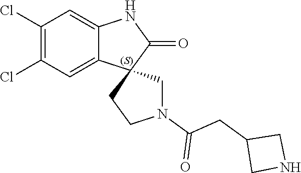 |
| 27 | 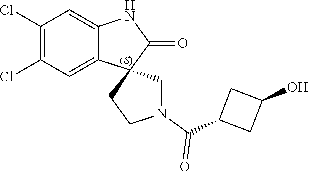 |
| 28 | 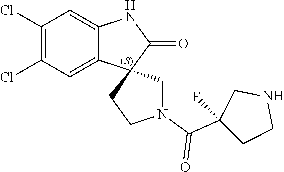 |
| 29 | 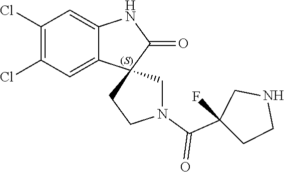 |
| 30 | 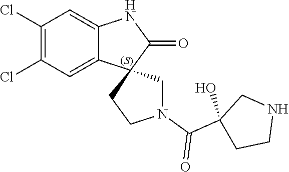 |
| 31 | 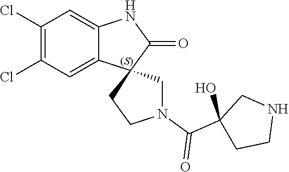 |
| 32 | 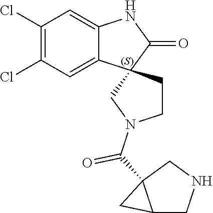 |
| 33 | 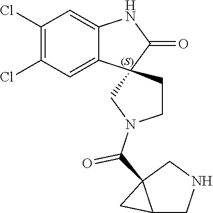 |
| 34 | 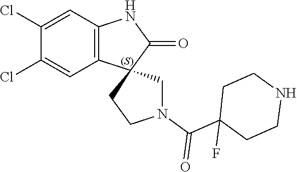 |
| 35 | 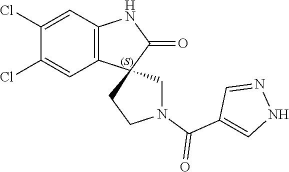 |
| 36 | 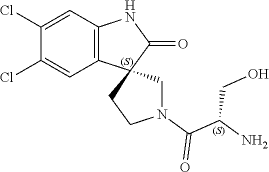 |
| 37 | 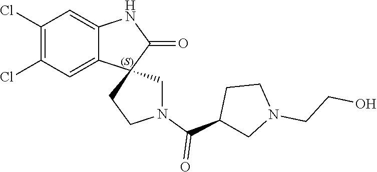 |
| 38 | 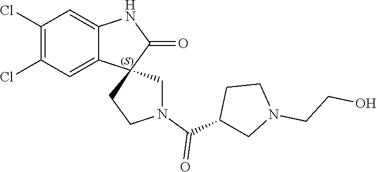 |
| 39 | 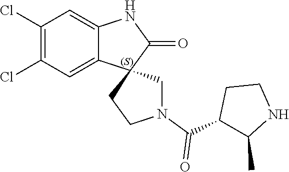 |
| 40 | 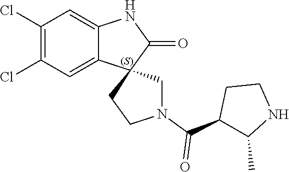 |
| 41 | 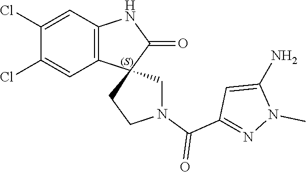 |
| 42 | 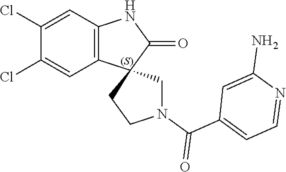 |
| 43 | 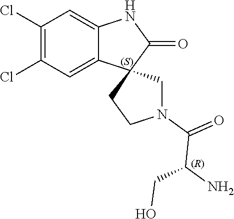 |
| 44 | 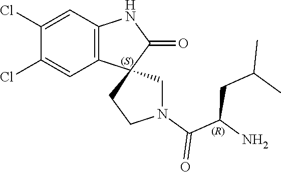 |
| 45 | 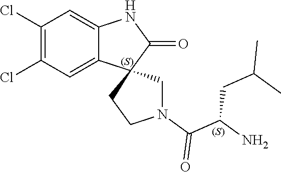 |
| 46 | 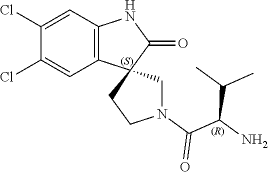 |
| 47 | 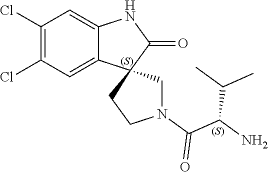 |
| 48 | 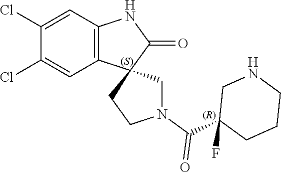 |
| 49 | 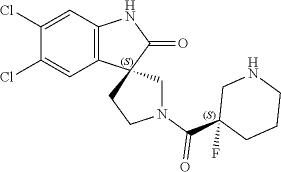 |
| 50 | 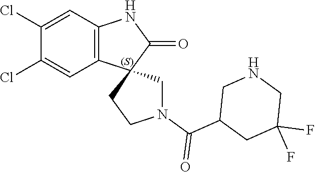 |
| 51 | 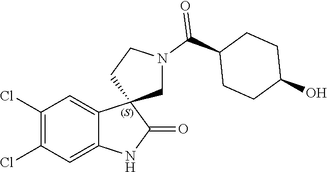 |
| 52 | 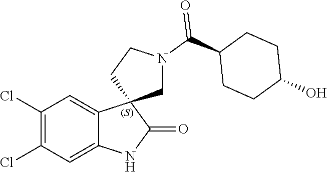 |
| 53 | 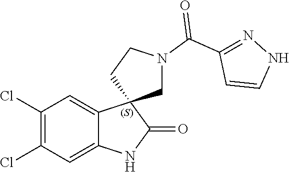 |
| 54 | 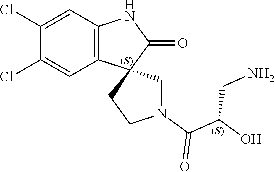 |
| 55 | 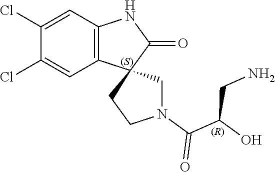 |
| 56 | 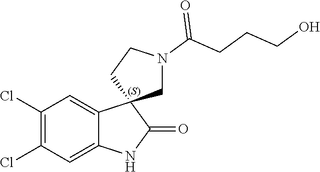 |
| 57 | 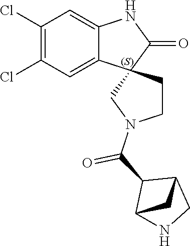 |
| 58 | 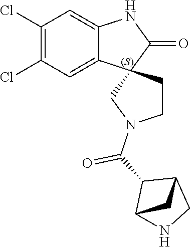 |
| 59 | 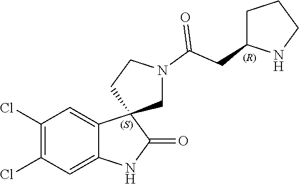 |
| 60 | 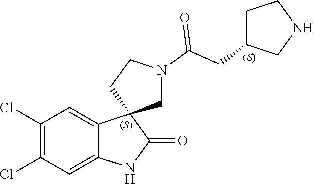 |
| 61 | 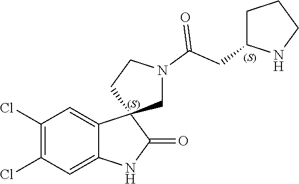 |
| 62 | 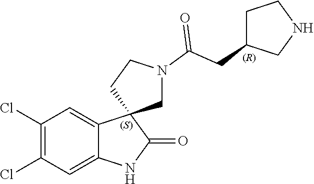 |
| 63 | 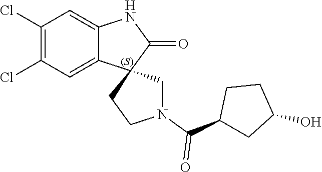 |
| 64 | 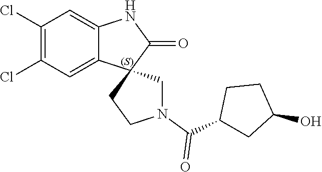 |
| 65 | 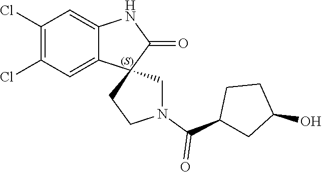 |
| 66 | 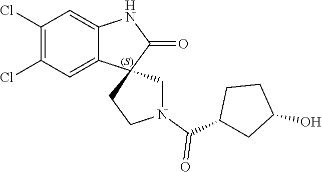 |
| 67 | 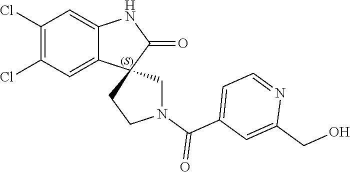 |
| 68 | 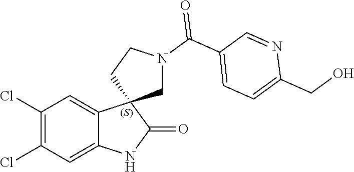 |
| 69 | 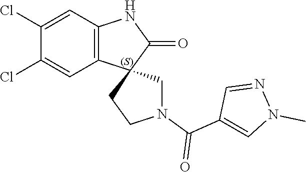 |
| 70 | 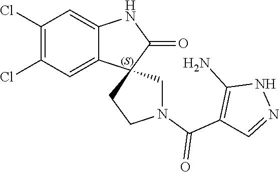 |
| 71 | 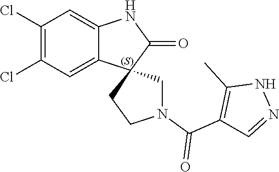 |
| 72 | 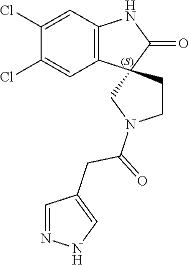 |
| 73 | 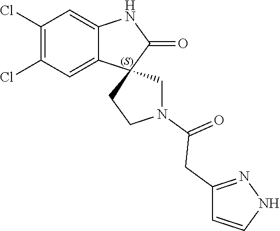 |
| 74 | 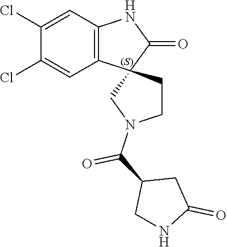 |
| 75 | 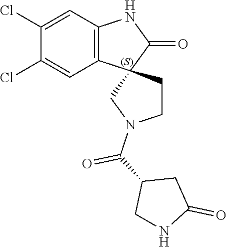 |
| 76 | 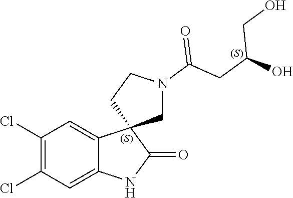 |
| 77 | 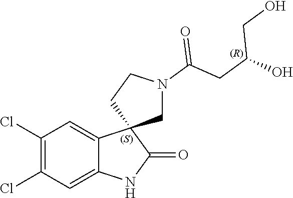 |
| 78 | 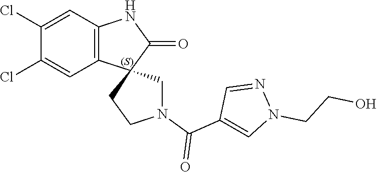 |
| 79 | 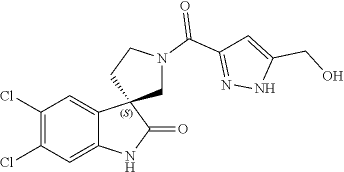 |
| 80 | 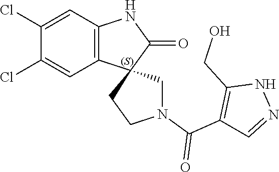 |
| 81 | 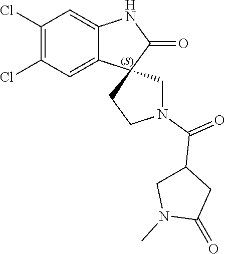 |
| 82 | 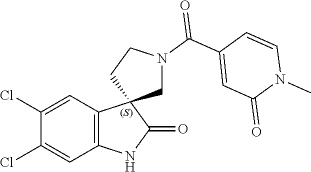 |
| 83 | 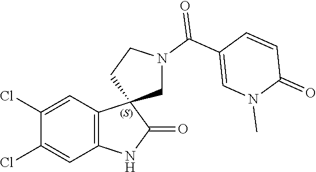 |
| 84 | 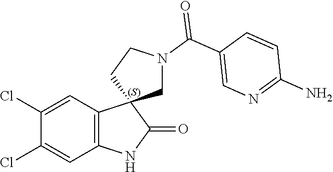 |
| 85 | 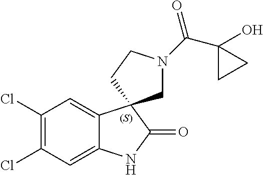 |
| 86 | 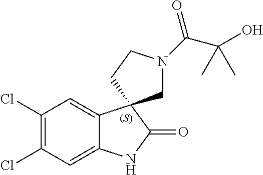 |
| 87 | 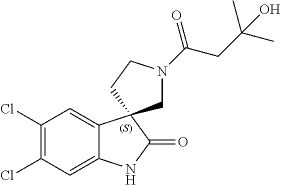 |
| 88 | 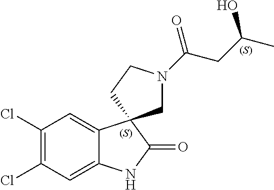 |
| 89 | 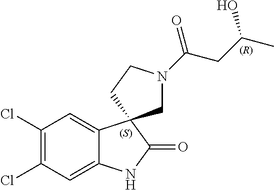 |
| 90 | 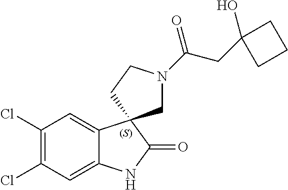 |
| 91 | 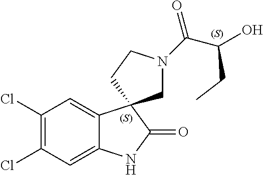 |
| 92 | 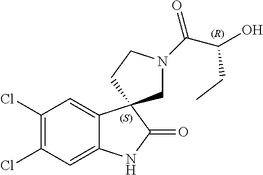 |
| 93 | 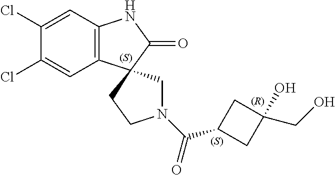 |
| 94 | 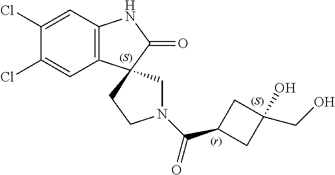 |
| 95 | 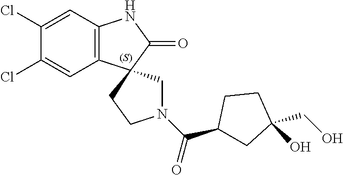 |
| 96 | 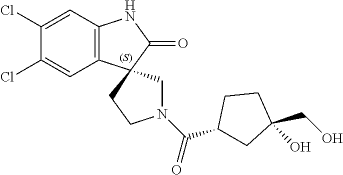 |
| 97 | 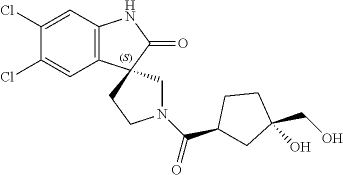 |
| 98 | 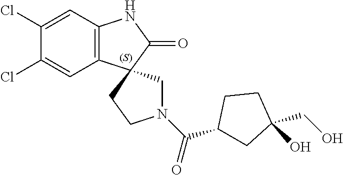 |
| 99 | 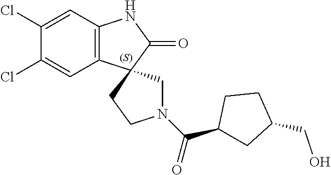 |
| 100 | 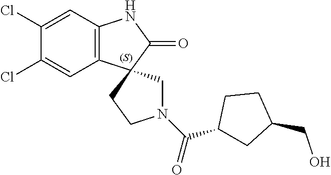 |
| 101 | 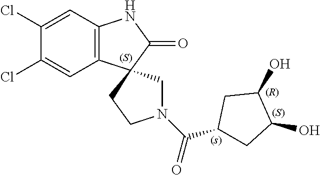 |
| 102 | 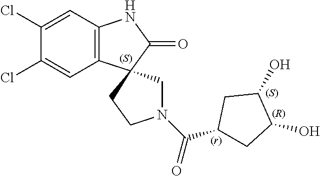 |
| 103 | 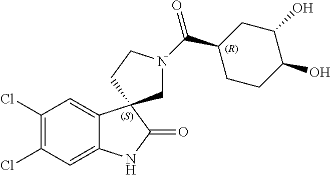 |
| 104 | 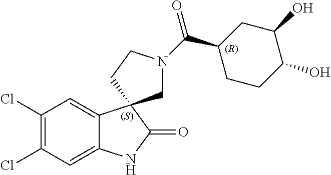 |
| 105 | 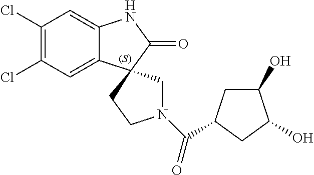 |
| 106 | 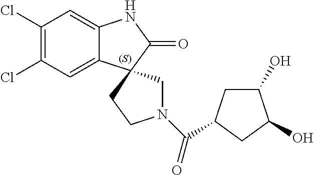 |
| 107 | 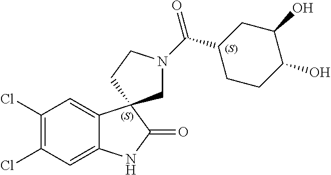 |
| 108 | 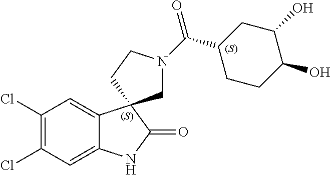 |
| 109 | 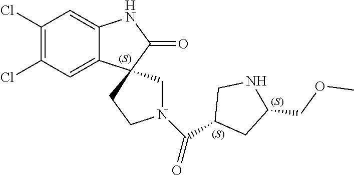 |
| 110 | 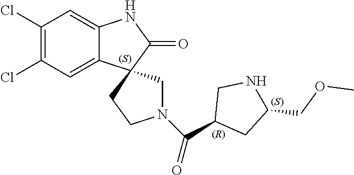 |
| 111 | 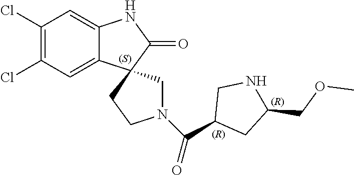 |
| 112 | 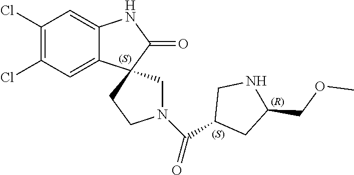 |
| 113 | 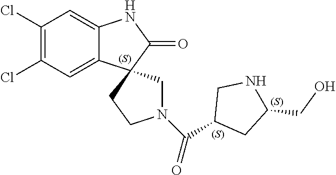 |
| 114 | 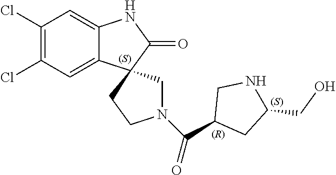 |
| 115 | 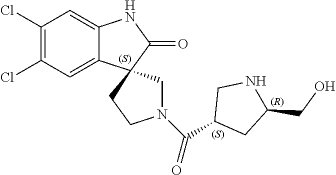 |
| 116 | 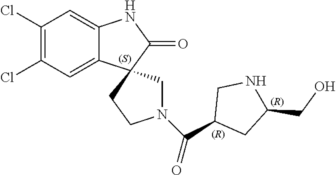 |
| 117 | 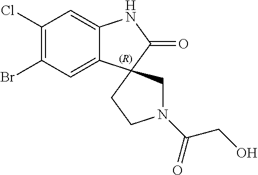 |
| 118 | 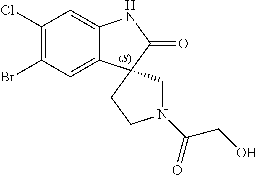 |
| 119 | 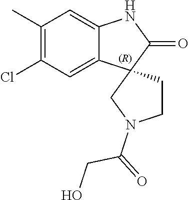 |
| 120 | 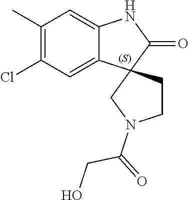 |
| 121 | 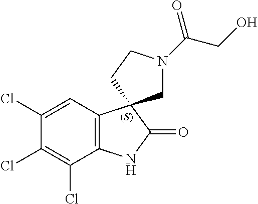 |
| 122 | 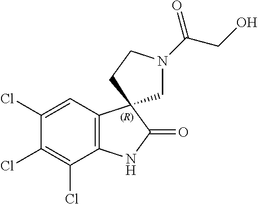 |
| 123 | 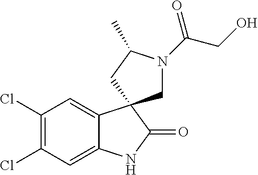 [SMILES: C[C@H]1C[C@]2(CN1C(=O)CO)C(=O)Nc1cc(Cl)c(Cl)cc12] |
| 124 | 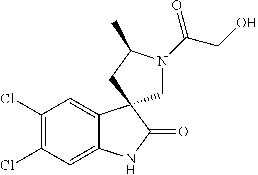 [SMILES: C[C@@H]1C[C@@]2(CN1C(=O)CO)C(=O)Nc1cc(Cl)c(Cl)cc12] |
| 125 | 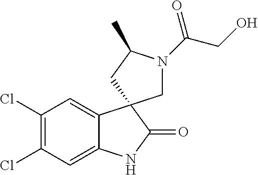 [SMILES: C[C@@H]1C[C@]2(CN1C(=O)CO)C(=O)Nc1cc(Cl)c(Cl)cc12] |
| 126 | 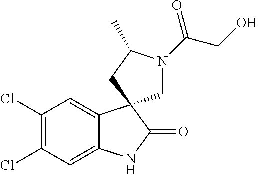 [SMILES: C[C@H]1C[C@@]2(CN1C(=O)CO)C(=O)Nc1cc(Cl)c(Cl)cc12] |
| 127 | 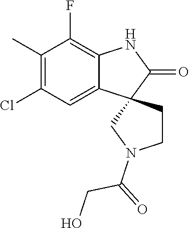 [SMILES: Cc1c(Cl)cc2c(c1F)NC(=O)[C@@]21CCN(C(=O)CO)C1] |
| 128 | 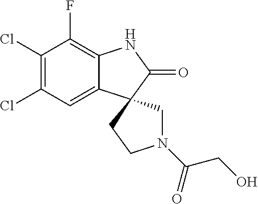 [SMILES: O=C(CO)N1CC[C@]2(C1)C(=O)Nc1c2cc(Cl)c(Cl)c1F] |
| 129 | 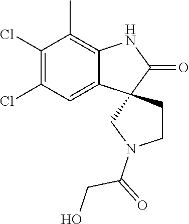 [SMILES: Cc1c(Cl)c(Cl)cc2c1NC(=O)[C@@]21CCN(C(=O)CO)C1] |
| 130 | 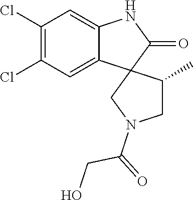 [SMILES: C[C@@H]1CN(C(=O)CO)CC12C(=O)Nc1cc(Cl)c(Cl)cc12] |
| 131 | 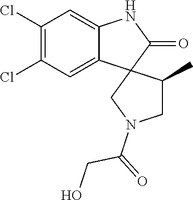 [SMILES: C[C@H]1CN(C(=O)CO)CC12C(=O)Nc1cc(Cl)c(Cl)cc12] |
| 132 | 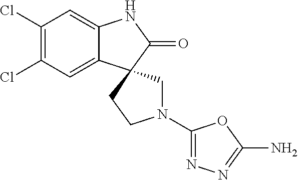 [SMILES: Nc1nnc(N2CC[C@]3(C2)C(=O)Nc2cc(Cl)c(Cl)cc23)o1] |
| 133 | 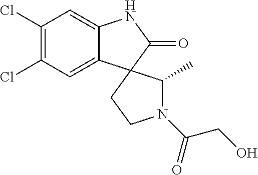 [SMILES: C[C@@H]1N(C(=O)CO)CCC12C(=O)Nc1cc(Cl)c(Cl)cc12] |
| 134 | 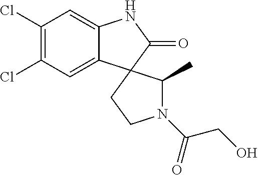 [SMILES: C[C@H]1N(C(=O)CO)CCC12C(=O)Nc1cc(Cl)c(Cl)cc12] |
| 135 | 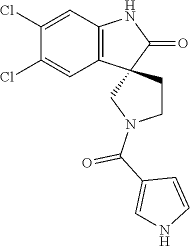 [SMILES: O=C(c1cc[nH]c1)N1CC[C@]2(C1)C(=O)Nc1cc(Cl)c(Cl)cc12] |
| 136 | 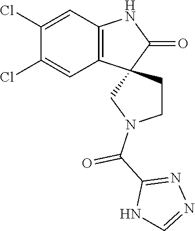 [SMILES: O=C(c1nnc[nH]1)N1CC[C@]2(C1)C(=O)Nc1cc(Cl)c(Cl)cc12] |
| 137 | 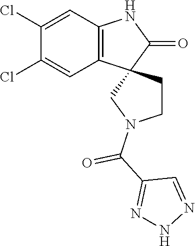 [SMILES: O=C(c1cn[nH]n1)N1CC[C@]2(C1)C(=O)Nc1cc(Cl)c(Cl)cc12] |
| 138 | 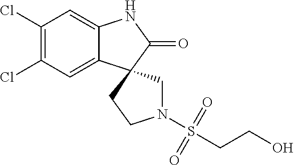 [SMILES: O=C1Nc2cc(Cl)c(Cl)cc2[C@]12CCN(S(=O)(=O)CCO)C2] |
| 139 | 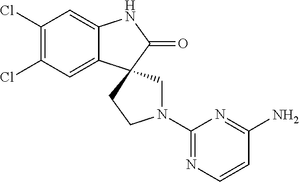 [SMILES: Nc1ccnc(N2CC[C@]3(C2)C(=O)Nc2cc(Cl)c(Cl)cc23)n1] |
| 140 | 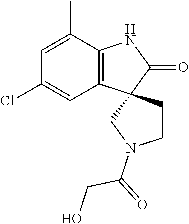 [SMILES: Cc1cc(Cl)cc2c1NC(=O)[C@@]21CCN(C(=O)CO)C1] |
| 141 | 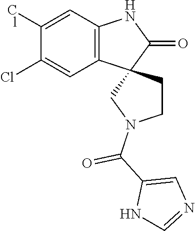 [SMILES: O=C(c1cnc[nH]1)N1CC[C@]2(C1)C(=O)Nc1cc(Cl)c(Cl)cc12] |
| 142 | 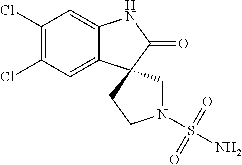 [SMILES: NS(=O)(=O)N1CC[C@]2(C1)C(=O)Nc1cc(Cl)c(Cl)cc12] |
| 143 | 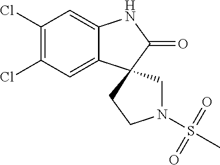 [SMILES: CS(=O)(=O)N1CC[C@]2(C1)C(=O)Nc1cc(Cl)c(Cl)cc12] |
| 144 | 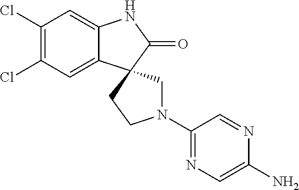 [SMILES: Nc1cnc(N2CC[C@]3(C2)C(=O)Nc2cc(Cl)c(Cl)cc23)cn1] |
| 145 | 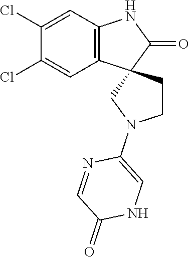 [SMILES: O=C1Nc2cc(Cl)c(Cl)cc2[C@]12CCN(c1c[nH]c(=O)cn1)C2] |
| 146 | 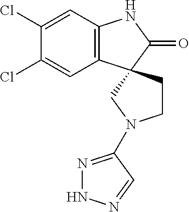 [SMILES: O=C1Nc2cc(Cl)c(Cl)cc2[C@]12CCN(c1cn[nH]n1)C2] |
| 147 | 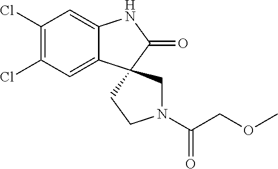 [SMILES: COCC(=O)N1CC[C@@]2(C1)C(=O)Nc1cc(Cl)c(Cl)cc12] |
| 148 | 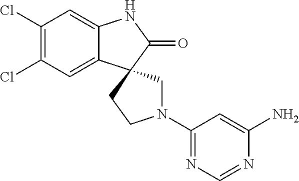 [SMILES: Nc1cc(N2CC[C@]3(C2)C(=O)Nc2cc(Cl)c(Cl)cc23)ncn1] |
| 149 | 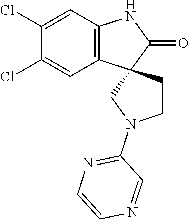 [SMILES: O=C1Nc2cc(Cl)c(Cl)cc2[C@]12CCN(c1cnccn1)C2] |
| 150 | 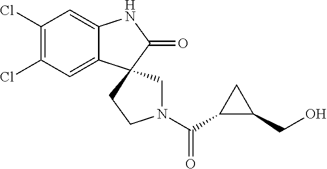 [SMILES: O=C([C@@H]1C[C@H]1CO)N1CC[C@]2(C1)C(=O)Nc1cc(Cl)c(Cl)cc12] |
| 151 | 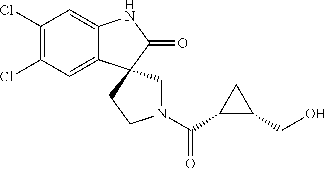 [SMILES: O=C([C@@H]1C[C@@H]1CO)N1CC[C@]2(C1)C(=O)Nc1cc(Cl)c(Cl)cc12] |
| 152 | 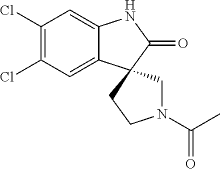 [SMILES: CC(=O)N1CC[C@]2(C1)C(=O)Nc1cc(Cl)c(Cl)cc12] |
| 153 | 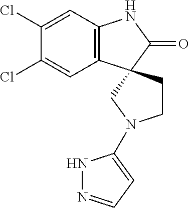 [SMILES: O=C1Nc2cc(Cl)c(Cl)cc2[C@]12CCN(c1ccn[nH]1)C2] |
| 154 | 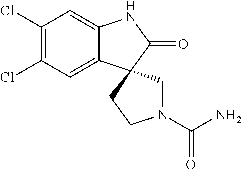 [SMILES: NC(=O)N1CC[C@]2(C1)C(=O)Nc1cc(Cl)c(Cl)cc12] |
| 155 | 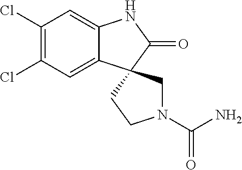 [SMILES: NC(=O)N1CC[C@@]2(C1)C(=O)Nc1cc(Cl)c(Cl)cc12] |
| 156 | 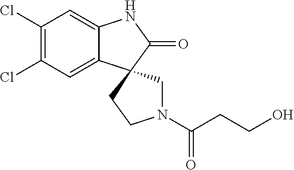 [SMILES: O=C(CCO)N1CC[C@]2(C1)C(=O)Nc1cc(Cl)c(Cl)cc12] |
| 157 | 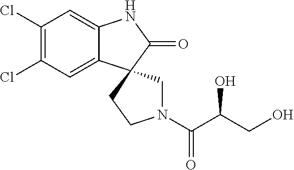 [SMILES: O=C([C@@H](O)CO)N1CC[C@]2(C1)C(=O)Nc1cc(Cl)c(Cl)cc12] |
| 158 | 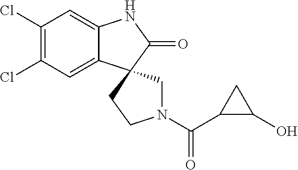 [SMILES: O=C(C1CC1O)N1CC[C@]2(C1)C(=O)Nc1cc(Cl)c(Cl)cc12] |
| 159 | 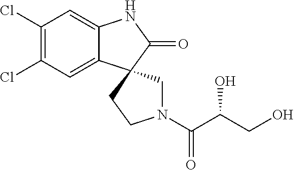 [SMILES: O=C([C@H](O)CO)N1CC[C@]2(C1)C(=O)Nc1cc(Cl)c(Cl)cc12] |

or a pharmaceutically acceptable salt thereof.

93. A pharmaceutical composition comprising at least one compound of Formula I or a pharmaceutically acceptable salt thereof: 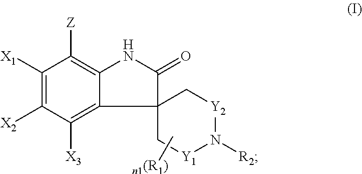
wherein:
the structural moiety 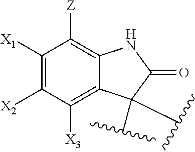
has the structure of 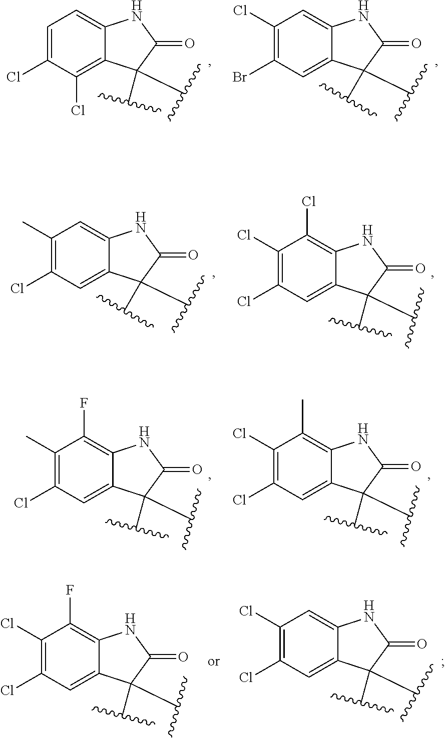
or alternatively X₁ and X₂ and the carbon atoms they are connected to taken together form a 5- or 6-membered aryl: wherein X₃ is H, halogen, CN, alkyl, cycloalkyl, halogenated alkyl, halogenated cycloalkyl, OH, SH, alkoxy, halogenated alkoxy, alkylthio, or halogenated alkylthio and Z is H, alkyl, halogenated alkyl, heteroalkyl, cycloalkyl, heterocycle, aryl, heteroaryl, halogen, CN, CF₃, OCF₃, ORₐ, NRₐR_{b}, or NRₐ(C═O)R_{b};
or alternatively X₂ and X₃ and the carbon atoms they are connected to taken together form a 5- or 6-membered aryl: wherein X₁ is H, halogen, CN, alkyl, cycloalkyl, halogenated alkyl, halogenated cycloalkyl, OH, SH, alkoxy, halogenated alkoxy, alkylthio, or halogenated alkylthio and Z is H, alkyl, halogenated alkyl, heteroalkyl, cycloalkyl, heterocycle, aryl, heteroaryl, halogen, CN, CF₃, OCF₃, ORₐ, NRₐR_{b}, or NRₐ(C═O)R_{b};
Y₁ is absent or C(R₁)₂;
Y₂ is absent, C(R₁)₂, C(R₁)₂(C═O), C(R₁)₂C(R₁)₂ or C(R₁)₂C(R₁)₂(C═O);
each occurrence of R₁ is independently H, halogen, alkyl, cycloalkyl, saturated heterocycle, aryl, heteroaryl, (CR₄R₅)ₙ₃OR_{c}, or (CR₄R₅)ₙ₃NR_{c}R_{d};
R₂ is alkyl, heteroalkyl, cycloalkyl, heterocycle, bicycloalkyl, spiroalkyl, heterobicycloalkyl, heterospiroalkyl, alkylaryl, alkylheteroaryl, aryl, heteroaryl, (CR₄R₅)ₙ₂(C═O)R₃, (CR₄R₅)ₙ₂(C═O)N(R₄)R₃, SO₂R_{c}, or SO₂NR_{c}R_{d};
each occurrence of R₃ is independently H, alkyl, cycloalkyl, heterocycle, bicycloalkyl, spiroalkyl, heterobicycloalkyl, heterospiroalkyl, alkylaryl, alkylheteroaryl, aryl, or heteroaryl;
each occurrence of R₄ and R₅ is independently H, alkyl, cycloalkyl, heterocycle, aryl, or heteroaryl;
each occurrence of Rₐ and R_{b} is independently H, alkyl, cycloalkyl, heterocycle, aryl, or heteroaryl;
or alternatively Rₐ and R_{b} together with the nitrogen atom that they are connected to form a 3-7-membered heterocycle;
each occurrence of R_{c} and R_{d} is independently H, alkyl, cycloalkyl, heterocycle, aryl, or heteroaryl;
or alternatively R_{c} and R_{d} together with the nitrogen atom that they are connected to form a 3-7-membered heterocycle;
each heterocycle comprises 1-3 heteroatoms each independently selected from the group consisting of N, O and S;
each of alkyl, cycloalkyl, heteroalkyl, heterocycle, aryl, and heteroaryl in X₁, X₃, Z, —R₁, R₂, or R₃, where applicable, are optionally substituted by 1-4 substituents each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)Rₐ, where valence permits;
n₁ is an integer from 0-4;
n₂ is an integer from 0-4; and
n₃ is an integer from 0-4;
and a pharmaceutically-acceptable carrier or diluent.

94. A method of treating a condition in a human subject in need thereof, comprising administering to the human subject a therapeutically effective amount of at least one compound of Formula I or a pharmaceutically acceptable salt thereof: 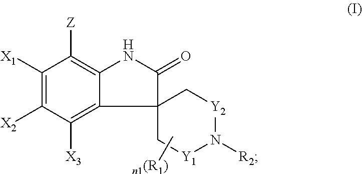
wherein:
the structural moiety 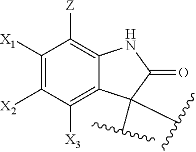
has the structure of 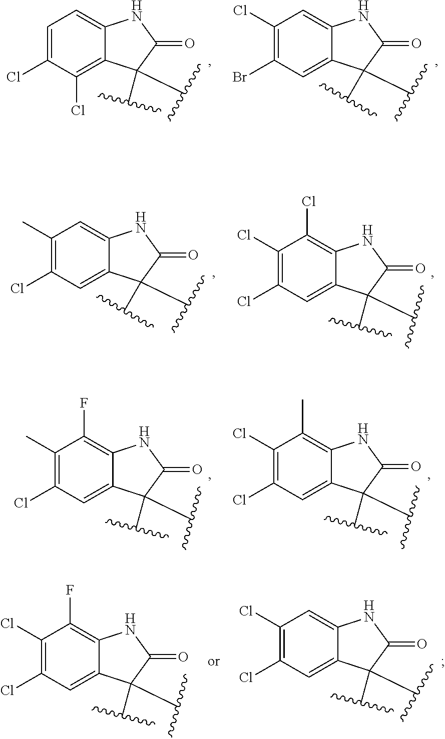
or alternatively X₁ and X₂ and the carbon atoms they are connected to taken together form a 5- or 6-membered aryl: wherein X₃ is H, halogen, CN, alkyl, cycloalkyl, halogenated alkyl, halogenated cycloalkyl, OH, SH, alkoxy, halogenated alkoxy, alkylthio, or halogenated alkylthio and Z is H, alkyl, halogenated alkyl, heteroalkyl, cycloalkyl, heterocycle, aryl, heteroaryl, halogen, CN, CF₃, OCF₃, ORₐ, NRₐR_{b}, or NRₐ(C═O)R_{b};
or alternatively X₂ and X₃ and the carbon atoms they are connected to taken together form a 5- or 6-membered aryl: wherein X₁ is H, halogen, CN, alkyl, cycloalkyl, halogenated alkyl, halogenated cycloalkyl, OH, SH, alkoxy, halogenated alkoxy, alkylthio, or halogenated alkylthio and Z is H, alkyl, halogenated alkyl, heteroalkyl, cycloalkyl, heterocycle, aryl, heteroaryl, halogen, CN, CF₃, OCF₃, ORₐ, NRₐR_{b}, or NRₐ(C═O)R_{b};
Y₁ is absent or C(R₁)₂;
Y₂ is absent, C(R₁)₂, C(R₁)₂(C═O), C(R₁)₂C(R₁)₂ or C(R₁)₂C(R₁)₂(C═O);
each occurrence of R₁ is independently H, halogen, alkyl, cycloalkyl, saturated heterocycle, aryl, heteroaryl, (CR₄R₅)ₙ₃OR_{c}, or (CR₄R₅)ₙ₃NR_{c}R_{d};
R₂ is alkyl, heteroalkyl, cycloalkyl, heterocycle, bicycloalkyl, spiroalkyl, heterobicycloalkyl, heterospiroalkyl, alkylaryl, alkylheteroaryl, aryl, heteroaryl, (CR₄R₅)ₙ₂(C═O)R₃, (CR₄R₅)ₙ₂(C═O)N(R₄)R₃, SO₂R_{c}, or SO₂NR_{c}R_{d},
each occurrence of R₃ is independently H, alkyl, cycloalkyl, heterocycle, bicycloalkyl, spiroalkyl, heterobicycloalkyl, heterospiroalkyl, alkylaryl, alkylheteroaryl, aryl, or heteroaryl;
each occurrence of R₄ and R₅ is independently H, alkyl, cycloalkyl, heterocycle, aryl, or heteroaryl;
each occurrence of Rₐ and R_{b} is independently H, alkyl, cycloalkyl, heterocycle, aryl, or heteroaryl;
or alternatively Rₐ and R_{b} together with the nitrogen atom that they are connected to form a 3-7-membered heterocycle;
each occurrence of R_{c} and R_{d} is independently H, alkyl, cycloalkyl, heterocycle, aryl, or heteroaryl;
or alternatively R_{c} and R_{d} together with the nitrogen atom that they are connected to form a 3-7-membered heterocycle;
each heterocycle comprises 1-3 heteroatoms each independently selected from the group consisting of N, O and S;
each of alkyl, cycloalkyl, heteroalkyl, heterocycle, aryl, and heteroaryl in X₁, X₃, Z, —R₁, R₂, or R₃, where applicable, are optionally substituted by 1-4 substituents
each independently selected from the group consisting of alkyl, cycloalkyl, heterocycle, aryl, heteroaryl, halogenated alkyl, halogenated cycloalkyl, halogen, CN, oxo, C(═O)R_{c}, COOR_{c}, (CR₄R₅)ₙ₃OR_{c}, (CR₄R₅)ₙ₃NR_{c}R_{d}, and (CR₄R₅)ₙ₃NR_{c}(C═O)R_{d}, where valence permits;
n₁ is an integer from 0-4;
n₂ is an integer from 0-4; and
n₃ is an integer from 0-4;
wherein the condition is selected from the group consisting of cancer, an immunological disorder, a central nervous system disorder, an inflammatory disorder, a gastroenterological disorder, a metabolic disorder, a cardiovascular disorder, and a kidney disease.

95. (canceled)

96. The method of claim 94, wherein the immunological disorder is an autoimmune disease selected from the group consisting of rheumatoid arthritis, multiple sclerosis, systemic lupus erythematosus, and type I diabetes mellitus.

97. (canceled)

98. The method of claim 94, wherein the inflammatory disorder is an inflammatory skin condition, arthritis, psoriasis, spondylitis, parodontitis, or an inflammatory neuropathy.

99.-102. (canceled)

103. The method of claim 94, wherein the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

104.-106. (canceled)

107. The compound of claim 1 of the formula: 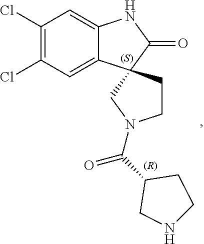
or a pharmaceutically acceptable salt thereof.

108. The compound of claim 1 of the formula: 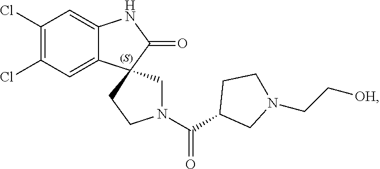
or a pharmaceutically acceptable salt thereof.

109. The compound of claim 1 of the formula: 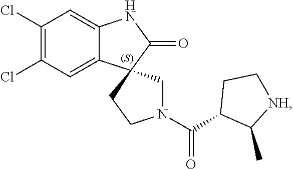
or a pharmaceutically acceptable salt thereof.

110. The compound of claim 1 of the formula: 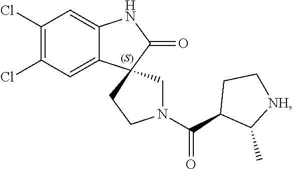
or a pharmaceutically acceptable salt thereof.

111. The compound of claim 1 of the formula: 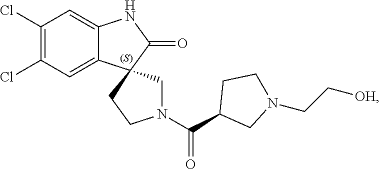
or a pharmaceutically acceptable salt thereof.

112. The compound of claim 1 of the formula: 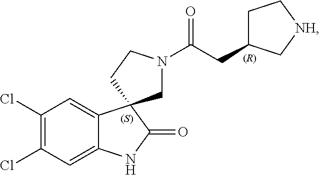
or a pharmaceutically acceptable salt thereof.

113. The compound of claim 1 of the formula: 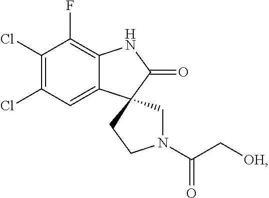 [SMILES: O=C(CO)N1CC[C@]2(C1)C(=O)Nc1c2cc(Cl)c(Cl)c1F]
or a pharmaceutically acceptable salt thereof.

114. The compound of claim 1 of the formula: 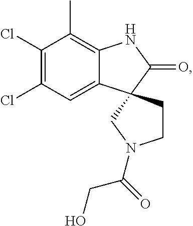 [SMILES: Cc1c(Cl)c(Cl)cc2c1NC(=O)[C@@]21CCN(C(=O)CO)C1]
or a pharmaceutically acceptable salt thereof.
